SEQUENCE LISTING (251 sequences)

SEQ ID NO: 1 - DNA, 1820 bp, Homo sapiens
tgaaagggatacgtttgcgtctgtcctgtttacttgctttgtccttcgctggggctttcactgtgccacatctcactgtagggatgctttctgtgctaagcttgtttcagtattcaaaccttcattttgtaagaacatgacagagcacctgccatggcattcacgcaggtagggctggaggcagccaccgacgtttgttaattgcagagttttaactcaagggggacagatgatctcaggacagaatgacaagctgagtgacagcaggagggacgtcaccgtacaattctctccacttttctgtaagtttgaaaatcctcacagaacacccagaggcacacagtgtcctgaagtggaaacggccaggacagtgtcctttctctttgttgggctgcaatttctggacttctgtacaactctgaccagctgcctgtcccctcccttcccagggtgaggtaggagccactatggcaggtcggggtcagggagaaacaaacgggggatctgcgtggagtcggcctcccccggctccccggggcgtcgggatgctgggtggggggccccactgtcaagaaccagtttagtgcgactgggaaatctggacacttgctggttctagggagaggaaggtggaattaggaattcccttgggattgggagcgtcaggaaaatatcctttttgttttaagaggtgtgtatgtaaagtctgtgggacaacgggaagggatgtcttttgactaattacctaaaccaaaattggagcaactatgataacagttcaatgctttaagacaaagtggggggtgtgcgggcaagcactccctcatcttggccgaaatttttctgaagaaacccgctaagtctcaatcagcagcatcaggactgacaggaagaagcagccgccacccgcgccccaaccctgccccgcctcggcgaggtcagaccctcacgcacagttccctgcctcccaccactacctccggccttctcagccctgtccacggctcctgcggtgggctcggccttcgatgtcagggacctccccgccatttcctctcagctcgccagcgagggtgcctcgggagggagcctccagtggtgattggagcaaccgccgctgggggcaggactccaggcagcgcgcctgcgcaatgcactcctgcgcgcgcctggagatgtgaggtaattctccggcaggcctgcgtggcactagtgcgcatgcgtaaaggcgcgagggctacaaacgcggcgggaagcccgccagggccacgtgcggccgtccaggcttgcgattggcccgctgccgggtgcccccgcgcatgtgcgctggcttccgaggggaccggccctggttctggaggccctccccaccaacgagcagtacgcatgtgtagcgccgaagcttcctgtgaagtgtgcgtgtctgacggatgacgactccacaaggcgctgtggccctggcagcctcatgaggttgcggctctgcgggaccacaccgccgcgggagtgcacgggccccagcgagtgaaatctgcggcagcccccgctgggcccgctgttcctgcgcgcgcagaggagcgtagcctgcccctaggccgcgttcccgtgagctccatgcccacagtggccgaggccggccacaagcccacggtcccttctgcacggtccctgccgcgctggggccaccgtggaggcccggagggccctgggaggagggaggaggagcagaggctttcgggagaacccagcccttcaccggccaggggaggccgcgatgcatcgcgactggttgtgaagagccaggggaagaactttaccgt

SEQ ID NO: 2 - DNA, 2052 bp, Homo sapiens
  misc_feature (1704)..(1803): n is a, c, t, or g
attctgacccttgcccagcctacgtctcgggcagcacccgtgaggacaccctccaggtgccggagaagcaggctctggcttccagctttgtttctaggaacacatttaaaggaaacttcctaagtgagagctgcacagaattttatctccgcagttctgatctttcatgtatgtgactgagagaggtcaagtgaggggccaaaaaaaaaaaaaaaaaaacacaaggcccaagaagcaaagcaagctgggacgtgagaactggggagggcttgctcattggtcaggtgttcacccacgtgcgtgtagaaacgtgctcttgcatgtgctggggatgcgtccagggctgaggaggaggagggccggcgctgtttataagatgccagttcttagcacgcctcccacatgtgctgctgggagccattcaggaaggggggcgcctcatgggacaggacaggtgataaggggagtgagggtgtccttggccagacatggggctttgtccaacagcacggcaggccggggtaaccggagggagggcacacgtgctgccaccgtgggaggaggctggctccagacatgctcttctccagtgccctctgcttcctcatagaagcaggaagctcagtgccagagagaatgcggcggaaggaggacgcatgagacaagtggcctctcggactggggacgcccagcagtgccagggcctgcttgagatgaggtgtcaagaaaggagaccaaggccacacagctccacgaggcgtctttctctagctgcatcccgccagtgcggaggggcacagtggcagggagttaagagccagccagggcgggctcattctgaacacaatgaggcaaaggtgtcaagttccattgtttgctttctgatctgaaataaacacatgatctcttggctactgtgtcctgatgctgttgtttgtacactacttcctgtggaggtctctgccattttcctggtgaaggacttctcagtaataaaagcaggaacgtggaaagcaaactcaagagccaagaaataaagaaactcagtccatacacattatgtgtttaaatcttttcagaattatttgaggacaatctattatacttccctaaggaagtgccattttgtaattgtgagctttcatggactcatttgagccataaagcttacctcacgctatttcccaggcaatcataactcactcagctcaaaccggtgtgtggcagatggagggcatgtgagcagttctgatggtgtcaaggcaagccaaggatacataacagaaaagtaacctggatctcggaggacactcaactcacctctccaaggtgtgagtcccccagcggtccttttgtttctgggttggcaattataatccgaacccctggaagtatctatttgggagaggaaaagtctcttgtcaatgggaggaatacagggagagactacacacaagccaacctcaatctcatctttatgccatttcctttcaagactgtttagaaagcaattaaatcaaaactatatgccacatagttatgacccattatacaaccacagcctcacaatcacagcctcacaatcacattctcactgtaactgtcaatattgtatgctgttatggtgacctcaaaattaaacattttgattgtcagtcatacaggtttctttagacccggagtgaggcttgcaacgctagttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaggaaacactgggaattttagttgtttaatgtattatttaagatatttacatagactaatattacatctcacatcatggcacacacatggatggagggtgatgcttgcagtaatcgctgaaggaagggagtcacatagtgacattttcaggggtaagcatggactcgaagataacccaaaatgcttttggcaaaatgatatagtaggcagctgctctggtggtgccagaagggaaagattgtgggtcaa

SEQ ID NO: 3 - DNA, 2527 bp, Homo sapiens
agatactggtctcattcttgggcagtttctgccaggtttttacatctgtagcattcaacaaggcctttaacaagctgcagggtcataaaagtggagttacatgtgtgagcagtgtctctgttacaatgaggaaaagataaacgggaagatagtctgtaagaaaaaatatttttctccttactctcattttacatgaaggatgcagtggaattctgtttcttgtaaatgtgctaattttcttactcaggctttaatgggaaacctggtgagtgagcagggccctctgcagagagcaggcttccctgggggaggtgcccagaatgggctctggtccccctgcctaccttgggcacagcaggcagtcacgggcaccatgagttttgcctctgccacgccctctccacccccctgcccaccctggggggagcccctcacaaaaccactccttctgggcatttcacatcttgtcctaaaggaaaacagctggaagagaaggagagagcaaaaaaagaaaagaaatcatctattaaatatcagtcttgttttgacaaaatcataaattaattgtatgcatattctaaacattgatcttccagaaattttattacctgtgtaaacttttagaatttaactatgttacctaaattctgaaaaggctttctgctttcctatcagtttctctcaaagatcacagtggacttcgtggattgacacatgaaaggtagcaattgttgttaataataataaagtcatagctaatatacagttgagaactgaaagggcaaataattgtatagagtctcattcccaaaccttttattcatggttaaagtcctggctagtgtccacaaaaacctacttttccagctccctccaccctctcaagctgttgccctcactgttcagtaactaaatagccctgaactgttgacgttgttatcctgaaatccataaatacaagaccattcagtaaaaactccagcaaacagaaaaatcagaaatacaagtggcttgctaatttaagaatttacttcaaccactggaaagtaataagttaaaatgaataaattaaaaacacaagatgttttctttttttcgtatctgcagccatgtctggggacaaacaaattcctttgaaagataacaatgttattgatttggaatgtcactgcaaagaaatgaaagagtaattccaaaggaaggtaatctctaaaagttgagaggaaatatctttttatcttgattccaatgatgaaatacaacattatttcattatttttgttacattttatcctacttgaatttaacattaagtttggaataaagtctctaagacaggatattacaagtaacagaacacaagaaaaatccttcattaagggtcactaccaatctgttaaaacatgagtgggtgtgggtacacttccagcccttctgtcaacgcttgcaagaagatagaataaatagcattccaccctctatactgacacatctcctgaaaactactgttatcatttaggtcaatttaacacactgaaatacatctttaatggtgatcacattctactgtagaatttgaattaaggccctgtctgtgagtttagagtcactaaagcagcagacaaatattggtaagtacttatgttactgggcacatgcattttatttacatgttggttttcactgagacataggaggggtttaccaactatattaagaactttaatcagaaatccagaaggaaaaacaccagggtgagagcatctggaaaactctaccctcaggcatgttttcaattcagcagaaatgtggcccctgtatcttataaacactttagtggcttctttgcatgagggaaaaggtaactaggagatgatgtttattaaggtaagaaacattgaacactgaagactccttcctcaattcaacaaggcaaagaactggtaattcctactgagcattaattttacagaggagtaaaaccaggataggaaaaaaatcacttatgatgtgtttttaattaatttaaacaatgtaaaaaattatacttttgcacatgttgctgtgtctgggattttgacatttgaaaactcaagtgtcaagtacgctaccagttaatctttgatttcatgttaagagtctgcttttgttttaattacatagtgacatggaatttgatggaaaggaatcccagttttttctatgttccataaacgtggttccaactaacgagcttagtttagtaagaaatgaaattttaaatgttattagtaaaatctaattctatttattatattttcaaatgaacacatttattgagagcatttatgggtacccaaaacccctaaatgctagtgcttatttggtacttagcatgtgtcaggcacatgcacatacatacatacatcatcatatcatgcagaagatgtcccttaccccaggacaaacaataaagtggcatggcgggtgctgaatggtcatttgaattacaatcatctaggtgagtgagtgaaagtcaaactcggat

SEQ ID NO: 4 - DNA, 3236 bp, Homo sapiens
atgtttctaactatacctttatgtgtttttcctagggcctggattccttctgaaaacattcaagatatcacagtcaacattcatcggctgcacgtgaagcgcagtatgggttggaaaaaggcctgtgatgagctggagctgcatcagcgtttcctacgagaagggagattttggaaatctaagaatgaggaccgaggtgaggaagaggcagaatccagtatctcctccaccagtaatgagcaggtgagtgtgtctccggaaggaagtgcctattcattattacttttaaatgcagaaatcttagtgcacactcctcactgtaatgaacagattttgacgttctccttcccttttttacatttgtaaagtgctctgcaaaactaaaccaaaagcagttcaaatgaatacatagatgtaacaatcaatgaccttgaccctgccagtaccaagagagttaagtacaagtgctcctctctgaaggtgcgctggctctttcaagcctacagttaccagaacagtaaattaagtcagtggtaactgagtggatggaaggatgcaaaaggtagaaatgtattcacttctcacctgtgggtccactatgagtgttttcagcagagaagtattttctagtgtctggaataatatattacttttataatgcccacagctaaaggtcactcaagaaccaagagcaaagaaaggacgacgtaatcaaagtgtggagcccaaaaaggaagtaagttgcccacctcgcagtatccaggtggcaaatgaaacaggaaatattttcaaagtattttgtattttcaaagtatttcaaagacagtcactcttggtggatacttgtgaaattcagctgctgtcagtcaaatcatatccatcaagttgaaaccagtcttctgacttccctgtcattatctgttaccctggaatagcgtacatgctccaagtctccatcttaattaagcagccgctgaccaaagcttggctaagtaggaagggcacattgctattaatacatttcctgggagctctgatatttttcctaagtatgattaaaaacaacacatttatccagtatatcagttgtgccaacatttaaaaacttgaaggagactgtggttgagctcagccgttttaagtgatataagccctgcatgttttaaaactgtaaatctgggcacatttcaaacacatattcagtgagaagtggtttaggatttgaggaaatgtgttaatgaatctagtccaatgaagtaattataagttgacaataatttttatattctataaatttctgtgtttagtttattttaaaaacaaaacttatagtattgataagtaaaattataaatgaagcttatgtttataattattgtagctgttaattgcatgttcttttcattcactaattgggggagatttgtttatttttaaattgtggcaaaatatacgtgacatctaccaccctaactacatttttcaaccagcagtttattctatggctattatgtatatcactgaatttttatccgaatggggtagttcttgaactggtgaattatgtggcttcgtttggcgtctaaactcttgtctcaccttttaggaaccagagcctgaaacagaagcagtaagttctagccaggaaatacccacgatgcctcagcccatcgaaaaagtctccgtgtcaactcagacaaagaagttaagtgcctcttcaccaagaatgctgcatcggagcacccagaccacaaacgacggcgtgtgtcagagcatgtgccatgacaaatacaccaagatcttcaatgacttcaaagaccggatgaagtcggaccacaagcgggagacagagcgtgttgtccgagaagctctggagaaggtaatgcttgtcgccactgtgggtgccctgctgcagccggcactcctgtcatggttaggctcctttcactcatgcatcaacccagtagcagcttttacatgtagccatataatgacaccagtatcttttacagcatttcaagtaataatgatactttcctcacctaaattttttacacatgtaatgaaggggaaaaaaggtacctcatgcaagttgtgttaagtttctgttccagtgtagatggtctgtgttaagttgtgtgctgacgcactgtgggttgtcttttcattccagctgcgttctgaaatggaagaagaaaagagacaagctgtaaataaagctgtagccaacatgcagggtgagatggacagaaaatgtaagcaagtaaaggaaaagtgtaaggaagaatttgtagaagaaatcaagaagctggcaacacagcacaagcaactgatttctcagaccaagaagaagcagtgggtaaataccagtcttttttagacccttatttctgaaaatgtaccacaggtatgatgcccgttaattcagaaggtagctgtggcacatgcagaagatgtttctgaaataagatcaaatgtgaaatggtcagctttagttttaaaaattttattaaaagtcctatgatctctcaaccccagatcccatattactgtgtactgctcaggattattttgttaaattgagattataataccttagtacatatttattacaattaacttatataatttctccatctatgcatatattttatttgggcaaagtggctggccctgacttttacctggtgatttcagatgggtaacatccaaatggtgaaattataaatgtaattatcacaataaatagtttcagatttccctgcacttaacatttatacattagattttgttaaagaaatcagttacttttactttatagtagtgacatctcattggtctctaactaccctccctcatacctgactagtatcatttgtcatcgtgtcctgctcgccagtctcatcctccccactagagtgggagcttctgagtgcacagggtccaagtgctcgtcctacagccgccacagtgctcagtgaattagggaaaagttttgctcccgaaagctcataacttggtttcagttttaataaatgactatataaagttttgtgataaactaattcttcattttatcaagcctatattatataaatacacataagcttttcatgaaagaaatatttttaaatctgtgacaaagatttggcaagaaggaaaatggaaacttcgaatagatgaagataacttggtaggaagagctggtgaataacaaaataaatattgttaacaaa

SEQ ID NO: 5 - DNA, 2133 bp, Homo sapiens
  misc_feature (405)..(504): n is a, c, t, or g
agttaagctcagctcactctgtggcactacctgggccgagcagagggaaagtaagggagcgacaggaatggcttgtgaatgtgaaggcgagccgtgaatgtctgcgtcttggagtggaacccagagctgctaagggggcggccaccaaaaccccaaccgtcaggccctgcgaaccctttcaaggcagcctcggcacacggacaaccgacaagggtcctgagcaaggaggacgcacagctcgagctggctttgacattcgtgctcagtgtacagacacgactgtacacacaaaattaaacaggaaaaactcaagtctgggtgacacaaaatacatattcacaccccccgcacctctgaaaaggaaaacaacatgcagtctgcaacagcaggggttgaagcccaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagttttccccggcttaaaaaaggaagcaataagggctcctattcaagagagttattgtaagtatgaaataaatccgtaaatggcatcctccccctccactaatgtcaggattttattcggggttatttatatatgtgccaacagaaaggtcatgaaaatgtactctctttctaatacaatatagatgaacatgaatagtgctaactttttcctatataaatacaaaacttaaaatgattgcacaattacttatgttacataaagttatcttgcattttgctttcctgtccaagctttatgcattaggaaaacaatgcaggacagataaatgtactgttccgttattgatctctgtgtagatgacagaaacacaaacacaatccatgtatatacaaagacatacacacatccaaagagtacaaagtcagttgaaattttatcaaaactggtcagatgattattccctcctagttacttggagctaaggactacttaatttaccatgaagatatacgtatcaaaatgtccttggtttaaatggagggaaatactattattcttacataatagcaattattaaaaaatgaaacacaacactgttaactgaactgtaaaatgaattgagcttagggtccagacccagaaatcagggtctccagggaaaataaaagtgagcggctaaattcaaacctaccttcttaaacaccagtatcaaataaagttaacatcacctaagatcttctgaacactgaacacttcagaacactgaatccacccaacaaaaaatcaaatttaggatctttcaagtagacccagtggaatgacaggcattgaaaatattttacattctggttcgttactgtctgtggtcgtggggaaatattcacgttaaaaagattttcatataaaggcagtttgtaagcttcaggtgacgttagattaaacccaggctttgttttggaggactgttttaacttcaccccatcacagatgtgccttcttagaaaggagtccctgtgggctcacagggcactgagctgccaagggagctgcttaccttgagggactctgtttgcgagcccagccccttggtgcacagctccatcacggagtaggagcaaaacgtgtctcggactttgtactgactcacggcaagaagccacaaggcggggttggtttccagctcagagggcgggatcaggatggactggtgcccagaatacacactgcagagaaagaagaggctgtcagggcgggagctcagcaaggctggagctcagcaaggctggagggctcagggcagcactgactccaaggaaaaggaggacttggaacagcccgtgctgccatctgtagaagggcacagtaaagccaacgctgcaaactgcaaccatgttcacgaaagccttctgaaaagcaaatacgtactacagaatcatggggcagttcctaccactttgaacacacatttaagactactaaacgctgtgatgctgtgatgtctctcagacctgcgacatcagcaaactggatcctctttcttagtagaaaacacagggatcaaatttcggtttaaaaaaaaaaagtccagcttcagaacaggagctggcaaaccacagacacttcct

SEQ ID NO: 6 - DNA, 2026 bp, Homo sapiens
tgagatcctattcaatgctagacctctttgcccccagtggcacattagatggtaaagaggtgtgtggcagcatcaacatccctgaacactggtaatatttactgacattttcttggttaacatgtattataacccgtgtgctgcttatatctttaagccaactagctcactgcaaatgcgtattgggaaatgttccctgattcctcatgggaccttctttgaagcaatgaagtagggatattacattctagtctggggcaggctgagtggtacccacatggccaggaggacttttccttcacatctccaggaagggcctctctattctccttttttctccatttgctttgggcttctgagaaacagcacacaggattctgggacctgttctctaactaaaaagaagatccagctaagtatcacccaaagtggcagaatccaatcttcacccttgggcttagaaaaagaattctggtgtcccagagacaggtctttcctcctccagggagaggcttgtctagatgcaggaaaggttccaccagaaaagccaagggaggaacaggaagaacccccaccgtcacactgtcctaggggaagccaggcattttggctgcagaatctgggtcaggatgttttattgtcaccataaccatcaaagtcataggcagggcaaatgcattcgccctgtgtacattgtgagacatagttaagctgggacgtccctgaatctgtctcctaggaccagaactgcctcattaaagggataaaagatgatatctgctgagctggtggaaagtggtggctgcatttttattaaagtatctgctgcagcaagtccagtccccaaaggttcatattccaagattctccacctctctgcctggagcatgcaagtgattctctgtaactcattaaggtaaaacaaaaagctctcctattgtgcttttcacacagaagtgatgttgttgcataaaagctacatgtttcctttccttggacccagtctgcaaaaataaaactgctgtcataatttacaatagggaccctaggagcactacaccaggtttggcacgagtgctgggtcttgaggagactcataacaggccgtgggctgacactggtaattccacagcctcacatttgaggtgcatctctgataagggctagcctggtggtcctgaggacgatcctgcctcatcatgtaccttctggcctgtgacagccatccaaggggctcaggctagccccccagtgtttcaaacccatgcactcatgttctcatcacggtgcccaagcaggagagaatctagcctgtcgtggcttcaaagaaccatggagtcccacacgtggacttcaaggttcacgcataagatcctggaccagcatagccggagcacaggacaaacctgtccaggggcacggcagtcggcacggcagcacgcaagcgggcgcccctcgggcctgcacaaggcccactcgcgttccggtcccccatggagccttctgccccctcttccctcctctccccagcgaccacagcccaggggctcggcccccgcggaaggacagctccctacctgagggtggcgctctccccctgccggaccgtcacgttgtccatagctttggggaaggtggcatctccgctgcgcacgggcactcctgtgggtacaaggaacagcagcctgagagacacgaccacgaggcacttccagggcaggaacaggtacccacagacccccattctcgacagccacaacttcccaggactccggcagccgcacagtcctggtcccccgccccgcgcaccagcgggctcgggaagcggtgcggggaggagggaaggggcagagttcgccaggagcagggggaaggagaagagaggagtccgggctctccggagtctgagaattcttcctcagatcctgcctcagctttccagcctagcagaaccagatgccccctcctgcatccaaaaagagctttcttgacgctcccctggggaggagggaggcggccaggaggg

SEQ ID NO: 7 - DNA, 2462 bp, Homo sapiens
acccgagagatgagccctgcgtccactgcaccagcatccagccatggactgccaaggaaatctacaccctggcccccttcccttggtggtcagcctgctgctggtgggcacccctcaggggctcagcccctatccttccccagggaaagccggtatctaccgtcctcctagaaaggcagctgacatggttgcaggttctgcgcactgcatgctctgttcattttctcacctcttctacccattattccatctccccacactcttcccactgcttcttatttttttggcaaacggtgagatcacacaggcttatagccctgggggaaggtattccacagctgcttttgagccccagcccttccagcagcctgggcatctgagcacaaattgaacaacattaatgagacacccaatctcagcattttactctccactgctattctaaaatcttcacaaaaaagttcaggtggttcttttcaagctgcccacacacatgcacacacaccaagcctcccaccccagggcctgtggccggcttgtgtgtgagaagccagctcgctctggatgtgcgattctgcagtctgtgaaggcacagtggtagattacacaagagaatggccttacagttttataaactatttattaggcccgtcctggagagctacatcaatatggccgtcggtgaagcaaagcagaagctataaaaatatcatctatcccaaacaagcttcataatcaaacaaagccccgtgctggctgggacaggcttgtgttctgacacataagggccctttccatctttaaaacagaccattaaaacaccagaacactttggctcacagaagtctaaatcaaaagggaggggaaaaaagagagatctcttttctccaagagtaataatgccttttccagctcctggaaaagctcattgcgatagagatgcaatattgcttttttcatagtggcttttccgtttctttccaatacccagaaaatcttctaggggttcaacatttccacttgtttccctctaggaatccctttctttttactccacgtgtacacagtagctatgcggcgatcccttcaatattattttgttgttttcccaataaataaagatatacagtttgatacatattccagaagggaaatcatcatcataataataacctgaagtagaatgttaccagcccagtactgtgctccaattccccaaggcaaacgaacacgggaggcaggtccgtacgctggggtttactgtgattaacatttccagccagtgctcctccaattggctccaaaacatgtcttaataaactgcattccaaaagcccttatatttccaccttattgcattctgctagaatgagatataatatgtggacgcaaggaaaagtgacattcagtgaatgagctgcagagagttatataaggaagctaaatctcactccctaccacctggcatactgcttgtggctcctcatcatgattctagaaatcagtctgcaactaaaattcatgcatggggatgctctgctttggaccgtgggctggggaagagaggtgtgatatgcttttgagagggcagaaggcaaaagagaggaagaagggctgcagaggtggttggtccactcagagttgcactcccatggcaaggtgctccataaagaagtctgagaatggagatatgcagaactgagtcactcagagctaggcagataatccagcacctcagtctgggagaagttttctatgacattttgattgtttttagatctgggtagaatttttggacaagaagaagagacacgggatggactgcagagcctgagcagacacatgcaaaggacagtcacggcaccccacgctctttccctatcccccattttcaacctttattttctttccatcatcctggagatgcacaccctctgtgacctaggaggttgcatagagaggaaaaaatagtatctgtgatcacattttcttgtatttacaaaacacaagaaagtacattgacggcgaagtccatgagccctgaggaaatgtgaatagctttcagactgaagagtattcaccctgagtatatgcctgataggtaattcttagaggtgtgggggccattcaagtaattggcagtaaatgctggctactaagtaataaataactaaatgtgtagcatctctccttcccatctgagccctgcacgtgccacggagaatcaaacacatgacagagagtaaacggatctgagttctggactcagcccacacatggtcaccttcagcatctcagtcaagtcagtgacactgtctggttccaatttaccccaaagaagaaaggatcaaggctgagatacatcacacaacagtgatcttaaggtctgatctggaagagaaacccacacagtaaatccactagcacacaggtgcccattagggcttgaagacgcaggtgac

SEQ ID NO: 8 - DNA, 2884 bp, Homo sapiens
tcctccccacacctgaccctgccctcacttctggctcccctcagccccctgtgccccagccccagccacaccaggtgcatttggaccctccaggtcgccgagttcatccccgcctcggcgtctctgcacctgctgttccctggtttacagctcaaccgtcatcctcccaccccacccagaggaccatcctcttttgttccttggaagctggtgctgctgctgcaaagtccatgctactggaagcctcgaagtaggggggattctgttctagtctttgtcaaatcccactgcccatggcagcaccaggacccagttggggctccttggaactggcaggaaggaatcgggtggggagacaggcagagaagggggtctgtgcaaagaccaggagaaaccagagacaggtcgtggcgggggctgagaccttcacacagggcaggggccgccccggggggttctccttgtcttgcagcccctgtgcagggcatcctcagagcaggggcagcccagggcaccgggacgcccaggtggaaggtgacctgccatcctgcagcttcacttcctgccgggtgattcggtacccctggttgtgcctgtcgctcagtgggccagggtctaagggctgtgaagactcaacatgcccccacctgctacttctgaacaccaggcactggctctgagacccccgggccttgctggacatctccccaggtgtactgggccaggggacaggggcctggccatcccaacacccaggagcaagcagcccgtcacctgcccaggtccccgaggcctggaacaccttcctgctgggcccacccagccctggacctgtcccgcttggtcacacgatgggaccctcggcccatcagcaggtgagcccccaggagcgtgcgtctggcctggtaaggcctccaccccaggagttggggggcccccgtgccagggagcaggaggctgccgaggtggagggtcccacacagctaccactccctatccccagcacagcctggggcctggctctgagtacacatcctggggcctggctctgagcagaccaagagcccatccctgctttgtgaccccctgggctgtgcctgacaccccaggtgtccagcgtggagctggggcccagctcagtgcctgggagctgatggaccctggggcccggctcagtgcctggtggctgatggacactggggcctggctcaaacctgcaccgctgtggtcgggggaggggagggctgagccacgtggggaccccagccccagtgacgactctttgcggtggccaagccctccaggtgtcccccagggctgaggggctgggcttggggcagctggtgacagcagatggtggccctgatcactggtgcctggacggcctctgaagggtctgtggggtcctggacgggtccccattcatggcaggattaacccccctcgggttctgtgtggtctaggccgcccctttgtctccactgccccctggccagaatgagggacagtgacccacccagggctgggcctggctcagactccgtcagagccgcagggcaagttcctggcacgtccgaggtgggaggctcctctgcgctccaggaggctgtgcctggccccccttcccggcaggaaccggctgtgtccctttccttcctttatcttctgttttcagcgccttcaactgtgaagaggtgaactcttcaaacacgctgagcaaacaggcccgactcccagggccgcatccgggatgtctcaatagctgtggccttgacgtccacctcggacccctgccccggacccagcccagttcccaatgggccctctgcccggggaggtgcctagtgggagggacgagggcaaagtcggggcccccacttgtttggtgtcactgtgtgccagcggccactggcgggcgaggctgttccagggtggaggcggggagggttggaccacaggcactgagcggggacagaggagctgcctgagggtcccagctctgccatggagaaaacgctatctcgctgatgcagaggtgcccggcccactcgagctgggggtgagggggctgctccccagtgggccgccagcccccatgaaggccgcgggcaccggccgtggtcagggagggcaggggacaggcagtgggggccagcaggggagacactaggcttggccccagcacccaggtgggcatcggcttgtgagctggagccgcgggcagggaggggggatgtcacgagggcttggctaaggtgggagacctgggcgggtgcgtcggggggacgtctgcagcagaggcccgggcagcaggcacacccctcctgccagtgcgaggaacgaggcgccacagcggccggtagccccccatttgcccagcctggcctggagcaggcaggaaggccggggagaggggtctggctggggcctgggtgcagtcacagccacgagcccaggggtggggactctggcccaccctccagaccatcctcaaggcccactggcccaggcatccccgcccacccctcccaccgtgccgtgctgcagcgggtctaccggcctggatgtgaaagagagcttggagaccccagagacctcggaaccttcagctttggaagtgacgtcggtggggtgggtggggggagcacaggctctggagtcccggaagtgagcggggagctacgctgagatctgggagaccccctgcccccacccaggtacagggccaggcagaagcccgaggtgtgccctgagttaaagaaaccgtcacaaagaacaaagggagaaggcgggttccagcctccaccacagccctcgcgctctgaggagccacctgggggcctcagccatgaggggtgacaggtggcaaaacgggccagctccg

SEQ ID NO: 9 - DNA, 2490 bp, Homo sapiens
cttcccctcctgataatgcaggcagcatcagaagcattcccaggtggacagaggggatgaaagggaacactattctgaagtcagtcaaggggattgttaaagatggtaactttttcacatctttattccccaaacagctgaattaatcctgaataaatggagagctgagtgtatgggtgggaaggtgaggacaccagggaggctctggccctcacagggtttgcatctgaaggggcaggggctggggctgggctgggaactgatggagtaagatgtgaataacagtgccaggggcccaacgttcagagctggcaggagagcgggaaggtgggtctggcctgggctgctgagaatttccatcaggtctgggcacagctggggaacacagggtggtcccggtgcagggcaggcgtcagtgaggacatgaaggctggtgagcagccgccagggggctggggcgcagtgagaagcaagaggaaagggcaggtgcggctgtggatccctggggactgcagcaggggtctgagctgtgcatggtgacaccagacaccacgaagggaccaggaggcccacacacctggagagagccgccacgcagctggggaccatagcgtcacctgcacctcctggctctgcctcttgtcttgggcatggctcactcaagccccacaggtgagtccccaccgctgcccccttactgggggatccctgaggccagtgagggtcacgaggacaggctggtgcatggctggacctgggaggtgggttcctagagccctcaggaggcagggtcaggtccagctggcttcctggaggtggtggccagcagaaaggaaggagagagaccagggagaaaccccggctggggcccagggtccctaaggacagcatcccgcgccccctcccactcccgcgggcctcgtcgctcgcccaccctggcctggccccgcagtctcaggacgcctggtacctgcttgtttgctcagggcgccccctcccctgcctgcctcgtggggcagggctgtctagacagcgggggctccttggcccaccggctttgtccccagagttccccgagcagaagaggcggccacagacaaaagggtgtttgcctttcccccacagccaggcagctcccctgtctccatggctccaggccagcctgtgaccccaggcccccacccagagggacacacccaggagctgggcctgtggctccctgaggggtggggtgaggaccgacaccaggacttgcttcccacaggggcttcctgggggtgcctccagccgagtctggggcacagggcagggctctgatgagtggaggttaggagggcgccgtgagggctggcaggagctcaggcagggggagtgaggaggtgggaggtgggcagagtggggtgtggcttccagcaggggccccctgacctggcaggtgtcgggcagaaagccaggccagctgtggcggatgcaggtgggctctggggtggggcagatgaggagggcccgggtagctgtgggtctgtgcccacctggcctggcccccaggcacctcctctgcttggcccccaggttctcccagcaccctgggcttcttcaagtccccctggcctctctccctctcatctcaggtggcttcccaggcagccctgcccctaaaaccagcacctagagcgtccctgcctgtgccagcaccctctccccacccggctctgccagcctgattccctcacgtctgagtttcctccacccgatttcctggcatattttatgtcacggtcctgcacggttgtcaggtgcccaggcctgtcttgggatggagggggctctgacagtgagcgagacagcaaatgtcccaagactcagtttctccgtttctgagcagggcttccccctgccaaggactcggccgaatggcacgtggggacactcccggtgccctggcccagtggcaaccctcccccggccccttcatctgtgtcccacatgctggggcgctcacggattttgtgaatgaacaaggaacaagggaggcagcgcctttgaaacccagggtaggagcacaaagccaccaagacccggctctcctgcacacccttgccccgagcccgccacgggcagccagatagcaggcagctggagcgaacccctgatccaggcccctggccctgcgccggctgaggggtgagagctgggcagagcgtatctgacctgggaacacccacctcacctaagcctgcccagctccacctgagacaacatccgggccctgataaagccagttgtgcaccctgggggcatgcaccatgctaatccgcttatctgctgggttggtctcagctgtgcccaaaaggagtccacactgggcggagatcaggggacaggcccagggtgggaggctggctctgcgtcccagcccgctgtgcagctgggccccgcagccttccccaccttcccctgtgttgggtctcaggtttcgatggcctttcc

SEQ ID NO: 10 - DNA, 3456 bp, Homo sapiens
cagaaggtagagttggaggatcataggcaagttttcagagaaaccgctttttttttcatttagattattataagatgttccagaggcactaagtgaacagaatctaatgtctttgtgcaatctgacgaacacttagtgtttagtagcagcattatgaaattgccatttttagataattctggcagtaaataccgtttaaatggtggtgaagaagactagcaacctatccttcacaaatatttcctgatagctctattttccctgctctttcaattacttacgtttacactttctctttatttacctatatgtctatctctgtttgatcttttctgaagttctgggcatactactcagatttcagtcacagctgtgaaagctgctattgataagattttttgaaacttcattctgttgctaaagaagggagaaatggccttattttattcaatacaggaaaaagaaacattcactttttttttggtatctttcagtttcagagtcaagtggtgagatcaaagacttttcaccaaaaaatgtcatttatgatgactcatcccagtatttgatcatggaaagaattctaagtcaaggccctgtgtattccagttttaaaggaggctggaaatgcaaggatcatactgagatgctgcaagaaaatcagggatgtattaggaaagtaacagtctctcatcaagaagccctggctcaacatatgaatatcagtactgtggagaggccctatggatgccatgaatgtggaaaaacttttggtcgacgcttttccctggtgttacaccagaggactcatactggagagaaaccatatgcatgtaaggaatgtggcaaaacctttagccagatttcaaaccttgtgaaacaccaaatgatacatactggaaagaaaccccatgagtgtaaggactgtaataaaacattcagttacctttcatttcttattgaacaccagagaacgcacactggggagaaaccttatgaatgtactgagtgtggaaaggcctttagccgtgcctccaacctcactcgacatcaaagaattcacataggaaagaaacaatatatatgtaggaaatgtggtaaagcatttagcagtggctcagaactcattcgccaccagattacacatactggagagaaaccttatgaatgcattgaatgtgggaaggcatttcgccgtttctcacaccttactcgacatcagagcatccatacaaccaaaaccccgtatgaatgtaatgaatgtaggaaagctttccgttgtcactcattccttattaaacatcagagaattcatgctggagaaaagctctatgaatgtgatgaatgtggtaaagttttcacttggcatgcatcccttattcaacatacgaagagtcacactggagagaaaccctatgcgtgtgctgaatgtgataaagccttcagccggagcttttccctcattctacatcagagaactcatactggagagaaaccctatgtatgtaaggtatgcaacaaatccttcagctggagctcaaaccttgctaaacatcagaggacacacactcttgacaacccctatgaatatgaaaattcatttaattaccactcattccttactgaacaccagtgaatttacactgcaaagaaaaactatgaatgtatggaattttttaaaaagaagtataatgccttacttcagagaactcttggaaagaagccttatgtgaaagtgatgactgtgaagtaatatggcccacactttattcaccaccctggagaaaaaaaaacccaggaatatgtggaaaagccattaataaccactcttttatttttttgcaataacaaggtgaaatcaatattgttgagaagattcttccatctggtaatgttgagaagacttcatttggtaggagtcccttactttacgtgtgtaaattcctaccaggaaagaatacatatccaatagattggagaaagccagagattagccctcattccgcatctgtcaaccaggacagaaagcatggacaagggatgagctttacaaagatgatgcactttggagatcagaaaattcatatttaagcaaagtgatacaaacacagtgatttgggaatgccttcatttacaatgcaatacttacattttaatactcttgtaggagaaaaagcaactgtataaatgaatgtagagtgactttctgcaatatttcaaacctatatcagagaattacactgtgggaaaactaccattgtaataagtgtagcaaaatctccttagatatctgaaaagtcatactggatggaatctgtaggaaacggttctattttgagggaagggggattcctttttgttttttaagtgaattcagaaaatgttataaataaatcttttggtttattataaaccttctgcttgctgattttttcccacagcatgtgattctgaaaatgtaactacaatattgacataaaaaataaacagtagtttttcttgttgaaacatacaaacataacaaagtgtttttaggtgttttatgattttaactttcagacagagtttggatttaaggtaatgctgacagttatccttgaatctgactatagacatttgttattcagtgtgaaacaaatataagatacatcacagaaaattaccaaggtattcttcctgttttgttccatgtacggtgaaaaccgttcttttgtaagcaggtatttaaaactgttctggcattaccacctgcccagctgacaaaggtcacaccatcagggttagtttgccttaatcaggaaggtaagcaattttattttgtagaaagagaggtagagaatatgaataggaatgaatttagtgagcattaatgtaatggctgcattgagggcacatttgtaggaggtgttattagataaatataagtaattttgtaagaggtgaaatttataaaagttttagcccaaaaacaccttatttacatgtactagagttctaaatacattatcagaagtgtatttcctcaaacctgccattggcatgccatattggtacatacatttagaagcttctcaagtttccataagagttgtttcagagaggctgatttatcttacaatagtgtacagtctgactcgaatacaagcagcatgccttactacgtatgggtatctaatatctgatttgattttctcaagcagcatgccttattacatatgggtatttaatatctgatttggtgtcctcaagcagcatgccttattacatatgggtatctagtatctgatttggttttctcaggcaggaatggtttgtatcagggtaaaaatcaagttaccctgtcagcaaaattaggatatgaaaaattcattatttatttatttaagagtatactcaatttctcccattatctgctccacatccactttccttcctactgtttactctgtggggatg

SEQ ID NO: 11 - DNA, 1914 bp, Homo sapiens
gtgtccccaggcagagttaagaaaagaagccaggagcctgtgtgtggagtgaactgtgcttgctggttatcagttttccgagggcaaggaatctatagtcttgtaaaccttctgtgtctgggcaccttcctgttcatgtttgtgacttagttttctcctgaacctttcagcagtttgccctccgttagcctgcccagatcatccatgggaggtcagagtctgtaggtctaggactctaggacttttcagagcatttctgaaaagccactggactggtcttcaaagttcgtctcgttaagattctgtgagactgaagggctgccccacactcagagtttgtgtctgctccctggccccagttgtgtgtcctgccccaagtccagcctctctcagtgccctcctttaagaggtcactctcccctacaccacctaccttcctgaaaggaccccgagtcttcaggagggtgatgacgacgaagagtgggacacagaccatggaggacagagccaggaaccagccaatggagtatccccagggcgggtacacatagacgttgttgtacttgaggggggtgtacttgctcaaggagaagaggaaagtggcctgggagaaggaaggggcagccatgggtaagatagggggcgactgaaaccctctccgcagctacgtacagccaaggacagaggacaagtcaggtgcactgcagcacgtctgtaaggtggaagagtaaaagcccctgcaaatcccaggccaaggcatcattcacatcacagacggagacaggaggcgatacaaaggaagggaggggctcggaagagcatcattcacatcacagacggagacaggaggcgatacaaaggaagggaggggctcggaagagcatcattcacatcacagacggagacagggggtgatacaaaggaagggaagggctcagaagagaagctcagacagacaggagaccaaccatcgagaaatcaggcagaagcaggaggcactgtgaggaagggatggagccggaagtaggaagtagaacaagattctacttatgggtggatgagatggccccagaaagaagagcagggaaggcaacatagaacaggaaatggaccaggccccacgggagactggacaggtggggaaagagccctgcatgtcagccgtcctttccctcatctctggagtcttctgggggcaggaaggaatagaggggcagctggtgggcacataccaggcaaagtccaggggtcaggaagagccaggagatcttcaccaggggccatggccggtagccaatcatgtcctcaatgttgtcatagaaacggtccgcccctgagcaggcatggcgtgggagagtgtgagagccagagggtgagaacagcttcccggtgtttgggaaagacccacttggctctgtgcccttccctcacccccgccctgtgcagggaaactggaacagggcacgtgagtgagacgcctccctgacaccctgtatccctgcatgagatgcattcgagtcacgaggcaggggctgcccccacacactgctgctgccatctcttgtcagtgctgtctcttgcctccctgtcttgtgatggagaccccactggtctaaccacaaaggagtggtgtgagcccaaaatggggctcaatggttagacaaacgcctgtttacccgggtagcagagatgaatttggttcaagccaaaacagcaaaacaacaaggctcccgctgttcagacacatcatagaaaactcatagagggctagagggctactgggaacagaacggtggtctagattgcagactccagaggaaccacctctgagttcccaaaaaagcatggtaagaaggttaatttgtgtttagtgaaaacattgactggctgtattttttgttgtttcactcctgc

SEQ ID NO: 12 - DNA, 3209 bp, Homo sapiens
cctgctgactgagggggatggccggaacctggccctgagaccgtccctcgaaggaagcagtgtggacatgtcctggaagcacctccagcccttcacatagattcccaataattccctagtttcagccgcctgttcccagctgttcattcccactgacttcctcagagcccgattcccctgaggccactgccaggccaggctctcaccagctggggagacctttctgaaggctgctcctggtggcagggccgagcctgggatgatggccaggacgccctccatgggggatcacagccatgcacgggggcgtccagtccgagacctatacacatgtgccgggtgcaaggcgggaggctcctggcctctgtaaataagacctcagctgttcaccagaaacctggagcccaaatcctccccagatgagtgcagaaggcccgtcccctagagaaggccactgtccccctgactcctgacttaagggcaagtcccacatgagagccctcccaacctccagtcagtctcctactcagaaaacctgtcttctgtgtgcaacagagccggctccttctgggagcttctgacctccaatcctaggatatctgtcccccctgccccagcacccccgtccctctaatcctaaggcttctgtcactcctgccccgggagacctgtccctccaatcacaggacccctgtcccacctgccccaggacctttgtgcctcccatttcttctgcctttgacaccctttgcccccaccccctgcttaactaactttgagtcaacgccgactacagcaccaggactgctcacttccagcttctgctgacacctgccctcgtttagtctttcttggtggctgcaggttcagtagaaactctatgccaggctttgtctccgggacataggagagtgctggtgctcagtcatgtttgttgaatgagtaataaatggtaaaggttgttgctgccccgagacgcttcaagaggaagcagccccctaaccccagctgggaggaggaggaagaatcctgggctggtcagttggggaaggagctgagcaggccgggccacctgggctgacacagcacgagcaccacgtggatgggatgcctgcagtcagctgcaggagggccttgtggggaggccacagggcccctcttttgtcttgaatggagacctccaaggctccaggacataaagggccttggccaagctgttcctggccacctggccacatctccagctgcaccagttctcacctccattccccacggccccagctgtcaggttttagggtggcagagagctccatgcaccccctggccttggcctcttctggggcttagagctccaggacttttgggcctgtgcaccctcagcgtcccctcttacgactccggcgaggacggccaggtgcctggtggactcttgcacgtgctcagccacgagacctcatgtgcgctgtcctgagcccacctgtgtcctcagatgttccaggtcatccagccagagcgtgcgctgtacatccaggccaacaactgcgtggaggccaaggactggatcgacattctcaccaaagtgagccagtgcaaccagaagcgcctcaccgtctaccacccgtccgcctacctgagcggccactggctgtgctgtagggcgccatccgactcggctccgggctgctcgccctgcactgggtaggtctgtgcctcggtgcccagctcgtgcactgtgcaggaaatgtggccaaggggctgagtagggagggaccagcagacagtgcatgcctgcctgtaagctgcacataaacagggctgccctcgcctcctcccaggagcctcccacccgaggggtcctccctcgagggagcatctggggcccagcctctggaaggctctgcgcagactccagggtgccacaggccttcgagggtcttcctgaggccctgccccgggggagcgggaggtcagggtgaagggggactccccaggccgtggccatcctgcttctctaggaggaggctgggagcaagcccctccctgaaagcttcgtctggcccaggacacccaccttgattccacatgacgcagcagcccgttgtcttcccggccccccatcagccgggtccccatcagccgggccccccatcagccgggccccccatcagccgggcccccccatcagccgggcccccccatcagccgggtcccccatcagccgggcctccccatcagccgggcctccccatcagccgggtcccccatcagccgggccccccattagccgggcccccccattagccgggccccccatcagccgggtcccccatcagccgggcctccccatcagccgggcctccccatcagccgggccccccgtcagccgggccccccgtcagccgggccccccgtcagccggacccccatcagccggaccccccgtcagccgggccccccgtcagccgggcccccgtcagccgggcccccgtcagccgggccccccatcagctgggtcctccgtcagccagccccccatcagccgggcccccatcagctgggtcctccgtcagctgggccccccgtcagctgggccccctgtcaggccccccatcagcagggccccccatcagccgggcctctggcagttgcacagaggcttgggtcatatctgccggtcctaaggaggaggcctgggtgcctggcggtccccctggttatgctccgtgagatgcacctcgctgttgttgtggccacgtgatgctttcgcataagggccctgcaggggatgagctgtgctccatgctgggccaccgtttaatcctcccacagcctcagaggtgggaccttagatcctgcttcgtggacacagaggctgaagctcaggaagggggcctggctgctgctcaggcatgcgtggccaccgccccagaatcccccaggagaggccagcgctctcccatgtcctcgcatcccaggacagcgggaagcattgcagcctgacgaggagagaaaacctggcctgtccccacccgcagccgaccgtgcagggaacacagtcccaggaggcttccttccaggccatttatctccatgagaacacgtctgccgagtttgctcactgccttggcagatctgtgggtcccaagaggctccagccgctgaggccggacagctcgggagcctcccctatcccgcacacccacagccag

SEQ ID NO: 13 - DNA, 1983 bp, Homo sapiens
cagcccagatggtcattacctgcttagttcaaaggagtctcacaaagactcatcctgccacccccaccatggcatgtagctggctacaagccagacctgctcaggctgtactgcttagatgcagaagcaggaacctgcaatcattaactacaggaaaaacagaaactcctaaaacgtacagagcaagaggcaaggtatagtttacatagcagaggggatgagattcgacagggaagttcacttacactaaaggagagataggaaaacttacctcttttcatccttatgctgagggagtgctgggagagtcttcagagcccattcctctgagctccggcccttagataacatcattgaaactttgcgtgttactgcctttgacgtgagtcagcctaacacaggcagcttgtttctttctcttttttgatttatattttctttctttaattttttcttttttctcgtgtcaacattaggttgacaacttgtgctctttccggctttttcacgtaggcagtagtcactataaactttcctcttaccactgcttttgctgtattcttaaggtttcaataacttgttaccatttaattaaggtaatttttaaattttcatcttatgccattgttaacccagatattactcaggagcagatttcttaatttctatgtatttgttcagttgtaagggtttctttgagagttcatttttagttttattctcctgtggtctgagaagatacttgatatgatttcactgttttaaaaattcattgagacttgttttgtgacctattatatgttctatcttgtagaatgttgcatgtactgattacaagaatgtttattctgcagatcttggacagaatgttctgtacacatctgctacatccatttgtttcagtgagttatttaagtgcattttttctctgttgactttcagtctcgaagatctgtctagtgctgttatgattgtattaaagtctcccactctgattgtttcgctctcatttttttaaatctctaatagtacttgttttatgaatctagttcctctggtgtttggtgcctataaatttagaattgtagtattttcttattgaattgatccttttgtaattgtatagtgatcatctatgtcttttttttactgttgttgctttgaagtccattttgtctgatatcaaaatagctactcctgctcactcttggtttccatttttgtgaaataccttcttccaaccttttaccttgagtttatgtaaatctttgtgtgttagggggatcttttagagacatcagatatttccattgtgattttttaatctattctgccattgtgtatcttttatatggagcatttaggccatttacattcaatgtgaatatttagatatgagttactgttttctttgccatgttaattcttacctagtttttttttttcactgtgttattgttttataggcctgtgagtttcaggctcttaagaggttccctttatgtgcttactgggcttttgtttcaaggtttgcaactccttttagcatttcttgtactgctggtttggtagtgacgaattccctgagcactggtgattctgaaaatgactttacttctttttcatttatcaaacagtttggcaggatacaaaattcttgattgaaagttgttctatttaaggaatttgaagatagaagcttaatccatctggctggtgaagtttctgctgagaagtctgccattagtctgatgggttttttgttttgttttgtattgctgctcttagaattatttccttcatgttaactttcggtagcctgatgactataagcttggtgaaggcagttttgcaatacatttcccaggagttctttgaacttcttggatttggatatctaggtctctaggcaggccaggaatgtatttctcaatttttctctcaaataagttttccaaacatattattttttttcttcttcag

SEQ ID NO: 14 - DNA, 2617 bp, Homo sapiens
catctcaccccgttgacacggttagtttgcatgcacacacagagcggccagccgccccgagcctgtgggcaggccagcagggtcagtagcaggtgccagctgtgtcggacatgaccagggacacgttgtacagggtgggtttaccggtggacttgtccacggtcctctcggtgaccctgttgggcagggcctcatgggccaccacgcaggtgtaggtctcccccgtgttccattcctcttcggacacggtcaggatgctgtgggcgaagtaccggcctggggcctggggctcaggcattggggcgctggtcacatacttctccggggacaagggctgccccctctgcatccactgcacgaagacgtccgcgggagagaagcccgtcaccaggcacgtgatggtggccgactcccgcaggttcagctgctcccgggctggtggcagcaagtagacatcgggcctgtgcagggccacccctgtgaacagagatggtggtgagggcggggcagtggggggaccagcctgtgggctggggttgagtccccttttccccagttgcccagacaacgggggagtgaggggtgctttccaccatgccccagaggccaagggaggtcccagggagtgcaggaagaggggcaagagtggggcctacccttgggccgggagatggtctgcttcagtggcgagggcaggtctgtgtgggtcacggtgcacgtgaacctctccccggaattccagtcatcctcgcagatgctggcctcacccacggcgctgaaagtggcattggggtggctctcggagatgttggtgtgggttttcacagcttcgccattctggcgggtccaggagatggtcacgctgtcataggtggtcaggtctgtgaccaggcaggtcaacttggtggacttggtgaggaagatgctggcaaaggatggggggatggcgaagacccggatggctgtgtcttgatctggagtcaagagaagggagtcagaggtggggcaggtgtggatgtgggcggaggcatggttcccacccaaagagtagcaactgcctctgccgagcccaggggtcctgccgcccgagcccctgcccttggccgctctgggaagccaaggctcagggagtagatggctgcatccggggtggcgaatgccagacccgagtggacccctgtgtgtcggtgggtgctgcccctggggacaggtcactcaccggggccacacatggaggacgcattctgctggaaggtcaggcccctgtgatccacgcggcaggtgaacatgctctggctgagccagtcgctctctttgatggtcagtgtgctggtcaccttgtaggtcgtgggcccagactctttggcctcagcctgcacctggtccgtggtgacgccagaccccacctgcttcccctcgcgcagccaggacacctgaatctgccggggactgaaacccgtggcctggcagatgagcttggacttgcgggggttgccgaagaagccgtcgcggggtgggacgaagacgctcactttgggaggcagctcggcaatcactgcagtgagggacacgtgtcagcccggtgcccgccactcccgcccccttcggctccctctctgtcccggtggctgggcccggccctcacctggaagaggcacgttcttttctttgttgccgttggggtgctggactttgcacaccacgtgttcgtctgtgccctgcatgacgtccttggaaggcagcagcacctgtgaggtggctgcgtacttgccccctctcaggactgatgggaagccccgggtgctgctgatgtcagagttgttcttgtatttccaggagaaagtgatggagtcgggaaggaagtcctgtgcgaggcagccaacggccacgctgctcgtatccgacggggaattctcacaggagacgagggggaaaagggttggggcggatgcactccctgaggacccgcaggacaaaagagaaagggagggtgaggagctgcctcctcgtgccctgcctgtcggggctgagtggcgttctgagtgccctcactacttgcgtcccgctgtggctgccccaccaaggccgagcccacctgcaggcctccaaagcccagactgtcatggctatcaggggtggcggggccgtggtgaggcctcaggtctttgtccaaggctgctggggctgcaggcctcggcccatcctgctgcagggcccagcactgaacacctggacagacctggggtctcctggagcaggctgagccatccctgccaccattcagctggctgccctgctgcactctgaggcctgactgcccctggctccctgctcagaatggctgagggctcaggtttgggtggaccaggcctgctttcccccgaggcatcagcacgtaggtgctgcacacactcagctcccagcacatgcagctggagggcccaggttgcatacctgaatgtgaagcctggagccacacaccccgcaggcagccaatagagtccctccagcccagcttctgctgcccccagctcagtcacactccagctaccctgaagtctccccaggcagacaacccaggcctgggagtgagtatagggagggtgggtgtgatggg

SEQ ID NO: 15 - DNA, 3839 bp, Homo sapiens
atacatctccgacactaggaaagacacgacaaagcgttaaaacgcagcttggtcactcaccacgtcgctggggcacgaccacgggctgctgagaaagctgggccctgccacctccccacgcacccaagcagcctgaggcaggcagggttgtgacgcaggacggtggactggccgcctgtgcccaggctccagagccaatgcggtggggtgcaggctgctcccaggcctgcgggagatgcacccagcgtaaccatggggcctgaggtgggcttggggtttgactgtctcgcagcagagcatgcatcctggcacttcaggtccctccacactggacccaacagcagttcaccttaacaacgcctttttagccctggtcctgttactggaaccaaagagcaacgccacgaagggactaggaaatccacagcaagagccaacctaaacccctaaaccagggaaggctgtgctagcacccacttcacaaacgaggcgagcatggggaggtgctgattctggggctgcgcgccagccggcaaaagcccaggtatctgagacataaagcttattattctagtttacttggagtcctggcgtgcgtgccctgacccccgcctgtgagggaacccctggaagcagctgaagcacacgcaggccggtgtgtgccacgggggcgggcgccaggcctggggacgccctgaagatgcttcctcagctggaggacccaggcacagagaagctgtaagactcacaagccagggctcacaaggctggactttgttggccaagagtgttctatgcacacagaatgtacaaaggtagacagaaacaggaaggtgactgggctcagggcccaccaggaattctgacagcacaagacctgggaactgggcaggtggccatggggctcactttccccaaggggtcacagcaggcctgaagccccatggcaaggtggtactgtcccggcacctcagatgcttggtcggcctaagggtaaaggtggaattgaaatcagttagaaataaaacagatttaagatgctccctgcatttccactgcttcacttgactagacaaaaaaacttgtcaccgaagcacagggtgcatttaccaagcacccagagacacacatgtggtggtctatgctgaagccccccactgacgctgggctctcagcccctgccaggaggccctcactgaggaggccacaagcccaaggtcacaccccactgtgggcagccatggccacccggccaactccttagaaaaaccagccgggcctccaagctcccgagggctgcagagacctcaggactggccacagccagcttctcagcagccccaaatggagcgtggcctggtgaggtgcctgctccgaccaccacagagcctgcttctgaggggcgtgggtcccagctgtgcctgccgcctccacttagaacagcaagccggatgcgttgaccacttgcagggggttcctagctcgaacctcctcatgaccaagggacgaagtcaccgtgaacacgctcaccctcagcaccaaaggcacggaactcccaaacctcagctgggaaggcctggcctggccgcctcctgctcactccagatggcagggggaccctgacgccggcacgagcgcagcacgaggacgccgccatcgccgccggctcccccgctctaacagcagggacttcagtccaaggggaagacattcagacctggctctgaaggaaatctgtgtcaccatgcattcttttaacagagtgagggacacttttgccacgaaaatggtccccggatttggtaagccggtacagcctttttcaaagctggccctcggtgctgcccacccgctccccagcaggcccttcagcagcgcattgggggctgcgggacccaggacgcctcgcctccctcagcttcatgagaacaagaccctcgtgctctggggtccttggtaaggatgaaacaaggtgtgacaagcacaccccgctttggtcctcgctgtcagagacctcggtggcgggtggtgaaccagaaacaggtgtgggttcaatgaaccagcgacggaacggtgggagtcaaaggggtcctcttgggagagatggagggtcttttggcttctgatgattaagggctcggctgaatattgaccaagaatcatccatgttctaagcacaataatcctcaaaagagatgtaagagaagaccttcgctccacgaagagcccccttttcccttctgggggaaggagggggcccccaaacgagaccaggaattacctggcgagcataaactgagggcctgaagtctcgaaaaggaggcagactggaggtggccacagcattaccaagccacacaagagctcagacgtcttatctaacgcgagagccgcctcagagctccaccaaggacagacgggctgtgctggcaccgacaagcagctgacagggctcggcccctccgtgggaaagctgctcccacacgcatggcaccgttccagcccaaccctgggccggcgaacactgctggggctgattccacaaggaggcaggcaaggcctgtggggtcaccggggccgagcaccttctggaacacaggcccctgggtctgagctggggtggggaccgcgcggccgcccaatcccccagcgcctctgacatggctgcacagcctccctgtggtctgggggcccagccacggatcctccatcaccccaccctgatcctctccctcataggcatggggactcttccctgccctgcaccccttctctgggaagtccaaccccttctctgagccccagaagacgctggtgtggaggagctgctctgatgcggtgccatcacagccgccaccctcaccatgtccccgccaccctcagcgtgtccctgccaccctgcaatctgcaaaggcaggggcctccctccagcctgcgggacccacacaggcagcacaggaagcctgcagcccctccacagggggctcggagacagtccacatcaggtgccaagtgcccactgtgcttagttggcaaaacagagtctggtggtcctgggactctgcagatgcttctggaaggagtcctatggggcccacagccacgtgtaccctcactgtaggaggacagaggtcccggttgtggcgcacatcaggggcccttcagacgccattctgcagcaaggactggcccgtcgcgacccacacgagggcctcatccctgccgagttccatgtcgccactgccccaactcaggcaggcaggtcctgagctttgtgagatcccacgaccagcctttttttgtttccctttgcttttaagctgcttcctggacttggaaaccaggcctggcccaccccagccttctggaagcatctaaaaagtccagctggcagctctgccaggggctccctgcccacgggctgtgggcgttggctggctgttccccgccctgattgtgcttcagcccagccctgccattgccctcaaatgggcctgtcggttctggaatgttctgcctgctgtgcggtggcacagtccctgcctctgtgtggtggccccttccctgaccccagacatccactagccacagaatccactagaatctgctagagaaagcttcacgggggttttaactctgagcttaagcaaacacgaggccacgttatcaccaggttccagtgagagtaactattgatggtctctccatggtgaccctggcccacagcgcccgacaggaggggagagggctctcaatattctcagcagacggtggtgaaagaggactgcttttcacatttactgtgcagtttgtgtttgggcaagctgaaaggccaattt

SEQ ID NO: 16 - DNA, 1866 bp, Homo sapiens
tcagacggtcgagtgacagtccaaacggggtctggtcacctggggcggggacttgctgaccagcatagacaatgacagctgtccccacaggacaccttgttggagtgtgtgaataagaaggtccccgtactgctgtctcggggcatggctcgcctggtggtcatcgactcggtggcagccccattccgctgtgaatttgacagccaggcctccgcccccagggccaggcatctgcagtccctgggggccacgctgcgtgagctgagcagtgccttccagagccctgtgctgtgcatcaaccaggtgagcaccaaggcagggttgcacccctgagctcgtatttttagccaggatgcggaagcagagccggtctggaggtggggcgggtggcagtgaggtggcctccggctcctgcgggtagcagcctgtgcctaaccatcgagaagaccctcagccgttgcagctgacctggactgtgctcttccaggtgacagaggccatggaggagcagggcgcagcacacgggccgctggggtgagtgcagccatgtggtgtgtgcacctctgtgcaggtgccaggggcacagctgggccgaagtgggcggggccaccaagcctgagcgccagcttgcctgcttcctgtttctcaggttctgggacgaacgtgtttccccagcccttggcataacctgggctaaccagctcctggtgagactgctggctgaccggctccgcgaggaagaggctgccctcggctgcccagcccggaccctgcgggtgctctctgccccccacctgcccccctcctcctgttcctacacgatcagtgccgaaggggtgcgagggacacctgggacccagtcccactgacacggtggcggctgcacaacagccctgcctgagaagccccgacacacggggctcgggcctttaaaacgcgtctgcctgggccgtggcacagctgggagcctggttcagacacagctcttccagggcagcggctccactttctcatccgaagatggtggccacagactgacccccatctgagctggggggatgttctgcctctccctgggtctggggacaggcccgcttgctgggtacctggtccccactgctgagctggcccttggggagaggtgattctcagggctggagcctggggtgtcctacagtgactccctgggagccgcctgcttcttctctccacatggaagcccaactggggttgcgtctgaggcctgccccctgggctggggcctcagaccccctcagccttgggaccgtgcccacgagggtctcccctcctgcacacagggcagtccttactcccccaccactcaggccacagtggggctgcaggcaggcggctcctcctcacccacctctgggtccttggctcccgggggccccacctcggcacacactgtgccccacaaaacttcagtgtggtacaaggtggagaaagcatatcccaccaacctccagtgtcagggtccaggagagcctgggggtggggggactgccttgtctctagtagtgtggcctgtgccagcaccacagccggtcagaggagcgcaggcagcgcagggctggcacgtgacaggctcgtcagccacctgggaacacagttctgggcaaagaggatccgaggttgagaggaaggagggtcccggtgtatcctggccctgggggtctgggcgtccagctcagccctggcctggctgggtggtattctggtagggatatggcaggactcctggcagggccacctgcaggaccctgtcctgcagtcccacactgtgcagacccagtcccacactgtggccaggccttacatctggctggaaagcagagcctcctg

SEQ ID NO: 17 - DNA, 1607 bp, Homo sapiens
ttttttttgtcacctagtatttgcaacacattgtatgggcaaactattgaaataaaaaattaaaggagtgatgatttataaccttgagcagtttataattctataggggaatagacatgtgaccaacaagcatttgggtatattggtgggtcctaaggaaggtttgataaatgaggtgctatttgatctggatattaaagaacaaattatattttgagaagtgtaaaatagggaaagaaaatttgtggcttgaacaaagaaatctgagtcacaagatcttaaaagtctatgtcacagaatagccctctttgtctgtctcgtatcatcattagttattactcctccagggagagggtggtgaatattgattttactgatacagcaatttgacatcaaatgcactttctttgtgatttccacaggtaaacacaggtaccaatctaccagactatttcaccatcccttaaattagcaagctcatgtggcagcttcgttactgtcacatgtaactgcagcagtagtggccaaaagaatgtcatttgttattcatgaggtgctcaggtaatatttgactttcatggttatatactttttcatagaggctattaatataatactattaattagaaatttctcatttttttttctctttaggtaacgtgaaagtgaacttatcaaatgaatagggacaaccagtctgtggtgtctgaattcgtgttgctgggactctcaaattcttgggagactcaagatttttcttttttgcttttcttgtcttttctatgtgtccggtgtgatggcaaacctcattgtagtggtcattgtaacctctgacccttacttgcactcctccttgtatattttgctggccaacctctctgtcattgatctcacattttgctccattgcagcacgcaagatgatttgtgatattttcaggaaacagaaagtcatttccttttggggctgtgtagctcagatcttctttagccatgctgttgggggcactgagatggtgctgctcatagccatggcctttgacagatatgttgccgtatgtaagccccttcactacctgaccatcatgcatccaagaatgtgcattttgattctagtggcttcctgggccattggtctcattcactcattggtccaattgtcttttgtagtaaacttgcccttctgtggccctaatgtgttggacagcttttactgtgacatacctcagctcatcaaacttgcttgcacaaatacctataaactgcagttcatggttactgctaatagtgggttcatttccttgagtgctttcttcttgctcatcctctcttacatcttcattctggccactcttcagaaacactcctcaggaggctcatccaaggctgtctctactctgtcagctcatattactgttgtggttttattctttggtccactgatttttttctatgtatggccctctcctccaacacatctgaataaatttctagccatatttgatgccattttcactccttttctgaatccagtcatctacacattcaggaacagggaaatgaagattgcaataaggagagtgttcggtcaatttatgggttttagaa

SEQ ID NO: 18 - DNA, 2567 bp, Homo sapiens
ttctctgcttcttccttgttttctctccacccttggagacctttttctgctgacaaccctgtgtggatggatgcatccatcaaaccaggctgctattcgctggatctctcagaacgcccactggagtccccaggccgctcccgttgccttggccaaaagatgagtctcaaactcccatcacctctctctcctcaggatgttcttgagtcgaagaacagcaccatcaaggacctgcagtatgagctggcccaggtctgtaaggtacggctgtgccctgccctccctcaggggcaccccctcggtgcccagactgttctaaatgcagacggtctctgaggaccccacctgtgcccacttcgtacctcgtttgacaaggcagctgtcactgtccccacgtgagggtgcagtcatagccgagagcatctggattctgtgtggtctggggcagtgcactgctgtctaggccatgtctctgctgggatgggtgtagggggggacctggacgcttccctggtcagccccttcccctgggcagggagtcagaaggtgctgtgcccaccggggaaggaaacagacgtcattcaacaggggaagggagggcgtgaagaacctgagtgggaaacacccagccagggcccagagccctcccagaccacagctctgccctgagtgtccctgccctctgcctctgtctcgtcatttgtggaataggaatagtgacagcctctccctgtcgtgctacctgagccaacgcagtgaaggtgcttggagctgtgtcccacacgggaaatgactgataagcctttggctttatccttctgcaccgtgatgctcacgctgcccctccatggagctgcactcagctctggtggtcctgagcgtggggaccctcagctccctgacactgccctgtctccacaggcccataacgacctgctgcgcacgtatgaggcaaagctgctggccttcgggatccctctggacaacgtgggcttcaagcccttggaaacagctgtgatcggacagacgctgggccagggccccgcgggactggtgggcaccccgacgtagctgcccccctggggggccacagcccagagaaccagcctaggaacactcgggatgacaccccttatcacaccaaggacagcaagttttttagattttatcatcagcaaatgaaagcttttcacatgttcttgccatcctctttcctggctctgtggaggagaaccacctgcaggaccctcacccatggtgtccctgtcgctcccttccctgggtgccgcacgtccagcctgtgtccaggcctactccctggtctcacctccgaccacagtcggcggcaccttctcagagtgccccgcactcacctgggggttggggcagtgccgcgctgtgctgcctgtcttcgcgccactgttgtcccaccgaatggacagctttgcaggtgctggcactaacttcattgacacctgagtcacagctgcccagtgggattctccagggggccgggacttccctaggaagtggtgagccaatgctccctgatgagcacaaagcccgctctgttgagggctgggtgggtgcagccagcgtgcgggaacgggcaggcagcctcccgctgccagtcttcgctctaactccctcggtaggtgatgtaggaccaggggcacgtggaacttctgggccttgctggtgatggttaaaacaacctgagatggagaggccaggagagagtataaggggatagcagcaaaccacctatctggccccaacacacctgagagaattcagcagcccagactgagggtctgggatggggtgaaccttccgcaccagagggacactccacagaagccacagcccagtaagtcaggcgcttctgcggcggctccagtgtggggtgaggcagtgaggttaggcccagagagctggagttggctcagatgaaaacctctgtcaacaaagaggggatgaatcacccttggcccagcctccccacaaagcctgaccctgggcaggtgagtgacgggtgtgtcctcgtagagtctattgctgcctggacacctttcttttgggagctcaaagcaagtgagctcacctacctgccaccgcccaggaccagtctgcccactgcctaaatgatgcccggccagcaggacctggcctgcagatcccagtgagtcatgagcctcagccccctccagcccactggggctctcacctccacatgtgggtagaagctttcctgccccctcttcctccagtagccctcagtgtcgaaggtgagcttgtaggtgcctgccttcatctggtccaggacagtgaccatctgggtctgtgtagctggggagaggatgaggctgcagagatggggaccagaagccccccaccccagctttcctgggtctgcatcccagtgggcctcagacactgccctgccacctgtcagacttgggtgagcagacacagtgaggctgttaggtcctgcagttccagagcagtctagggacaccactgccctgtctttaggaaatcacaacacagagaagcaaaaagggaaa

SEQ ID NO: 19 - DNA, 2082 bp, Homo sapiens
  misc_feature (1774)..(1873): n is a, c, t, or g
taagggttagggttggggtcagtggttaggggtcatggttaagggttaagggttggggttgggggttagggttaggggttagggttaggggtaagggttaaggctaaggctaggactagggttagggttggggttagggtttggggttagggctagggctagggctttgaataaacttatatggtagccaagttgtggttacagtgggccttgggtgagaccaagttctatgcctacttcaagtgtgaaccagcacagtctcagtggtcgtggcctcaggggtgcttatgttaccccaactccagctgccacatgcctcagcagagaaagagagactgctggtttcagagaaagaaagggaagagaacaagatctctacttgaaaaatcaagagaatttttcttgatgttaatccaaggccaccaaagcagcacctctacgtgtttgctactatgtattgggcttgggacctaagtctctttgaacacctggaaagtgttcccaaaaataatgggcaccaacaagcccagactgtgaagactacaataaagactgacctcttcaatgcccacatatagatgaacatctataagtatcaaggccatgccaggaaaacatgacctcaccaaacaagctaaataagtcaccaggggcaaatgcctgggaaaatagagatatgtgacctttcatacaggaaatccaaaatagctggttgaggtaattcaaagaaattcaatataacacagagaaggaattcaaaattctatcagataaatttaacaataagatttaaataaaaagaataaagcagaaattctgaagttaaaatgcaattatcatactgaagaatgcatcagagttactttaaaaaattgatcaaggagaagatagatttagtgaacttgaagtcagactatttgaaaagacaaagtcagaggagacaaaaaagaataaaaaataaagcatgcctacagaatctaaaaaatagcctcaaaataggaatctaagagttattggccttaaagaggtggtagaaaaagagataagagttaaacatttattggcccggtgcagtggctcacacctgtaatcccagcactttggaaggccaaggcaggtggatcacaaggtcaggagatcaagaccatcctggctaacacggtgaaaccccgtctctactaaaaatacaaaaagaaattagctgggcacggtggtgggtgcctgtagtcccagctccttgggaggctgaggcaggagaatggcgtgaacccaggaggcggagcttgcagtgagccgagattgcgccattgcactccagcctgggctacagagcgagactccgtcaaaaaaaaaaaaaaaaataaacatttatttaaagaaataatattaaataatattaaacaattccccaacattcgatatcaacattcaagtacaaaaaagttacagaacatcgagcagatttaacccaaagaagaccacctcaaggcacttaactgaactcccaaaggttaaggataaagaaatgattctaaaagcagcaagagaagagacacaaataacattcagtggaactccagtacatctgacagcagacttttcaggggaaaatttacaggctgagagagtggatgacatattaaaaaagctgaagaaaaaaaagactttactttagaatatgtatttggcaaaagtcttaaattgacagagaaatagaactttttcgagcaacaaaactggggttctttacaaccgactgtctttagaaatgtannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctggtgagtatgtggttgcattgcgaagttctcgatgtgtgtttctcacctccatcaggtcagttatgttcctctctaaactgaatattctggttatcaccttctgtaatttcttttatgatttttagcttccttgcattaagttagaatgtgctcctttactcagtgtggtttgttattacccacctcctaaagcctacttttgtcaa

SEQ ID NO: 20 - DNA, 3362 bp, Homo sapiens
gacggaggcagcacatgaggatgagaagctgattggagaagaggatgactgcagtgctaagagcagcgtggtcaggttgccaaggatggagcagtgggcacagcagggggacttagggtcggcggaggagtcggtgaggaaagggaggtttggcaggaagtgatcaaaggggtcatgtttttgtcaggatgtgggacttggatgtgttctgtgtgaaggagccagggcacggggctgtggtgatgagggcggccaggctttgactcatttgcaggcggctctgtgggggctcagtgagacaacgaggggcgtgtgccctgcacccacagggatgtagagggtcctgctcctccctactgaggtgggtcagggtgggcagcaggcaccccacctggtgagctggaagcagcgtgggaatcacagaatggacgggaacttaaaggctttgcttggcctggattttatcttgaaatacttttgacagctggctggttgagggtatctgctcacaggaacgccgcatttgctggctttgtccactagtgctcgcccctggctgctgatgcggagcctcacgtggccgcagcccaagagtagggactggcttggccacctccaggctaagcttcggactcccaggtggctgggagggccaggggtgcacaggtgcatcagagcaggtgctgccttgctggagggccagggctcttctggccagggtccaggtcatcattgtccccagccaggaatccaaggggcctttccaaacctgcagggcagagggaattcgggtatctgtgcttgagtgagcccctgggcccaggagccttcgcttgctgtctctgtttctcaaggggcctggcctggtgagggagggggctaggctggaggagggatcccaagggaggtgagggggctttgtcagcctcctcctgccctgcctgtgcagggtgttgcagtcagtccttccactgagtcattgcatgggctctcccaacatccggtgcacactggcagctgctctaagccaactcctagcccccaccacttgaccaacacaaacactgagtgggtgaggcagaaggggagcgctggggcctggctaggccaaggcttcctgcttcctggctgaatgatcgcacccgaggactggctctctggagcttcctttgctggctttatagctgctgccagtcacaagaccaggggaagccaggtggaaaggaactgatacccagcatttgtcatgtgtttttaacagtctggctttgtgggggcggccacagtgggggaggccctgcctggtggtggaagccagaggtgcccacaggaggcacacctcatggtgcaggcttggaggatggcaaggtaggcagaggggtctggacacagtgaggtgcagccccctcccaccaggtcagacccaggagatggtgcaggtgcacagagcaggtccctggcccaggcaggaaggcagctgcaccctccctgcagcacaggatgtctggatgtgtactagggcagagaggacaggagcctagggaggctccacttccaaactgtccgtcccacaggggacggggcttgcgtcttgctgcgagcactggagcccctggaaggtctggagaccatgcgtcagcttcgcagcaccctccggaaaggcaccgccagccgtgtcctcaaggaccgcgagctctgcagtggcccctccaagctgtgccaggccctggccatcaacaagagctttgaccagagggacctggcacaggatgaagctgtatggctggagcgtggtcccctggagcccagtgagccggctgtagtggcagcagcccgggtgggcgtcggccatgcaggggagtgggcccggaaacccctccgcttctatgtccggggcagcccctgggtcagtgtggtcgacagagtggctgagcaggacacacaggcctgagcaaagggcctgcccagacaagattttttaattgtttaaaaaccgaataaatgttttatttctagaaaactgtgccttagccagagctcctctaggtgatcaacccatgtctggagctagctcttcctccaggacacgagagctgggggcctgagtacgtagcgccaggcccggtgtggatgctggggagaatcatcagtgtgggagccgaaagcccccgagggtggggtcctgcacagtgggccatgcctccaccagcaagatgtgcacaggtgacagggcttctccagcctagcagggccagcccaggccctcgtgccccagatggtcaggaccaggtcacagcttggctatgagcctgtttgcggcttctgtggactgtggtgaggactgggccaggaaaggctcagggtagcctgggaggaagaagcgcatggcagacagaggtgctggggagggggccacagggcacttcacaaatagaaggctgtcagagagacagggacaggccacacaagtgtttctgcacattcttcagggtggccacagactggggggtccaaggagcaggtgtagggacagaaggagggtctgagaaacgcacagcccacatgggccttgaaggatgcggcctcacccagagacaggagtcctggcaggcccccctccagcgtggagatgcctacgcgtgcggcaaggactggagggaagcgtaggaacacagagggcagcagccccacagcggaaccaccaggggcaaggacagcggggctctgcaggcttcactgggccacggccagcccgcatccacccaatgccaggcctcagggccaagagggctcagcctcagcacggggggagccctggggtggggagacgcgagcgcccacctgcgcaccccagcagccttccgccctccgcctgggctcaggggagcagagcctggaagacggcaatgacagggtcctcgtgggtggtcaccaccagcacgctgcggaacttgtcaaacagcatgagcagctgggagcgccgcgtgttctcgttgtacataatctcctccaggtggtggcggccgcggaagtagtgaaggagcctggaagggatgggtgggtgtgagcccaacctgacaccagcccccagaggcctctgctgaagagccactgctgggaatcagctctgagctgcccacaggcctgaacagagctggtggtgaaggccagggaggcagccaccacagccccccaacaagggtgggcaggcctcctggaccccatgcccaccacggtcccgctgaccaccaggtgggcggagtgggttcaggacggcagacggctgttcaaacccagaggtgcccaagcctgcgtcctgatgttgggaccagggttctgctggtggcttctttttcgtgcta

SEQ ID NO: 21 - DNA, 2219 bp, Homo sapiens
cagctgttcagaaaatccaggtgtgtttccacctgcaacaatgccgagctgtcagcttagacttggaaggcgctaagagctggggaaggccacatttggggtctggttccaggccttgcgggtcaccatccctggctgtattagtcctttcctgcactgctataaagtacccaaggctgggtaattgataaagaaaagcaaagtaatgggctcacggttcctcaggctgtacaggaagcttgatgctggcatgtgctcagcttctgaggaggcctcaagaaacttacaatcatggcagaaggctaagggggagcaggcatgccacacggtcagcgcagcagcaagagagtgaggcgggaggtgctacccacttgtaaatggccgagctcgtgaggactcaccaaggcggacggtgctcaaccagtcatgggaaaaccgcccccgtgatctagtcgcttcccaccaggcgccacctccaacgctgagggttacaattcgacatgacacgcgggggggacacagatccaaaccacgtcatcagctctttcagagggagatggctctggaccccactttagagtctggctgatttgctctcccaggtgcgcctggcacagctctcaggttctgcaggagccgctgggcttggacgaagggccctcccgcagtgtgaggagcctggcgacctggcccggtctcaccccacagcctagggcagagatgccacaaagtcacagactttcagggccaagagaccctggagtgcgtctgactcggcctcgtgtttcacagggaatctgaggcccgcactggccaagtgacctgtctgtacttacacactctggaggcagcagagtggaggagagtggtgctatggcctgagtgatttattttagaatgcagtcatgcattgtataacgaagtttgtcaatgacaggctgtatatccagcggtggtcccataagactacaaagcagctgaaaattcccgttgcctagtgaggttgcggcgtgtaatgtcacagtgcaacacgttatcactcgtttgtggtgatgctggtgtgaacacacctattacactgccagtcacatacgagtggacagtaatgccctgggccctcacactcaccacacactgactctcccacagcgactccagtcccgcaagctccattcacgggaagtgctctatacacctgtgtcattttaaaacatcttttataccgtatttttactgtaccctttctatgattagctacacacataattccacggtgtcgcagttgctacatgctgcacaggtttgtagcccaggagcccaggctctcccacatagcctaggtgtgctgtaggttctgccacttagatttacgtccgtgctctctatgatgtctgcacaatgatgaaattgcctgacaacacatctcttggaagtatccctgtcgtatcctggttgttaggtgacacatgcctgtacttctgtgtgaatgagtttgagtaagatctcatctgcacacacattaagggctggctagccttattagcataaggaatgtggcagtgggttttctttcatttatttactgtttttgaatagggtcttgttttgttacccaggctgagtgcagtggcgagatcatggctcactacagcctccaacttctgtgctcaagcaatcctcctgcctcagcctcccaagtagctgggactacagctatagtgattttgatagggggggaatttgttgggggtcactgaggcgggctggggcacacagaccagggctccccacgagggcctctgaggcacacagaccagggctccacacaagggccctctgaggtacgcagaccaggctgaggcacagagaccagggctcaagagctgctctgcccaggattcctgtggctgctgtgaactgagtgctcctggccgaggacccacagcttctgggaagtgtaggttggggctcctgatctgctggcccctccctagggatgcagagcacacaggccctgggcctggagtgtttccatccatccacacatccttcttcccatcaggacactggtccatcctctgttcatctgtccatcctctcagatgtccttcagcacattggtccatgcagaatatctatgcacctgtctctccatccatctgtccaatgctccatcagtctgtccatcatccatcctcccatctgtcctccacccaccc

SEQ ID NO: 22 - DNA, 4984 bp, Homo sapiens
tcctttccttttttgccttcttcctcatctgccctgtcttctggcccacacactcttaaccagcgttcacactcagtgtacatggcctggaggcccgagtgtttgtacatgagtgatgatgtcaaacccagctggtaacaccttccttgggtcatgtttgccattttcttggaatgaatgtgagttcctgctcagggctcatgtccttttacagtgaattctatataacgcccctcccagtctcacagctaggaggcttcatcactgctaggccagttggagcgttccctagagctcagaacaaattgtttcctctgctgtccctaaatataggacacctacaagcactctgaagcaagggcagacattcccacctggtacctgtcaaagtcctaggatgcctgggatcttccatctttcagtctagcacgtgggaccaaatacaagagatgctgccctcacaacagccttggaaaagatgagcgccagggctgtcagtacccatcggttcagtaagcgaggcattgtccacgctgcctattcactcgagagatgaatagtttcctgttttcgatggctggggagccagtatgagctcataaaccaaacagcaattttcagagacatctgttcctgatcttcagaataaactcagtgtccagttgcttcggctggtgggagccaatattcacgccactgactctctcaaagggagggtgggccctcggagacccagcttctctgacaagcagattagaccaaaaggctgcctcaaagatatgccactttgaaggaaagcgtagagaagcgtttacataaaagaagacgcttcctgttcagtggacaacttcatgccactttcaaggcacaccgatggccaggtgggacatttgtactgtagcagcacatggcaaaggtgagccagaagcagcctggatgctggctgatccggaggcctttgtgaagagcaaggagagggctccagcccacctccccgcagctctgccccagcccccgtgggccacagggaggctcaaggggagtgaactaggtaaacagattcctggaaactcacatctggatgcagctggaagagttaaatatttacattggtggcttccctggaccaccgcgaacacaaacatccacaccacagggctgagttttgtgcaaatgatggggctttgcattttttattaacattttcctctcacgtggtttacatcaatttataataatctacataagttgaaacagaacatagacaaaaaaatatatccttaccaacttattaaagtcagatattcatgaagggtcccatcctacctgtgtatcagcagaaactggcagccatcagccattgcccagcaagaacaggcagacctggcgtttcttagcctgactcctgctgggcacagcccaccctgctgggcacagtgactggaggttccaggctgcacagtccctggctcctgactcctgccgggcgcagtgactggaggtttcgggctgcatggtccccggctcacaggagaccctgctgggtgtttccttggtgcagtttagtccaggtctggcacctgaccctccccactctgggggtgggatttataaatatgagcctttgcatttctcagcctttgcagccttcccatagcctgttctcacgttgcctcagcgagcttggggctgtggggctccctgaggctgagacgcgaaggtgcccagtctgggccgtgactcactctgccccttcctgtccatcactttggaagcaagcaggagccttctgtgccacacaccgacactcggatgccaggcagggaccttaggaagggccaggcactgcatctttagactcaagttcaccgcctttcccagggagcaagggctccttgctaagctgctcacaggcagccgatggtcagtacttccttcctcttgggcatgtctttcctccgtgcacagagtatttactgttctgcccaaggccacaggagtaaacaggctcaaaaagggcctctcaccgcgcacgcgctgcagcgttagggccggcaaacccttctttaagactcagccctgagcacaagcaatgggaactgagctccccagccctgagggcccggaaacgacgctctgccacacagaagagccggggagctgtaactggctataagtcgagcccctggagctgcatctgctctcctaggctgatggcccgaggctggcagccgcagctcgtgtgggaagtgtacggtgggaacacacctcactccttcctagtaccgggcaatgcgtctgcaagtcgggtccctgctccctggcgggtgcctacagcaccaacaaggaggccccagcagaacccagcccctagaggcggctgtctgattccccactctccccacaacttctggagttcccagtgtttacccaaaaggctgtatccagaagctggggcggcaccacaatggctggccaccgtgggcctgtgcctttgcttcccaggtcctggaggaccgtggcagtgcttggctgtggagtgtgtgtaaaatctaaggcaagagtaccacgaggtcctgcggtgccagggagctcctggctgcagcctacctgcctggacacctgcttcggccacatcagtcaccctccaggaagcctggcccctcttgaaaagcccccacaacttgctcctaagagctgagctgcctccccgcgacccgggacacccagcgtggcatgtgcattcctcccccgttcagcctgtggtgtttcctcagcagcctgaccgcctcctcccccattctctcctgaccctctggctatctcgatagcaggtcacctgtgagtctttacactcaaaggaaatagaacagcagggaagggaactgaaaagcagtagaagaaacagtcagagatgcctcactgatagacaggaggccgaacaggtaaaccccagaagtggagattcccaaacggaaaattccagaaatgggcgctccagctctgtgctaagctggggacgagtgtgagtgtgtctgcttgtccaacatttgcacaggcagcaaggcaaagcaggtgtgctcccaaaggcggagtctgaggaggggccggcagcggcaaacggcagcatcaaacagaccactgctgccgcggcaacccagggcctcttcagagctttcaaggcgatggagcgaagaccaagggtgcacatgcatgcaggcaggctgggaaggaagagcgggtggaggaagactgaggggaggctgccaggagaccgccatctgggagcagggccaagagagaagctggcagcagttacacagcgcaaaataaaaggccttgggctggactcaggcggaaagaaagtgctggaggaaatgaaagaacaaagcgggctgtctgtgtgcccacgccgggccggtcactaccttttctgcctgacaagtgtacataaaacaattcccgaacagcacggagcatcagacacaactagaggtatggagggcaggaggtgggatgcggtggtgaggctggggctgggcagccggctttgtacaaggtggcacaaaagacgtacgcattccagttcttggaagctggcttccctcgagtctggagtgctgggtttgggagttttctattgcagtctttcaagtctgagttggaccccaggctggaggggctggttccaccacccgcccgcagccaccctgcctcgggctacacgtcggtggagaagtacagtgtgttccgcttgagttctgcgaaggaaatgggggggtgctgcaggtagtagaggaggacctggacctgtggggagacaggaaggcggaggctgggctccctgtcctaggcctcgtccttgctgactccagcctgtgttgcccctcccactccctagactggctccggccaccgccccttcctggggagcccaggtgtgtttgcctttctgcagccgtggaaggtgctacggggcagagggtcgggggcctagggccacttccccaacctggccataagcttctgctctgtcctgaggcggccacagtccggcccctgctctgggtcttgcaggaatcccagggaagcctcccgcccttggaagcaacctcagagcttccacccatgaggacaagggcccagcatctccccacccctgggcttgctttctgagactgaggccctcctgagaatgcagccagcatctctgggccctggtctaggctcacatgtttgttttggcctgggaggggcagaagtgtctacagtcctgcctccctggtgacaccccatagcccatcaacccagcttcccacgagggaagaggtgtggggactctgagctgttctctctcctcctaaggggctggtctcaccctccgccagccacgggcccgggcggtgccagggtacctgcgccatgacgtcatgggaccgtcaccctccgccagccacgggcccgggcggtgccagggtacctgcgccatgacgtcatgggaccgtcaccctccgccagccacgggcccgggcggtgccagggtacctgcgccatgacgtcatgggaccgtcaccctccgccagccacgggcccgggcggtgccagggtacctacgccatgacgtcatgggaccgtcaccctccgccagccacgggcccgggcggtgccagggtacctgcgccatgacgtcatgggaccgtcaccctccgccagccacgggcccgggcggtgccagggtacctgcgccatgacgtcatgggaccgtcaccctccgccagccacgggcccgggcggtgccagggtacctgcgccatgacgtcatgggaccagatgtccgcagccgaggtgaggtgtgctttgctctccacttctgagggtctcagtaacgtgggtccaaacacggtagccaggttgtgaagtgacattttgttgatgggctccttctcggcaaccctaagaaggagaagatggggaggaaagaagc

SEQ ID NO: 23 - DNA, 2593 bp, Homo sapiens
cggataaaagcagaagcagagagagcaggcgccctggctgaagaggggacgtggggcccactggctcacacctgcttttccaccacccctcgcctgccttggggctcacgtccctccccggaattcccacgccccacaggcagaatctgaggcacacctcagcgccccgccctcctttcaggcatctacagctcaaaccttaggttcccagcagctcctagaggcagttctcccgaaggcctcgctctccctcggggtgggggacgtgggggtctgagagattaggggctttgtaaggacacctctgggtcagacgctgaacctgcagctccagtcgtgtctctgcttctctccctcctttgggaaactcagggcttttgctcagtggctgtgggttcgccctggcagcctcgagaggggacagcacctgtctagtgggtcaggcgggtgtgtctgggtcatcttgcgtctccagccgcgctagggtctttcctgaagccagggcagctcagcacttgcctccgagggcgtgaacacggtgtgcccatccctccctgccccagcccaaagctacaggctacactggggcttagaccctcgcccagcaccaccaatgtccacgcccccaggccacggcaagggcggggctggccacgaggggctgctgtgagtctgcggtggccgcaggcttgagggaggccagcagagcccaccctaaaggtgacccccgctcagcattcatctgcagcctcagccctaactcaagaaattctctggcaacccttctgtggcatccttctcttgaagctttcagaaaacacggaaagtgggacaaccctggagctgatcctttggattcctaggaggaagcagcagcctccgccagcagggaggttagcggctcacggggaggaatctctgtctgcggctttcgcctcggcgagttcgctgaatgccacagacccgagaggacactctctgaagggtcacccgaggttggccggctaagatcaaacccaggtcccgtgcctctgagtctgggagcccggcacccagagctgagaacacctttttttggtctgtcgggaggctggatgttctcagggcctgactgcatcggctcctgaggtcctgtctggaccggcttctctgcatggtgcccacccttcagaggcgggtcagggggagcgggcgccaagcctgcctgctgaggcggcacttcccaggggtggaggggagcggggggagccgactcacacctccatctgcttcctgctggatgcttcctgcccagaatccactgggcagagtccaggctcccaaaatcaggaacacctgggcgatggaggcagctgagcagggctgacgagagaggttcgtgccccacgtttggaaaagctttcgacggcagggcaggcactctcgagggaccctcccccgacttcccccacccaggacaggctctgctgcccactctccaaggagaaccaggcgtctagacctgccttgaagagggacagcaggtgggagtctgggctggagaacaaatgtgcccgaaacagctggggtgggcagggccagagcaggacaatggctgcagtcacggggccctgggaggaagtggagagtcagcaggaagtagaaccaggcctggggctcagcctccacggtccctatgtgcctggggaactggcacaggggtgggggtggcggcagagggaagagccccacgtgggccagctgtgagggtggcaagcagcagggaggcggaactcctaagccaggagccgaggcggggcctgacatgcactcctggccttggcgggcgccgacgcgggctgatcttccagggagaggtcactccggtgtcccacgacagggagctatgggggctgtgagtgccagggcaggggttggggacgggagagatggaaccaaagggaaaggcctgtgttccttcccagttgaatcaaggcctccctcagggccaggggcccggctgtggtcagtgtggcccacgcgtgaggcctggaacggggaagcactgaggacccacgttaccggccgtcgatcatcttcctgggaggggtcccagtaccaccatgaagaacgagagggggccggagctggaaggggctctgggctcacaacccagggcccccaggacgcacgcgcaggaccctcaggcagggtcgaatggggacaagacaccccttgggggtcagagggagggaagtggggcaggggagcccttgactcctgccctggcgggctccggccccacgttctctgcaagcttcctcgtgctctccagagtaattgaaaccagaagctgctccccagccgctgacaaaggccccttgtttccgaccacaccaggccaagctcagagctgccgtgctgggtcatggcagggaaacctcgggccagccggcattgagggccccagccttgacttccccgcccctgctatgaggttggttcagcaaagccagtctgaccccatcagcttaagaaaataatgctgcctcggccagccaaaggccccgacccaggggaccacttataggtgacagcctttaggagggggctg

SEQ ID NO: 24 - DNA, 6190 bp, Homo sapiens
aaactgtgtcctgacacccccagacctgctggccagcagggaggggcctctcagcatctgggctttctccttgctcagggaacaggagcacagctctgagaactaaggatgggggtaagtgagctaggccctcaaggcagggcacttactaggtggaaaaaacagcctggaagctcatgggcatgaaaatgaggtccatggagagagcttcctctgtggcccagaaactagaagctggaacagccatgtggaactgtgcagcagcccagaacaggatatgggggcctaagtcacagcagaccagtgagaggagaaagctgacctcagattgcagatctgtataaagaaaagtagggtggcgggggagccttgggttcaaattctggaacaggagggacaaagaagggcagggaattggtggtgatgagtaggtaccacttctggggaagatgacagagcaactggacctgaaaaactctcgacttacctaaaatatcaattacagccagtgacaaagaattcacgccacacaactcattaccaatcaaacaaactactatggttatctcaaaccaaacgtcactttacttttttggtaacttttcattataataataaactctattcatgaatatgcagcctccataatcttctcccttgtaacaaacgtgcagtccgttcacaagctgtaaaaacaagcccaaacccaagacatcacaagaggcaagagcagtggcagtgagaagggagcctgtaaaggatgtttcaaaggagggtcccaggctatgtggccactggatgtaggcagtgagctgagtccaggctttcggtctgggaagtggcagaggctgagacaatggccaaagaggagttggagaggaaactatgctcggtttcactcctgccagcccaacagcctattccctggtgtgaatcaactggtgtttgatcaactttgatcgctggctgaaggctttcccacaagcagcacagtcatagggcttcaccccagtgtgaatcctctggtgctggatgaggaccgaacgctgactgaaggctttcccacactcactgcatttgtaggggcgctcgcccgtgtggattatctgatgctgaatgaggtgtgagctctggctgaagcccttaccacattcaacacaggtgtagggtttttccccagtatgaactttctggtggtgaatgagatttgagcttcggttgaaggctttaccacactggttacattcatggggcttcagcccattatgaatcctctgatgctgaatgagggttgagctctggctgaaggtttttccacattcagtacattcatagggcttctctccagtgtggactcgctggtgaaggatgaggttggagctgcgaccaaaggtcttcccacactcgtggcaggcgtagggcttgtcgcctgtgtgcacgccctggtgctgaatgagggctgagctgtggctgaaggccttcccacagacactgcatctgtacggcttctctcccgtgtggatgatctggtgctttcggagcactgagctataactaaaggcttttccacatacattacacacgtgaggcttttctccagtgtgaattctccgatgctgaataaggctggagctctgactaaatgctttcccacagtcactgcacttatagggcttctctccagtgtgaaccctgtggtgcttaatgaggttggagacccgactgaagggcttgccacaatcattacactcataaggcttctctccagtgtggaccctctggtgcttcctcaggtgtgcactctggctgaaggctttcccacactcgccacactcaaaaggcttctctcctgtgtgagtcctgtggtgtttgatgaggtttgagcttcgcctgaaggccttcccacactcactgcacacatacggtttctccccagaatggattctttgatgttggatgaggtttgagctccgcctaaaagccttcccacattcattgcattcatagggcttctcactcatgtgagacttttggtgctttttaaggctcgagttctggctgaaggcttttccacattcattacacatataaggcctctcactgctgtggtgactctgatgcctagaaaagtctgagtgccctcggaaggctttcccacattcgctgcactggtaagctttctcactcatatgagatcgatgacggtttttaagaactgagttctggctgaaggttttcccacaatcatcacacataaaggaagcctccccagtgtggactatttgacgctgaataaggtcaggatttccttggaaggttttcccacactcattacatatgagtggactttcagctgtgggaaccggctggccgaggccccggcatgtcaagccatctcaggttgggcaggaatgtggtccgtgttcacatgtgtctctgtgtgtgtgagagagaggggtcagctgggacgctggggtggcagggacagtcctggctcacccctcatcctccctcgacctcgactccctccacatgaggagcccccccttcctggctatcctgtgagttgagcttcctctgctgggagggctttgtcagaggttccctgcggttccagaaggaaagctggctgcagggagggccgggcactggacaccgtgtggctgagcctgtggcgggggctgcacagctgggttcccagcccccctccttgtccccaccccaccgcactgggaggccctgctgaggggccagagtccggctgcaggtcccacgggtgggggtggggcccctcattagcactgcagctgacactgagggcttccacctcgctaattgattaaactgtttagaaaccaggccggcgtggtgggaattggccccggccgggctgtccgctccccttctgtgcaggcagcggcccccggagttcatcagtcaggccggttggtggggtcccggccctggctgccctcgggaacccttctttgctcctttgtgcggtcaaaatggtgagggtcctgagaggagctggtgagaccccggggtcctctcctccctgaccactcactgggcgagcatggagggaggcctactgtgcacgggcatgttcctgggaacctgcctgctgggattaaacccgcccttgtgaaggacggcaggtgggtcactcaataccaggaggggcacggggctgtgagcagaggcccgagagccttctgaggcggcaccgggtgctcctgggccctgctctcctgggatttgttgtgcctgtgacctcagcctcttccttcctctcctgtgggattcccccaacaccccctcccctcctgccattccttcccccaccaggccccatgcctcccctccccagtgccccctacccccaggtcttccctctaggacatcagcctgggctgtgggtcttggtctcccacagagactgagtcctgggagaagggcagagccttggttcccagtgcagcccctgtgccagcctgcagtgggcaccggttcagccggtgcacactgggtcctgcccccacctgaggagcggcctggggcctgatcagccctgctggtgtctggcctgcagccagcaccggctctgctattcacacttggttacaggtgggtgcccatcccagcagcctcggagcagagtgggtcgggctccggaggtgggggcggccactaacagcaggaggtcgtggcagtgcggctatggcaggggttctgaggggcggaaggcaggggcgggacgtggggacgcagacctgcagggaggacgccggctcacccagcagggaggggatggccgcccagggacccccagcctgcccgctctgcttccccgaccgccggggcaggggccccacgggggacgccagggaacgtgaggaatccggagtcaacactgggccactgtgtgctgccagccgggcgggccgtgatttataaagacagcggaggcttggctggtgtcggggcggtgaggtcacggcggccgggggctctggaatttcttcagaagaattttgcttaccaagccacatacttttctagccatcagtttgatcagaggcaagatgaaaaatatgctaaaaaacaaagaaacaaaaatacacccggggggctccggtgagggggaggggcgctgcgggaggggtggagggcccagggaagggtgaggggccgggagccactctgcccggcactctccgcccagaaacagcccaacgcccctttctttccccttttagcactgctgagctggactaaaatgcccaacaaggaactttactaaaaactgaggcaagaaagaaaacacacatgacataaaaatagtcaagggcacattcttgatggtagataactggtctctggccacagcggctgccaggttgggtgtcggccggcgggtctgccagtcccacccataggcactgcacttccctgggccggacagggggtgtggcgggtctgtgggcggggggacaaggttggcaggaccgtgaggggggtggtgggtctgtgggagggggacaaggttggcaggaccgtgaggggggtggcgggtctgtgggcggggggacaaggttggcaggaccgtgaggggggtggtgggtctgtgggagggggacaagggtggcaggaccgtgaggggggtggcgggtctgtgggaggggggacaaggttggcaggaccgtgaggggggtggcgggtctgtgggcaggtggacaagggtggcaggacctgtgagatgatgtgagtgcagcacagtggggctctgtaagaagcgacccgggcagcttgagcaggggcaggctgggcggtgcctacgggtctctgtccaccggagcctctgttcagcccacctcagtgtcgctccggatgtggatagaaggagacactgtctgggccacagaccaggtgcttccttcgtcctgaccacacctgcttctgcccaggagacgctgcaggggctgtgctccccgcccggctactcttgagtggtccccaggctcctcctcctcccggttccacctggagccgtggggctgtgccggggatgcctcgctgcagctgcagctcagggagaactcactgctggagcttctgcctctcccgtgccgtggggccgagccgagctccaccagggtctggacttctgcacgggcagctgtgcttcccagggtcgtggagaggggtccttggtcccagccactgtgtgacctcgaccaggacacttgactttcctgcccccagagggtcttgtctggacctccagagcccccagccttgctcacttggctctgcttctgggcagggtgccctggcattgctgttgctggcacctgccgtgccttggaggggtctccagtgggacctctgagcacggctcttcctgtacttctcagaggtgagcagagggcatttgtgggagaactggaacctggggaggaaaaaccccaaggctggcaaagactccctgcagtctgtccagtgatccactgaggctgagtggtggaggacatggaggccggcccgggaccaggacatggaggccggccagggacctggggaagagagggcctcagtctggtgagaccagcctggtgggtgcctggggaagagagggcctcagtcctgtgagaccagcctggtgggtgcctggggaagagaggccctcagtccggtgaggagaccagcctggtgggtgcaggccacccttgcctgctgtcagggcctgcccttctctccggcctccagctgctttgccccagcgatcaggcgcctgagcttcctcccccgagcctgagtccagctgagctccgtgtggctttcccggtggagcagactctgtctgatttcccaacggctggcgcctcccagggcgtgctccttgccacggaacagccccttggggccaggtgtgtactccaggcagtggcccggcagtgctgggaagtgccggtcatggctgctgcacgtgggttgctgtctgggagagtcctgtggtgtttgctgagggcggaggacaccgaggacagagaatgggcaacttccagggagggcccagatgcagccacgactggggtgcatctgggatacctcgtccagggacactccccaccatggcctggtgcctgtccagcaggaagagcttcagggcagtaggaagggggag

SEQ ID NO: 25 - DNA, 1689 bp, Homo sapiens
aaaattgaagagcttccatcaataagggattggctaaatacagtatgcctcacctgtacaatagaatactgcacaatcattaacaaagatgagtgtgctgatatggaagagatattgatattctgatgtactaaatatcttttcatctcccagatttattgttacaaagcaagaggcataaaaagcatattccctttgtaaataaatgaaaagatatgtatacacatgcatatttgtatgtatatgcgcagaatacctctgaaagaatgaacaggaaactggtaaccacagttcatctgggaagagcactagaggacagggaaacttttttgctctgtgaattcttaccacgcatgtgtattagcctgttggaaaaaattagccctagaataggcaaattcgtagagactgaaagtagaatagaggttgccagaggttttggggtagagaatagggggtttttatttgatagatgcattttctgtttgagatgatgagagagttctgaaatggatagtggtgatggttgtacaacattgtgattgtacttaatgccactcaactgtacacttaaaagcggttgaaatgggctgggcacggtggctcacacctggaatcccagcgcttcgggaagccaaggtgggcagatcacctgaggtcaggagttcacgaccagcctgaccaacatggtgaaaccccgtctctactaaaaatacaaaaattagctgggcgtggtggtggtcgcctataatcccagctactcaggaggctgaggcaggagaattgcttgaacctgggaggtggaggttgcagtgagccaagatcacgccactgtactccagcctgggcaacagaagtgagacctcatctcaaaaaaaaaaaatgttgaaatggcctggcacaatggttcacacctgtaatcccagccctcagggatgccaaggcaagaggatcacttgagcccaggagtttgagaccagcctgggaaagatggtgagactctgtctctacaaaatgttttttaaaaattagctgggtgcagtggtgcacaccctgtggtcccagctgctggggaggctgaggtgggaggattgcttgagcctaggttgtggtcccagctgctggggaggctgaggcgggaggattgcttgagcctaggaggttgaggctgcagtgaatcatgttctcagcactgcactccagtctgggcaacacagtgagaccctgtctcaaaaaaaaaagaaggaaagaaagaaggaaggaaggaaagaaaagaaataaagaaagagaaagaagagaaagagaaagaaagagagaaaaagaagaaagaagaaaaagaaagaaagaaaagagagaaagaaagaaagaaagaaagaaagaaagaaagaaagaaggaaagaaagaaagaaagaaagaaggaaagaaagaaaggaaagaaagaaagaagaaagaaaagaccaagtacagtgactcacacctgtaatcccagcactttgggaggccaaagtgggaggattgcttgaggccagggattcgagaccagcctgggcatcacagtgagaccccatcactacaaaaaataaaaaaaaaaaggagtggggtatggtagcatgcacccatagtcccagctactcaggaggagtggggaggatcccttgaactagggagatcgagactgcagtgagccat

SEQ ID NO: 26 - DNA, 2530 bp, Homo sapiens
agaatgtgattgccgttctgaaaacacccagaggccgcagtgtgcccggcagagagcaaggacccctgaccaccggctgggttggtcctgggagggccccggtgatacctggggggtgtacaccatggagcagagcctcctccagtgtagcctgggagcctctgtgaggccacagcccccaggaagagcacagtgctgcattcccaggtgctgccggctgcgcccctcccagctgcgtgtcctcacctgccggccccagctgtcgctgcccacgccctgcctgcctctcctgacaggaacttcccaagcagaggcctcaggtagcaggcgctccttgtcccctctgccacctgggctgctgagggtgtatcaccaggagtgagctcaggacctggacacccaagcccaggtgagcagctgacacaccaatggccattcccgtcccgggccctggttcacccagccaggcctctgtgccacttttccacgggacattcagcttcccctttcctctcctctctgcagaccactgaactttcgttctgaggcacaatggggcgttcccgtcaggctctgcccccctagacagaggtgagaccagctacggcacagctcttggcagctgggtgcccctctgagatgggccaggcagcacgctcatggcaccttcatgtggcttcaattctctggccattgcattcctaaccaaaatataaactgcaggatcgttttggattttgcattacccaaaccatttgcttttgataataacagtgtcttggcagagttcttgctcttggactccgtgtggtgatggtgaccgcccgtgcacggaacaccatggcatgggcatccgcctctgtgcttgttaactgaggaggaggtgcagtcgctgcccggaaggcacaggcagtggccagggacagcagtgagaccacaccgttgtgaaactcatgctcataacaactcgcgtgcacctctccttttggctgtgcaagtctttgcatggaacagttgatttaacgtgggcccagggcagcaggggcccataaagcaagcctcttgggtggggggaggcagtggcatgtcattgggactcccctgtcctgttgcccttctgtggtggatttgggggccagtggcccgttaagggcaggacacaccttggcaagggagcgggcgtgggcggaagggcatgttgctgcagtttagggcatgtgagcttggcctccagagatgagctcatcctccctgggccttgctgagcgtctgaggcttcttcaccgaggctcacctgagtgacttcagcgccgggggtttaccaaggaaaaacgttcccctccagtttgaaaaaaaaaaaaaaaatgactgcagccaaccctcaggcccttcctgtgaaggtgctgtgggccacaccacgtgggcttggctgtgggcactgggccggcttctggtgctcaccagctgatgcgtcgggaggtgtcgggggcagtgagttcccactggcgctttgtgacaggctcctcctcttcgtggcctcggaaaaaatatatgaaatgggaaactgtcagtggtggttagtgctctccctgggctctggcgtgtccttctctgtctccctgcaggtcgccacccgcccagtgagttcttctgcctgtctcctgctcttccttcctcactccctccccagaagaggagctactggcttgacaccttcacactgttttgggtggacctgctcctacacatgggaggaagtgatggggcagggcaaaggaggggaccttgccatgctgtcggcatgtgtccatctgcccagattcgtggacgtctgttttctgcctcatgtgttctgtaaagacacttgtgccatgtgaaggtggcactccttcaaactctgtgagctccaccctcccatcctggcaggaaccatctggggtgagagtcggcgttgctagggagactgggggctgggacatggttttaccaaagtgccatggtcggaggccttcctaaagcaaaaatgatcagaaagccaggctggacactggaaatgcgcttgagggaagatggctgcaagctgggattctccagggatgctcctctctatgggttctcagcatgcaggcacagaaggctggaggattctccctttcttgagaggagacactgttggaagggcaggtgcagccaggagcaggagtcggtggtgaaggagtggggttcccctcagcccagcagcagcggacactgagctcggaggaatctggctggaaggcccaagtttacaaagcctggaccagaggcatctccttgaggagtcagacctgttctcctcttagagtgcagcactgaacctactgggagcgggtggttgagatttttatagagatcactgcagcttttccaatgatatctccactgggacagacatggggatgcaatccaggtctccccatctcacgtgtgctgggtgggtcttaggagcaaaccacagctgtatctgcaagaatcaagcacagaaaagaaa

SEQ ID NO: 27 - DNA, 2094 bp, Homo sapiens
tacctgccctgccacctctgttctccctgcccagctcctgccacctttactgcacaggctgggcacctggctgtcccaggctcacctctcctggatttgccaccaaagggcagccaaggcacctggtggctggtccagagtcggggaaggactctgattggctgagccagggttaagtcccagggaaggactctgattgggtggtcccgagttaagtcccagggaataactctgattggctgatccagggttagtttccagggcaaggccaattagtgggtcttgaaaagcaaaggactagagtcctccttagaactcaacactgagagtcgaggactctaattggctcaacttgggtagggaagaacgtagccaatcaatagtggccaagggctttgaatcctgcctctcctacttgggggacctgagagccatcagccaagcataggagtctgcttcccctgctctcccctttgctcttcaggaggagaaggtggaggagggccccagcgaggagattttcaccatggagcccttgcctcatgtacaccgggagtctcgtgcccgccgttccagctatgctttctcccaccgtgagggatatgcaaacctcatcactcagggcacaattctgcggaggggaccaggggtcagcagtgacatagcatctgaatccctagacccatctgatgaagaggcagcttcgagcccaaaagagtcacagtgacacctcaggaagatgtccttcctggggaagaagaagcaccagccacaggggcaggtgtcctcccaggaagtacagctcccccctacacctagctcatcattttctatggatagacaatccgctcttcatccagaaaaccaacctgccctccccaaatatgtgctcaccagcagcaacaggctatctgagtctttccaagagcaattgccaagggcacaggagaggtcattgtcacccaagcagaggccaccttctcctgagaagttgctgttgaccaaggagaggtcacattcttttcaggagaaatcactgttgcacagagaaagccagctgtcgtcatttgagagccagccacagcctctggggagccagtcatttctttcaggccagctgacgttggagagccagccagactcctcggaggagaagtcagcatttttgaagccctccacaccgttccggaagagctggcaaaaggagcctcacacccccaaggaggggacggtgccacttccagacaagacccacaaatctcaggtggagactctgccaccaagtctggaagaatcgtccacgtccacgagcgagcagcctatggaggtggagctgtggcccgcggagaagcagtcatcatcatccatggagtggctgctggtgcccggggaggagcagctatccttgcccccagaggagcagtcattgccctctgcggaggggaccagggttcagcagtgacgtagcatctgaatccctagacccatctgatgaagaggcatcttcgagcccaaaggagtcacgctggcatatcaggaagatgtccttcctgggaagaagaagctccagccagttctgctgcaagtcaaccagcatgcagggggccttcctctaaagacaaggactccacatgcttttctttttctaataaaccagggtccatctgaccccagcgctaattcaggctccctctttccctacactttttttgtgatggaatattccttcccggtttttaaaatcaaaacactgacctctagtggtccagccgggtatttgcagggaaaactttccttcttcatgctggggtaagataatgtgggtaaagcttcattgctctcaaaagttgcttattaaaagctgtggctcccccgctgcctgacagctggcccctcccaagaaagtttataaattccagttcttgtaccatctagcttcttcctctatcgggaagccctggtttctcccattcaaatacaccttcattcactggggcctccgttcactttagactccagaaagcaatgagcagtgatgtcacagaagcaggtcctgacaaggtgtgcatcttggggcttggttgactcaaaggc

SEQ ID NO: 28 - DNA, 4137 bp, Homo sapiens
gggagacgagaagggacacacacacgcacacaaggcttcagggacacgagaagggacacacacacacgcacacaaggcttcagggagacgagaagggacacacacacacacacacaaggcttcagggagacgagaagggacacacacacacacgcacacaaggcttcagggagacgagaagggacacacacgcacacaaggcttcagggacacgagaagggacacacacacacacgcacacaaggcttcagggagacgagaagagacacacacacgcacacaaggcttcagggagacgagaagggacacacacacacacgcacacaaggcttcagggagacgagaagggacacacacacacacgcacacaaggcttcagggacacgagaagggacacacagcaagtgtgttccatgtggcacctggcacagagctgggcgcacacctggcaacacctccaacatctccacccgggaggctcatcccacagagagcttgaggctgtggccactgctggtgatggcggaaaagaccccctcacctggacatgctctgggccaactaacccaccgccacccagaacgaggatgccccatgctcaccgctgcgagaacaacgtggggtcctgcctgggggcgagaccgagacaacctccctgcagggcaaacctcaaacgcacgccacgagggagctcttctgtgaagggccagggtgaaatacgcactggctcaggctgaccaacgtgtgctggctacacacggcccctcgcggctgggccaggacctgcccggagctccagaaacacggccgggagttacaaaaacgcggccctgagctatagaaacacggcccggagctgcagaaacacggcccggagctatagaaacacggccgggagctgcagaaacacagccgggagctatagaaacacagcccggagctatagaaacagcccagagtccagaaacacagcccgaagctccagaaacacagcccagagctatagaaacacggcccggagctataggaacatggcccggagctgtagaaacacagcccggagctacagaaacacggagtccatagaaacacggcccagagtccagaaacacagcctggagctgtagaaacacggccaggagtccagaaacacggcccacaactccagaaacacggcccggagctacagaaacttgacaggggctccaagtgtagcctgggagcaccacactccagccacacctcgccccgctgtctccaatcaaaacaccacgtggtgctggagtctgacaaggacagtccatcgctgctgcgcacggcaccgcacagtcacctgagcaatgtcctgagccgtacaaccagccccgggcaggtgcctcctcacccaagcccttcagtggacgacatcgggccccaaatggagcacggtcccaggacacgaggcagaagcaaggctcggcaacaaggccacagcccactggtcctgaagggactcagtgcccaaccggggcgtggacagaggcggagaagccactggtcagagccatgggaaggttttcagccagagatgtctgactgccaagaggctggcttggaagttaccactcaagaagccacagggcagagggcactgctgcagacatgcagagacccacagaggacgtggggaaggtctaaggaagggcagaaggccccggcacttggcagcacctgcctgtcatgagggtttgtcccgggtggcaggacctgggtccctggaggagggaaccaggagacccctggtctccaggtgtcaggggttctgctgtggggccaatgctggacactgagccagcaggctctgctcagaggacacagacttgaagatgaggtgcccagggccctggggtggaatgtgaggcagaaacaactactagaattcagcttttgccacattctttcccaaagccagagccttgttcttgtggggacaggaaaggggcccacagcagtcagtagcaaaaaatgcagaagacagcaatgggcacacggtgaggaggcggacacaggacacggggctccaggcctccagtcggccgtgtgctgtgtgcctgcggaccctgagcccctccccagatcgagaagcccccggtggagcctggcagtggagtccgcaccttgttggcctggatcaggtgaaagttctttccatgcacacggaagccgtgctcaaagttcctgcactcctcttcactccaagcacagagcccatctgcaaacacggccggggagaacggtcagtggtgcccagggcggggccgcagcggaaggaaggcccaggccggggagaacagtcagcggcgcccagggcggggccgcagcggaaggaaggcccaggccggggagaacggtcagcggcgcccagggcggggccgcagcggaaggaaggcccaggccggggagaacggtcagcagtgcccagggcggggccgcagcggaaggaaggcccagaccgctgctcacctcggatcaccttcacgttgaaccgcagccttcgcagggcctcctccacattgaagttgcatttcaccaactcgtacagcgcctggggagaggacatgttggctcttccatgggctcagcgcaggagccgacagcaagaactgtctataccatccagcgagtggcatcaggggccgtccacaccaccctcctgggcgatgtcagagccacctacacctctatccagggagtgacatcaggggccgtccacaccaccctcctgggcgatgtcagggccacctacacctctatccagggagtgacatcaggggccgtccacaccaccctcctgggcgatgtcagggccacctacacctctatccagggagtgacatcaggggccgtccacaccaccctcctgggcgatgtcagagccacctacacctctatccagggactggcatcaggggccgtccacaccaccctcctgggcgatgtcagggccacctacacctctatccagggagtgacatcaggggccgtccacaccaccctcctgggcgatgtcagggccacctacacctctatccagggagtgacatcaggggccgtccacaccaccctcctgggcgatgtcagggccacctacacctctatccagggagtgacatcaggggccgtccacaccaccctcctgggcaatgtcagggccacctacacctctatccagggagtgacatcaggggccgtccacaccaccctcctgggcgatgtcagggccacctacacctctatccagggagtgacatcaggggccgtccacaccaccctcctgggcgatgtcagggccacctacacctctatccagggagtgacatcaggggccgtccacaccaccctcctgggcgatgtcagggccacctacacctctatccagggactggcatcaggggccgtccacaccaccctcctgggcgatgtcagagccacctacacctctatccagggactggcatcaggggccgtccacaccatcctcctgggcgatgtcagggccacctacacctctatccagggagtgacatcaggggtgtctacatccccttgcaggatacccggaggcgtctacacctcctccctgatacgtggttttaattggccccccttctgacctgagtagctgttccagtgccctggcccccacacacctgacccctgccctcccctctgccctccctggcccctggaggcactggggtgtgagctctggcccacgccacggcagccctcagcccctctgtccccggcatggcagcccccacctgctcactgtctttcacggcttctccctctgggagctgaggcccggccatctcgtgccaacgccgcttcaccgccctgtacaggaactcctccacctccctctcagggaggacgctggggtcccagagcagctggtcttcgttctcgtagactgcacaagcagagggcaaaggtcagcttgcaggaacccaatctgcacccacacacgccaggacaagcaaagcagccaactcagcccctgacagggaggaggcactgtccgtcctccctttcccaagccctgggccgccatccctgtgctcctcctgggcttggtgctgctgtgctcaattc

SEQ ID NO: 29 - DNA, 2400 bp, Homo sapiens
ttcgcctcctctccccaggccctacttactcttctcacagtgccggttcaagtgcaggttgctgaggtcagcttggaactgaggtcccaccatgatctcctgcaaagcaagcacctgggaatcaggacactgaggagcatctaggccgggcgggaggctggctgcagcgtgctgtggcaggcttacggggaggggccactgtccagaccccagacccatctgtgccgtctacctgctgatgcccagttctggggtctgaaggtgggaggcagaggcctgggtgtgtgaggggtgaggctgtgtcctgacgcctggcctggcagaggcccagacaggatgtcggaggacaaacactctgggtcagcagcaggggcccaggctccggtccaaagcacctgtggccggtcccagcccaccctggggtcgagcagcacgtccctcctctgagaaggggcacaaacccagggagagggctcagcaggacccggctgcggttactgaggccgagataccaggttggggagagggcagagccatgggagggatgccaggttggggacacggcagaaccacggctgggatgccaggttggagacacagcagagccacggtcgggatgccaggttggggacacagcagagccacggttgggatgccagtttggggagacggcagaaccacagtccggatgccaggttggggacacggcagagccacggccgggatgccaggttggggacacggcagagccacggccgggatgccaggttggggacacggcagagccacggccgggatgccaggctggggagacggcagagccacggtcgggatgccaggttggggagacggcagaaccacggccgggatgccaggttggggagatggcagaaccacgtaccttcttacatttgttggcaggaagagagtcctcctcggtgtcggaggaggcagaagagccaggctctctgtcttcatcagccaggaaacgagctttgggaaaacagaggcaggtcccccagggtctccactgcctgcagcctatacaaccccttctctccactcccattctccatccacctgatccccaggccataaccctctctctggccagacattgggtaaacagatgggcacaggacccaggaccagggatgcacctttgaagaaagaggccttcccttctatgcagctgctgcacctctgggccccgagccctcagttcccaggaaagccagcacagaggcttgtgaaggaggccggttctgggaatgctgtccctggatctgctaggggaaccaacatgttccctacttgtttaaaccaaatcgctctgagagtccaggctcactggccagcgtggaggagaacaaagcacccccagggctactgacgcttcccgccaggcagacgccctcatctgtgatgagttcttggcctgcatcagcccaaggacccttcatcaagcatcacgactgcctggcagggggcctggctgcggtggagtatggggacagagtcacctacatccactccggttagggaagaggtcggaggcctcgtgggaggtcacggacggggtgaggtcgtcagcagatgattgcgtctcttcctcttcttcccctgaaagcaaatccttcgctatttgttcctttaaaaaaaaaaaaaaaagtaaagaacattttacagtttaacaatctcgcaataccactaatgataacaacagtaaagacactgggagtgccctgaggctcacatggggctgctattcccattctgcaaagggtgcacagcgtggggggagcggggatgggaaggagacacgtgggagcccacacccagccaccagagctggagacagttagagctgccactgggcacacgcccggagtgcatggctctttctctgactgtgcatttggttttaaccttctacaatgcagcccgcccctgctcccaacacccaagccttgacctgtgacctctgggtacggaatggcagagagaccagtcctggggaggccccgatgtgcccctccacccaccaaagccagaatgacatgtggcctggggttaaggctagggtccagccccatgcccatggccattccaaccccagggtagtggtcacaggtacattctacttattctgggggcctttgtgcctcctctcactgaacactcccctctgcagagaggcagcgccaggcccccccaccttcagctgtgagccagttccaggaagggccctcacttactttgtccagggtcatgtctgggaggttcggggccacgtcaccaccctcactctcccggtctgaaatggggtctgacgcctcgtagccatagagcgcaagcagctcatcaaagggcatgtcgttgctctgagttggggaaggg

SEQ ID NO: 30 - DNA, 1815 bp, Homo sapiens
gggagaaggggagtttgctggggagacgaggcgtgtgggagaagttccaggcaggtggagggatgccggggcgtttgtcccgagggctgggggttgcaggagatggctggaccccggtcaaggtggccagcagatgtgtcacgtggtgtcgagtgcggggctaggtcggcttggtggaagggcaggggacgggggagtgggctggtgtgacccttcctgtggccccctcacgtcagagcattcccgacatctccacgctgccctggttctcgctcagtacccctatggtctgcctcctcttcatccgtgccacccgggacctggtggacgacatggtgagtgctgttggatgcagctgcctgggggagggagcggggccggtcgggggggtctcttgatccctgggcgagagtgggaggagggctgggcttcctggagcattaggggaacgtgggcctgggagcctcagctgctggggctacattgtccttatctgctagcacccacattgggcaggtgccgcaggtggcgttggctctgtcggtgcgtggttttggggccattgagctttggtggggggtggtctggcaggcactctaggtggtgggcagcacgcctgtcttctccccgccaatagcagtgggtccagtggcccccacgtccgggatccctgagcagacgcaacgtggcgtggggccagcggacagggaccccgtgttgcgggcgggcactgctgggctgcagtgcggcagcggcctgggcgggggcaggagaggctggacggtctctctgatcctttccctcctggcccaggggagacacaagagtgacagagccatcaacaacagaccctgccagattctgatggggaagaggtgaggctggggctgcagctggggatccgcggggacacgggggctccagcccagcagggtcatcggcctcggcaagtgtccatcaccttccgtgctccctgatctcccggctggttgagtccgacaggaaccgggcctgcattcattaggcgtttggccgggacgaggacagaggccgaggccctgatggcgaacccttgcagagcttagggctcgggcgatggggaggacaaggaaagtctgaagaggacgtgggtgcaggaccctggaggtcactgggtgggagcgtggacccgcggggagtggggtgggagcccggggaaggcttcctgagggggcaaaggcccggaggtggggactgcagctgcgggccccccgtcatcccgtgcctctggtctcccggtgtggggagggtttgcagagggaggggcctccttcacaaccccctctccccgcagcttcaagcagaagaaatggcaggatctgtgcgtgggggatgtggtctgtctccgcaaggacaacatcgtcccagtgagctggggttgaccccgaggtcccagaaccacgcgccccctcaccgagagcacccctcccagggtggggagggctgccgcacccccaatttgtcttgcatcccctcttgcaacgctgccccccactccacaccaggccgacatgctcttgctggccagcacggagcccagcagcctgtgctatgtggagacggtggacattgacgggtgaggagctgtggcatcgctggggaccctggggggtggggagcatggcccggaggagcccccttccccagtcaccaaggaggcggccagccaaggtcgctcagagactttggtcactcaccccatgagtgtctggggcgtgggtgctgccaggcactgaggggaggaagacgcccaccctccccattgtttccattgtg

SEQ ID NO: 31 - DNA, 2721 bp, Homo sapiens
gatggagacactctccctgggaaatgcccgaagtcccttctctcctaggggtttcttcagaggccacctgttaggcctggaagctcagcttgaggcctcttctacctggatcgcttggttcccaagtgtgggtagcaaggtcttttcctctcccggctcctctaacaactccactggggagcttcagcagcaacattgctggttgagatgtgtttcgaggctaagaagtccttccaggctccctccacagccccatggcacagtcagaaagtgaggcagggtgggtaggctgcacttcccagtgtcctcacctccagccagcaccatctctagctgtggctcctcacagctgccgccttcctgcccctggacttgccacagcttgtccctcaggattatttttcccaacccagcaaagccccagatgatgggactcaggcagcaaggagggctgacccccaatcagggagttcattcctcgataaagtcactcaggtccctgtgatgctgccaaacctgccctctgagcaggatggtgtagtagagggggatgagtgctggcagcagcactggtcaggtgatctgaaggagaacctctgcacttaacaaacacacaccttgagatcattctcagcaggaggggcagatgaggcgtaggtaacctgctgactcttccgggtaataggtaagaatgtgaaccagacagggcagggaaggggtggaaagacgcctacagtgatgggccacatccgcaggaggagtgggggctgctggaccggtcacagaaggaactgtactgggatgcgatgctggagaagtacggcacagtggtctccctgggtgaggaccagccagccccaccccgcccctctccctggggcctgcacccaccctgcagcaggcctagctgggcagggcctctgtgctaccagccctacccagctctcccaccttccagaggaacaccctgtcacctaccagaaccgaccccacccctccttcatgcaaaccccatgcctaactgtgccccccacccgggcagggttaccgccccaccagccagaggcacaggcccagtcagagctggggatgctgctcacggggacaggcgtctgcagaagcctgcgctcgggtgagtgccccacaccatccagcctgaatcacccctcctgtatcggtgggacctgagccacccactcatggggggacgggagcttgtgccacggccacaagcctgagggaggggttgctgagtgccgggactcacctggtttgcccctgcccccaggaaatgagagtgagggtccacctggctgcccagaggcccagccgccccagggcccagggccggcagcctgggagggcttgtctggggctgccactcctgcccccactgtgcgcccagggacaccgccagtgcccactcagcccacacctgcagagacgagactggagccggctgccacccccaggaagccctacacgtgcgagcagtgtggccgcggcttcgactggaagtcagtgttcgtcatccaccaccggacacacacgagtgggccaggtgtgcagtccccggggctagccaccggggaaagcacagagaagccaccacaaggggaggtggcctttccgcaccacccccgacgctcactcacaggcccccggagttacccgtgtgaggagtgcgggtgcagcttcagctggaagtcgcagctggtcatccaccgcaagagccacacaggccagcggcgtcacttctgcagtgactgtggccgcgccttcgactggaagtcgcagctggtcatccaccgcaagggccaccggccggaggttccatgagcagccagacagcacagtccctcggggcctcggtgttctcggggcctggatacagcctctggggcaccagcagaagactctggaggcagcaggggatgccagagtgaacaaggggtcccaagccagttccctgcccctggtctggtctcccccaaaagacctgggtgcaaggaaaaggagctgctctctctcttcttgcccctgcctcctagagggaggtctgggttcccttctatggctgaccagtgcctgtggggtgactgccaagcaccaggctccctccctccctgtgacatggcctgggctgacaacactccctctcctgggacctccttgcctcaggtgggtgttcaaaaactgtgccttcccactcgtctgtgcagaggctgggcctgaggtctcagtgtggagagcagcagaagacccaggaaagcacagttggcttccgtttctcctgctccctgtgtgtgttagaattttaacataaattccactttcataatatggagtttctgaataagaatcctgatttctggcttctgctggtcgggaaataggcagtttgctgtctctgcccagtagctgcagcacagggcagttgagcccagaacggccaaacctctgttgccacagaacccaggtcccaggtccccagcctcccttgctccttgccgcccacatcactcaccagcctcactggccttggaactcatcagtcccggcttgagagacacaaaggggatttcctttcgaagtacggctggacaagggggacctctgagaagaggggctgcaagcaggggttgcgccaaggccatgggtacttctaggtcaggccgcaccctccatagttagctggtcatgcagcaggaaggcaaaagg

SEQ ID NO: 32 - DNA, 2399 bp, Homo sapiens
ctctgctccacctctggctttgacgacgatggagtcctggggttcaggagactgaagtcagcccatgatgcacacagttggatcatgaaagccctggcctctcaccttgaggaagcagtctcagaaggtgaacccagaggagctgccattggcctaggagcctggcaggtcaggctggggtatggcctggggccataccccactccaccagctccaaatccttatggcagggcacctaggctaggagccactattgtgctgaagaggagaggggcaaagagtggctgctctctccgctggatgcaggggcctgggacactggctggccagtaggggtggtgtccccaaccgcccagcagtcagccccaggatcccacccctcactgtttcctgcccccaacacggccatcggagccctccctgaactttgcccccagcaccaagggcagatatatgggggcttatataccctcagtgcaacctggccccaaagatccccctgggctccccacaagtaaggtgctcagccatgtccatcaaggtcggggaggggaagtcttaagtccaaaagacccttagagcctgactggaagatctatgggaggggccttaaaggtcgtggacagcagcaaccaggagtatgatggggctttcacgtggcctccctctcggagacccacctcagatgtggcctgcctatcctactccccacaggactgagggatccaagagaaccaagtgctggttatatatgcagcccaccttagcccctacagaatagaggtcctagatggcaaagtggaccatcctgttcctgcccaggacagcctgtgggccgcatggatgccacccaagaacagggacgctgaaccctgacactcacatcttgtctatgagggcaaggcacgcactgatccaggtgctcacagcttcgtggtttaggccccatggcctacagtcctttattagagcgagagtcccgaggcccagcccccatatatgatgggtccacttgagtctccttaggcgccccatgagggagtaacagcttgggtagagagctagggaccttgcccagcctgaccctggggcaggcaagcggccccccagcccccaccaccaccccaggagagggcggggtgagaaccggagtcaaatcttgggccgggtccaagcgcctgagcgcccggtttacgcaggaaatagtccagttctcagaagtggtctaaccagccccagccccagcccggcaccacctggagggttcaagtacatggaggagaggagtaaggcggacttaggccctggtatggagaaagggtgaagggagagagaggacctgcgctcaggagggagcgtggtctagtggcgggaaccacgggtcccgcagcgggcgtggccgactgtgcgggaggccccggatccaccgtgggcgaggccaggccccagcgccatcagggcgcagggtgcgccgccaggtggcgctccagcagcgcgcggtgcgagaagaccttgccgcaggcggggcagggcgcgcgctcgggccggtgagtgcgcatgtgcacgttgagcgagctcttctgcgtgaagcgcttggcgcagacggcgcactggaaggcgcgcacgccggtgtgcgtgaccatgtgtttgagcaggtagtcgcgtagagagaaggatcgccagcacacggcgcactggtgcggcttctcccctgcggaagacagggcgggccgcgaacgcaagtcagactctacagctccccgcccccaccccaccccacccccacctgggctcctggacctagcaggggctcccctcccctcccgaaccaccaccccgggatcccttgcctatcagagaaccctcccctcactatgggatcttcctgcccagcagggacaccccctcctctccaggacctcccttcacgttgggactttcctgcccaacagggatcctcatacactgtgaggtacccctctcccatcccttcctggcagggaccccctttctgttatcctgggatatcactgtgacagggcacccctaaatccagcaagcacctgtctgcaaggaacccagcctgtctggaacatctgttggccatctggactgcccactgggatctccctctaccctcaggtaccctccccctcaacccctacccacccggcacagggagacactgggtcctggcccccctcgcctatgcccatagagtcccctaaactcagtctgacaaggccagtgccctttcataaggagggacctgggcacatctgccaccttcctgcaggaagccccagttgcccagaacccctgcccgctggccactataatgtccttggtgtgatagagagagctcctcattctgggttaggggaggggaggcagtctga

SEQ ID NO: 33 - DNA, 2533 bp, Homo sapiens
ggcagcagccaggcatggtgaggagacagtcctggacccaggtgaccacagaacccggcggggcgagcttcggcctcacctctcacaagccccggctccaggcagccccaaccccacccccatccctaacttgccggcgcccggagttcatgggcctggcctagacttcggtcaccacagggactgaggttctccagatttcaaaagcctgtgatctgcggttgtgttgccccgttccccccgcggcagacaagcccagacacacacagcccagacaccccagaggcaaaggaattcagcaaacatttattgacccttggtcctcatcaaggaggcagtgagagatgaactggaagtgaccaggggctgccagccacaccccctccaccgagaagatgactttcacctactatacagcagaaaaccaaaagccaagataaaaatcgctggggatgggcagggatgggggaccgggccagaccccagctgctgagcagccgccacctgaggtggggaggggcaggaaatgtctggagagtagggagggcaggggagggcagaaaggacccccacgtgagggggcaccccacatctggggccacaggatgcagggtggggagggcagaaaggcccccccgcgggaaggggcaccccacatctggggccacaggatgcagggtggggagggcagaaaggaccccccgctggagggggcacctcacgtctggggccacaggatgcagggtggggaggacagaaaggaccccccgctggagggggcaccccacatctgggaccacaggatgcagggtggggagggcagaaaggaccccccgctggagggggcaccccacgtctggggccacaggatgcagggtggggaggacagaaaggaccccccgctggagggggcacccatctggggccacaggatgcagggtggggagggcagaaaggaccccccgctggagggggcacctcacatctggggccacaggatgcagggtggggaggacatcagactctgccccaggttccaggaatccgaaccccggagtgctgacgcggttccccaacttccgccttaagaaaacaggaccagccggcaccaggcccgtctctcacgtactttaacacatccttgaaagcccctcgtttaatgagaaaagcgaacactgcggtccttgccaaagtaaaatgaagctgccccaggacaaggggttaccatgagctccctggagtccgacgcgggttttctctctgggggacctgggtggtccccgctgtggtctttgttgtcccactttgggaccgggtccagtctggggtctagtctcgagcatcagggtcaggctcggggcagggctgggttaggctccgggtcagtcttgccatgggtttgggagcaggtttgggttacttgcgtttgaaggcagcagtggtctcaggaggaagaaacgggggcgggagagagtggtgatctgtggtcagtgggtcagtgacctgcacggtgattctcccacctccaaaaggtaggggtgggactggaggcgtccctaggtcaggccgttgagttcgagctccgatgggccaccttgaatccaggactgaccgcccgtgtgtgcacagtttgttcttggacgaggactcgtgaggatcgagggctggggaccccggtgtgagcaggatggggccctgccctcccgtgggagttgtggactcgagcccaggggctgcccgtcacagcggtgtcccaggtccctgccatccgattttacctgggatgtcttctctggagtttggaattgcttgaggaaccctgcgtgtgcttggagaggccagagggcttgctgagaaccccatggacagtggagagcgggattcgaaccaagggctggactcccacacctctggcctgcgtcgcccagttctttgtggctctgaagaattggccgctgtggaaaagagcaaatgtccgagacccccaacaggaagagtctaaaaatccagtttgcaaccacttctgacctacaaaaaaatggaaatttagtgtttttcagcctaagacattaaatttcatatcagaacaaagcctgccccaggctgaccctccccagccgtaccgtggtgaacgggttcagaggatacgtgggctgaaggctgggcctcgggagggctgggggcttccagagccggggcagctgcagctctctctggtctcacctggaacttgccctgtagatcctccctgccctgcggctccaatcgaccgtgcacgggccgtggcatccgtcccccaggcgtccttccctggtcttagcttgtacagctccccacccacccaggtactcggttcccggagaccagggccaaaccaggaggccctcgggagatggggggtcaccgaattcatttccatgtgggaacttgggatacaaaacagccaactcttcctcagccacacggatgtttctcctctagtggccccgagaacctaccatggaggggacagtgtcagggctggacgggcacg

SEQ ID NO: 34 - DNA, 3930 bp, Homo sapiens
gccaaggattgaggaccctccacccccaccccaccaggcaaggaagggctctacccagagtcaggagcgtggcctccagggctgcgagggaagacgccccgtccagcagccccaggatgccagcccagttccctgtgcccggcgctcttcggtgcagacgcaggcaggggctcctgcaaccttgtggcatcacagacgcccagcactgactgggcccagatctcctccccgcagggctcagcacacaccctgttcccggcaggcctccatcagtccagcctgcagcagggctgcccccgcggcctgggtcaccccagactcttccaccctctccctggctgactgtcccagctcagagtcctcaggtctaagggggtcacggccctcctgtggccccaccggccccaggctccccagctgtggcactgtgagaccagctgacgttgcaggaatggaagccccagcggcccagacggcttggggagtcctcgggagcaggtggccagagacaggtgcgtgccaggccctccgcacccagagcggggccgggaggagagaggaggccccttgttcgcgcaaggccctgcttcctgggcccacagcagcctgtcagaagtttccagctccttggactggctgtgtggggcctgctccctggtttcaggggcctgggaagggcttggcgctttttcctggtttcctactctgaggtgagctggcgtctccctctcccactgtgggctgaggggaaagacctctgtgtccatcccacaggcctggccaatctctggggtcctcaaagaggaggcttttgagggggcacagcccaaacccctgggcctccccttgaggtctcctcccagcccccacccagaggaccttcccacagccttgggagctgaaacccaggccaccccatcaagttggcctctgtgggtgtacacactcctttccctcagggccagggtgggtccccacccccagcactcacagcccctccttctctggcctccctgccctccgcaccctccctgctagatgctggtgccgctagccctgccctgatggccacactgcaccacgctggccaggtcagaaccacccgaggagaagaaccaagatctggccccaccctgtcctcctcggaaggtctctctggggcccaccccctcctccctccccaaggatctgagcctccctcaccgaggttcccagtggaggtagacagtggatgagtgatcccaggagagctggctgcagccaaggggctgaagggaggtggaggcgggaggggcaggaaggaggatctggaaggccccaggcgctccccacccatccagcctcggcctctgtcctggtcgcgttgcccagcgaggcctctccttgggctggggctcgggtactctgccctggtcggggccacagatgccgcaaagtcccctcaactcagctagccagggtgcaagaccgcgcccacagctgagaagccaggggttacgagtgtggccctgccaggacctcctcagctgcatcctccagagtaaacacaggtggccgcagatcttccagggccggccgggcaggcaggacaggagcccaggagggccgcagtccagctcccctccccgctgacccagggccggacccagcccggtgactggagcagaaggaaacccaagccccaggccctccctccggtggcatccgaaggtctcagcggccccagcctcccccaggggccccgcacccgccaccgcccacctcagaccggagagagagtgagggatgggcagagccaggcccaagtccccgccggggcgacggtcacggtgcctcaccctcaaccgcctcacccagaccttccgacccaggaacagctgaactcagcctaaaaagcacccgtcccgagggcctgagtccggccgtggtgcctcctgctgcagagatgtgttttgcacactcctgtgtggcagggagaggcccgggcgtgcgggctgggggcccaaggggtctggagacgcttccctgcggagacggggtttgcccagcccccacctgtcacgcttctcgtcacccccaagtgagggccgtgggcgcgggcggggtgggcaggaggccctgctgggctgggtcacacgcatgacacctggctgtcgcaacacagatatcatcacgcccgggcacccgtgagtcactggcccagagcaggggctgcccccagcctcccaaacaaagaccctttgtccccaggcctctggtgccaggcccacctgtacagcagtcagatgcgcaggcggacagacacgccggtggctcggcaggcacaggcagggccagggcgtgttcccgcaaccagacacgctgccattcctgggtcagggtcaggctgagggagacccctgggggacaggccctgaggtcaccatagctcagagtgacctgaactgggagtccaagcacagactggccaagcccagcccgtgagcgacggccccaggacgcggcgccgagctctgcccccagctccagctcccagcggcgtcggagcacagcagatcccagggcagcgctctgcaggcaggaaagagcttccccttgggacagcgcgctgagcagcccccagctgagggtgggagccccgtccctggaccccttcacgcagttcagggagccccacatgccgaagcagccgtcacagctccatgggcccctctgctgtccctggcaggaccgaagctatgtggcctcccggacgccagggaccccggccacgcccgctccaggcactgagtggccagccaagcgctcgggcccggggtcctggacggctgttctgggtttgttctcaagggggccgtgctgctggctctgtagagagtcccagtcccagggcagagacccacacagatgtgcagacacgtgggcacacacgcaccagtcgcagggacacacaactgtcaacccggggtcaacacggggcacctgggtacatagatttttacaaagcagggcaggcaggtctgtttggaccctacacagcccctacatgcccccaggccattcttgttccaaggcccagatgacagtggtcaccaggtgtggtgtggtctggggtctgggacaggccccaggaacgccctgggcttactccagagaggctggcaggcagtccgaggggcctttggagcagacaccctcccagctgcagggcggcaggggcggcaggggtgacagaggcggggagaaggatgcgaagacaagatgccaaagctgggcctccagcgcctgcctgtcctggctgcagccccagggtccacacccaggcgcccccaggggccaggccagggcagccgcatctcctacgtaccccaacagtggggcccttgaggcaccggggacggatgggcaatggtgtccacacctgacaggcggggccggagcggggcccagcctcctcctcacagccaggagcccccagccctgcctcccctggctcctgctgccccctcagggtggctgccgcacctggccccaagaggacttcctggctgccctgagctcccgtccgcatttctgtccattcaagaccaggacagcaccagggctgggaatactggctccgacccagccgaggcagccccggggcagggtgggtcaggcaggtccagcgctgggactctagggaagggctggtcctgtgagcagacgagctggagggttggtggggggagtgtccccgcaccgggcatggcccctcccaggatggcagggagcccacggcaggagtgtccgatgcccccagccccggccaggcagcagggtcggcctgcggttctgggaagtcagccctggtggaggtcacggagaagccggcagctccctgccgctcagggcatggggtcaagggtcaggggtcaggggtcgggttga

SEQ ID NO: 35 - DNA, 3512 bp, Homo sapiens
tggtgaggccccaggcggtgttcagaaaggcctggctgggtgctgcctgatcctgggtgcctgcccccagcccgttcttgcccagggttggcccgtcagtttggggaggagccactgaaaactggaagcaaacaggggagtccgcagcccagggctcacgccaaccaggaaggtgcaggccacgctcctgcctctgcctcctcagggcccccacactgctgtccccgctgacccagctccaggagggcccggcacaaccttggttccccctgtacagatgcacagctgcccgactctctggaagggagcactcttgagtgctgtggccaagcagggcaggggctgcagaagggagaccccccgttccagatccaggccccagggggcaggccgtgcccacagaaggggtgctgagggcagagaggagcccctaagccggggccacagccttggcaagtgaagcagaggcccctccagacagccccagcccctgacgccactctggggggcccagggagagaggtggggacgggtcaccacccaagcccacctcgtgccgattggcgcctgcccacacacctcgtcgcagggctgggctgtcccgcctcactgcccagcaagccttggggagggccccttctgtgccagccccggcagctccaggtcccaggggaggggtaacagccgtgggctctggcctcttccaacctccccaaccccaccagcgactaagggctctggatgccaaccagagatggcatctccgcagctcagcagaggcctggacgtcctgaggccagtttacactctttggtgtgggtttgccagagccaaaatggggtgggggtggggcccaaatccacaggacctgccagggagcagcagcatgatggtcacatatggggcccaccccaccctccatggggcagttctggcccctaaggcccccgagaggccctggtcattagagtgcggccataccgagagcaggcgaggagaagcctgctggttccagccctgctccacctgggtgccccgggcacggcacggtctgggcgcacctgagcccgcaggggtgcctttcagctccacacgcctgcggcggccagcacatgcaagcacgcggtcccgtgtgtggcatgcacgtcctcttgccctgcacagagccccccacaggacgcaggcctcccgagggcccagaacagtgctgctctccaacctctggggcttccagtgccccacggcctgctgctcccccaaggctggacaggccgtgggcagagctgagtggggccggcacggacagtggtccttgtcctcagggtcgacgtggcccctgcaggggctaccagggcagcgcccagcctcttgccatcaccataatcccgggccaggtaagtcggccccgagggaggctctacggcccataccccaagctaccgggctcccctgtgaacagcacccttctgcccccacccatctcccgccgacctcggcagcctggcttccacccccagtgaaacatccaggcagcactcgaaggcagtggggagggtggagggctctttattgtggtgaccacgggcatcagtaggagggtccccgggatccggcggcagctcctcgccagcccccctgggcgccctcacgtgcccaggagcagcccggagaagctggagcccgcctggatggtgaggacggccccggagccattgtccacaaacacagaagcgtactgtccagcctgtaagaagcacggggacgtcacaaccgcagccacagcccagccactcggtggccaacgtctgcccacctgccctgcgctaggaggtgccgaggccccagaggtctgcgccctgagtgcaccgagctcacacccggcccagcccgagtgcacccgagccctcccgctcacacccggcccggactcacctgcagctgcagcagcccctgcacctgtagcgtgaagaccctgctgttgctctccaggcctgagacggcctccaggcacctgaacacagccccacagggcaagagggaggcgttgcaggtccagggggccaagacctgctccagtgcccagagacccctgtggcctgtgagcccctccaagggtggtccgggggctgccgcctggagcgggggctgaggtcactcacgtgtggcgctggcacagggactcaatacagatgagaacacacaccacgtcccgggcccgcagccgggccttgccctgcagctcactgtggtctgcggagagagccctggggagggtggtgcatggggggcggggtgggggctggtggggaggggcttcagggcacacatcccaggacaggcccaggagtggctgctggggctggggagggggcgcctgaggccaggcgtgcagcagggaccccatgcccagtccaaggccccccatggggcaggggataggtccctaacaggacccgcacccggggccggcgatgccaggcgcccccagaaagctcagccccagccccgtcacagcacacggcactgccccatccggctcacccacgtgcagactggcagagaactggaagatgccggacacgggggccgtgaaccgacccgaggccaggctcagaccggagcctcgcaggaaggcaccttgggcagcaggctgtgaggggcagtgggtgagcggccagcgcagggcctggcccccaccccacagaccccgcctggggaaggtgcctgcaaccgacagcccctcactcggagcagctctcccgggaccctcacgctcactgtgggcaccagcaggactgaccctcgagtccacacccaggagggtctccctgcctcccggctaccggggacccacgctccgtctgggcataaagtgtgatctgggcccccagggcctcccaaccctgacccgaggcagcccctcgccctccgagccccgcccccagcccccaacccacatgctgccccatgagtgtcaggcggtgtgtgtggtcccgtcttgcctgtggggccccacccaacaccccgctctaagctcccggctccactcacagcctggaaaccatgcagctccaccagcgtccgcttgtccacccggcggggaccctgcagccggcagtgaaaggcctcgcccaccagccgcaggcccgccccctggggcagcagcgggtccagaagccctgagaaccggcgctccgtggcctctgtggggaggagggcacaggcggccagcagggtcagcacagggcccaggcacgtctggtctctgggcagtgcagggcggctgacctttcagcagctcctgaaactcgtgaagcagagtctccgcggtcacttctgcacctggaggtcctgggggaccgaagagatcccgctggggggagagagaagcaggtgaggggcccagtgggacccggtgggagctaccaccacaccctgtccggggctcagaccctgcagcagcccgggcggggctcaccggcttcttgtccctgcttccgcaccgcttcctt

SEQ ID NO: 36 - DNA, 1632 bp, Homo sapiens
gcagtgctgtggaggatatgatgactgtagtcagagtacttgtatgtgcagtgggtagtgctgtggagggtacgatgactgtagtcagagtatttgtatgcagtgggtagtgctgtggaggatatgatgactgtagtcaggccctttcctccagggacctaacatttgggaaaattggattccagactaatacatcacttttaaaaagcactgagtatcttctgtgtgcccaagtccttgctaggcccagggaaggtgtgaaagaccttatagtcctttctctctgatctggggggctctggccactctgggcttcaatgttgcctgtgtctcagaaggacaggacaagctcccactatgtatgttctctccttgtctacatcctgttgcctgtgtctcagaaggacaggacaagctcccactatgtatgttctctccttgcctacatcctgttgcctgtgtctcaaaaggacagggcaagctcccactatgtatgttctctccttgtctacatccataccttctctatacttcccagatttcacaggaaaatctttgtgaaaccaaaactttcaaaagaatatatttgggctcggcacggtggctcacacctgtaatgccagcactttgggaggctgaagcaggaggatcaactgaggccaggagttcaagaccagcctgggcaacatggcaaaaccccgtgtctgctaaaaatacaaaaattagctgtggtagctcgagcctgtaatcccagctgcttgggaggctgaagcgcaagaatcgcttgaacctcggaggcagaggttgcagtgagccgagatcacactgagatggcgccactgcactttagcctgggagacagagtgagactctgcctccaaaataaaaagaatgtgttggctcatgatcagacttgagcacttgggctgagagcaaactgtcattcctatttccaccagctccttagctagagactgaatctgaagctggaaggagcaacttcttttgaagtattggattttgtttctttatgggggaaggaagcaaggaggggcaattctggtgctctgaattccgttccccatccgcacctcctagaatagggctgaagtctgtccagagtggagaggaatccctgcttcctgttacattcactgactaatagatgctccttccagcttcagattcagtcggacatgtctaaggagctggtggaaacaggaataacagttcgctcctaccccaagtgcctaagtctgatcgtgatccagatacattcttttgaaagttttggtttcacaaagattttcctgtgaaatctggggagtgtggagaaggtatggatgtgaacagggagagaacatacatagtgggagtttatcctgtccctttgagacaggatagcccacgctgaagcccagagtggccacagcacccgagatcagggagaataaagctgagcaatgagtacgagggaggtgtggaggcaggggtggcctctctgagaaagggtagagagtcttgaatgaaggagtgagagagctttgccagtagaaggaattgtaagtggcaaggccccaaaactccctcctgaaggccagggaaacttctactccacaccctatctagagt

SEQ ID NO: 37 - DNA, 2502 bp, Homo sapiens
ctgcttgggccctgatctttgagaagggggagcagcagaacccgggcactgacgctacagtgccactcacacccacagatttctccacacaggcatcagtctcggtcctggccacctcctcctggacggcttcagccattccccgggactcacgtggtccttcctcacacgcggctctggtaggatgcattgctctgtacccagggacctctgaggtgacaatggccacggtcatgcagagtgcaagggcacaggctgggtgcctattgtggggaccgtgactgcagcactcccagactatcctcgggcatgttgcccccaggcttagctagggcaccagcggtaggtgcacactgctccggactctgcaggaggaggacaactgttacctgtgtctttatgttctcctgctgctgtcactctgtgcttctcatctccttgtggtaggattcagggcagactctctgaacaccttgtgggaaatagcagagtccagcagggaagagagaagcccagctgcaaaggtgaaaaaatggcaggtgtgacaaggacccccattcagatttaaatgaggtcctcatttaatctctgttctgattggataacacttcaagtgtgtatgtgtgtgtatattttttgtttgtttgtttttgtttgagatggagtttcgctcttggcatgcccaggctggagtgcaatggtgcaatctcggctcactgcaacctccgcctcccgggttcaagagcgtctcctgcctcaccgtcccgagtagctgggattataggcatgcgccaccacacctggctaattttgtatttttagtagagactttggggtttctccatgttggtcaggctggtctcgaactcctgacctcaggtgatctgcccgcctcggcctcccaaagtgctgggattacaggcatgagccaccgcgcccggcatatatacatacatatatatatatatatatatatatatatatatagagagagagagagagagagagagagagagagagagagagagagagagagagagagagagagagtctcgctctgtagcccaggctggagtgcagtggtgtgatctcggctcactgcaacctctgcctcctgggtcctggttcaagcaattctcctgcctcagcctcccgagtagctgggattacaggcacacgccaccatgcccagctaatatttgtatttttttttttagacagagactcacagagtgctgtcacccaggctggggtgcaatggtgtggtctgggctcactgcaacctctgcctcctgggttcaagcaattcccctgcctcagcctcccgagtagctgggactataggctcctgccaccacacctggctaatttttgtatttttagtagagacgggggtttcactatgttggccaggctggtcttgaactcctgaacttgtgatccgccctcctcggcctcccaaagtgttggaattacaggcatgagccactgtgtccggccactatgccccacctctactcaaggtgataagcaagcctgggtgcctcctcttttggtgccagcagaaaaagcaaactactacacaaggctcttcttcagtacatgcatatacaaactctcaccctggccccaaaccataacaaaaacctaagctattctccttttcttacgctctcaggccacttttcgcctgtttgagagtcctgccctgctctccccaaagacctcaattatggacttgtggctgggggccacctgcctctgcagatgaccataacagctgtagaaaggtaaaatggtgtaaacattgcaatatatgttattttcaattgacaaatcctgcaaatcttttcatatcaataaatgctgcccctcatttttaagtgtgtatgatgaggccatttatccaatattttctaaataggtacttgaattatttctaatcttttgctattacaactgtgaattaaaactcacactgtcaattcagagaacaattgttcctttccacttttatggtgctttaaatatattaaaaatgaaaaaatatacacatacacacaacacaaagcacacacgcacacatacacatgtaaaagatagggtttcgctctatcacccaggctgaagtgcagtggcatgatcatatctcactgcagccttaaattcttaggctcaagcaatcctcctgcctcagcctcctcatgagtagctaggagtgtaagtgcgtaccactacgtctggctaatttttaaaattttttgtagagacagtgtctctattttgcccgggctaggctgtaacacttggctccaagcaccaagcaatccttctgcctaggactcccaaagtggtgggattataagcatgaaccatgtgtccagtctgaaaataaaaatatataatatcaaaacttctggaatgcagtgaaagtattgcttagaaatttacaacgttgaatgcatacattacaaacaaataaaattatacacccaatgatgt

SEQ ID NO: 38 - DNA, 1853 bp, Homo sapiens
gatgtttatgtccagattttctcttccctgttatattgattacataaggagttatgaacagagagacattgattattaacattgttgaataatgaggtctactacaatacccccataatgtgcttggctaccatgctaggtgtataaaattcatcacagggatattaagtgattcaggataaatgccaaataaaaatattcggaagcaaacattccgacattttgtcatctattattgaaaaaggtgagtctactttcagttatgaggcctgtggttcaaaacatacattctagcttactaaacaaagaaacctctcttcaagtttttgacctaatgactttgttactttcttttctttattgtaattttgattccatgaaactaggcatacagaagactaacatgaaacatgaaaacagcttctaataaattttgcaaagcatgaacatctgcagaaacaaacaaacagaaagtaatacaataagcaataaacaaacagaaacaacttaaatggcccttataaaatgcaaaggtttgggggagggtcttggagtatgttcacttaccattagtccaataccctggattcagcagaggtaattactccaaataattataactgagaactaggccaagaaaaaacaactcacaaaaaaccagtacctttttctttgcctgtagaagctcctgataggcactggatcttataaaacgtgggtatgaatcacttttcatcagtttgtaaatgtgctcctaaaaagaaataatggttgaggtgcttctttatgatttcttgggaaaagtaaaatatcatgatgtcacacatggctaaagaacaaatctagtagcagcgaaaaatagtaataacaatgctgattagaataccttctatttacaggatatttagatcttcaaattcattatctcattcatagatcattgtttaaattggtttaggagctactgaggaggcaaatcacatccagtcattacaaaaatggaatttgattaataaaatgtcataaaattacctcaaatcaagttgttgacttatatagatcactagagaatataactaaatttgctgtctcttaaaactactccaggcctgaagtgggtaatgttgactcagactgagtaatcatcctggatacctttggcctctacatttactgggagggtgccaactacccagaagaatcaaatcatctctttggtacaaattgcatggaaaattgtcttccatacccactttgggtcagagcacaagtccaaaaataaattttgtgatatttaattgctaaatctccaaatttgtgtgctctttcttattacttacccagtgacagattaggtaaatagttgatcatttgcccctaagaagtttgcaatattctgttttgatgatgaattttgatagacaagtcaaaaaaaaaaaaggaaaaaggtactcattcaattcaatctagacccaatctagggaggttccactctggtctaccgcagctcagggagctaacatgtgccttgatcttccaactctagtgaaatatcagttaggtgtagagcttggaactattggagagcattctgaatgttccagttttcttttctttctttttttttttcttgaagaaaatagatgtttcaagaaatgactccagttctctggtcttaaacacaacagcaataatttgaagttactttaaattcatttaaagacattcaggattaaatctcaagacttagcccaatggtgatcttcaaaggatgttaagtttggaactgtatgggaatttgtttgaaaagtagagcaatggctggttttggagttaagcattttgagattcac

SEQ ID NO: 39 - DNA, 2616 bp, Homo sapiens
gtgcccaggaaagaccaggaaaatacaagtacatggctgcttcataccatataccccaattctttaaagcagcaaaaggcactttttttttcaggccagagtgaatctaaaacaaacctggctttgcttacagggaagctgtcccagaaggactgagtgatgcctcttgttccctaaggtctggagagtctttgcaagtttccaacgacatttccaaccaggtgggagagaccagcagttgacgagtcaagtcagacccaaaaaacgacgccaaggtagtgagtgggtgcctatttgggagtaggatgatttgaggaaaacaggaagaaaaaccggtcagaaagtggcactttggaagtggaaagctgtttgcaaatagcaactctggctaaagcgaaaatgttaatcaagtagaaagtaaaattcaggatcttagaagctcatccttctgatgagaactatttttttttccgtgaaggaactattattactttaaaagtgagggtaatttacatatggggtgtatatattctaaaaatagtaataaaagtaccttttataagcaatgttgtgtggcttgtagaagaaagcagggaggaaaaaaaggcaggcaaaactagtctaggtctaggccctaaaaatgagcttccttcccacttgactggaaacgcccatgtgatttctaggctgaaaataggtaggatttaacgagtaacctagttcccttctgtctctgatttctgatcagctgatggagctgctagtaagaggggccgatcatgctcccagacgagtcctttggcctcttgctctccatcccaagcctgactccttcagcagcagccccctccttctgtgtccatctgatgcaggcaagcaggagcagtaagagggcatcccatgttccagttcaccttctatggggtgactaggaggttcccggtaactagggcagcccaggcccagcaggttgcaaaagcagctgcaagcttcagaaacccacttcctccaacaccagggaggtggcagagagcccatccaaaagcccactgggagaggcataagattctgtgccaggcccccaggtcccctctgtgtcaggtaggctctgctactggcctctgaagtaaaggcaaacacaaacgggcagggcagggtggcaggaataaaaaactctggacagaaacccttttaataaaggaaattccacccctcccaatccttccatggaagggtgagaccttaatgtgatgtaagaggaaggtcttctctggctttcagggaaacagctgcagctgaaacttaggggcccattccagggcacttttcaccacagccagtgcagccgctccaagtgccactgtcagccccatcactgccaatttcacaaagcggttggtccttggcttggtcaggacatcttttgttcgatcttcaggccgcagaagtccccgaaaccgctgccgcagcaccatatcaggcctctgctgggctgatgccagctcaaagtctttgaaagtagaggctgccgtcctgcaggggaaagagacggaaggaaggaagtggtatgaaagaggaggaggaaagcaaaactacaccacataggctgcgggcagagcctttcattgctgggaaagctctttatgataaagacccatatgtctacagtggggattccactggcctaagctcagatctctggaaacatgccccaaccctatcccaccagacacaaaccttccctcgcttctgctcatttacagccacccccattcaaccagtgtcccagccttgctcacctctcagcttgctgttgggcagcggcctcccgagcaagttcggatgggggaaactgaacaaaaaggtctcctgctctgctgatcagtgtctcatagggcaagtcctgagggatctgggacaacaggtggtggaccgaggccatgtcacagtcacagtccaggacttcctgctcgcgatacaacacaatctgtggggaggtagtaaagccttgcagtcagaggccagacacacagggcctgggccacctgcactccattatccttgcagatgaatttaaactggtaacagacaggactcagcccaaatgttgagcaaactcttgtatccatcaaggaagtaataacatatatacgctcagtgctactcctactctctggcccttcctgcaaacttccaccacatgacatgaaaggctgaccagttacaatctaagtccttcgggcatgctgggctgctcaggtgtccctttaagtcttgaaagaaatgaaggagattcttttaggagaaagtaggagaattattgggagattcctggagctccagcatagaagaaatggttcaaaacagtagaaagaacagtcttgctccctttaagcatcttccttctgactgttggtccacaaatccacagatgctcaagggaccagtggtcattgaaggacttccctgaattcccatctccaccccatccctcaagacccttctactaactgaagcccctaccctccaccgcaagccgcctcccttgtctgtcatgacaccagatctcttcttttcttaaatctggagttgacagcttacgctactatttcccta

SEQ ID NO: 40 - DNA, 2997 bp, Homo sapiens
tcagtgctctcccgctctcctgcttctcttctgaggtcagtcacagacctggacatccggcttgtggggagtattgagttgcagtggctgtgtgtgcttttgtatgtgaacacatgtgctcatgtgttgcatgtgtgtggtgtgcactgtgtctggatgtgatcataggcagcattttggggtatttttgggtgtcagggtactcactgggggcattgaagatgcagtggcaaagcaggtgtccaggagtctgagctcagacttgactttctgcctgggtcagcctagattttctacatggaagtgaggtgaaagggagaggaatatttgggagcccttctctgtcccttaggtccctaggagcccaaggatggtgagagggcccagcccttggtttttgatctatttgagaggaaccgagtaatcttctggggtctgctcttggcttcttcagtacagtgaaattagctgagcagttcctctgggcagagcctctgctaacattcctttgaagcctccctccatgctgggaatccagcaatgtccagtgataagcttgggaggaggacatacttgcagtggaagagacaccatgcctgtcccaccagccccttcacttttggggtcaagcattattagagccctgccaatggattgtgtgtgtcgtgacagatgtcagctgggaggaaaagacactgggcccctcctgcacaggggccttatttctagagaaagggaagactgaggtgcaacgtgggcctgtggttagggagactgcattctgaacaccgtgggaagaatgctagaagctctcagcctctgccttcctctgccatgctcgagctggtcagtcatggtccccgaggccctacagcagcctgcagggatcagggcagcaaaggtgctgcaaaaccagcaagaccaacaggactgtacaagaccggtgttccacggcgacaccttgtggttgcaatggcagcagcactgcctgtggaaggacaaggctctcctgcagctcctccctaccaggctttggactaagcctccagcatttttggacagttggcatgcatgttggaggagagtacttgagaaggaaataatgggctgggtgctaatagaggatttggaggctcacacactaaatggggaaggactcattcatacccattccttcttccgaaatgtctccttccatgtcctgccctcgtacccattccttcttccgaaatgtctccctccatgtcctgcccaggcctgctctttgggtctcctggctggtgggggaacagatgtggcgtaatcacgtcgagatgcagcaggtgcaccaagcactgtgcgcaccgctgttagccccaggacccccagtgtcagcactggtggggctggtgtttgtggagtgtgtcagtggactggcaggcccgtggattccacgtgtgtaagagagactgacagcccttcctgtctcagagcagcccctcctgggtcccatcctgggtcccatcttggggttggacatgcccttgtttgagcttggccccttcttgctgggccaccagccctgaccctaaatctgagagggggcttggctgggcctggggtcaggggacaaacagccaccctggctgaggccctgggcagctgaggaacttcagccagctttgggcagctcttgggttgggagatgggctgctgttttctcggacaacgccctccccagcccctcaagactctgttttcagtcagttcaattagtacaactttaaagcaattagggagaattagtggccaggctgctgcaggcagatgctgaatacactcatgccccctcccccaacctccctcaccgaacctgacagctgctgcggggagtgcctttctctgctggctctgtcctttctcccagagatccagcccccatctctccttctctcaagggtctgaggaggggagggtgggcagtctaggggacagacccagagacaggggccctgggactgggagggtggggcaggcccggggaaatgggccaacttcccctcaagaccccaggcctgggcctgctctaaggagagaagggatgggtgctggttggaggctcagcccctgagtgagggtgagggtactcagcgcggattgggaggactgaccaggattgtggcccagcctctggccctgtggcctccaggagcccccagctctggtgagggcaccctttggtggggctgggggctgttcttcagtgggaggcctctgagaggctgggcctctcccactaggtgtggggtggcagcgaggccctgcttctgagccagtgctggagccacaccaccttctctgcctggtagtgaaggaggtggccccgtgggtgctgcagaccctgggccctccctggtgccccttgggctgctctgtggggagagctccaggtgcttgcttgcgtggatggggcaccagggcaggtgcagggctgacttcgcagatggagccctttgtgcggggaccctgtcttccggccttgcccctccctactcccccagcttctcaaagaaggtctgttttctgagcctcctctgtgatgcccccaccagccgcagcctccctcagatgtgtggggggtgtccgcggtcctaaccaatgtcttttctgcatgtgtccacgtgtatctggcactttctctgagcaggctctgggctcagcaccgggtaaggcagatccatgcagcccctcaccttggccgaacactgaacagatgatgacatgtacttgtgcaattccagcttcaacaagggtcaccagaacagctctgagcaattccagcttcaacaagggtcaccagaattgctctgtgcaatcccagcttcaacaagggtcaccagaacagctcggagaagggctgtgacccggtctgaaagcttcccagagactggcttagcgggatgaccctggggaaggagatagtgggtggagcagagaggctgattagaggctgagtct

SEQ ID NO: 41 - DNA, 2166 bp, Homo sapiens
ctaccccagatcctgaggattcacatagcgctgtactggcatgagatcatgtgagcatgaacgttacttgacttgaggccaggggctctgcatgcagcgttatctacaaatgtctggtgccatgtcaggggtgggtcggaagacttttgtctccccctggcccagacatgacaaactcagagagtttgggacctaccatgacaacccatggctgttcaaagtgctgcttctgtgaacaaagccagggacccgtgcccaggttctcgtggcatcaccagctctttcatcactgctctgtttgagggtcatttcccttcttttcttgcagatagggccgagtgactgctctgaatagagaagctaagatgaaaagtgtgccagagaaggcgagaggatgagaaagggtcgactgcctagaggacagtggggcagcaggtgcaagtagaatctcctgactaagaggctgaggagggtggcagcagagggcataagccgtggtcacagtgtgagaatgtcacacagccacagcagcatcggggtcagccttccagaggctggcttcggacaggagatgggtggtgaggagccagcatgggagggcagtgaacacacaaaccctgtgcatgggaccgtcacagcctgcggcgtgcctctgagttcagcaccaggcatgtggacagctcaggaccggttggaaggggctgccagaagtcaggtggtcgtgtgtcggggtatgcaggagctgatggtagctcctcaacccccttcttgccaaatattcagagatatggaatcaaggaaaagatcagttgcatggccattcagccaacccttcttcctgccacccagggcaggaggtgcctctggcaaggactactggacagaggctcctgcaagggaaggagctgccactgggtatggcccttctggcctctctttatgttgttggattctaccctgggtgggtataaattccatttatgctggagtttttaacagacggttgcagatatggctgcttcatcagggtatccattatgtagctctaatttttgatttgggaatgaagtgagccagtatcccatgcttagagctgtcaagagaaccccttctcagacatgtgttaaataatgccccatggaggtgtcctttctataccccaaggaggaggctggtctattctgctgaatttgttgggagaatttcagaatttcagacatgcaacaggacatcacccaatgtgaggacagaactatctctgcaaggaaccaagggtactgtgatggctgccagtggggatcaggggtgagggcatatggtttagcctcagagatcaagagagtggaaagcaggatgtgtgctgaggtcaccgactttctatatctgttctgtgggctgagctggcaggcaggtccatgcaccaaagaaagggaaggggagggctgtggatgcagcagaagatcctcctgggatactcgggaggggagcaacacaaatgcttgaatgctgctcttagatcgttgagtgggagcttggatcttccacaatactgtctgctgtaatggcttcacagcagtgacagggaagttgatgctgccctcagtacataaatgagagaagaaaacaggccagaccatggctctgtctttctcccctcccctcactgcagagaagtgagactgaatgtggtgtgaggtactgctggagccaggcagggtaggggacagccagtttctggccacctcctcaccccccactcttcactggccccttccttctgggaagtggctgcctatggtccgctgggactcagcaggtgctcttcctcttcttctaggtctctgggaggaaaaccattatgcaagaggctcaaccgtcccaccgagacactataacctatgtaattttatggatttttaaagaatagttgtaagtccattctaattctccagatttgctggctgtcagaacacattttaaataaaataaaacactaccgtgtctccttctctggcccagcgctggggtgaatggcccccgtggtgtcagaatgcccggaaccccccagctcagcgttcccacatatggcctctctgcagcccctctgaccacggctctccacacaccccagccccagggtttcagagatgtttctgactgtcccca

SEQ ID NO: 42 - DNA, 3695 bp, Homo sapiens
ttttccctcctggcctcactcttgcaacttttctatctgccactggggtcaggatccatcctggggctcccacccttcctggagaaggagaaaacacccacgtcctggtagtgttcagttcttccaggcccatcagagctggccgtggttgcagggctggcctggtggtcctctgtgctgggctctgttcttagtccacacttaagttctcgtagcacccagcaccttggaggctgtcattgtcagctccttcttaattccactgattgtacactttccagactgaagtcattgcttggtccagacaggaacaaagaaagccatggctgcttgccaggatctcctcttctctgagctgccaggttcagaagctcctctgtgcctgtgtggtcaccagcatctaccaccagtcttcctgcccctgtgccttctatgccagtttcttcgtgccatcttttgtgcatgtaaaatcctgaagtattccaagagcattagtggcagtgaactgaatgcttgcagtagctttttcgtggctgttgctgacccttccaacagttccttgagggtccacctcaacacagctttaagaagagggcagctgagggctgagtccctggctgaatgaagaagggtcaggcctggccctgaggccactcctcagaaatgcacctgatacaactagcgtctcctgtagattcctcagcttcctccttgctggggagttctaggttatgctgccttggagtgtcttgctattgtcctgggctatgctactctttggccctgcctgatactcactccagttgcagctgagctgtttgaaacctgctctcctaagttctggggaaaatcttaggccctcctctatctgatgctgtcagcaggacaggccattgattatttgagggtcctattgcttcctccctgcaggccattcttcaccggcctgctctgggagcccttgaccctgggaggtggaactctgcccagctttagtggtggaatatgcaggggtagtgtcttcctgagtctccttcctcaccagacgctgtgaggcccctgcctgggctgcagattggggttggggagggtggcacgggatccccaggtcccatctcactggctgtgcatccctgtactgcaccccaggcccatgtgcttcgtgaagcagctcgaggtccctccatatgggagctaccggcccaacgtggcccccgccacacccagggccaacttggccaaggagctggagaagttctccaaggtcacctttgactacgcaagtttcgatgctcaggtttttggcaaacgcatgcttgccccaaagattcagaccagcgaaacctcacctaaagcctttcaatgtaagttggggagaattgttcttgtttctcttctgtgttgctcctgggaggggcaggattcaaggggcagtggaggagggaccctctcgaggagctactagggagggaaactctaccctcatgggaggaccacgatgcaggctggaggtctcagctgtcccagtgggcactgtggtggctttcttggggcctgcatctcactcctgctgccaccttcatgttcaccattaacatttatgtgtctcctagttatttgtgaaacaaaacccagatccgttacgggcgtgtgtgtccaaagacttcagagcaaccccaccagcatggttcacactgggagacgccactctccccactgtcctcctgctacctgtttaatcccagtgcagccggctgtccatttcccagccctgcctctggggagggtcagactgtgggctgggtggggccagatgactgcggggctgggcccagtgccctggcaggaagccattgctctcctggtggggaccatcttactggatacaatgtgttatctgtgacattagtaacaaattttctgggtaattgtactgacaaaaatcattcctacaaatctttaagaacaatcctttctgtcttgtcttgtcacttactgccctaatttgtggaataagcccattagccctggaagtgcatgcgaaatggaaaagcattcagtgtacacatgagattgggagtggcatcgcggggcagatgttgtcagccccaaacatgacgtgacgagtttcctacatgagaataataaaagtactgattgatgcggctgccagtggggtgtgagcctctcttcctaactttgacagaacctgctctttaggatggaggacttcctgcctccaggcacacatgcctacttggatgagggaatgcaatggtgccagtggagagggggacctcacgataagctttccaatatatctagacctttctggatatactggtgacatcgtgattgctgagaacatcgtgcatgagagtgattttgcagctacagtacaattgctagaaaagataacattctgtgccttcatttgtcatgttcatttgagcaataatgttacttttttaaggcagtgatggttaccggggacaccaagtcagcctaaatatgggtacacccttttgagatcatgggacaaaattttcctatttgggcgatatggcaaacactcatcctattcacagaatgcttcagtttctgatagacaagttatttttgtttgaaatatcagggctgctggaatgtcttggaggcttttactccttttgcccaaattttcactgagccagaaacaagattgtctcctcagtcccctagaggagggtgggtgggagtgaggtgtgtgaggacttgggactgggacgggtggccaagcccctggcccacttcgatatagctgtgccctgggccctcccatccctcccaaagtgccccctccccactgacttgtctgcattgctgcctcttttcaagttgtatatcagcctggtgttgttccctttttgcagccaaacctttcccaaaggcctcttcccccaggcacagcccctccagtagttatgtgaggagcacttcatcctcttctgcaggctttgactactcgcaggacgccgaggctgcacacatggctgccactgccatcctgaacctctccacgcgctgctgggagatgcctgagaacctcagcacgaagccacaggacctccccagcaaggttagtacatctgccacagagcctttcttgggagaggtgagttggtggaatttgcagtcaggcccacctgctctctgcacaaaatgtccctaggaatggcttgtgcctagctggcaattctcattcttaactttttctccctcctggccatggccccaaggaccgcagagcttggatgggtccaccaggagaacctggtgtgctgagtgaagggggaccaagggctgcgaacacaagttcccacgtgttaggttgtgtgcacaccatgcgcccgcgtgtctccctctgagcctgagggtggtgcacacacatgcccatgtgtttccttctgactccagggcggtgcacgtgccctgttcacacgtgtttcccgcagtcttgtggttgctgacacactctccttgctcagaggacctagtcttacccgtgtttatgacatgtcctgagggactggtttttgtgctgttgggaggcaagaggaattgtagggcccccttcatgggaaatcaggaaatggcagctggattttttccctctcgctgcctgtctgtccccgttgtcctgcttccttctatgg

SEQ ID NO: 43 - DNA, 3164 bp, Homo sapiens
tggtttcgaggttactgcgattgttgtaatttgtatgttattaccctcgttgtgccatctcatcttcatggcatttcggtaacacttatttagtgcctactgtctattgagtgccatccctggctctgaagggaactgtatcctgatgtttacgctgcggagtgatgtggcggagggaggccagggagggtgtcaggagcctgccacactgggcagcaccaggcctcatttctagggcaacgcaggacctctggctgaagcaggggagggatccagcccctcaggggtgttgtcttctgtgttttgctggggggagttaagtcttcctcccttatccagaagataggagactccgggagatgcttctgtggacactgtcctgaagggtccctctccctcgcccactgggttgggcgcccaggcctccccgccagccggttaaaacatcttcctgctggttttttgcagtcagagccagcagcccattcttttgcttcttctgaagcagatgaccaggaagtgtcggaagagaattttgaggagcggaagtatccgggggaagtcaccctgaccaactttaagctgaagtttctctccaaggacataaagaaggagctgctcacgtaagtccctgtttggctggcacagctcctaggggaccctctgtggcctggggaggaacaggccctggtcccaacccatgacgaccgggtctgctcaggctttccccgacctgtcctgaccacctcgagccaggcagcctgtgacaggagccagggtattcagaggtttcccaacacctttgtgttgtgctgggctttactgcaatcttctaaaagtgattaagaacaaagaaatcccctggccaagctcaccaagcaggacagagcagggcaggggcagagtggaggagagctcctcagagagctctgcaggaagccctcggggcacccagaggcctggccctctccctgaggccgcagctgggcacgttctgccctgggctccatggccaaggcctggaatgtactgccttagggctcaccaccctcaactctgtcagcctggctggcccagaggctgcgtgtctgagctggtccgcatggggttggaacagacagagttgctgatggatatgaatcagatgtcaatgaccttctggtcagccttcattgccagccacctgtcctaggggactgtgagaggctgtgcctggcacctgctccacaggtgatccagctctcacatgtgctcagagtacatttctggggtccctcttctccccaacctgaacccctcttgtaccctcacacttgtagcttgccctcctgggagtggctggatccagggaaggccttgcttcagggcctggagaagggaaggagctcctctgcctaaatattcgtgggcacatacacgtgcacacacagcacatgtgcgtcagaggcatcctaactttaagctcaactttaatttggttactttttcttcttgagttaagttgtgtgggagaaacttccagcctgagaggcaccggctgtcctccaaggactgagtggaggaggggccaccgcttggctcgcgggtgagccaggagtgggcaccagtctccctcgcagagcaggctcagcctggggggcaggtacacaccactctccggtctgacactctttttcctttgtccagctgtcccacccctggctgtgacggcagcggccacatcaccgggaactacgcctcccaccgcaggtttgtctcctgctcgggtccgtctggcctgggtgcttcgtggtgggtcttcctcctctcctcctcctctgctctccctctttggcttaccccaatatcccatctcttctctttcagcctctctggttgccctcttgctgacaagagcctcagaaacctcatggctgcccactctgctgacctcaagtatgtttgcgctccctgacctcctgtctcttgggcggcaccctcgctttgctctccttccatgaggctcctgccaaaatcagccttctccaaggtgccaagcctcagctggccccagctctcctgagatgggcagaggggcagggccgtggaggggccgattctgcttggctggggctgctctgcctgtgtgcacctgctctgagctctgctgtttgcctctccgctgggggctaggggtcgctgcaggctcctgcgctgctcttgacccatcccccaccctccagcctctcctgaagatccccgacagggctgtctgggcctgctttcttactgccctagagatttgggaaaagcccagaaccgaccagggaacgtaagccctgccgtggctcggcaggccacaggctgtgcggctcttgctaaatgaactgaacgctgataatgaagagaaagctccttcccctcccctctcctgtcacgctccagctgcttctgccttggccctgatgccctccccccatgctcatgccttctctttgctgggctcacccgtttctgcttctgtacctccctgcccctacctaacacatgggcagggcaggccctgcaggcaccagctatagcttgctggacagtcctgcacaaccaggcgcaagcacccagaggtttccaggggtcagtgtcctcctggggctggagtcagggactgttactgcctttggttttcatgcctccagttgtgctgtgactcctcagcctgtgtgaccctgagccatggggagctcctcctgggcaccggggccgagctgaggccttggaggaagggggtcccattcttgtctcctcaggtcacctctctccaggggtgtccctccctcccataggcctctgtgttgggggccctgaatccaggtcaacacaccctggcttattccattctggggccagacaggatcctgggcactggtgcctctaagatgaggaaatgaacttgctgaaggcttctagggaccttggctggctcagacctggacagaaagctctaggtctcccagagcccccaccagcagccttgtctctgttcccctctggaggctggtctggccccagcagccaggaggagtgtgtcatgaggcccttcagttcccacagagtggggtgcagcatctaagtttccttcctggaagttaatagcttcaacataagcattttctgaggctgagatc

SEQ ID NO: 44 - DNA, 4370 bp, Homo sapiens
atgtatgcccacaaatctccagcgaccccagcctcagttactggacagttcccttcgcgttgatgtgaaacggtgcgtttgtcctgctctggatttcaggggtctgctgtagaattcctgttgtttcactggtctgtttactgcagttcccagtgcttcgtcatttccatcactgcacctttgtaggatctgcaagagctaggtctccagcagttcttttttttttaaagcattttcctcattagccttgggcacttactgttttgaaactaattttattatcattttgttgtgctttctcctttagtaggtactgcatggaatgtttatgttaattttgggagagctgacatctttataacattgactctcagtctctgattacttaagctttgtttaatatctcttagtattttaagataaggacaatatctctttgtcatacatggttgtgcacctttcttgttaaatttgttcctaggtattttttgtgtattattactgttataagggggtgggtgaagtgttctctaaataccaatgagattaacttggttgacagtgatgtccaggccttccatagtcttccataggggtgttggggtcaggggtcatcagctgtggctctgaccctccatctcagtccagacctcagcatggctctaggtcacaggcagtgattctgaatgtgcatttcttccagaaactccacttggagatgttggcagaccagccacgaacaactaaataccacagtgtcatcctgcagaataaagaatccctgacggataaagtcatcctggacgtgggctgtgggactgggatcatcagtctcttctgtgcacactatgcgcggcctagagcggtgagtggggtctcgagcgcatcccgggtgtttgtgccgaggctggtgacgtccgaggtggcctctgagtgtgctgacttgtgaccctgagctgttgggggctcaccggtgactccatggtcttgttgagcaccctgcacgtggggctcagggtcggtaaaatagcagtgcgtggagaccgcgtgctagaggccgtggcgcccgcgtacaatgagtcgcagacagcacagacgggagtagggcagaatagacaatatcccgtgaattgcgtggggcggggtatgttctgtgagacgtttatttcagttgagtagagaaacacgtgcacccacatgtctgtgctgggccttgggtgtggttggtctcatggggttgggagggatgcacacgctgggccccctccccacccctcttaggccgtctatactgtgctgagctgagccgagctgcagccttggagactccttacacagtgggtggggtcgcagcacagtgtccacccaagtccaggctctgcaggacccaggacccagcgcttgggtgcttcccaccagacccttccctgagaacctgggtttgaaattgtctgacaggcctcagatgtggcacagaccagcattgtcacttgggtgctaagaagttgctgtgctggtcatggattaagattgctgtgcgtgtggcagccggctcgggcatgcgagtcttccatccacttgcagccctgcgtctgtgtcttgtccgggaggtgggggcagttgggagggttagaggcggctcctttctgggtgcccctggaggggcaggtgtggccagtcctcgctgcctctgctgtctggaatgctgcttccctcttgtgtcattgaccatttctcgtgatgctggttgtgactcaggagagtagatgacgggccgtgtgccggccggatgtacgctgacggtgcctctgctgctgcaggtgtacgcggtggaggccagtgagatggcacagcacacggggcagctggtcctgcagaacggctttgctgacatcatcaccgtgtaccagcagaaggtggaggatgtggtgctgcccgagaaggtggacgtgctggtgtctgagtggatggggacctgcctgctggtgagggcgggcgtgcgggcagctgggggccggagctggggggcttctgagcacgggctcggctgggccaacctcaggatctcaagggtcgtgcgtgattcattttgatgttttccctaatgtgaggtctaattaatttcttgtgtggacattggctcagtgtcttgaattttcacctgatttaaaaaatgcctttatgagaaatttaagtcaaagttcatgtaacattttcatgagtgatttacatgaactgtgttctcctcggggatctgtaaaaatcctgtgcctaacaggtaaggctgtttctttaatgccagtagggccttcgtccctggccagggtctcctcgccttagactggccccagtgatgctgtgaagccacttgggcatctgtagggccagcatatgcctgtcctgtcagggttgctcaccctgagtttcacatgtgggtggaagtggactgttttctggttgcctgtgaatatgccctgcacaaacgctgtctgcttggagggaagttgacgggagtgtggctggatgctgtctgcccgcgctgtcttcctgggctcagcatcctgggacacaggacattgtagtggagcatcccaacctgaaactttgtctcagtgtagagacccagaaagatggggtctgggtgaaggagtgtggagtatggctgctgctttccaggaaacggtttcccctggtaacagatggcattgggcttttagtcctgttgaaattttgttgtcagaagataaatgtaaatagactcaatgtccatgctgtgacttggcttattaataacatctgtggagccataagatgacacacaggagaaacgggctccactcctaccccctgaaggggcatttgcctttgccctgaacagcagcgcccattcaataagtatctgttgacagctggtgccccggccacggggacaaaaagaggacagagcaggagtgaggctgtggtgaggccaaggttgtgtgggcggtgatacggggaagcctggctgctggagtgtccggctgtgccctggattgggtgagagggacacaggagggacgtggggcagagggaggggagaggagtagccactgtgttcaccgtgttgccgtgttccagggctgcccagtggccggattggccagactgtgttgcatcagggaggcagaggccagatgtagggaactgtgtgtctgaggactttgtgccacgtcctggacaccgaagggagtgccactggtgtgtgagtgatggagtaagaggtgggctgtgttttggaggcccctgggtatgtgtggccgggactggaggccagggactggctgtggtccagccccagcatgcagagaggcctgggacattctgtgtgaggggaggcccctctgtgtgggaggtgcacagacttccaggactgaccatggctttattgtcaggatgcaggagccagggcttggcatggggcaggtgtgggggatgcagagcagggccagcaggcaggatgtgctgatgggggcctggcgtgagcaggacggtgcctcccagccctgagccgcagggagtgggccaccaggactggctgggggccggggtagggagggccctggggagggtggacatctgtgtgggtcttgaacataggatgcccatccgatgtgcagggccagctattggttgggcagtggggacatggcctggggtctcggtgggcgatggcctggaggggccaccctgagcaggacatttggaggagtgctggggtgagtcagacaggaccatgtggtggttttctccagtgcaggcagtggagggggaaggcggagctttgcaggtgagggcttgaggcagttccgacttcagactcccccccagggagactgagggaccaccaccatcattactcaggccaaggaggcccagaacagggcagacggggctgcaagagttcctatggcgatagttgttggggcacagggttggtcggatttgagggagggagggtatgaatctgggagtcgttggtgcggttgtacccaccttcactttccgtccccaggctgcgcctctcctgagctgccgcattctcccctgcacctgtgcgtctggccctcttcacgtcctcctggcctgctgtctgcctctcccctgcacctgtgcgtctgtccctcttcatgtcctccttgcctgctgtctgcctgttctcagagcccctcagccctcaggccttcatctctcctggcccatcttcctactctgacgctgacatgtagtaaaagtctgaagacagagaagagtgcatgtgcgtttagcataggaggggcagctttcagtcagtgcagcaagggcatgtagttgttcagagatggtgctggaac

SEQ ID NO: 45 - DNA, 3550 bp, Homo sapiens
ggtaagggagatgagacctccagacaaccaggaagaggtgagaatacctccagacctcagggggttgagatgagaactttggacacccagaatagaggagatctcatgatactctagcagaggagatgaaagctccatgccatttagacagggatatgagactatattcaagtagagggtaggacatgccctggcacccagatgggggcaatgagatctcccaacactctggtataccggtggagacttcagaacattcatataggtaaaatacaacctcttgacattcagctggaagatgtaagacctcttgattttcaggtagagaaagtgcgacagggtgacacttgggtggtggaggtgagaattcttaacctgtaggtggaggcgatgagggcctctggcactgaagtggaaaaacagagttgttatttctttcaaagaaggaggtgatcactccctgatactgggtaagatatacgagacctattgaacattcatttgaggatgtcataagtacgacattcagttagagaaaatagataaatcaagatcatctgataatctgaaaactcaacactcaggaataggagatgagatgtcctgacactcaggttggaggcatgggaccttctgacacccacttagatgatgtgcaacctattgaccctcgggctggttgagatcttacattcaggtagaagaggtaaggctgccctcatgcaggtaagagtgtgacctcctgacacttgcaggcgatgggaaatgttttaacattcaggtgtttgcaataagcatttgtcacactctggtaggtgagatgctagttcctgatgatcagatgggaaaaatgatgcttcatgatattcaggtagctgtatgaaaactcttgacattcaagtataggagaaaacaccttgctccacctcagtcacagaaagccgatctggagacattcaggataataggagaccttgtgatattcagcaacggacaggaaggtgggctttgcagttgtaaattaggaaaattcaaaatgactcttggaaaagtgtgttgatagcattcacttggaagaggaaaagaaaacttccccaacaacaattaaggatcaattaatctgctgaccctgactcctctgatccacaaacatgttgcaccgtctcatcactgaagggctgagccgctcctcagtctgtgagtctgcagtggtcacagcacgcatgagaggcagactctgaacctgcacaaagccagagccttgggtgatgtggggacctcgcaagagttactgggaatggagatcctggccttgggacagagggagtggggctgcacaggagtcccccatcatcctggtggtgggggagcctatgcaggaagtcaagaagtctcttcagcacaaaccagttaaggcgaggggctcttacctggcctgactgctgggggtggggtgggggtcacccctgctgattggccaggcagccacggagctttgtgaggtcactaggcttgcaggccaggcagtgccaggagtatggttgagatgctaccaactgccattctgctggtcttggcagtgtccgtggttgctaaagataacgccacgtgtgagtaagtgtcggggcaccttggtgggggaaggatcttctgaggagcaggtaccaccccgactccctctgtccagggctagggaaaaggaggctgcatccctaacctggaccccccctgctcccagaatcagcagcctggagcccccagaccctcagctttcgtggtttcctccagagatggacccctcagcacctcaggctccttgtgcctctcccactcccccagggactgaccccactgtcttgaagacatgaagtcctgattttgggagcccttatccccccacagacagctgtcccaacccgtggttgcccccaacagccccaggatatcatcgcttcacaccgcttgcacccctaccccccagtaggctctctcactccaaggtaccccgaaataccaacacctcccaagctatatgtggcctcccacccgtgacacagttcccagagcctccacctctagacctccactgctctcagtgtgccccctacacctgtgggccacagtatctgcccctggctgctatccctcctcccatcactgtcaacgacccccttcatcacctgacttccctgagtctcccacccaagattggttataaggacctcaggccattacacccctctgtccccaggccccgcatccccacctctaccctcctgttctgcccagggacgggccatccctcagggcccatgcagcctgtcctggcttcctatggcctcctctttctccatctgtgactgcacccacaagacctgagaagtcgtggccccagaaccatttcctagagcctgcggcttcctacatagcgcaggctgcccctgctttcccagaacccggaagctcttccccacttttcccaaccccatgtccctgcctcccctcagttgtggagttacaaggacaggctgtgctcatgccaggtttgaactgtgctctggtctctccccagtggcccctgtgggttacggttcaggcaaaacccacagggtggtgtccgcatcgtcggcgggaaggctgcacagcatggggcctggccctggatggtcagcctccagatcttcacgtacaacagccacaggtaccacacatgtggaggcagcttgctgaattcacgatgggtgctcactgctgctcactgcttcgtcggcaaaaagtacgtgtagggatgcactgagggaggtcttcagaacggctcttctcagagaggggcgttccccggggatgctgtgcagcgtctccctggggctctgggccaagtggctgcaagactccgggggctggtccagacctttgctaggggaaggccctgagggtcgctgtcaccaggcttttgtccagccggttgtgacctggcttacctttgtgcccacagtaatgtgcatgactggagactggttttcggagcaaaggaaattacatatgggaacaataaaccagtaaaggcgcctctgcaagagagatatgtggagaaaatcatcattcatgaaaaatacaactctgcgacagagggaaatgacattgccctcgtggagatcacccctcccatttcgtgtgggcgcttcattgggccgggctgcctgccccactttaaggcaggcctccccagaggctcccagagctgctgggtggccggctggggatatatagaagagaaaggtgagtatgggagcgcctccaaggggggacgctgctggccattctcctggtggtctttgaggtgcagcggtcacttgttgacacccagccaggctgctttcatcctcctcacggcgctacacgtagagccatcactgtggccttccacagtcccctgtgccaggtcacgtgatgggtgactcgtctggctgtctacgggggggctgacagcaggtgcaggcagagcgcagcgttgcttagaatggggttgaggctgtgtctgtatttggca

SEQ ID NO: 46 - DNA, 2653 bp, Homo sapiens
aaagacaatgcaaaaaacactttacatggttaggagcctgctgtagtcaggcttcattttaaaaaattacttctgccaaatctctgccagttttataaaaatttctctaaaactcctctaaaatacctgataatagagaattccagaatgaggagagagataattattttctttttctccatattctctgctcctaaaaatagacaagtctcctgttggatcctcttgttggcctttgcacatccactagtggtttagtttgtgttttggacaagatgctgttcctcccttatgtgaacctgagccagtttctaactgtctctccccctatattcctcactggtgtaagaaacagggttgtggtgcaaatgaaataaggcttgggattcaaactgttcagcatgatgattggtgcatagcaggcatctttcagtcttagctattgatggatcatctctgctttcaacattcttgtttttgttatgattacttaaaaagtattagttcattatttcagtgaattaatacacttaacattgatcagggcactagaagattcaaactaaatgacaatctatttctattagtctctcttaagtgatttactatgtgcaaattgctgagagtattaattttatgtcagtgcatttatattgctgattattttggaaagcagacatttgattgtctttatttgctcttttattgcatccactttctttaaactcaatgatagttggaaatagaaaattatggagaagaatcatcagaatcttcaccccaggacttaattccaatccattcaaaaataaatgtcaaattatttaatggatttaaatgttgaagccctaaatcaactactgccctatgatggttgagggttctgtaaacaaacccatgacatccttgacatttcagaagacagataaccccatctttttctcagggaggaaaacttttacaccaacggctcctaataactaaatggaagaccaaaccatgttaggacgctccgaaattcagaatctatggattatttctggaaaatccacctgcttatggcccatgaactacatagaaatcccctgcccccatttgtatatagaaatgtgctgctaataagaagagaaagagctagatctttcctgatgagtgttccccacacaagggcctttagtggtcaaaattagggcttttatagctgcagtggcagaaaatgcatacaaataacacatttgtcacctagatggtcaattaaatactcacatgaggtcagtgcaaaactgtttaccaaacagcaccaattgcaacttgtgagacctgagactacaggactcagtgatattttaaggattaaattataatcaatacatgcatttcttaagttttgcacccccttgaatgtcaactacatatgtttttaattccacaaatatttgatgtcactgactgcgctaagagaacaagaagatgaaggaaatgcataaagtattaattgaactgagccttaaaaatagctacaaaatacatattagttcaaacactcattaaaatgagaagagttaaattcagagaacgacatttcccagttatgatcacactccccagtgcaaggtgttctatagcaatgtttgcctaacggcatttggttgatatctgagcactagcccataagaatgttactattgtcacttctaaaaggtaagctttaaaataaaggattggcaggataatgccttgagatgccttcagtttcatgactcaggacaatacatatctacctgaagagacagcctgcctgaggctgtgagggcttcaaaggccctaagaccgtcagagccacaggacacagagacagcatgaggtcaaaggctgacccagggtgagtggtgactgtatagaaagagtttaacactggcccagaacagtgtgaagagaagtttattagccctaaaaagaagaagatccaggtggcgctcctctagagcacaggtaattttagtctgaaactaagggagaatcatgttaaaataagcaagagaaatgtgttgggcaatgttcatgactgcaatgcatgagtaaggatcttggcacacaagttaaactcccttattttgttttgagcagaaacatcatttagcaagtgccaactctgacagttttctttgaagaatgtcctggaacgtcccatgctagttaccataatgactgaaataggataccacaaaattaagcaatgagagaggaggggatattctgatgaaaagtggtcaaaactaagggtgaaatgtttttcagaataaatgacataagattttatgggaaaattctggtgacttagaaatattatctgcattacaaacagaggagaaggatcacatcatctattctgataaaaagaaggttcacctgcgaacatttaaataattcaaattttatggacagttctaggtttctggaatgtgggaagacccctttattctttcaaattgtccaattaacaccaaagtcttccataatcatcataatcatcatcatcattaatgttattgactgcttaccacataactaggcacagtgcatttgatataactatttatttctcatcatcagccaccttctgtagctctctgaatatacctatatcaggcag

SEQ ID NO: 47 - DNA, 2093 bp, Homo sapiens
ttgtgatacaccattcactcaccatgtgactgcttacaaagagggaaaaaatatggagccctctgttccaagggaacactcctttccccctcccgacacttcctagagatcttagacccacatgactgtgagaaagaagagtgatgtgagagtgaactttggcaaggctgaagtgccctggttttgtctggagcgagaataaaagtgagaggaaggaggcgtccagttggctgagaatactgttggctaagattctttagcagggtgggcttttcggatgcttttctcctctgatctatttaggtttatccttactcttttccatttatctgggaagtgacttgggtttaagagaaccaggagtatcttagcagagtcaaaagggccacggtgaaccccaaatgtcaggaaacaaggaactgactagattactcaaggcttcactcttgaggagggagagaaaagagctcctgcatttccttctatttattgattacagccacaaatggaaaaggaagcaggctttctgccctgaaataatgatgatacatcgggctgcagagctcctatacctataactctcaaaagcaaatggaaaggagactagcgtgtggctagtaccattattctcacatcttcctgcagtgttatgagagcacagagtaggatgcagggtgaggatagacagcagtagagctttcttgagctgcttattcctctccaaattctctctgaaagtggatgaagaactgctgccatgtctggtgtgggttcaatttgtgctctcattgcttctacttctctgtttctccagatcctaccatcacgttcttccttctgtggcttagccatttttctctccacgcttaggaaccatacatactatcattcttctacctctgaagcattatcccatccttctgacaaacatgagtagatgttttcccctcacagtcttgccaaaaagcacttataaagtattgcaccgtagttttcatatttcaaaaacacttcaacaggcaaaatgcgatatacacaaccccaaaatgctgtgctatgatgaatttagttctgtattggtaatactataaattgcttttgaatgaaagatacaatgtctatatattatttaatttgatacttgcagtaactagctatttaagcaagataggtatcagtcctctttagcgaagttcagtggaaccaatggaacaaacgtgtgggagtggaactggaactcggatgtctgattttgtcttaagttattttaatgacaagtcatttagccaccgataaaaagttacttattcagaaaattcaatcttctggacaagttttatttttacatgacataacctaaaatgttatatatgttaaattctgccgttttagatttcaggaaaacaaatgcagagtggtagaggctggtggtgagaatgagctgagaagggtggtaataaactgaggtttctacaacgagtttgcattaaaaaaaacttgttgggggttctggaacccaatcaattctcagatgtttccatagtctatttttatatagcataatacatttttattatgatcaggcaataaagcaagactgttcaccagtcttgctttagccatttaccatttcctatactctatgtatgtcctttgtctgcttttacactaccataaagcctgcttcaactttcccctcaatacactgagatttatttcttcactcaccattctggaaaattccttgttcagccttctaatcactagacacctgcaacctttccttcactggatttctgcctcgaacagtcactcttctccactaagatctacatgtcaccgctaaaatcccctttcttgcttgtcactttgaccatgatgtcacttacttcctgaaaatttcccctggctccctactgctttgcaggccaagtaactgtcacatttcgtttccactttcagctggagtcagccttcattattcccctctccgtccctgtatccttagagaccctctcctttgactcaacagctcactgctcttgtcttctcaaagctcctgtcttttcacacacagttcctgctgtcttttg

SEQ ID NO: 48 - DNA, 2953 bp, Homo sapiens
gtggtaaatgcacatctatccctctcctgtccaggcatgtggggcctcgttaacaatgccggcatctcaacgttcggggaggtggagttcaccagcctggagacctacaagcaggtggcagaagtgaacctttggggcacagtgcggatgacgaaatcctttctccccctcatccgaagggccaaaggtgagtgggaaagggagctccctcctgcccctgaacctgccccacgtgttcatctttgctcagaatggaaatacctgtcccagcagctccaatgtccacaactcagcagaggtgagctcgtgaatcccagggactatgctgggcctggggtgatggtgggcagaggggctgtggccgggtaggggaggaggaagcagagcaggtaagaggtcagtggtccatgcagcaaaagcttaaagagttgagcagccatccactctgcacacctaatctatagagagaatcaccctttgcacaaagctgtgtgtacacatctttgtatcagtcaggtgtggttagtaaaatctggcatattcattctatgggttatttatatcgtagtttaaaaaatgagatcattgtggtattagggaacgatagtaaaaatcaagattagaaatttggaaaaccaacaaaacacccaaaccatgtgggtggccaaatgtgagcaaaccactttagaagtcattgacttggatttttttctctggcatagcaaacaattgtggcaaaaagggtaagatccatacatctatggtgaagtcctagcaacaacaagcatgaacacagactgcagctgtaggattttagatggaaaccccaacccttcagtgacttcaaatttagagctttctgaaaggtgcctcccccaggatgggctgagttccctcccggggacacacctggatgggctgagtgccctcccggggacacacctggatgggctgagtgccctcccgggggcacacctggatgggctgagtgccctcccgggggcacacctggatgggctgagggccctcccgggggcacacctggatgggctgagtgccctcccgggggcacacctggatgggctgagtgccctcccgggggcacacctggatgggctgagtgccctccccgggacacacctggatgggctgagttccctcccaggaaaactggtcccagatccgcctcggcttcccgggctgggccaaatgcaatccacttccaacccctctgttcccagggccaggaggagctgtgggaggcccctgatgcccccaggctgggcctgtggcctttggagggggatcaccacactctcccagtgcccaggactctctcctcatatcctagccctgaagtcaggttcagaaatcctgcccctgcccctgcctgctgctctgtttgccaggcggtcctggtctccacccaggctccaccctaccagggtggaatggagttggggagttgggcctaacagcacgggtcctgtcctctttcagggctgtcccggggctccctcccagctgcagccccaggtacttcctcgtctgcactccaacccccatcgccagggctgctgtcagtggctagacacttggccctagtgtgctacttatctgcacgtcgtactactggagctggactttaagctccataaggggaaggggaagctttcaggctgtatttctccctcaccagcaccagaccttgcctatagtgaaagctcagatccacacagacagctgtctcgcctcccacttctcccctcgtgttttcaccccaaattatcaccgcatcgggcttgatctggtttttgagtcagttgcgtgttgcccattacactgtgccctgctgcttctcactcacttgtcctcccctgtcctgcctggcacagccaggttcccagggaagaccaggggtgccgatgctgatgcgtgggcctgagctggccttgcctattgactgagaaggctcctgggtggctcagaagtggttccagccaagcctctagagacatgccagacttctgcccgctgtgtcatagggcagtaacggcttagcaggtacctctgtctccctctgtaggccgcgtcgtcaatatcagcagcatgctgggccgcatggccaacccggcccgctccccgtactgcatcaccaagttcggggtagaggctttctcggactgcctgcgctatgagatgtaccccctgggcgtgaaggtcagcgtggtggagcccggcaacttcatcgctgccaccagcctttacagccctgagagcattcaggccatcgccaagaagatgtgggaggagctgcctgaggtcgtgcgcaaggactacggcaagaagtactttgatgaaaagatcgccaagatggagacctactgcagcagtggctccacagacacgtcccctgtcatcgatgctgtcacacacgccctgaccgccaccaccccctacacccgctaccaccccatggactactactggtggctgcgaatgcagatcatgacccacttgcctggagccatctccgacatgatctacatccgctgaagagtctcgctgtggcctctgtcagggatccctggtggaaggggaggggagggaggaacccatatagtcaactcttgattatccacgtgtggattatccaccatgccaggaagacccataactggttttaacactaactagagggaatgacttctttgcatagtgagtgacttgggccttcacaaacagggtgtggagtggcaggcagaggcctctaaatctcagggcaaacatggtgaatctatctctccggagataatttcatacagagattttaagaaaacatctttatattaaaaacagatctcatttgatccttaagccagtctcatgaatgaaaaggacaggtttttttcttttgtaaatgaagcatttgcagcttaaagaggatgcatga

SEQ ID NO: 49 - DNA, 1834 bp, Homo sapiens
tgtgttatcgcagcaattttataatggctcattaacccctgtgagaggccagtaatatgggatagcaacggatttctatcaactccatgagggagataagtaaggtggcatcttatgtagatttctaaatcctctactttgaaatcagctcaatggcatattttaaactcaaaatagaatgtcttctggttcctaatggttgatttaatggtggatttgaccatatgtgtatcagatgtaaaaagtattgtccactaagtggagtaaaaaatgatcttttacagaaggaaaaaaaaactgatttaaatctttagattctcatgggatctcattaaggttctctttctttaatacattgtgcagcctaatagttatcagcagccctgcggtgtgcattgctgataggttagtttacacaggattaattgtgtaattttgcaagcaaccagcacagtgaacactgatttttgcattagccccatgtgttgtttccaaggggactctgctttctattttaaggtggtgttacatttcacttcttattaattataatttctgctagcatgttttatgcccaatatgatttattaaaaatccttcataatgtttttttcctaattgttatgtccttcggtaacttcattaattttgagcactgatgtgtaaaaaatggcaggagaaaatggcattcacagaaggttctctgaccagccagtttccccatgcccccgttgataagttgccacaaatcttttgctaaaatacagacacaaattcagttgcagccactccaggtatgcgaagtgaataatcagtgcaggcaacaacctgacaatactacattcctcaaaccaaaagaatgcgaatgttcaaagaagtgttggctaagcagaactcagtccattttccacaatacgtagcttagtattttccagaaatacttgtgtattcggaagaattagaggaaggaaacttttgtttgaattttccacataatagcttagttcaatactcagctactacattttatcgactcttggtgggattatgaaatgcctattgaggtttcagtggaatctttatagctggacttgatattcttttacatggttttgaaaaaacaaaacaaaacaaaatgttgactgtgcacagtttagaacttaatctttaaattctttttgccttgaacttgaaaatcaattatctgtctgtgccccaccacctcttccctcatctcagccttcacgagataaaatttctctccctccggagcacatggtctctcaaaggggaagagtcacatctccttgtctgtgcagctgttgcttcgttttgtttagggtggatcttctctccttatccccgtgagtttctatagtattataaaggcccaataaggttctgtacaaagtgggtacttaaaatgtgtcctgagtgacaaactggcccccactggaagaactctttaaaacactctgttaccagagcttcaaaaagggcttgtttctgaaggatcaaaggatctcttgtataataaattctgagcattcagtacataatgaagagaagaaaacatgtcttttaagctcctatatgatgcctggattatgtgaagagatgaaggaagtggtgactctttctggcttttgtgtcattcacattaaacaggaatagatgaaagcaaaggcttaacactgacaaaatcccaagtaggcaggctctgcatccacagcctgttcacacattcataacaaaccaccagctgatgacttgaaaaaaatatgattttctttctagtgaaagactgactttgttttgtgttttgtgccttttt

SEQ ID NO: 50 - DNA, 2426 bp, Homo sapiens
ctgactcaagaactgtagcattgagtgtaagggtgcatcattttcataaacacagaggaaaatgtggctggtggctgatggcagagctgagtcccgagagctcagccctgagctgcctttcatctggtcaccatgttcaggggttcttctccatgtaaataaacatctgtgatgaaaacctccacaggtctcatcatcaaagtgggtcttctagaaaccaatttgctttcaaaacaagagatcgagtgataatctatctaatgttctagaaatgttggaggcaccctagacaaatgtcaatcttaaagttttccttttgccttatttctctaagtaacaccttctcaaatcatgaaagcaagagtgatctaaattttttttaaaaaatccatattagaaggaagatctattaaggatctagtgagtaaatgacacttttggaatgtttagaacttcaagggggaaaccacatgttttcacatcccactatatcatttccataaggatgaggaaaagcagtacccctatttgcagaagagagactgccgtgaagtcagtggacactatctccaggtcagaatccaacctaaaggcctttaatcaatggtaagtgctctgaggcacaaaatcctatgctcctcatcagtcatgctttatgtcctctgaatattctgaattcaccagaacctagtagacctattttaagtttctccaaaaatgtcaaaactctgttttatagaaaaccagaactttcatgtcaagtgttcctgagaacattaataacaaaagccaaaacaagtttcttaaagtctgtcagccagttctgtaaatatgacacaagtaaatacttctggacatcatttagatattaacgtaacatgcataagctagaaaaggcagcattaaatttggatgtttttgacttttgtttctcaactttttaaagattaaatcatgggattttattctcttctattccctctagggaaagcaatgtgctgatatttttctgaaagatgctaacagtggaaggaactattgaaaacaattaggggaaaatcgcaccttgaacttagtagaacgtgtacaccatgttctcacaggaaatctcagacatgatattaaaaattccagttgtttcatttttttgcagaacagtctgtagttatgtactgagtgcactgtgcagggggcacacagggcataccaaaggcttcttttgtttatgatacagattcccactgtactcggaaggttttctttcaaatgcctcatcacagtgtgtccaaacttcttgtagggagcaacagggcctctatttaagcctcttgttagccgatccaccagccaaggtcatgttgctttcccttaagaatcagagccccggggatcctgttctatctgttctttccgccgcctcctgtctttcagcagggcagatgcctcccagaagtaaaccagatgccaggactgtgggggactcttgagcagcatcagccaaactgtaggagctgagaagaggaagctttgctcagggtaagcgccctgggataatgtctttaatgtcaagaggatgcacactggaaacgtggaaagccctccaggctgaaagagggagtcacacaggtggggagtgttgccaagcatttgcgagcactctcttcggtgggcagacagccggcttgctcatgattccgccttttctgttattgtcaacaagccgccactggaaatttgtatccttaaggctttgaggtcttgcctcaggtgggggtcccggaataagctcattaagtttttgcctcattacctccaggctccaaatcactggtacaaatttctcagtctgacttaatgcttagggaaatgtcgtatttttggacccttcattttaaaaaagtatatatatttaccagtgctatctccgccaattccgaataaaccttagacttcaggtcatgagtcactaggagtctgaatatgtcttttatttggattcaaataagattttaacttcctggcaccatggttttctgaaggtgccagtgtgagacctgggtcatcagaatgacttggtgctgggaagccacagaatggtgcagtaagatcttgctgtctcggtttctgccttagaaacaatatcatacaccctctctcatttcacagaatgctaaaatttagcatatgttatagtatttattgacaataataaggcaggatagcaaagtggttaaggaatgactacactcaacaaccataacctcctatcgtgccagggacggcaggcaaataccatgcacggaagtcagtgtcagcagagatcagcgggcattctcagaacactgtgggaactaagggtctgagccatcaggactgtccacagatattccactccttctgctcatataatatgcttgcattcccca

SEQ ID NO: 51 - DNA, 1796 bp, Homo sapiens
taaacctttgttactgtaaaccaacaccctctccagggaagtttcctatgtccctcctacatttacacatcaaagccataatctgagtagtgatctctctaataatcattgcattaacagttgctcttaacaagcatctcaatttggccctattctgaaccatgcagcctaatgttctctggtcattactcatactcttttgttgttgttgttgcactctgcaggcaactccacaactactaaactctaccaattcttcctatgcctcaaacctgttagctagtcatgaattcctcttcattcagggtgggaatggcctacttggccacaatacaagaatgggcaacttctcaagcccaacttagcttcacctatcatcaggacctctctatacaaaaaccttccctctgctaacataatatttttaatacaacctaaagcagcttttaaagattttcttaaacccacccccattgattcaagccccttgttctcccctgctaccctcattggccaggcactcctatacatctgtgctactgtaaattccagatccattgtgggtgctttagacccagcacaatgcaacacaacaagcaccattattgatatttctcaaaattttgtttcactaaatattctcaacatcaaatgagattttctattctccctccaaatgttttaacacctggaccattcatccaaaatgatgcctctgagttctgcgtcagtcacccttcttggagtcaacccaacccatggtgttgaccaagccagtataaattatgcaaaaggtttcaagtctttaatttctttcagaaaatccttttctttgacactactagaaacatgcctatgtttaaaaaaaaaaaaataggacccatgtctggctcccctggcagcagcaactttagtggcaggatctcacatgtcgggtagccaacaaggaccctggtcaatgtttggaactgacctcaccttctgcatccatttttatcgactacagaactttacttcctgtgtgaaatgcaggcttatctctgtctctctggaaacttgacgagcacaagcactctggcttccttcacccctaacatttccattgtcccggttgatgcttccttgctgttaccctttactacctcacaccagatcgactaagcagtttatcttttttttttttttttcctgagtttggcatctcaggtgccactataggaatagctggcataattattgcctcctcaacttaccaaaacctgtctctggaactgactcacaaaataaaaactactgctcagactcttacagagtgacaccaacaagttgattatctcgtggctgtagtttgaaattgtagaggtcttgctgcagctcaggaaagaatctgccttatgctaggagaaaaatgctgtttctgggttaacagattagggaaagtccaggaccatgttagaggttttacaaaccaggcctgtcaccatcagaaacatgccactgaaagctagttctcttggggtgccacttggttccaattctcatgacatcccactttttggggatccctagcctttgtcttcctttctctcttttgtgagccttgctcactaaatctagtaaccaggttcgtttcctctcacctagaaactctcagacttcaaatggtcctgcaacaggaatatcgacctattttcccccaatctgcacagccatgtccctacacatttcctctggacaatgcaagttcaaccttctgggagaacatggatggaatctttttctgacaaaaagcaaga

SEQ ID NO: 52 - DNA, 2633 bp, Homo sapiens
acactgtgtaaattacaagccatgaccccctacattcttacattcataaggtatttcttccatttgagttcggagagacttggtaagctctgcctgctacagaggcatcctcatcctgcccccatccagggcattccctccctcataggttctcttctgggatgtgccactataacttcccacatatatcacatttaaagattcctctccagtatgggttcttttatgcttggtgagatttgatctgatattaaaagccttaccacactcattacatcggtatggcttctttccagtgtggatccttttgtgctggtcaaggactgatctataattgaaggatttcccacactcacaattatagggctgcttcccctggtggacacttttatgattgataagacttgagtgtgagatgtatgccttcccacactcatcacattcatagggtttctcacctgtgtggatccttttatgcactgtgaggcctgagctgttcctgaaggccttcccacacctatcacacacatagggtttctcccctgtgtggatcctcttgtgctgagaaaggagagagctgtaactgaaagatttcccacactcaacacacttgaagggtttctccccaagatggactcttttatggcttataagagttctgcttgagaaaaaagcttttccacattcatcacatgtatggggtgtcctgccagggtgggtactcttatggttaataaggcttgagtgtgagatgtaggcttttccacacacatcacattcatagggcctctccccagtatggattcttttatgaactttaaggcttgagttgtttctgaagaccttctcacacctgtcacattcatagggtttctctctagtgtggacccttctgtgctgagaaaggagcgatgtgtaattaaaagatttctcacacacatcacatttgtagggcttctccccaagatgaacttttttgtggtttgtaagggttcggtatgtgatgaaggccttctcacactcgtcacacttaaagggcttctccccagggtgtacacttttatgatttataaggctcgagagagagatgtatgctttcccacattcttcacatttgtaaggtcgttccccagtgtggattcgtttatgtactttaaggccagaattatttctgaaagctttaccacactcatcacacccaaagggtttttccctggtatgaatccttttatgctgttcaagggcagagctgtagttgaaggatttctcacaatagctacatttatagggcttctccccaaggtggattcctttgtgatttttaaggctagagcgtgagatataggctttcccacacacatcacacttatatggtttttccccagtatggagcctcctgtggactttgaggcctgcattgtttctgaacgttttcccacacacatcacatacataaggtctctctccggtatgaatagttctgtgttgaagaagtagtgagttataactaaaggatttcccacactccttacattcatgggctttcttcccagggtgaatgcttttatggactgcgaggcctgagctatagctgaatgctttgccacagacatcacacttgtaaggtttctctcctgtgtggatccttttatgcactatgaggcctgagctgttcctgaaagccttcccacattcatcacattcataaggtttctctccagtgtggatgactttatgctgaatgagaagagagctataattaaaagatttctcacactcatcacatttatagggtttatctccaaagtggatgcttttatggttgagaagtgttctacaagtaatgaaggccttcccacactcatcacattcgtaaggtttctcacctgtgtggatccttttatggaccctaaggccagagctgttactgaaggttttcccacagatgtcgcattcatagggcttctcccccgtgtggatccttttgtggactctgagcccagagctgttcctgaaggccttcccacactcaccacattcatagggcttctccccagtgtggatccttttatgctggtccagaacagagctataattgaaggattttccacattcatcacatttacagttcttctccccagaatgggtgcttttgtggtttataaggctggagtaggacatgtaggctttcccacattcctcacacttgtacggcttctccccagtgtggatccgtttgtggacccgaaggctcgagctgctccggaaagtccctccacagtcatcacattcatagcgcttttccccagtgtgcataattttatgttgaacaaggcgggaattatatttgaaggatttcccacattcatcacatttatgtaatttcttaacagcattggttttctgctgtagactagggtaggaggttccattaatgttctccacacgtttgccttgctcactgcctctctgtcctataggcatagtctggtgtgtgatatgctgtgggctcagatgcaagctcttctcagatgcctcaccttcctgttctgtctttatatttgctgtactcttggctttgctgattgcttccctgatgctgcttttgtcctccttcatcctgttttccacag

SEQ ID NO: 53 - DNA, 1752 bp, Homo sapiens
tagtgcatctaatgaatgactgaatgaatgcatctttgcctttgccttacccccgggcctgaaacatcgtcttggtccccttctcaataccttggatccttggagatcaaggtcctggttgttctggcaagttcaacacaatctggcctcatgatcagagtcctgtccctgaactcaagacaagggagggatgggcagaattacctcatgctgtgccaggaaatatgagtctcatggggcatggcctgtgtgcctgggcaaattcactgcctcactaccctgtgctgagatgatctctttttttttttttttttttttttttttctgagatagagcctcactctgtcaccagactggagtgtagtagtgcaatctgggctcactgcaacctccctcttcccggttcaagcaattctcctgcctcagccgcccaagtaggtgggactacaggtgcgcaccaccatgcctggctgatttttgtattttcagtagagacggggtttcatcatgttggccaggatgatctcgatctcttgacctcgtgattcacctgccttggcttcccaaagtgctgggattacaggcatgagccactgcgcccgtccaatctctctttcagggacagatgttcactctctcttgcagctctgcctgccagactaagcctgaaaatatctctgcatctggcattcctttaccacctatgtggggcacaacccagaacaaagtccctccaagtgtaccctactctctttccattatcatttctctggtctgagatagatgtttatgacctgccaataaatgcagtgactcaaactccagtgcccatactcctcattcatacagccatgtttagggaggctctagggagaaatgcacagtttgacatcgttcatgaagagcctctccacggctcctgcgcctgagacagctggcctgacctccaaatcatccatccacccctgctgtcatctgttttcatagtgtgagatcaacccacaggaatatccatggcttttgtgctcattttggttctcagtttctacgagctggtgtcaggtaagcctttcagtttggactgttgtttttctccttgttgaataatattttgagttcattcatgacaatgatctcagcacagtgagatgcaggaatctttggtgcttgcattctccagcttctcctggcctcaggctggaaactaccaatgccaggagctgtgggaagcacagggcagcaggaattgaggaagactccttgggctgtttctcaaggacttgggcactatcacagtagctcagaataatgggagcaggccctgggagcagggagggaacacattgagaacgccaaggtaaacacattgttctccccaggtgggctgtggggcttaggcaggggaagtctctaataaaatccccaggtttttgacttgggtgcctgggtggaaggtggcactgtttaggatgtttggagaaaaagacaatgtgtccagttatgcacatgctgagttagaaacacctgtagttatggggtagagcaccagacctttaagtgaggagtaagttggaacctggcatagtctaggcagaaacccactcttctttctccttctagtaaccatcaagacaaagcctggtgtataggatattcagtaatcaaataaattttgcagggagagataggggctggagtagaacactggattctgggtggtcagtgttaagccacaaaaagttcatttgactg

SEQ ID NO: 54 - DNA, 2795 bp, Homo sapiens
ccagccccacctgctcaggcagcctctatggcccctgcacgctgcccccagggccaggagcaaggttctaccttcgccactctgcctcccaaggcctccccaccagcccacggtctgacatctggactgttgccataggcccccgttttggctgctggctaacaggacagcgaccacccaccaagacagacatccactctctgtggccacgccctgctttctctgcagctcggggccaggagcactgtgactcctcaaggcaggatgaaggctgccgctgtgcctgtgagctctcatgtcccaccgctctgcccgagccatggtctcagggcactgcctggagctcctttcacagaaagggtcagatgcccaagggggcccgtagggcagcagcgggtgggtgaagccagctaagcagggccttccagcacacaaggatgtcggccccagggcgggcatcttcagagagacccagagcatcgaggctggggtgtggagctgccggtgcgccaccgtgggtggtgtcaagcagaatgcatcttgccgcgagatctggcatctgcactgcctgcttctcctgccgcaggctgccacctccctgacacagggacccagcccagccggtgttctcacatgagcctgggggtggggggcggctgttgtctgcccctccaggacacatgtgcctaggcctgagcccctgcttggctcctgccgcaccctgtgggctcaactccgcacagggcagctgttcttcttgacattttccagataagtggatgtttttattctggaatttgggagcgacctttatctgctgtctggaaggaagcatctgtcaccagtgtaaagcctcccagtctcccagggctccactcggtggcccccgcatgctggaaccagtcctcccagacaccacggttgggggcagggccggccctggggtcaggcaacaaccaggccgtcagctactctgggacgcagcccaggccgggaggaggcagatgcaggcaccacgggacctgggtgaccggcctctgttcactcctcccatcccttggtgcccggcacacagaggggctgaggagcgtggagaagggaggggcagggagcagccggggcaggggcctcccggctgggcctgaggaggagcaaagcctgcctgggacccccaggacccccaggatccctcttcactgccagcctggccatggagaggggcccagtctcccctggagcacacggtcgcccgacggctggtcacaatcgggtaggcagcgtgtcctccctctccagtcctcaactacagagggaggactcaaagtgggacaggcagacaatcatccgcccagggactgtgctgggaaggagggtgtggtctcaaggagggaggcctgggcgctgaggcatttccaggtaggaagcagacaagctcctgggtgggtggaagaggcctcccctagggcatgtggaccccgggcaaatacattctaaggcgggagtcctcgtttctataaactatcaggttttcctaaaatcaacaagacagcaccatgctggccgcccaacctcacgtgatccaactaaaggaagcccacacaggctagcagggaaccatctgttcctaggccccctttccaggactggaccccagccacacagtcctcacaaccaccatcagcctgagttccaaagctccttcagacatgcaaccaactttccacactgggcatggggccacacagtgctccgtggagaggaacaggggccaccaggccccacatggttccccactcaggcttggggagctacccctcggcacctttggcagtgctgactggtctcaggcactggagggggtcttggaatttctgagaacggtattccaaactcgggggcccaggatcccagggcagggcacccaccacccaggtctaaagcaatactgactacaaagaccccaggtgacaggaccgagggcatcccaacccttccctcccaagagccagggctgagccagacacaagggacagaggaagggctggcctgggatgaaagggacactcaagggggcagctccctggagcctggactagccacccaggctcaatctgcaggcagcatcaccccacacaccccagattccaggtggtgcaaagctcagatgctgccaccacctgttccccgtgcccaggccaccccactccaggccagggtgggagccaggccggcctcctttgccaacctctgggcccaggcagactccttctctccgagactctgctcagaaacaccagaggctttctgagcctatccaagaccagatggcgttcatctctcagtgtcaataaatcggacgtctccagggaaatgacttttacttggtaaataccaagcaagaagagacggcggcgcgagcccccagtctaggagaaccgcagccagcaggcagccacctattgatttcatctccctccaaggccagggtgctgcagggaggagcagcttttcctccgacacgactgcgcccgcagggacaggaggagcagccgtgcttctctccagctgcatgaggcggtcttgcaggggagagacagccctcccagaagggacctcggtagggctaacggcagctggcacaaaaatccaccaccaaaggtagaaggagctgcgccaggctgttggcagtgggaggggagagagtcctggagacaaggaggggaccaaagggaaggcagcaatccagatggtcctgcggggtcggacagggctaagacaggaggctgtgctggctg

SEQ ID NO: 55 - DNA, 2661 bp, Homo sapiens
aaaggacctctttaatgcttatcagccacccctccgcccttggctgtctttctggtatcagcatcctcctcctcctccctcccagactccaggccctgggctccagaaggtccatccctgtggcctcaaggcaccaggcacatccatgccagcttcatcctctccagtgacacggctgtgcagctgtaactgaaaatttaacagactgtccctctgactatttctccttcactttcttgtagcaaaacaaaaagggggaaaaatgcatcccaggggtttccagctgccaccttttcaagccaccgttaggctggccaacccccgccagtttcctcccatcctcctgggatgcctgggggactccatcaccactttctagaaactgcctatagtcagaggtggcctggggctgcccacacaggcatggagacgtggaggacacagcctgatgctagactgcacaggaccctcttccgccaggttccccggacacctccatcccctcttcttgcaatcatgtcattgcatggtagcgcctgtgtcctaatgttcccatgccacaagtctggagcccttcgctcctgtctcccgaggccaggattgagcctgcttggcccagaggagggggcagtaaatgtcatggacagaagcagtgatgggagagtggttaatgtggagtcgtcacagtgacacagaggctgaggcacactgtctggcacagcccagctaggcgctgcccacagctgagcttccagaggacaccttctgtgtcaccatattccaggattcaaatccttccagtctgggacaagttccatggggtgccatgaggctgccccagtttgattttaaaatgtacagtgaaatgcctaccttggtggtggccaagccctgaccctgccaaggacagtctgggagaggcagggccagcctgaatgccctgtgctgatggacacacaggcacaacacccacagctcagggagcccgctccagcctgccgtggagcccagggccaggtggtgagccatgagcctgctcgggacagtccttcctgatcctggaagggagcggcccaattataacagctcccggccggcaaggctctcagtggagccgagcccagagagaaggcctgcactgccagatgggcgagctcattagaatgggagtgtggtatttcttatgcaaatgagggcaaatacatccatgggagaaatgtgaacaacagacatgcacaggagcacggacttcaccgggtttcaagaggagagggagctgggacgggagaccaggagagatctctgcccccagcactgccctgcagtggcctagcccaggccttctggatctgcctacatggaatgctcaagagagaaactgaggccccaggggccctgcatatgggtggaggctggcctgacctgcatcctggaacagagagctgcccgggcacctataggcaggcaggaagtcactgggcagagggacaggtgcaaggccaggtccacaatcctggccaggctccaggggagggagatgccccagctaatgggacacgggccagatgtagactgtagccaagggacccagaacagaagcaccagggcccagttttagggagcacccctcaggaggcagggcttgtcctgcgcctcagagactccacagctcagcactctgggctcacccaggttgggttaccggtcagatgcacctgctccatctccattctgccacatcctatgacctacagtccagatctaggactgggctcacaccctctgagccctttccccggcatcctgcccctcagggtcctgcaagcccctgctcctacacatccacagtaagccccttgcctctcccatctctgcccctccctgcctcacgcctctgcagacctcagatctctttccctgtcccttcccagtgcactcgcggcctgctcaccctgcccaccatggccgccttcagccccctctctcctccctggcagctgcagctccctcaaggctgccgccctggcccttggtctgtgctgccttccactgaccagtccctttgccccccaaccctgtccaatcctcaagttccagcatcctcctggggctccttcccactctccagtgacctgccctggctcagggcgcgcagggccttctcagcactgtcatcgctgatctctgcaggcatcgccctctgctccgccagctcccgtctgtccaggttgcaccatcataacccagacaccaacaccctcaaccaggacttgcagtccaccatcatgcccgtccctgctgaattccactactgtgcctctcgacacgctttccactctcattaggcaaagccctgggcaaagccgaaggcctgggtaccccacctctgccttccagcaccctctgcaggtgaacagacaacacccaggccaggcccagggtcatggacccataccttagaacccctggcaggcacagggaagacacacaattgcctgacctacccccggtccctcccactctgccgtcccacctggcgactgaacaccctctgctctgctcagctcccaggacctaacagccacacacacaacctcagcttcggacctggccgcccagctcactgcaacaataggagaggctttccatagctctcaccca

SEQ ID NO: 56 - DNA, 2189 bp, Homo sapiens
gaactaactgaaccagagacaatctgtcatcctgttggcttttggactgcctgttatcacttgtcctaaaattatttatatcttttctttataagatatactaatattccttagaaattccattgaatgtaaaataaaacaccctaaaattccaccaacagagggaagtaggtgttaatcatttttagtaaatacccaaattcgtctatgtaaacatgaaaaacaacaacgtatatctacatttactgtcatggaaatgacacccctgacgcgccgtttccggagagagacagggcgcagagcggcaggtgccatttcccccatgtgacatcactcacaaatacacagtgtcatcaggagattatctttcggtgataaaattgttagctctgggttgagagaaggtctcaagattcaaaagcgtcacccccaaccccctctgacctcactcacctcacactgcaacacaccccataagatacactgccccacaagcacactcacacaacccacacaaacactggcagtccccagggtcaagagctccacaccccacgctctgaccctgtccctcctcacagatctgtcctgatgtgcatgctctgtgggcaccttgcctcagacgcaatccacacaaaacctctcacccccatccccttctgcagaaagcaccagtgtgcaaaaagcatgcagaattagaaagaacagaaaacgaatgcaggtaaagcaaaaacaaacaacaaaaactcaggatacacagctcagaagaaagcaaatacaagaagaaagattgagtccacgtgggcgggctgggaatgcccaactgtgcctggcagaagaccaggccacttgctgctccggagccacagggagctcctggagagcctctgccccgactccaggcccccagtgtgccaagcctccaaaacgcccttgcgtttccaatccccaggcaaccttaggcccctcacagccccaaccaacagccagtgcagacgcaggtcctcgggctgacatggccgtcctgggaacagcgggcgcaatgccggggttgcagtgactgacccttccccggtaacaccggcgtggacgcccggcttttcgcgcattacatgctggaaactgttcacggtacttacatttccttacacggcactgcaagatgcctacgttttgtgattcagtcacatcgcctacagaagccatagggaggcgggggaggccagacaagccgcagtccagccttccctggggcccctggcaactgaaactcgccacaaatgctcaaacatgtctgactttgttcaaagtgttaattttccaggcctttgcacaggagttcatgtggcccaggagcctcatttgcacagaagcatggcttcgggtttgaagcacaggcctagggacggtcatctgtccactcccaccccagttgcaaggaaaaggaaatctcccagaagccggaagtggccgggaggcgaccctggtcctggccagagctgtggtctcttccagagttgatgccccccacctcccagcgacccccgcacaagttgcccctcctacctgagaggcttaggtgttaggtgtgggcagagacttccccacagatgtcaggccatgaaggactgcatatgaggggcgtgcctgtgaacacgaggggctgcctatgaatatgaggggttgcagatgaggggctgcccgtgggcccggcggtggggggcgctgcctggcccttcacgttctgcaatattcatatggacctgacttccattaccctgggggtgcccgggccacggcggccccttcctcttcctcctcctgggtggggtctgcagtctgaccaggcccctctcgcacacaggagcgtgggggctaaagcaagtggaaacagaataaggcaattggggtttggggggctggggcggtttttggttgttcgtcctggacgtagccacagaggaactgctttctaggggactcaccaactttaggggcttccctagaaggcgcgggagcgtaggacccacggggcgctcagcagtcgggccagggttccagggctcccggttccgcgctctcctcccgcagcgccgggcagcaggtgagtgtcccggggagcagcggatctccggcgtccccaggcgccgcccccggtctcagcagctcaaatcctccctctggaaact

SEQ ID NO: 57 - DNA, 2554 bp, Homo sapiens
ttccttatgacttcaaagcccctctcaccttctgtttggtcttttccatttgagaaagaagttcacaagtggctgttaatgaattattttcattactaatatgccactcaaaagggctgaggcttctatttgggcaacttttactttgtatcattgcagatgttgttactcttgactcaagaaacactaattactagtaatgaatacagaaaggacatctatcaatgtagttatagagaccagagaggaatcttagaagtagtctaactcaaagagtgaataggcagaatagccacctgatatggaatcactttatacaaatcctgtcacctcaatttggacattgagagctttggcactaagaaccaagcagagttttgtgtatggtcctcataattccttttttacccaaagaaacaaaccaatattagctatgactttggtaaggttagtgaatccatagctcaagagcatttccaccctacccaaatggattttgatgctaacaaatccttttgggcagggaaggacatttatctttaatgcttatatccattttttctaacaaatccacaaaccaagattaaacagtaaagactcctctcataaagtatatagtcaaagactttaattactagaacaagaaaggaaggtatacattatttaaaataacaaaagttaacagaggcactaataataatgacataaccacactggaggtggagagcagtgtagatatcctcattgtcacagaagtcagtcaatagaccgtgtctgaaaactaggaaacagaaaaaaacaagacagttccttccagggaactagccccaaggtgaggcaggaaactgatgattttcattatagggtacccttccatactgccatgttgacccatgtgcacaaattaccttggtgaagtttttaatgtttaaaaacaatcatggtgattacacactaaatggtccttatttaaggtcatacctggaattccaatattctcttggcaccacaggggcaatctggaatatccttttcttgaggaatattttcaccagaaatccagatgggggcaatacctctgccatatctaagaatctaaaatcaatgaagatcatgttcaaataatcaataccttacctataagttgccaatggtaacatgctatctactccatgaatgttcctactcttgatgtagcactgacccaaaaggcatgtcacagttcccccatcagacctggctgtaccagtgtgccactaatgccttctcaatcacctcaaagtgattatttcagtttatctgactcagagggcatcaaaatatatctcccagatgatgcttttactacctaatgttggcaacttaatcctatgaatatattgtgaagggactaagaatgagcctctgctctaattgcagaattctgcccagagtctgtgcctaccttcatagttaaaaaattttaggagggacaaataccaagtgaaacatagtgttttgaaaactactacaaacataagtaaatttcactgtaataagcttcctacagcaactgagtggttttctgtattttgtctaaaagcatatgcattgctaaaaactgccttagtgtttaagacctagatctattcttcctgtgtatttatttgaaccagtgactggtttatgggagtttagttttctttcgtgatttacgtttatggtaggggaggttaaggagaaaaatgttaacatgtcacattttacaagccaaagttacctgttggaaatgggcaaaaataaccttttttctttctggcgggggggccaatggtgcctaaacctcatgtaccttaggcaacatctcattcatctcccatccctgatgcttgctttagaaaatgaaccctgtatgataaacagtataacctttagtcttttagtaactattaaatggatcagcactgcaaaacacctttctacatggcccatctgtgtgaggaactcctctaacaagataacaaaagcctgcttttataggctcctaaggaacagactaatgttactatgaagttatttcttacagattatactcataaaacatggcctgaagagaacacgatgaggagctatgagctccactttacctgttctggttcaagggctatctgagttttaaacttctgaaaaattttatcttccctggattcatgttttgccatggaatccagttcttcctcaagtgcttcacctgaaaaatcaacgtaactattatgaaaaacaggagtaatccccacaacttgacaattcacacatggagaggggacccacttttaatcagatagctttccctatttattcactcattcaagttggaccatctgaatttccaggtactccatccaactctattatatggacttccatttagtgcatctccttaaagcttcaaaataacagaatggtcaagggcttaggactgcccagcacatcacaggacacccaacaaatgtgagcccttatcattagtatcctcagctggtaggctcactcactcagtcatcaagtgttcatt

SEQ ID NO: 58 - DNA, 2599 bp, Homo sapiens
ctatcttcatctctcttcctataccccccattgacacgtgaatcagcgtttctcagaatactgcaggtttggagtgtgtgtggcggaggagggcggagcagcgtggaaggtggagaggtgggcggtgtcggggatatcagcagggcagtgggcattggaggggtgcccttggcctcagccacagggccgttccagagccctgcgtgggcgaggccagggcggcgcgtgatggtgccctccgagaagcactgggaccagcaggaaaggctgcctgccggtgcgcaggaaaagggaagagagccggggaattgctttttgacccgtaagggagcgtttcttggtggatggggaaatcaaaaaattgactacggtgtagtcagctacatcgtgtaccaattttcaaataccggtgagatcagtaaaaagagaaagggaaggagatcacagatagcatgaaaccaagccatcaataatgaaagtaccactggttactgagcagcgtctgcttctaactgactttgctgggggaggggcgggacaggtacaagcaaaaacagcaacgacagcgcagcagttgcttcatgtgagtaataattgaatggtacgaggctcttccacattcatgtattgaaggcccaagtgcggccaaggtctccctggttcctgaggtttgtttcatgctgggttccttatactccagatgtcgggagggaccctcaggggccgaggtgcccacacctgtgctccctgcatgacagacttcctggggtcttggctcccagtctgtcctcatcctctacacacacccaaatgtggaagtcacccccagcttgagtgaatcccacaccctcagaccattggccatgatattacgtgtgttgcaaaatatcaaggattcagctgagaggctctcgcagtggacggctcagaggccgagtcacacactgcccaggctttccctggggggccctggcccgggggccccctgccttaagatgcccttcctctcctccctcagtctcccactgtcttcaactcgggccctcactctgcttatcatagaccccaaaatgcctctgctcaaacaaatggcttgacctgttagcgatatagaaaagtgagcggatcctttgaacatgttcgtttctccttttctccacccaccctgcgccgtttcccatttctctaagtgcctggaatgtgtggagagtctcctgatgatatgatgccagctgtgcccagctccctggaacacaacatagggaattaaccagtgtgttcctctttcctccgttagtgaaaatgagtactatttaataatgcagtgacacaggatttgttgctgttgcagcacttgcatggccatgctcaccttcacaccacgcggaggccaaaggcattgttccctcagctgcggccctctcccctcagcagccctggccattccaccatggtgtagtcctcctgcccttctccatccttctgaatcccattctgccagctccagggctgcacgccctctggaatgaccacccgcagctagcccaagctgctcctgctgtttattttctttgcactttgtttaattatttcccacatcttggtcctctctccttgatttcagatggattgctgaagacagagtgtatttgtggctccgctcaggctgtacacagacaggggcactcagcatccgtgggtcgtatttcattctagggccaggagcgcgggctactgcgtcagtgggaaagacgtggagatgagttcatatttacctatttcatggtgaaatctgcaaggtccctaaggcaatggctttcttgaatggtgacagcaactgatgagtctgaaaaatctttgtgtctcacttaggatttttgcacagctggtttcataattcagttattttgatacaaaagcgttctgctctaattagtaaaaaaagaccaggcgatagtgtttgcctcttgttaggtggctgccccatccatgcctttcatttctggagtaggtgcccaggaaatgtttactgagttgcaccagtgaatgaactcatgatgccgggattagaaggggaagcccttggagcctccttctgccccagttctcagcgtccctggtgttcagtaagtattagctggtcagtggagtgcaaggctgctggggctgcaggcctcggcccatcctgctgcagggcccagcactgaacacctggacagacctggggtctcctggagcaggctgagccatccctgccaccattcagctggctgccctgctgcactctgaggcctgactgcccctggctccctgctcagaatggctgagggctcaggtttgggtggaccaggcctgctttcccccgaggcatcagcacgtaggtgctgcacacactcagctcccagcacatgcagctggagggcccaggttgcatacctgaatgtgaagcctggagccacacaccccgcaggcagccaatagagtccctccagcccagcttctgctgcccccagctcagtcacactccagctaccctgaagtctccccaggcagacaacccaggcctgggagtgagtatagggagggtgggtgtgatggg

SEQ ID NO: 59 - DNA, 2347 bp, Homo sapiens
cccacagtaggctgcaagccgaggaacaaggaagccagtctgagtcccaaaacctcaaaagtagggaagccgacagtgcagccttcagtctggggccaaaggcccgagagcccctggcaaaccactggtgtaaatccaagagtccaaaactgaagaacttggagtccggtattcaagggcaggaggcatccagcgtgggagaaagatgaaggccggaagactcagccagtctcgtccttccgcatttctctgcctgcttttatcccagccacactggcaactgatgagatgatgcccacccagattgagggtgggtctgcctctcccaggtccactgactcaaatgtgaatctcccttggcaacactctcacggacgcacccaggaacaatactctgcatctttcaatccaatcaagttgacaatagtaaccatcacattaagtaaccaattagtgaaaactcataatgaatccattatgctaatgaacatcaaggattatgttatgttcataacataacatgttacgaaaataactatattttctttagaaactggtgacaggagtagcattgtttagatgtgtgaatgctcctgctgcctggctcctgggaaacaagtttcccatgtggaattctgtattcagtctgcagtgacatcacacgtcagttgcctctgcacacttgtgagagaacgggagtggaaaaggcactcaacacttcagccatgagaggaaacctgtttgaactaagagtcccctaagaggggagccagcaccacttaaaaacctttaagtactctcaatagaaatctttagttcacaagatgttttacaaataccttatcctagtctccatatcatttgtggaagggaaagtttagattttattattattttttaaaaaattattatagatatatttattattaaattttagtcaattttattaatcttttgatcatgtgatttttctatgtattttgcgaaatccacaaaatgtattcaaaatatattttcttatattttcatctaaagagtcttgctatatttataaagtttctcagtccacctgaaaataacctttgtgtatgtcttgaggtatagatctaaaggtatcttttttcaaaatgaagagccaattgcccaaacgattgggcactttatttgttttctaatagactaagtttcaacacagaagagggtcttctttggtgctctgtactcttttcctttggtctatttttctcttctaccaagatatcatgtggctgtaattgcaatggatttatatggtgtgcttatatctggtgtaatgtatcctcgacttactttttctcctttaaaagtatcttggttattattgtcctgtattgtttttggagtcagccagtcaagttttaaaaaacacgtaaacagatgcaggtgaacgtgtccccatgggtgtgtgcttggtgggaactgcatcaaattcatcacctcacttggggagacttcatcgctttaccatgcaggtctcaccacacctccccatttatagacatctttaaaaatattcttcactgatatctttattttttcataaagttattacccttgtcttagttgatgtattcctaggtaactgataacttttgttgatgtcaaatgaaattgctttttataattatgaattgggtactgctgatagttttgtttactagtcttgtgtccagttgaactctcttatttgttatgaccttttaaaatgtagatttttatagggtcaataaagaatgatggtttccttttattcctgacccattgttccacatttagttcattttcttgcattattgcacaagccggtaactctacccgaggttgcatagaaagggtacatagaaagggcatatctttgccttgctcctacctcccaaaggcagtttctgaagcttcactgtcacatgtggtggctgctttttctagtctatgatttagatgctgcttttgcatcaacttagctgtggattttttttttaatgaagtttcactctgttccccagcctggagtgcagttgtgcaatcttagctcctgcaggcctaagtgctctctataaaccccaagtgcagcaggcgggaggagactctggctatgcacaaagtttgctggtgggaggacagagccaggaactctgtgtgtgtcagtaaaatgttggggtgacagtcacctggggggaaagccatcacagaggcactgacatgagctgtgtgcattgggcagtctctccacctccaagggcctcagtgtcctctcaggtgtgagggtcagtggtccccgtggcctactgccacattcattgaaatgcta

SEQ ID NO: 60 - DNA, 2574 bp, Homo sapiens
ctctttctgaacaccccccggcagacacagcgcttacatgggagtgcacgaaggacacccttccctcacgctgagctcagcacagagcctgcaggagttgcccgcagcccggcggctgccatggagatacacacaggacacaagtgtctgtgatttctgtggccacacctgtgctggctgctcccgacgtccctggaggccagctgttccggcagggctggggcacacacacaatctccacagtgcagccgcggcctcctgctgggaacgtccgccccgtcctgcctctcggggcggctaagtcgctaagtcacgcccgtgtccggctctgattggaaaaggacgccctgggcttggctgggaggaaaggccagagggtccacaggggaaaagctcagctctggggggcatccctccctacagctgggcctggagaggagcccagcacacctgatggccatcgcagatcaggaaaccgtcctcccctccctcctgccctgggccaagcaggtcctgccagttactataaaataaagcggggggtgtgggtggcaccaaaagcacagcaggcgagacgcggggcacaggaaggaaggaagccacagcaaggcttctggtctctgccgctcatcagaaacctttcttccgccctcagccactgtccctcttaatccagccacattcacggtttctgtatcacccaaaacatcatgtttgttggaacttattttattttagattcaggtcttgttaaccattgctccaggatgctttactttccttgtcttaaacgggaacttcccaggtcatgttattaagaagtgggtgcccaggaagcacgggtcgcagctccacacggacagaggctcctgggacctgggactggctctaggtcatgacagctcagcaggattccagggaccgacggattcagtcctgaggggcagaccaggtcctggtaggtacagcaaggaggactcccctgcaagtctggagcaacaaggccccatgaagggagacaaaaccagggaccctgacacggtggctacaagggcagaggtgagagcagaggtgtgaaggccacgcagcccccaggacgcccccaggacaggctggcctatgctaagccacgcggctccccagactcctgaatggagaagagggtgctggcctcagaggctctcgtgagggccgtggaggggagcggaaagccaggcaggcagctgccacccgagcctggtgtttgctcggtcaaggtgccacagcccccatcaccccggggtgggggccaccaccatgccctgaggaccgagggccttctctgaggccagccagagggtcgatgttcctctgcgccttttccaaacagcaggatggtgcagaaacctcaggagggtaaaacccgtcagctattccccttggggcactgtctctctgtgcagggaagagtcagcagttctctctgttggagcagacgcgacctccagctctaaccaagactctcagaccacgttcaagttgcagccagcaaggagcccggagctggtatcccggagcttgttctttcctggggcgctttgtttcagtccacaagccaacgctccgtagcgcggcccccaccctcctgccgtgtggggcaaactattcaaagtcccctggccgtcagaaggttccagagggtgtgcagtcactttcctccccattctcacagcagcaggaccaatggggacgtggctttgtctgcatccctgcggcccctgccactgcactcgccaccatcaaaagcttctcctctcggagctcaaggacacatcaaatgatgtcacaccacttcacgcccttctcccagcagccccgcttcagtgcctgggaagctgcacaaaataagattctgttatcaagcaacgctgcacttcccacatctggatgcacgccaagacaagacgtcagtcatttcctggtgaaatgaaagaaagccacgcttcctccacgcccattgggtcacgaaatccttgctaatcctggccggggcactggaggatgctataaacaatcacggatctgagcaggtggatgaagggaacgtagatgacacgttgagggtgtggtgcgggcaatacacagactaagagtgggaactggcgaagtgagctataatcccaagcataaaggaaaggaggggaggtggcctccagcgcctctcctactagttaaaggagagagagggagaaaaataccactggaacctccaggcaggtcagacgggcacttggggcttatgtgcattatttgatggaacaagcagtgtctttgtttcttaggatggccatttttatctttttgataagtgtggaggaagttggcttagtataatttaatttctctctcctattaacaggtctcagtaaaacaatggggaatataccaaaaaagagagagagagagagaaagccaaaagaacataaaactagcacattagtcttttaaataaaaatgcagaggaagatagggaaggaaaagaatactacccaatattagtccagacctcgaatacgaccaggacagcctgccaca

SEQ ID NO: 61 - DNA, 2872 bp, Homo sapiens
cagctccagagcagggaacccacctcaccagcgacacagcggcgacgagggccgggtctgggagggcgtgggcagggaggggcgacggaggcggtctcccttgccggggtgctggtgacacagcggctgcacctgtcagaacacgccagggtggagacaggagatctgtgtgcttcccgagtacagatcacggctcagcatctcatgggaaagggacagggctctcttcaggacacgcagtaagatttcaagtgcgggcacttttaatactccgcgatccaaaggcagctccagggccagccgcggtttccggcctcaagggcaggctcggttctggagctccctccagtggccgtcggggtgccgtcactttcagggccccaccaggagagcaggggccccgccgaggaccagagcgcctggaccagagggagccctgcgcggccggcacggatgcctctcaataggcggcatggggccgacacgactcggtgagttcccgccacggctttcgcggcagccggcggctggaggacaaggagaatgcgccggttctgttcctggacaagctccatggcgctgcggggtcccggcccagaaagcccaccctcccccagaatttccccaggcccacagaaggggaccggaatgggaaaaataccgacaaacgcagcaacggtgcggccgtaggtgtctgcgcatccggcggggctcctacgggacccccacgccgcctggacgccgcctagcagatttggggccaggctaattggggcccatcgtggcccacagatgccagctccgggccatgctgagggacaggggagcggaggatactgcctgtttcccggcggggggccctgctcaacagcctttcccttccctacaaactgtcccaggatcccgggccattccttccagtaagttgggaagtccaggaccagacctcaacgtggaaaaagctggaggagagaaggggggacgaggggttctacctgccctctacctacctgccctcctacctgtctgtccacgggatgcccagaggctcccagaccaccagccccagacccttggtactgcgtccccagctgtctgccaggggcctgctggggaggccgatgcccatccctaagcctgagcctccagcccggcacgagggaaggccccacatgccccaaaggagagggttcggggcacaatcttcacaaaggctggagtgcaccccagaggtgagggtttggggcacagtctgttggcggaggcaggagtacaccccagaggtgagggtttggggcacagtctgttggcggaggctggagtgcacccagaggtgagggtttggggcacagtctgttggcggaggctggagtgcacccagaggtgagggtttggggcacagtctgttggcggaggctggagtacaccccagaggtgaggatttggggcagtctattggcagaagctggagtacatcccagaggtgagggtttggggcacagtctgttggcggaggcaggagtacaccccagaggtgtgggtttggggcacagtctgttggtggaggctggagtgcacccagaggtgagggtttggggcacaatcttcacacaggctggagtgcaccccagaagtgagggtttggggcacagtctgttggtggaggctggagtacacccagaggtgcgggtttggggcacagtctgttggaggctggaatacacccagaggtgagggtttggggcacagttttcacacaggctgcagtgcaccccagaggtgagggtttggggcacagtcttcacacaggctggagtgcaccccagaggtgagggtttggggcacagtctgttggtggaggctggagtacatccagaggtgcgggtttggggcacagtctgttggaggctggaatacacccagaggtgagggtttgggcacagtcttcacacaggctgcagtgcaccccagaggtgagggtttggggcacagtcttcacacaggctggagtgcaccccagaggtgagggtttggggcacagtctgttggtggaggctggagtacatccagaggtgcgggtttggggcacagtctgttggaggctggaatacacccagaggtgagggtttggggcacagtcttcacacaggctggagtgcatcccagaggtgagggtttggggcacagtcttcacacaggctggagtgcaccccagaggtgagggtttggggcacagtcttcacacaggctggagtgcaccccagaggtgagggtttggggcacagtcttcacacaggctggagtgcaccccagaggtgagggtttggggcacagttttcacacaggctggagtgcacaccagggaggcttcccgcctctggcagaatcaccgccatgctcagtcacaaacccagagctgcgtttggacgctgcagcacacgctgcggccccagcaacggtcctgcgcaccaggctcctctcccagtaaggtccgcttctctgtggagctcaggggtccctgcagtgcccaccttagcagagggcaaagccttgagacacggatgctttgtcctcaggtctccactggctcctcagaacagggcccctcagcgctgcagtgtgtcacatgtccccagtttcccctcgtggtgctcacgccacacccctggcacggaggctggaacccaggtgtcagtcctggctctgaccatgaccttggacaaaccacccctcagacctagagccctcatgcacatccccatggtcactgccacccggcagggagcaggacagccccgggggtctgtgactgtccccgggacatcagtctgagaaacagcgctgagttggacgctgcctggtgtggacactc

SEQ ID NO: 62 - DNA, 2856 bp, Homo sapiens
atttctcagaataatgaatggcaggaaataccatagttaattaataattgactggtttgtaattatgtgctatctacacccataaagaaattgagaagctcataaaatgcacatataaataagagttaattatgtgaataagtttaaatgtttttatgacaatttaaaattattttacttttataagacttccatgtaggtactagcactttcattaatgtgcttgctatttttcacttaaatttttatctctatgaaaacctaacaccttcgagaaacggattcatgtgcacgtttctgttgctaaactgtggcaggaacatcagaccttaataagagaagggtgaggaaccacaactgcatatgtagtattcacagtaggagaaaagtgatactaatataccatgtagaaaaaaagcacaacaaaataagataccatttagcacacacagacaaacatgtttgctgctttgtttcttgtgactgacagacgctcttacttactccgagtctttgaggtaataactgcttggaagatggccgaagaggaggtgttgacatgcaagagtggctattttaaaggagcacgaaccatgggctaataagcgcctgcgatgtggccacttcaagcccacatgctgccagcaccatgtcctcgtctggcgtggacatccaagggcggaggaagagctgaaccctccacaaaggttccatttgtatgcagaaacaatgtccacagtaggcgagggttttctttaaaatcattagcgtagctaaatttcaaagttcaagtaaaaattgttttttacagattgggaagtcctcttccgttgtacccatcagcagaaggtgtgtgtgttcaaggcaaagcgatcagaattgagtgcagaattgacctctgtcggaatgttccgcatcctaggtctcctgtccctcgctgccactgcgaagtttgctggagacagactgtgccttcacggtcagacaatgccctcctggactcttctggctttgtaatgtgcctgctcttcagccagacggggccttctggaaggagtgaaggccagtagtcagagatgctggtgcaaacctatgctctgtcattcccagactcggtgttcttgggtgaatcctctccctgtctgttttctgggaataataagaacctgtcacttctgtctttgcgggctgctgtgaggatggtttgctatgctgtaatatgaaaggaccatgcagatgataaaatgacccacagaaaaagctggtattctcattatcatcatttaaaatactacaggtgaactttctgtgtaagtagaggttctttgcagaaacatttttgttttaaatttttgaaaagactttatccttgaacagaatatgtggcagagggatttgtccgtattcatgtctcattacaaacatctcttctggttaaaaatgcaaatgcagctgacaggagaggacagatgcttggctagaagccttctgactgtcatcctcagctgcccctcagcagtaactacaaagcctgcttcctcaaaagctactcctggtatttgctgggttgtgccctcttcttttttttttcttctttttttgctttatgcacaaagtgagcagcacaaaggcatgatctcatggccattgtagcatgggcaactttgggttaaattgctttggtctctatttaatttggttatttttctcccacatgcttttgcactgtccggaaaatgagctttttcatgattactctcagtgtgctgagactagtcagcagcgttgaaagattctttgtttttgcacagccagcccagggctcacggacacactttaatatcctgcatccacactcccttttcctttgtgtgtaaattcccgagaatgaaggaaccgttttaccccctcatgtttcaggatgctttgctaaggcgagaacctcacagtacatgaaagcacctgtagggctcctgtctgaggagccacccacctatgtctgcatccagtccgctcctttacaagattaaagtggcccggctgagacactgctttttagaaggtaagttacactcagaaaagtcttatctgaaaaatcgtgtttgactgttaacagatctaatgttattctttaaaaaaatatagtccaacttatagaaatttctcattgagagactatctaaacagtgaacagtgaccaaacacaagtcctctgttagggtaggaacagccgcacaatcacaatctgagaatgtcttgaaacatgcacacccctcatgaccagttaggtccacactgtgctggaaactctggccacccatgtcatatggatgtggcctctcttctgtagggatttcctgacatgccatcaggtttgggctcagactgaagcgactgtcaaaaccattacagtccagatctttctcccctaaggggcccctaaggagccccatggcagctggtgtgaagtccccctcctgggagagggactgtggcagcctcctgccttcggggactccccagtctctttctgatacatcatcacacagatctccaagctcgggtacctgggaaacatcaccagcatagttttctgatatttctgcctgtgattccaaatcttcatgaatgtcttccttgtgaagaaactccttgtcttcagtcctggtgtcacaatctgaaacaataaatagaatatcacttggaaggcagtgctgcagcaggagcaggaacatagacagtcacagttgcacccactaactgtggaggaggcaaggggagcaggggatcctctggggtggcagtccagatcagagggcatcagggaggggtgggaggagcactgggtgattaggc

SEQ ID NO: 63 - DNA, 2154 bp, Homo sapiens
gagcggcctttgcaacatctcacttcccctgttgactgttatttcttttcttcctgctttcctactcccttgatcccaaactcactaggggtatttagtgagcacttactgttgcagtaagactctagccaaggaagacgaagagacagttggagaccaaagagaacttcaattcgggcacccgagcctagagcaggctcatgcccaaaatggctaccgacccagacaaagaaagcaggcttgcttatatgtcgtttcaggcgtgaaaaacaaggcaggatacaagtttcagacaaagacagtaaattattcaacctgtgacaattctgagaaaacttacatttagttatcttgaccagtcaaccttgaagctggacagagctggggtaagggaaaacaggaattacggaagtatgagggagtcgcgaggccggagataagcttggaaggttgagataagctcgcaggtgcaacttcttagcaatgctgagagtggctgcttaaatttcttagcctatgtataacttctaaatagcctacactaaatggtaactattacctatgttgtgtttgttattttaaactttaatgttatttattttatttcattttccttccacattacctctgctgttagcagctttgagaaatgctgctataggatgtgggaagtcattaaaggatttaagcagggagaggcaagatcagattaacatttcagaaaaatatttactgttttccagctgaaactagtagagtacaatttactttctggtcacagcacacagcagtcacatcctggaggaactgtacttctctaagatctagtctgtcctgtggtttaaatgacctttagcaaattgtctttattactttgtacactgctttcaccagtctgctcttccatggctaacggggcagaactgttatttttagggttttccacatccagtatgttcataagatttctaccctgtgtgaacttccagatgtcgaataaaggctggatgctgaccaaagacctttccacattttttacatgtgtgtagggttgctcaccagtagtattcccctgatgcttcataatggttgatcccagagagaatgccctttcacactcattacattcatagggtttctgtccagtgtgagttctgtgatgggaaattaggttagaactttaaacaaaggcctttccacgtttgttacactgataaggcttatttccaatgtggattctttggtgttacacaagattagagctgtagttgaaggttttcccacactctgccttcatagaacttgtctttatagtgaaatctctggtgttttctaaattgtgttagtccttcttaaggcttaccatgttcactacactacacaattcctctccatggtaactatttgggtgctcattaaaggctgtactctgacgttctgcatgtttttgagatttcattaggatgtgggctttctggtgattgttaaaatgtgagttatctgaagctgtgtccagatgaattacgttgataggttttctcttttgtgggaacattcagatatgctacagggtttgaggtcaagtctaggatgctgtcaacattgttatactcctggcttttctcccatggaatgtttttatggatcactgtgatttatcttcacatgtacttgactagtactttcttaaacattttcttagttttcctctacaaagatttccctgatatttctctagtagactcacaactctgtaggctttaaaaaagttgggtgcttagtcaatatctcctttttaacacataccacccactgtggtttcatgctttgggggttccttttggagaggcaactctttgttatctgcctcacaacctgaagcaatacagcaagcaggaaacatggcataataaaaagaccacagccttttaattctaaagaccaagattctacatttcctcttctcctttccagacaacttagtcccaaaggtataaagtaaagctgagcaaggtagcatccataccagggctgggggaaccaaagcaggaaagagcagcaaggtggaggccatccatatagcaagactggcacagtgtgtccagcctaagcaggctgaagatgtcttcatggaagggcagaggcagaagggca

SEQ ID NO: 64 - DNA, 2079 bp, Homo sapiens
tgctctcctgtgccaagcgtcaatatggatttttgatgaaattttctacattggcagggcaagcccctgcgtgtttcctcaagtggaggcagtgacagcaaaagcaaacattttggatcacacacaaatgtttacaaataagatatgtttaatgagcatgatgcttcatgcaataatagcagtggcaaaaatggccaacagctacattattattacattcccagtgctgttcccagtgctattcccagtgtttctctgtcactgtatttgctggtttgctgagagcactatgagattcagtgttccccagtgacttctcacgtcgcctaattaattcagcaaagcacttattggcgacttcatatggcctaattgtggcaataacttagtgtgattaaacttaatcaaacaccatgtcagtaaatgacatgatgtcactccaccgatgacattcatgaaggaaatattagggcccaaatattcctataggtgactttccaggacgctgctgctggtgtgttcacaaggctgcatgatcaggaaattaaccgcaccacatgctccacaatttggagcaaatcatccacctgggacctcaccagactctccccgtcagcagcggcttctgcctggaggctgcagatgggagcacagagggcagtcagtcattccattgccacgtcctaaaatccagtcctgacttcttaatcccaagccccgttctcagattcaaggccccgtcttctctggcgttgccattgccatattctagaatgttatttacactaacaacttagggccgaagacgcggatgataataggacccaaggaaaaatcaatgccgagcaggggtgcggggtgcaaggaaggcccatgaggagcctgggctgagtgggttttccgataggagcacacacttcaattctgaggtttctgttagcaaaaaaatcattaagtaagagaacactgagagctatactttcacagctaaaaaaaagttcatttctttagagagagcttccccacagccctaactgctgcagaccgcactccccaccacttccacctctgtaaatcctgcacactcaggtggaccctgtctccgaaacttcccccgtggagaaggacgtgtcctcctcactccagtgagagaccaccacgcccgtggccaggcactggggctggcatgaggctgccctgaacaccgggaacagcgtcttgaccagttcaaattaggtcacgattttgcacttcccaaagcaggccttcgctctgtttctccagtcccaagggcttcctgaaacgtgggggcccttctgtcacccaggctcccacttccctgaaactcctccagatgtgactctcgcctggaaaaaggacatcttctcctgttaccttttagcttgttacaaccggagaaactcactcaaaaggctctggacttgtacctgccccctgagaggccagcggggaagggttgtcccttggccctgaacctctgcagggcctcatttcctccgcagcccttccgctgctctgataagagaaccaccaattagacccggcactccagctcccaggagactgaaacacatgaattcccaatgtcggcttctgaggcctcagcatttcttcctcaatgagcaccgtatgcacatggagagccgtcttcacctcaaatttcagatttgcccgttttacttcctgctcactctgccccagctctgctctcctgcctcagtttcccagagaatgtggaatcccccgagaacacagtcacctccccagcctctggacaccatcacagtcccttcttcctgactccccacagggccgcctcttctgccactactttctcagcacgaagcgggagaaggaggaggcaggcagcttcagacagtgagaaagagagacagacgcgagccgcaagcacctttcgatgcccaagaggggaagctgttctttcctcttttaagtgggagccgctcaccactatctctcctgcaggttttttggggggccctggccgtgctccctgaggaaactgcagtgaggagggagagagacccagagaggtag

SEQ ID NO: 65 - DNA, 2707 bp, Homo sapiens
gagcagccaccctggatgctcctgcacggagtctgttcctggacacagccagcaccgggggcttgcagggtacaagtgggtcagaggcctgggtccccacctccgtgtgtctgtgtgcgcagccccaggcgtaagctgggcccactcctcactgatgacagccggaggcaggggggttcctgcagggctgctgcttcaacctgtgctgggcctgactgataagggtgttcccagggaacacgaagttcagggagaaacagaaagctgtgagaccaaaggcctcaaaactaaggctgacttcataggtttgccttaagtcttccgcggcatgaggcagaatagtaataaatgatgagataaaattaacgcagcagctaaagcccagccaaacaacatcatctggggacagtgtcagcctaagggtgcttgcttatgttatgcaaagaaacaagagtctaagaggtctctccaggcagctcagcaaagcaggtctgggtctgagctcgccccagcgcgcatctgcaggcagggtgggctgtacagcagcccagtgcatttgcacacatggactgaaatggcaaatccctaaaagagctccttccttctgtcctaggctcgtgagtgataaactgtgggagactcaggaggcaggaaaacatgttcacccacctcccttctgctcccaagttcactctcaaaccaggatggcccatagctcctgttccgcgcccaggaacagcagctgatgctgaggcctctcctggcacatctccaccaggagatctcagaaggccccgaagcttgtgccatggcctcttggcccctccaggttctgcctgttacttggcttggctggatccaggagcccagggaacggcagctcccatgagagatggtggaaaataaaggtgtgttcagatcggcagttctggtcagttgggttccttgggccactgagtagctacaaactctgctggtcagttccccctgttgccctactgccctcgatcccaccaatccctgtaatcaacaagggcgcaggtggaaagctggaggcccgcacttcaagagagcccctgctaggcacctctgtcctcccagacctctgcctggagcctcaccggaggctcccaagctgtcgccagggagcacagacgaggcagcagaggccggcctggcccagggctcccaggatgatctccctcagggcttcccttcagcctgttctgagactggggcagatatcaagagcctttggaaaagaggagcagagagaggggaagaaccagaaaggcctgctgaggggaagccagtggggtcggggaattagaagtgggtggtctccacggttgacacccagccttcttcatcctgagtaaagcagcccccgacggaagagcagacattggcctgggctgaccgaacaacacacctgaacagcagcatcagggcttgcaaaaacgtccggaagttgttgtggcggttgatgctggtgtcatcatccagggcaatattcccaaacacctgcaatggagaagagcaatggcaccggaccctgctgggtctgcaggagccgcgccaggtggaccgagccacgagagggcgtgcgagccgtacaaggaccccacgtgagatgggcgactgccccacaccagagaactcccacccgggagaggccagtgtgcattcccagtatagacgccctctccgtagctacacatgtgccggctccagctctgaacctgtccacagatgcaagtccgaaacactcacaagaacggccccgagctaagtttgtgaggcctctgccacacgtaacacaggaagtgttttcaagtgggatcatcagcgactccaaagcaggcattatgttggtaacaggtctgacagatcatgggaaaatgtcttcttaaaacatatgcaatagtacaacgggctttttagccattttaactgacttttccacagtaagaaatgcaaatgggtcagtaattgtactcagcccaaaatctggaatctggctgcaaatttatgaactatgacacatccacaaagatcagtaacgtatgtgctcttgtacatccacagaccaaagcaggaaaaaaagatgtatttatttaaacagcatcagatctctgcaaattttaaagcaagagaactcttcaatccctgaaatagagtttagaaatcagttttccgtgaacctttgaaacaccggcaccttcgataacaaattaacactcgggtccctcttccgtccctgctgttggaaaagtggtcagatgccaaagatttataactgggacactgctttatgtttttttaaatgctttttcccaaatagctgacaatgtgttctttctaaaataaagaaaatctaaattatgcaagccaagtttgcccagcgcaaggatccgatgcgctcctgctcacattctcaggcagttttccccagtagcatgtaacccccgccgcgggtcaggcctgtccttcagcgcggctgccagactaacaggataaccgaccgccactgtgcaagccactcggcaaacacagctgtgctccaacgcgcctccaccacacagccagcacccattctgacctgtgccccgacaccctaccatcactgggcgggagcccatgcagccctcaagaacaccacggtgcatccacctgttgaggtggcaatgggccgagggccagagct

SEQ ID NO: 66 - DNA, 2232 bp, Homo sapiens
ctccaggtaactctcaggccagcagcccaaaagatctttgagaaccactttcttattcaagaaagaacatctgctgaggtaacacccaatccctaaactccacccctggagcgaagcctccacatgtccagggggttctgcggaacccaggaagaggctaacacagggcctggagatgcactgaggggagcaggctctagaaggaaaccacctggggaccctgaaggagggacagaaatgctacttaccgcaatctctgttactaaaatatcagtaatacttcccaacacagtgacaaagtcaaagacattccaggcatctctgaaatagttctagagaaaaagaagagcagttagtgccagcggctgatgagggctctgttggcaaagaggtatatataggtggtggccctgattaagaaagcggtgagggtgatagaccctgagcacagggcagacaggccaccccagggggcacagcacaaggccagaggtaagcagatgtcaaagccagggacacagatacctctgggcctgggcagaggcaggactaagagccatgtgtccaaagaggaagaacccagccctgcctccctcccaggacctaggctgggggcagagcttatgtagccaagagtctcagaacagccccttccccagggcccctgtagcattacatatactctgggtactcggagaattcccagctccaaattgtgagcccccaaaggtcgccctacagatggggaaccagaatataggttgtcaaaaggcaaagcagggaccaaagcacgtaccagcaccccaaaggcgatgatcttcagcacgcattccatggagaacatggatgtgaacacgatgttcaggcatttcagcatcagctcgtactcatagggtgcatcatagaactgcccggggaataggcactgttggccatgggtttggcagccccaagacaccccatctgggaccactatgaccaagcaaagcgggcagacagaactcgatgcctgcctaggcctgggacaccccttcctgctctccccgagtcctcccagaacctccccactgtcccagcccacagacaacaaagggaaacaggattccacaggcatcccatgctggccaggatgcaaggccactactgctttggctcatgcagggaggaagaaggctgactctccactcagcctcagggttagatcccaatccctagcagcgccactgccctctgcgctgagccccacacaccttcatcatcagcaccacagtgttgagggctatcatggccatgatgaagtattcaaagggcggggagaccacaaatgtccacgtcttatactggaacgactgccggttttggggcatgtaccgtgtcaggggtttggcgctgatggcgaagtcaatgcaagccctctgtaaggggagaaaggagcacagagactcagaagcagaaaacctacccgacggcatctactgcacccagccctgtctcgccaggcctcacagggagccctgacaacagaagacaaattgaagcagcccagaccttctccaagcaggcctcaaccagccaaatccggtccctctgcaggagaaaggaggacctgcccctgtgttggcagacggtggcagccaaagctgacccagctgtgagtgatttgtgtgcaggagggaagcctgatggcgctgcccacgctgtccactgcaagactccacagagcgtccacctacctcgttcttctccaggctgcattcagacatcaccttgtccccctgctcctggaaggtgatgatgatcaaagccacaaagatgttgacgaagaagaagggaaagaccacaaagtagaccacgtagaagatggacagctccatgcggtacccagggcttggaccctgctcctcataggtggcatccacggagtgtttcagcaccctgggcaaagaggagagcaagtgtcaggggaacccccaaaggagacagccctaagaactcaagacctgcaccacaagggtgggtctgcttccatgcctgagcccagggatagagggaggaagggaggccgagctcaggggctgcctgccccagctacggagagcaggatgagcactcacatgggccagccttctcccgtggacactgtgaacagcgtcagcagagcccagagcacattgtcgtagtgaaagtcgtatttcttccactgcctgggctgagcttccacttcctccttct

SEQ ID NO: 67 - DNA, 2278 bp, Homo sapiens
  MISC_FEATURE (1473)..(1572): n or x is a, c, t, or g
agaagcaagcagaagtacagaaccagagggcctcaatcagggcccctccaagaaaaagccaggacagacccaggcagctgcctctacctgtcagggacgcaggaattagcaggttctggggactggacctcccacgaccctactgaggccgggccagcagtgtctaggagagatttcctcctaaggcggcccccgttctcagaagcaaagccactctacttggtgggaggtgagggtgggagctgaggactcaggactgagtgggattcactcacacatggaacccttcccaccctgctcagaggccacgtcccaccacgctccctggggaaggcctgcttctaggggtggccctgccccctgtgctcttcctggggctccagcaacacttggggctgagcagggagagtgagctacacgtctcaggcaccctggtccccttcttctcccctgactgtaggctacactccagaatcagatcaaactccccctgaaacgcttccaggtgggaagaacccagcctcctgtctccatcaccccagtgctcccgacacccactctcaaaccagctcctccgccagctgagggaagaggggacaggagcaggagggaggggatactgttttgtcacccagtaaatgaggctttctgggggagcgtccatctggggcctgctccttttctcctgctctgaagccgcctggatgggcccaaccccttcccctcctcctgactgggggacccctggctgcagtgttcccactcccaaggcctaagctgatgctttggcaaagctctcattcctttatcacagaaagaggaaatagtgggaactgcagggggctggagtggagaggaaacagaggaaagaatgccgctcttccagagaggagctgcaccgggagcgcctcgcgatgtccccggtcctcgggctgtggccacaggtggcagttccctcccggagcccttgctgccctccaggccaatggccccagcctccagccctcgctggtgacagcctgctcaccagcaagctcctcaccaagggctgaccatgcccagctccagcccagctccccgccccgctcccagaggcatggcaggaacccctggccggggacttggctcccggcagcatgcagccccgatggggtgaaagtggatgggcggggggtgaggctggagatgaaatgacccaagaggggctgctggaatgctgtgatgtcaggggcagcgtgtgggggagagaaggcattaccccacgaagctcctgccgagtccagcaagaggaagacaaagagaacagagtcagtggcaccaggagcagccctcccagccgctcagagagatgtggaccctccctcatgtctgtcgtcactaggggtcttccttgtctctggatctcaccccacaaccttcccggcgtatttcccattcccagctgttgctgagccccccgacattgccctaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtagctgagcccccgacaatgccctccacgcagttatctgaggaagccaactccatttccagccacagcagggccaagtgcactgctcaggagtcagaggagacgtctaatgccccaaaaggggaaggcgaccaccagttctgccttgggcgaaacaccagaggtctccctgggtcaggagcagacttgcctcagagcaggtcaaggtgaagttgcctccaaggaccctgggaggcaaacgctggacacaccagaggctgtgcgcccggtcccagagccaccggccgcaccagggcctcccagccccacatagcacccacccctccaggcaggcggggatggtcccagggccacaggcacaccagggccctacctagtttgagagccacagacagacctcatgccctcctaaccccacgcagccccgccccaagcaggcagggacagtcccacatggacagcaacagagccacaggcagcccagggaagcccacaagagggcctcttccttattccctcaccctcacgcccatcgtgattctgggggctctccctagccagagcagagcgaacgttacttacgagaaagcaaacgccaccagggcgccactgaccacaatgaagtccagaatgttccacaagtcccggaaataggctccagggtgaagcagcagtcccaagtcgatcatctaggagggagaaaca

SEQ ID NO: 68 - DNA, 2376 bp, Homo sapiens
actccatccctcctggaaaaggactggaccccaattcccaccattgcttttttgggacccattatcttccttagcttcctatgcatctacagggtagtctgggcttcacttcctcagtgtccctgtatgaaattaggtggatatagattagtctgatgtaggaatatcacactgtactaaggtttagtttgtatgttattctctcaagtaactgatctttcaatccaactaaacacttcctatgtgctttaaggtggtgggaattacaagcatagcaagttatgattggtcacggatttctttcctctttaaatggtgacctactgcccattgtacctactcaaagcaactttctttaggaaaaaagaccacagtctactttcctaagcataaactcagttctcattccacctctaccacctgcaagatttgttaggcttaagcagtcccttaacttctttgagtgtttgttgccttgcctacttcattggaagtaaggctctggaacagggaaggtttgcctccataagactaaaagttatgctaatataagagactagcaaaatgggagacatattcagctctcttcttgtggggaataccttgcccttgaccaaaagccttgtcccagaaagagccgtgtgggtgttggctttgtgcccaacatgtggctcctctgccatgattgatggcttcatttaagaaacaggttttaggattttttcccctaaaatcttattcctgttaattatcatggatcaactttaccttagctcgtttaatacacagtcacctggtataaaagcatgtgaaaacccccagggatcgtaaccacatttatgcattgagaaaagagagtgaggccaagattttgagatgtgttcaaatgcaagaagcttttaaaatgcaaagtattctaaaactgttgaaagttgaagctaactgttgttcccttgttgaaggtaaaaagtaaagcatttttaggaaagcacttttccttatgtgtctaatatttgggaactgcataggagaacagtttaataggaaccctgatattgacagtaagatatattcttaatgtagtaaccagacccagggcagaatttgcaaacccatggtaggcatacaggtggctgaagaagaatcgggacagcaagatctcactgagatgcaattccattcctccatttgatacagattaagatttctgaaaaagaccatcctcctaaaccctcatggactctgcagataatatgaggccagaaaatgaataattcccaactcttgctatctcgttactggccagtgtgtctggcttcgctgagtgtgtgccttctgaagcgtaccctataattattcagcaggtatagtccagttcgtcctacttactttagcaagattacctttcttttatttttcctgtgaaaatccttctcttccttctttcctcctttgtctttcctctttgttaactttttaaatctaaagtgccttgaaaaacttgtttacatagtagtaagaaggaaaatgttgacttgtgctatcctgggaaccttgaccttcctgcattatggataaatcatttccctgcaggtggaagtggaaaattgcagatagaaccacattgactcacattctccttctacttccatttgagtgagcaccaagtatgcatcacgacttgagattataaagttggcttaatgatgagacaggtttctcagtcgggttttccattggctcgaagttcacaagcaaagggtgcacagcgtggggggagcggggatgggaaggagacacgtgggagcccacacccagccaccagagctggagacagttagagctgccactgggcacacgcccggagtgcatggctctttctctgactgtgcatttggttttaaccttctacaatgcagcccgcccctgctcccaacacccaagccttgacctgtgacctctgggtacggaatggcagagagaccagtcctggggaggccccgatgtgcccctccacccaccaaagccagaatgacatgtggcctggggttaaggctagggtccagccccatgcccatggccattccaaccccagggtagtggtcacaggtacattctacttattctgggggcctttgtgcctcctctcactgaacactcccctctgcagagaggcagcgccaggcccccccaccttcagctgtgagccagttccaggaagggccctcacttactttgtccagggtcatgtctgggaggttcggggccacgtcaccaccctcactctcccggtctgaaatggggtctgacgcctcgtagccatagagcgcaagcagctcatcaaagggcatgtcgttgctctgagttggggaaggg

SEQ ID NO: 69 - DNA, 1896 bp, Homo sapiens
caggaaataggcaaacacacactggaaggaggccacatggctgttttttaacattttaatttcaacgtgccagcatttgtccaaatgagatgatacaggctagaatgcacggcggaattccagactggactcactccataagccaactcatcactgcccgtgaacatgaattctggtcctcagagaagctgacattgtttccctgaacattcccgtggtctccttctgaaagccgatgaccatccaaccctgactcacctgaaatatcctacgagcctcgccctccgagactgacgattattaaccacccacacggaaaaagaaacagcccctccatcacccacatcttgtacacaaaaaaatgccaccactaatgccataaattcaggcaggttcctctatccaaaggctaaactgcttcaggtgacctaaaaagtggccacgcctctccacgtaaacacatccagctgacacaggctaggatcgagttctcccacggccttcctatcccgtctctaatttactctctgcttttccctggaatgtgcatgagaaataaaccttccaaacatttcaaaagtcgcactttcctcctttattacaaccatgcccatttttaacgacactctcggtggcccctgacagctacctggtgagatacacagcatattgtgcccattgaatgaagatacttctgacaatgaggctttctcgtgaaataaagtttcccgtctcataaaactgagaattctctggaaagagctgagtggaaatggctttgaggagggcagtgattcactaagttattgagaactgaggtagtgagggtagagaccaagccaagagcagtcaagggtggaccgactgcaccctgacttttgttgtcaagcagagagcatctctagatcctgttatcctctaaacgatttagagcaagccctcgttgcttctcaaccaggaagtgaatcggtttagatcctctaagccacccacattccccaagccacctacaatctttcttcccaacgtccacgagtagaatttctgtcaacgctctaggaagtcctgttaggatttaaagcagagagaccacagccgaggtgtttctcagatacacttcgccaagtccaaatgaaagtcagtcaccacgtctaaatgtttccttagccctacagaaatgggtctccatggcaaagcctcagaggtgctaaatacgtatattagtgttgttagcttcgtgatgggaggaaatttgcagtgaggtttaattctgaatagggtaggtctcacagcacctgtacaacacagctccagcgtacttcagaggtccttcgggcaagagcggagaccaccatcgagagtctactagaatgttattactgctcgcttttgccgacagcttcaagggtagaagtgacctctgaagaaagcccagaaggcgttggtggagaagttggggcgaggggctttaaggtggatttctatactctacgttttttgtgtgaggcactcaaatggattaagcataaatagaggcacaaggttcaacagcgtttccctttgaaaggaccagaggagatctccacgcaacaggaccacccaacaggacattgtctaactacacacaacgcccaccagctgccggattactgcaggaaccggtccagcttctcctggatgcgagcaaacgcgtccttccccatgtagtcgatacggccttcctcccacttcctcctctcttcctcggggctcaattccttcaccttctcttcgatggagatctgggaaacagagacggccaggtcgacctagggaagacagtcagtgggagatggtttttgcagctgtccattatcgagggaaagactgctaaaacccatccagtgtagggtcccg

SEQ ID NO: 70 - DNA, 3700 bp, Homo sapiens
tagacgagagatggaacaaacaacacaaccaccccatgcgggcacagaagatttacaagcttaatctcatggacagaaatagactcggccccagcacagctgcagagcacacattcttttcaacacacacagctcacttgggaactggccacctctcgggctgagctgcaggtctcagggggttctgaaggaatcacagggactgctgccctgccccaaacgtagccggtgaggccaggcatctacggtaaacacagaaggagcaaaaacagctgcatgtatgtggaagaagaattctaaagccagccgcctgttcattaaaaagttcagacaaaccagaggggcctgtggcggccaggtcatccactttaaatcctcctcagtacgtgtactttaaaaagaacttcgtaaagagcccgtcacccaaagcgcactgataaaggcgacaccctgactcccaacaagctatttctgttgagggcactgagaaggcagctccctgactcatcacaattccagaagtcacagatacatgtgtcgcccttccagagtacacccacagttttgcaaaacacgtccatatacgaccaaaaacaaaggctgagcctaacactgaggctgcctgtttttgcgtagaagtgcgtgcgcttgatgggtgcaggtgagtgtaccccgagaacacaggccacgtgcaccgtgacacatcctctcgcgacaccagcctcgggcagacccccgcatgtgcagagggtgcgcacagcaggcagggcgcggtgaccagcagaaatgaccctcgcccccacggcagcaggaccggacaccacgatcaaagccacagaggaggtgccggagcagcagggggccggcggaagggacgctcagtacgggctgcaacgcacagccgtgcccccaggagcccccgctctgcagcggcccccactctgcagcgggaggcggaagcacgggaggctgtggtatggaatcagggacggggggtttggccgggacgcacactcatggattccagctgagcccctcgcccacccagatgacggccaccccctggaaggcagggcctgctgcaagctctgagcattcttctcggcccagcacttgactcccagggaccctctgagagggctggtagagggctgccagctacacctgcaaaccgcacgctggacggctaaacacaggagtcaaaaaggtcggtgtttacacagaggagccgaacacggagatgagaggccccacgtgtgggtttaaaaatcccctctctagcaaagagggagaactggtgtggaggggtcaacacagaaacgcagcaggtgcaggtgtctgagtaggccagagctcacgtgggctaacattcactcagacacatgactgcagccgagcaaccgggcctcaacggacgctgagagacgtcggctggggcctgcacccacacctgcagcccaggcactggcgcctgcagccacggctgcagcgaggcgtgagtctccacagagctcggaaggctgggctgggggacgtggggatcattctgtccaccagccaaggggtgacggtggatgccgcgcaacacagcgaggggaggatccggcaccctccctgcgtccacaagcccctggcggatgctcctgagcttggtcttctgtgtggacgttcccacccgggcttctgtttcccgttaaccccccttgctgcagctccctgccaggtggggaacccaagccctgccttctccctgccactgcccagggagtggcatcctgggcagcgtcctggccaaaccaaaggctgcaagggttttggtgaccactggccttgggaggggaacggcacgtgccctggcggtgagagcaggaggtgcgtcagggacgcccagagcccaggctgtcaccacgctgaagtcagttccaagtacagcggggctgccgcgtaggggacggcgctttcagccatgcgtggtgccgtgtagggtctgtgcgtccacccgaaggaccccgtggggacgccggacagtgtctgtgtgaccaggacaggtgaagaggggcgtctgtgtgctgagtcagtgtgtggggagcgggagagtcactccccaggcggggagggccaggctaggcagcacagctgtcctgggctgggaacaaggtctgagctgtcctgctgttgcccggggacagaaggcccgagaatccctgggcaggaggcgcaggcagtggctccggcaagaagagctcagccaagcagctgcacggccccactccaggtacatgctgggtcctacagtgagagcatgagccgtgtaacacgccatcgtcacacgggagcctccccggacccacggtgagagtacgtgtaacacgccatcgtcacacgggagcctccccggacccacggcgtgaacgcatgctgttccgttcccaaggccggcggtcgctgaacgcccccaccccccgagtttggtttgtcaaggatgccggtgacagggaagtgggcagtggcagggaggaggaggagcttgggttcaccatcggggcaggcagcacccgccagggggttagtgggaacagaagcccaggtgggacgtcgcacagtcagaagatcaagctcaggagcacccgccaggggctcgtgggtgcggccaacgttggccgtggaaggctgtgcccgtcagaggacccctgaaaacagtaccgtgctgcccggccgggagcgtccgaaggcggaggtgcggcaccccaacacgtccagtggctccaacacgggtgctccctgacaaccctgagggtgtgtccaagtggggtggacccaacagacagagcccacactcatgcgcggagtgaaagcagccaggaaacgtccccttctcccccaacaccacccccacaaatacccccaaatatgcctgtaattcctccaccacccctcagacaacatgcatttcacacgtctgtcctcactccctaaaaacgtggaaacctattttctgtaaaatgaagcaaacttctgtaaacggaattcatgatttcccagaaactgactttttaaaaataaacagtcctcacaggtgcatcgtcaccacagccccccacagaagagccagggccccactgcagggctgaagggcttcctcatccagccacgtgcgagctaatcacctcattgactctgcgaccagcgagcccgcaccgcccagcacctcccaccatctagagcaaatcccgcacgaggctgatctcgctcttcgcaggttaagaggattttaaagacaccagcctcgcccttacccacttacaggcaaaatgtcaaaacctggaagacagaggtcaaaaactccgaaggagtgcaaaagttgatgtgagatcttacagaaaaaatttcaattaaaatatcaacagaaagaagtgggtcttcctcccccttcaagcaggatgccttggttcaccttgatgttaggccactagttccagactcctggaactgagtttgaaaagcgcgtctgatgtgccacgtgggtgtgaggcgcccgccacgcacaccctgtctggatgaaattcggatcagattcggccgcagccaaaccctaaattctcaaattatactgggattgtcacaggaagactcttacacgtttaaatcacatggtactcgtaaaactaactcatacaatatacacggggtacagacacaa

SEQ ID NO: 71 - DNA, 2529 bp, Homo sapiens
ccacagctttgatctagggaaaataaactgattcagtctaagatgggtgtacttggaaaatctggaaaaaaaatcctatttggtcattgcctacctgtatatcaaatatccacagaaggcaaatagagttgtcacacaatcaactaacacataaaattatttgaaaaccataatcaagaggcatgatcctttataaactgctcaaaaatactgtgcacaccaggtctatcctttttgatgtgactacagctaaatctgacatcagacaagagaggaacacaaacacaagtatattctctagttgaactttagggcataatccatatgaattttcatgtgcagatgagatcctgggccatcttctcctaaccaaacaagaaaagcaactctgtgcacataatacgtgaatcaatttctccagccttggacacttccaatctcaaactggtaccttctcacaactggtcatacaagcagttctccctgagtacagaaaagagtatgaatatataggaaatatggttaattagcaggcctaacgatgacactggtcatagttacaaaatttcaaaataaaaagtgtgaaaatgaaacttttagttattgcctagtttgggctacaagccttaaaagcttgcactctgcaatgacttcataggttcactaaatttataacatcacttggttttgagttgagaaaaacgttttcagatccatttattaaggaactttggagattaactacttggacctcctggctgattgtctttcacctaacccagacagaaatgtttccatctgacccttaaaatttactgaagacaatattactacattttctgcagttattagctaagaggccttacaaaaggaactgaaaagggaagcaggccaatgacaaaaactgggccatgattatgcaagattcaacaggttatgagtgaggtgtttcaaatccctttctcttttaagatttggcactgacgttggatagctttctagcttggttcccctggaaacctgacgaagggagaccaccagctgtgtgacgagagactgcttctggtaaaacgctcagcgaagtatcctgtgtccaagctaggagagctgcaaatgaatgtaaatacctgctaagagtcacagcttgggctccaagagcgcagtgtacaacttgttcctgggctttgtccctagccggaacccaaggatgctacatgcacagggaactgttaaaaagagggtggtcccttatggcttctaaagccaaggtgactcctatgtccttttgtgcagtctgtggggactgccaagataattctcatagaactctgcctaaagccaccctctggcatgctgtcttgcctgtccaatgtccttcagagcaaactggtaacagaggaggcctttccatgttgtgggagtttgtgtagttgaacccaacaccagctgtgacgggcgctgccctagcactctggagtgtcttcagaggcaaccccatcccacattggcaccaaattgtcatagccatgactatcacaagagtatgggattagaaccaatgaaggcaaaccttcaaaaaatggtttaagatctttaaagacatcactgaagtttaaggctgtgaatagcaaatatataaaggcagagtgttcactcattaaaaaatggaccttaacattttccccaaacttagctattactaagtaaaggagcaaagtatcatggtatagaggggtaaattttcccagaagcaaggaaatgtggctgtcattctggctgtgcacatagccgctgtatggccttgaataaggtgcttctccctacagatgtcagtgtctttatattgaagaggatgggtagggggagcaggggatgatggaaagcacaattgaagtacaggaaaaacacgaatttagaaaaatgttacattaataacagctggaaaaaagaaaacaccaatttggcttgtgtgttttaaattgtaaaacctgcaaacaaacacctatgattctgggcttttaaggtgagaacaaaaacaatttcttaagtttttgcctgttgatgcttcactcaattctcaacatacctgttcgaaaactcatcagcctcacagcctctgtgtcaaacaagttctatctaactaaacaatactttcagttaaccccaggtaatgatatactatgatcattgactccataattccactggtaatctagtctcagaaaaaaccctaaatataagaaaaagtcttatgtaaacataaactgctcagttctctacttacaatagagaaaaagttttaaaaacaacccacaaatttcatgctaagtgaagaaagtaggattaagacaaaatcatttcagctatgttttcaaaaaacctatgcacagaaaaagaaacagaatacacagaaatatcaagggggactgcaaatagaacatctttttttcctgttttctaaattttcttaactgaacatccattttataatgaaaagcagttcaatttaagttgcatttccaacacatt

SEQ ID NO: 72 - DNA, 2446 bp, Homo sapiens
tagacacgtacaaagtagctgaaagaccaatgaatacacggtctagagaggactgcttaacacgctgcatatagaagtgtgattttttttggtacaattttcaagtgtgtttctcattagagcatttaaagtaagccacagtgtccgtttgtatcaagttagtactctgacggccacaaacataggcaggctcacttctggatgtcttatttctttgcatgttaatcgtgttgacacaacttgtcttgaaattaagtttaaaatgaaataccagtaaaactgaaatgaataaggcctttattagccagagaaaagaaaacaatattgaaactaaacataagaaagtgagggctgtaagttatcgtaaaaaggagcatctaggtaggtctttgtagccaatgttacccgattgtcctacagctttgtccagtggctgtagcggtcccgttgctgcggtgagctggctgcgttgatgggcggtaagtggcctagctggtgctccattcttgagtgtgtggctttcgtacagtcatccctgtacaacctgttgtccagttgcacttcgctgcagagtaccgaagcgggatctgcgggaagcaaactgcaattcttcggcagcatcttcgccttccgacgaggtcgatacttataattcgggtatttctctctgtgcatggcctgtaatttctgtgcctcctggaagaatggccatttttcggcttcagtaagcattttccactggtatcccagctgcttgctgatctctgagtttcgcattctgggattctctagagccatcttgcgcctctgatcgcgagaccacacgatgaatgcgttcatgggtcgcttcactctatcctggacgttgcctttactgttttctcccgtttcacactgatacttagagttacagctttcagtgcaaaggaaggaagagcttctccggagagcgggaatattctcttgcacagctggactgtaatcatcgctgttgaatacgcttaacatagcagaagcatatgattgcattgtcaaaaacaaggagagtgcgacaaaattgaaaggtgccagagttcgaaacttattttactatccaaaactcacttctaccagattctttgttacgttaacttttgtaatgaaacttgcatttctccgccctcaacaccccctcaaccccgcccaaccagcctaccccctagtaccctgacaatgtattcattctcaagcaaaacatggtaattcagtaacgttgactacttgccctgctgatctgcctccctgactgctctactgctgtcctgaaaaatgcgaatttgacttaatcgccaattttttcattgaccttttatgtcacaaaacgagaggacacaaaaagctatatgaattgtttatcattatcaatatatgtgtatgttatctttaaaaaaacaaagcttaatgagaacctaattgtcttaaccacacacatacatacataactgcatattgaatttatagtaattattatcgctttttcttcacttctatttaaaaattgaaaattctatacacatttttcacaggcattaagtatcagaatattagcatatacttacaagtattttatgcccaacttctaggatggctaacatttgacttttagaaaagtaattgtttcgtttagagaaaaaaaaatatgacctaagaactcaaaacagtttcagtgaagtgttaagctacactaaaaaggggacacaattcttttctttgcagattgtatagtgggatattttgaagtcattctcttcactgtcacacaattagcaatttaaaaaacaatcttttacaagtctaaattaaatttccattcacaacaaatagagccatcaatttatcatatttcaccttttagttcaacctccttcaaaatttaaaggtcacagtttaccagactaaacaagtgaataactctcctcaataaatcttaaagtctgaagagaaatgacaagatttctttgctgaaataaaatgggaggaaagtccccccactcaccaatgttttaatgccatatttgcaaaacaggagtaacaactacaggttgcatagtacacagaacctattaataaaaataaactctcagcaaaactgaatgatgccacaattcctaagacaacaaaataaaaatcccgtaaaatatgaaaagagttcatagaaccaaatgtggttggtttgtccagtaaatgttataatgaattaatatcagaaactttaaaaaattatattccatgaaaagaaaaatatgaaaactgtaatttgtatcctagttatctactaaagtttagtatctaagatacaaaatttagtattcattatacaaagtggaaatatagttggctcaagttaaaacatgtatctggatagcaaataaaatggttaaattgcagtcatacacagaaacagatt

SEQ ID NO: 73 - DNA, 2000 bp, Homo sapiens
tgctaaattcatgggccatattttcaacatctaattctcaaaaagttagaatagtcttctgatttggtaggtagaagttaatgctcactttaattgctaggttctactgtttcaagacttaatcagataaatcacctagcaactgatgcatttaaacatgatcaattttactggcatctttttttcccagggataatctaattatttgccagtgggaggatgaagtagggtgcagtgggaaatagaatgatctcctacctgagccgaagaaccttacaaatgcatatctactacatgtaaattaaactataagtaaacaaaatagtttacaactttaaaataatgctgcctgtttttttctctaacttcacctgaattatttttcttttactttattattttgattttttcaaagtataggaaattgcctgtaaaaacaaggtttcatacttgggaagaaatttctcatagagtgaagcattttttttttttttcaaatcagttgtaactaaccgtcttaaaatcacattgtggctatccatgcctgaaatatgtaaacagaaaacagatgacatccacaattttcctttcttccttaaaacaaagaggtaacttcactctttcatttaccttctgatgcacaagtatgagcttctctttttagttcttctaatcagcttagatactacatgttatagcttgtttctctccataaaatgaaggtcacttttgatcttttccagggtcttccttcagttcctttttgtccaaggctaactacactcctctttgtctagtgagccagcagctgtttgaccaagaaccattttaggaaacagtttttaaagatacctcatggaagcattctgttgtacccttccgtacattattttttctcagtctgttgcattaagattagagactgctttctttttattaatgttttgaaatattttgtttagtgtccaaaggcttggtcaaatcatgaatagttctatttttcttctgaaaaatattgttcctttagtgatttatagttaagagatattatcctttagctgtcatacatttcaaaaatactttcctgattttggacttaaaattgcatttatcctttttatcttaaccttcaaaacaataatataacaatgattattataatttgtgcccgtttttgccttctttgaatgacgatggctttagtatcttactgctaaaaaatgttgcttgtttgtaaaatagcctttatgcagaaacctgcagcaagtatccaataaccacaacaggaaaaatctgaggaattccgggcttttcaaatttttgtattacctagcaattatatgttatttgaaatttgattagaaaaaggctaaaacaattgtttgagtctggtaattaaaaagtggtaagtctttgtctgatctatgatggttagtagtttgtattttgtggtaaaaacaatacttactttccattttcaaataattttaattgttataagttattataagcgtcttgtaattagtttttactgcctctctcatagctttggttatatctaatttctcatttataatatcacttacatttgctttattatatttgtatttaatctataccagcaagaaggcacttaatattgcaagcttttaaaagaaatagggcttcttcttttgctaatcctctttgtaattccttttggctttttgggagaagttatttctactcaaaccttgttcaggtcacaaagaagctacagatgaagaacacgaaaaaattgttggttaaaataaaactataactaggcttatttacggtgagtaatttcttttcatgctccatttaaatgtttttaccctaaagtaatgatgtaggagaagtctaaagcaatggtattaatatacaagtcccagtgaaaatgtgattcatgaaactctttgttatttttggctgcatgtacattgttacgattgtgatgtgagatgaacattttgcatctt

SEQ ID NO: 74 - DNA, 1865 bp, Homo sapiens
tcctgaaggagtgtatgacatacgtacaaggaaaaaattgaggaaaatgagatgaaggtctgcaggtattgagaggtggaagcaaatcaataatgcaagattttgggtccagtttattaagttctccagctatgttcaacagcctcggatagaatggaggaaagcagatcttgggaaggtgaacgtggaagacagacaagacagtgaagtgttctcagcgtccccagggacatcatgagactgaattgaagaacaggtgaagatggggcaggggtagggtagttagtcatgatgtggggaggtgagcagaggttccagatcctctggaaggtgtatttcaacaaggctgtgggtgggtatgagcaagtttgtaagcgtgaatgcacagcagtttcaaaccatgacagggcccgaagaatgctgcaggctgcagatgatgcagctcctgtggggtggaagcaatcctatgcatgtggacccctcgggtccgactggaaaaggagtaaacgattgttcgaccaaagcctaagcttcaggaggaagagccttgccttcctcatcctaccttattatcattaaaatgagctgctggttaagaatttgaaagccaagaatattctctgatacttgtcagaacttagtggtttctaaatttgtagcagcgtaagcaccaaatgcacctcattcatttgcttgactaaactgaaattctcagcaaaccaggcttcccacctctcactcctgacaaccctcggggtactgccactgcagtaacttgggctggaaaaccttcagaaaactgtctgtcttcactccacccctgcacagccctctcttcctccaaagatctgtggtttgggacaggctagtacagaatttggttctgggcaggtacacttggcttccatttcaaagcacccaagtcaacctggcaacctgaaggaactagaaaagcttctgctaatcagttgttggtcagcagccctgattcttgtggacggcagggacgataggctctcctgggaagcagcggtctttggaactgtggggaccacaaaagctctccctgtgccggcaccacggccctcccacttcatcactgccgtctaactgccctcaaactgtcactccttttcctgaatcattagttttcttggaaaaaaataatcagacccataaggaggaggagagtatgaaggaaaaaataaaaccaaaatgagcaaaattcttccagtcaatgggggtggggaaataagactcatcagcagcccctcaaaaataacatgattatcttttattcctttttacttttggagttctgttgtaaatacttacattacatataaaagcagtttaaaaaaatttccatagtgccacaactacttactggggataatgtgggtataatcttgcctgcaggcaagagagagattattacacctattttcaagctttctgtgactctcaaaaatagatgttgacataggtttttgaatgcttctggaaatgttaaaatcattatgtgattattcaaaatatagtttgccatgtgatcaaaagctaataaactcttctatgtttattttgttttaaggcataatcggcacaaatgcattgttccagtggcttaacattgtatgtaaacggtataaacagaaattgtggaaatgtgtgttttcacttgattcaaacagagaaagagttccaaatacgaaaatgaactaaataaaaaatgagattggattgctgcctgaaatttgtaaatttaaaaaactaactctctaaagtaaattacttagggaccttcatatttaccaaatcttctgcataataaacttagaattaaacttagccctcctacatgc

SEQ ID NO: 75 - DNA, 1517 bp, Homo sapiens
agcttctttgaccaagctgactacaggatgcccttgatggagagaccagggatcatcaccttcaagttcctggtccttcttcttgaactaaagactccttggctttgctcatgttggctttagccaccagttgctttacagcctcccacactcagtctctcagcttaggtatcagaagatacttccattttttaaaaattatttagctctctcatgacctcctgtcagcagatctacctcgcacctcatttccttaggctgatacctaatgatgctccaaccccacggaggggcatctagctaactggtactaaataacagtcacttaaaaggtagtttaaatttcacacattaagacatacatgtttgtgcaaggcagaggttttctttcttgttgactgtattttcaggttgtagttacagatacccattaacaagcctgccttctgaaataagattatctcagtcaagtattctctttgttatgtgtggcatcatcagacacatctgcaatgatcccaaaaaaagatatgatcagaaccacatttatttaaatatgcaaaatgctgcaggagagctattggctgatgcataaatacaaattctgtttccatctatgagaattggagtgaggacggggagtcacaaccatccacaagtgacactgacttaataacatagaaaatgtttcagatttctcatgtactggggaagacaagagtggtgagcacaatcagggtaataaaacatccctcagctcaaagagataattctaatatcatatattgtgcatggagtagtgaaggccaaatacaagcaacttcacatcagtacatagcctacacaagacagccacaagtcaggaaagggttgtattgcattagcaaatgattgaattaatagctaatgatctcctagaagaattatattaaagacttttaattgacactttatcaaccataatcaactcttttttttcattgctctgctcatttatgttccaatgaataagactcaaaatcctgaggcagcttaaagtatattttacatcagtcaccatggtcagtgtagcatacattttatgatttgaaaatttgtaatagcctttcataggctaattgctgagccctctaccagagctaagaaaagagtgcacagttttgtacattgaaagaaaaggcaaaacacagtaaggcaagcagcagtaaaatgagacagctgtgtccagctccccagcaacccctgccaagaaagccctttatatgaaaatgaacatttgacaagaaagcatattaaagtattagctttttcattcagcatagggcatctctttattttaaaaaaatcttaggattgctctaataataaattgcctaatgtgtggacagcatgattccatttgtaaaatgtctatttagcattgcttttcaaaggcatgtcattgctttgtgagatgtactctgaggttaaaagatgctttccctaagaaacactagctatggagtaactgtcctaca

SEQ ID NO: 76 - DNA, 1634 bp, Homo sapiens
cctgcttgtctctgctcagcacctcataacttcgtcttcctaagatcctgtcagccacattctgctgtgttttctccggccccaccactcttctgtgcctcatcttacacattctccattttggtgacaaagctggattctgtctattggcctcagcaggctattctctgcctcggtatctaagtggcttcttgtcacttagataattaatttcagcttccttttctctgacagtgataacctcaataccaaatctgaaaatatctctaactgcatgtctcttttcccctcaagtcacaaatcgaatcggccagatattttagcacttaccgtaatttagcagcctcccaatatctgagttctttagtaactgagaaactttggatgctattcacagaaatttattttatttataaacaaaatgtggccccaatttgtcaacgttttaattgcctttgcaacattgttcctcactccaacccaccatggaaataagtgctggcttaaagagaaaccaaggaggacctgcagaattagaagcaggcaacaagaagactgatgagtattaaatgggactcccaagagaagttttgcatgggtcaaccgtcctccatgtctgcatctagctagggcttagctggcttttagatgaatggaattctgagcctaacaaccaacagatacctttctctgtcccttaatgtcagcagaaggaagtggaaatgtttaggtgaatgagaaaataaaaatagcacatttgaaagaaatgatcaaaattaagaccagatcagtatattttttttcaagccacaccaagtgtcagatgactggattagtttgcatctggttttgaaaattctgtctcaacattcaacagccagcacctgtcgtgagcagtctgaggctttttcaagtaagcttcaaatatctgctgttgaatgcatttggttaaaccttgtttctcttgaatgcacgtgtacagtatacactgggcagagtccacagtgtgacacacattgttgagtatgtctcctttaagtgaagagtcaaccatgtgccacttggtggaggaagatacactctgcacagtccatgcttatgcaaagccactgaccccactctggaactttttttttttgccttggggtgaatatgctaagcttggttacgatgagaacacagttactggttttctagtctccctaaccacaaaaatcaataccagcttagtttgcaaattttcttagcaaatcaagattaaatgcatggcttggtttgaaattggatatggtcatgaataaaccctaagttttaaaatattgttaaacaactgtcttctcatctccatacacatcatatctgaccaatgtctttatatgtgtattctatcatatctgttcacagaattcttatttcccatttggcagaagaggaaagagatctgccaaagaacaaatgatgtatcctggtgatggggccaatctttgaatccaagccctgtcccaagatgtttctattctaaatacagtggaatcaggagaaggataagctacaattttttctcatgtgtatatatggagcaggtaactgacagattctcaggtgagattactgacaagccaggggttgcagac

SEQ ID NO: 77 - DNA, 2920 bp, Homo sapiens
gctcactcaggcccagcgcccgacaagaacccccgacctggggcctgggccacccccttcctcagacttcgcgtgacagtcttgtgccacccccccccactagggattcacgtgacagagacacgtgcccccctcgccagggcctggggtgacaaccactcgctgtcggggcacaaaaagctcacgtcaggcaacgatgaggagagggaccggggtcctcgcaggggcaatggctgccgtcaggcgcctgagccgtacgtaccgtgtgactgctcctgagaagatcctgtctatcatcttggtagaaagggctggaaaggaatgcggttgatgggcagcccgcaccgtgcctcggccccgacgtcaccaccccccggagccgagactggatgcggtggggaccgaaaagctgagaggacgcctgggtctgggagagccccggggccccgatgcccctgcacggcccatcctaggggcccaccacgctttcccgtcgagcagagccaagtccagcatgaaatccacagagcgcaaagctgaccgcggctccaagaccgacttgtaaagagcagaatattcaggcctcaaaggtacagctttcagacggagagagagacctcgagtgtgatcacggaaacaaacacgtttcaaccaaaggttcaccaacgggagacgggagtgagacctcagcaacgggaggcgggagtgagacctcagcaacgggaggcgggagtgagacctcagcaacgggaggcgggagtgagacctcagcaacgggaggcgggagtgagacctcagcaacgggaggcgggagggagacctcagcaacgggaggcgggagggagacctcagcaacgggaggcgggagggagacctcgccaacgggaggcgggagggagacctcgccaacgggaggcgggagggagacctcgccaacgggaggcgggagtgagacctcgccaacgggaggcgggagtgagacctcgccaacgggaggcgggagtgagacctcgccaacgggaggcgggagtgagacctcgccaacgggaggcgggagtgagacctcgccaacgggaggcgggagtgagacctcgccaacgggaggcgggagtgagacctcgccaacgggaggcgggagtgagacctcgccaacgggaggcgggagggagacctcagcaacgggaggcgggagggagacctcagcaacgggaggcgggagggagacctcagcaacgggaggcgggagggagacctcagcaacgggaggcgggagggagacctcagcaacgggaggcgggagggagacctcgccaaggagaggcgggagtgagacctcgccaacgggaggcgggagtgagacctcgccaacgggaggcgggagtgagacctcagcaacgggaggcgggagtgagacctcagcaacgggaggcgggagtgagacctcgccaaggagaggcgggagtgagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagggagacctcgccaacgggaggcgggagtgagacctcgccaacgggaggcgggagtgagacctcgccaacgggaggcgggagtgagacctcgccaacgggaggcgggagtgagacctcgccaacgggaggcgggagtgagacctcgccaacgggaggcgggagggagacctcgccaacgggaggcgggagtgagacctcagcaacgggaggcgggagtgagacctcaccaaggagacgcgggagtgagacctcagcaacgggagggggggagggagacctcaccaaggagacgcgggagtgagacctcagcaacgggaggcggtagggagacctcaccaaggagacgcgggagtgagacctcagcaacgggaggcgggagggagacctcaccaaggagaggcgggagggagacctcagcaacgggaggcgggagggagacctcagcaacgggaggcgggagggagacctcagcaacgggaggcgggagggagacgtcgccaaggagaggcgggagggagacgtcgccaacgggaggcgggagggagacgtcgccaacgggaggcgggagggagacctcaccaacgggaggcgggagtgagacctcaccaacgggaggcgggagggagacctcagcaacgggaggcgggagggagacctcaccaacgggaggcgggagtgagacctcagcaacgggaggcgggattgagacctcaccaacgggaggccggagtgagacctcaccaaggagaggcgggagtgagacctcaccaacgggaggccggagtgagacctcaccaacgggaggcgggagggagacctcaccaacgggaggcaggagtgaaagcaccgtcgccgtcagcttgggccacgagaaggtcccgcagcctgggcggccatccctgcggtcaccggtgtccctgggacgcacgagccaaggtgccgccccccgcttcaggccgcagtgcgtgagaaacagcgcagcccggccgcacacggcatcctgccctgggaccgagagtgggctcc

SEQ ID NO: 78 - DNA, 2419 bp, Homo sapiens
ctcctttccccccacaatccctgcacacccgtgggcacctatgctctcgtgtggtctggatctgccctctgtgtgcacagcctgtgcctggcccagcgtgagtgactcgtggatgctctgcaggtgagacctgaggtgagtgtcctggcaccgcccgggcctggctatcgggaagctccgcccagacggccgcctcctccctggcgcgggcctcttccctaggaggagctcgttagcttgtttttccatcggtattctttgtccccagtcacccggacctggggctgggcactgccaggggcaaatgtgccatgtggagaggccaagcgggggacaggggcggcttgtccgccaggtggcaccgaggcggctgcgtgtggggcagtgttcccactctcgtcaccagcccgcacttcccgctgcctctgagtattctgtgggggctgccccggctgcagccccaggtgtagcctgctggaaatctcacggtgtccaggccccatccctaaccggcccggggcatccctgatttcgtgctcaccgagaggggcctccctcggcctgcccagctaagagccttgcaggagcccttctccagcctcacactgccagcccctttgaattgcagcactcaggtccccaggaaaggtgtttttatccagttagctgttttttatacttatgaaaaagctccgtcgcttggagcaaagcagagttgattttcagatgtgatttctgcaggcagagcaatgtctggttcctgctgtttcttctgatgggcgcggcggtgactgagggtgtcctgcgagccgtcggtgagcgctcagctgtcctggtctgcaagttcctactgacatcacaacctgctgcttctctctgtccttaagggtcagaagatggagaaaaggttcatgtttccacccctgtattctgttaggttcgggtttttgagagaggcttgtggggaaggggccgtgtccccactccttcctttcttcttgtacacatatttacatccactgattgagtgatttacaatcactcaacatgattgacggaacttctggcactgcggaagctgtgctaaggcctgggcattcatgggacatggagcgtgcaagagctgaagttttaatgacttgcttgcagaaaaagatcaagttttacaacagaaaattatggggcataatttctattgtggcaagggaccagggccgtctcctggaggaaatctggagagaacatgccacagccaggccggcgtagagagaggctctggcaggggcccctcccaacccacccctgcatgcgtggggcttctgctcagcaacaggggcgcagctccactttcaaagtgtgaggggcaggggctcaggtctcggatgccttcaccacctgcctgagtcgggcatcgggcagggagcgtgcgggggcctctgcctctgctggcccagatgattccctggccctcctcaagtgcagctcccattaaatagatagagccgggctctgagccacgaattgggccaagcatcccaagggggtggaaccgagtcaggagtcaagaccagaggccaggaactgcccacgcccatgttccttccacagggccagcctgtccggtggcaacactaataccatcccatgaagcctgtgaaaattaaagggaatggtgcatgtttagaggccacacacagcaagtaaccaatgaacacccacccttcatgcttggttttcatcactgggccagcaggggcggaggccccagcactctccctgcctgatgcccgactcaggcaggtgggcttgagagcccctcccggggctccagggctctgaaggcatccaacacctgggcccctgcccctcacattttggaagtggagctgtgcccgtgctgctgagcgaaagccccatccagctctccgagaaccagacgaggggcaagggagatgaagtcttcctggaaacttggactccagctggtgtgggggtcagagcagcaggctgagccttcagggggcctccggcaggctcccaaggctgcgctgtgcgtctcttccaccacacgcactggggcatgaggccaagggcatcgtctgcagagcgagagggaaactggggtggcagggcttgcgggcgcaggacagcgccaaggggctttcgtctcccagcattaggacgaccttgtcctctgcccctgtctgggggccgctgggtccctcctcacaggagcgaggcaggcagctctggtgcagggccggccaacaggcctcagatctggagtcacagacccaaggacgaggacaagggccccacacacctccaagcaggccctgaggtactgacgggcaggcaggaccctctgtgacccttcctcactcctcacccagagaagccaggagagcgggatgccgag

SEQ ID NO: 79 - DNA, 3355 bp, Homo sapiens
tggggcaggagtcacagtgtgggaattaaggaaaaaacaagcaggtagggtagagagccggactaccatcaaagcatgagttttctgctgcccggctccgccgtgacgccactcctcccaccagaacgagcgcgtttgtctccacactctcccctgcttgtcattgagctttgttcggtttaggaagcacgaacagaaaggtggctgtgacaggcagtgggctggaaagtgcatttccactggtctgccctctcctgggacaaggtgagcttggtgcttagcactgggccgtcccgactccaggagcaacgccagtcctccaagcacgggaggcttttcctcctctcagtattgcagcaggcagcgcacagcccttctgtccaaatctgggaacctgaaagaccttcggaatcttgctgttttagacgttgtaagaggagcgggtaggaccccacgtgctcaggccccacgctttggatctaccccctctgcagccagagggacaagcagctgctgtgctggtcatggcctcatcccgtgtgtgacgatggccactcacgtcttctcattcaacagaagttatcaccgtgcgtcagacttttatttggattttgtgcgtcttgcatgtatggtggggatgaccggccccacctccaagtgtaggcgctggagcccctggggacgcagcgctgcttgttcctgacagatgggttgcacccgtgggaggggtccagatgtgctagctcttgggagtcagtgatgggtgtaccgggaatggcctggcgtgcatttccattcagaaactcccagtccctgcctggaacctggctccttttgctgtttttttccccctttcctgtccctttcctgggtggctggtccctgctgtcgcccctgcctccctggctgcagagctttcctctggaggactcgacacagagcctgcgccgtctctgactccgggctctgctgccctgccccactttggtctctcaggttggagttgaggttgcatctgctgagagccgtgcccacaggtgaggtagtatcagggtcctgagccagagtccactgtcccctggccgtgggtttggagctgccagccatccttccctgagaacccagcctatgactcggctccccttgggcctgccctatctttccttcctgccctggtctgtcctgcggccccctcagtcctcatggccaagtcagccaacagcaacccacacacagaggccacttctggatgggtgtctggcaaggtgtgggtctgaattcagccttttgcctcgcgtgccaacccccgtgtcctgggctctccaagagccaccttaggaagatggggagtgggtctggaccactgagcaactggtcattctgcatcagctcctgaaagtcccttgtggaccagctccctgatgaggacaagctcttagctcagaacaacacagaatccagcgctgaccataggacggctgtctaatggtccttctctagaaacctctctgtgccattctgaaagtggaaaatgccggcattggtcatgcgaccttgcatagctgtctattttcatggtctctccacccactctggccccttcatgttttgtggagagaatagcagacctcgccccccgccccagtgttaagaggtgacttagacaccctcaccttgaagttttcacatattttctatccatagtatttgtatacttcacacgaagacttattagtggataaatataataaactccttcctattgaaataaaatttgagaagaacatggtatgtgccagccaaagcccaaattcaaatgaacccttctgtgaaggggaagaatcagtcttgttgagagaaagtaatttagatgcagaaggaatcccagctgcctagaaatccccgttgccaacagcaggcgaaaggaaccacccatgggagggaatgtcgcagggcagcggcaggtcgggcggcagtgcagcagccgtgagaacgcaggactcacacttccgggctgtgtcgccaacattggcaaccagtcgtcacctgccaacccacttgggggagcatggatggtattggtcgggctctatccagctgtttgttagcagtgagtacaaaaaaataaaaaaatgctattttttagctggtcagaaatgacttgaaagacctcagactgttgagttaacttaaaacagcccctcctttgcatctaacaaagtaataaaattgtgtgtgttcatccaatgggtaaatatgcagcctctgctgtttcaaggaaagtgaaaggctcagcagtatgtgttatcttgccctccttaaggcatgcttttcctctgaatgtccttggctcagaaagctggttgtcagggagcttcactggggtctctgaggggacttctccagaggagctggtgaaggagcgcgtgaggacacaggagagcagcatctctggctggcactctgcccagccgggcaggttgagcccactttcacaaccctgaggcggtcacagcccgaccgtcagggggaacccactctcacggtcctggggtggtcactcagctggcctggcaggtggcacccagtctcacagccctgaggcagtcacagcctgaccgtcagggggaacccactctcacagtcctggggtggtcactcagctggcctggcaggtggcacccagtctcacagccctgaggcagtcacagcctgaccgtcgggggaacccactctcacagtcctggggtggtcactcagctggcctggcaggtggcacccagtctcacagccctgaggcagtcacagcctgaccgtcagggggaacccactctcacagtcctggggtggtcactcagcggtcccggcagggggaacccactttcacagccccgaggcggtcggtcactcagcctagcccagcccagcaggtggaacccactccccactgtcacagccctgaggcggcgggggcgtcctccacctcgctcttcctggagagacgccagtgtgtgggtttggaagcggagtctattttaagtttgcagttcctgaaggagcctgtgttggctgtgctgtctccacatggtcacagccttgaagcctccagccttttaaggacaagcctctgcctggctgcctgtggttggggcaagccgctacttacgttcgcggtgcctgttgcgttttcccacctaagagggcacaggaggtggtggaaggggagtggaactaaggtgggggacttgagagcaaactgtgagtgtccagagctgtaggaggttcggagaagacaccgagtgctcctcctgcagggtgagaaaccctcctgtttctgattgcctcatgcaccacc

SEQ ID NO: 80 - DNA, 2503 bp, Homo sapiens
tgaggcaactcgtagatggagatttgggaaaagacgatgtggcctcctacctttccagtttctgttggcagcccttcacgtagcctcctgcctcgcctctacacctactaccctgtcggcccttttgccatgctgtcctcgtataactcggattctctcctcaggtgtaggtgcagggagtcagggaacccttagactcccctgtgtgcaagagcccaggtgttggtgtgtccctttaatgctactgtgctctctggtgtttctgattttcctgcctttattctgtcttctcttgtcctatctcattccagcccacatcttctcctttcctgattacttttgttgtcctgcctcttcaggtaatggtcacagatttggctgtaggcacgttaccagccctgtggcttcttgactcttggttccctgttaactctgtttctgagaaatgtgggtatggaggtgggtgggaaagctcacttccatgaaggatgtctccatgctaggagctgcctgcaccctggcagaggtggccagtcacgtgaaggtgggcagggcccttagcatggccacacatgtccccagggcagatcaaggggcctctcagaaccatgttccccagccaggtgaggaccattttcactgggacccaggccaaaaccatgtgggtgcacaaagccaggcactgccaagtggaacatgaggttatttccaaatcatgggagccaccagcagggagagggcaggatggaaaatcccctggagccggtcaactttttgctcatggctagtgaaataaagttgtttgagtactagatgccaagtgccgcctttatcaaacctaaggctgctgaccagagtttggaagtgatctaagaacaggtccattcagttccaaggtctcttgtaccttcccagggcagctcagtgatcttgcatggaggaccacttgattccacactaaaaggtaagacttcaaggcctacatattgggttttctctgttaatggcaagtacaagatggctcaggatcatatgcctctatttctgctccagccagtcggccaggagtgacccggcagtctccagattatccccgcctgctctatttgagtgtaagggtgtgtgtcttactccacaggaaagggctgcaaactgtcaaagtgagtctggaaagggtcagaggtgagggcctgcagagagagaaacaggacctgcacctaagctgcattctggtacatggtttcaaagggatccaggatttctgcacctcaggtgccaaaacacttgctctgcccacacatgcctgcataaaatactgtttattttgtcctttaggaagactaaagtagtccagctcccctacagcccagtcttgcccccaccctgcactctgtcgccttagttcctggggaccaagcatctggcatttctcaagcagaccctctccttgttgctccttttcagtccctggagtctggcttcccaaagccaaagctggaggagagctcattgctgaggaagcagggttggagcctgaggagatgcagagggcctggacccctcgctggatcccagaggcccaggggcagagatgctgggacagggctctaggggaccactgggtgactcttgaggggctagaagcagggctgggtgacttttgctacggtgggctgcaacactgtctggcttctcaaagcgcttgccgcagaattcacaggggaagcgcaaggcagccaccgtctctgcatgcttgcgctggtgccagttcagggaagccttctggcggcaggtaaacccgcatatctcacacctggagtcagggacagaagagggaaggaacaaggcctcaggccatcatgacttccctagggggttcctcctgctccccactgcctaggtgtcctatatgcctagcttccagactccacctcctcccttctagcccctggccctcagaccccaccccagcactcactgcaggggtttttctccagtgtggatacgtctgtggatgacaaggttgctgctagtgcggaaagaccgggcgcagaactcacagatgtagtcccgggtgtctgcaggcatatgagggacactccagcatctgcccccaccctgtggcccctccttggcccaccccacccactgtccctcaccagagtgcaccgtattggaggtcaggaggctcaggttctaattagttgttatccaaatcatggagcccgtctggacctcccttacctgatgggtcatgacaaccaagtaagatacgaacccagctaaaagacttcattattgtccaccccagcccctgcccgccaatcccactcaaaccaatgaactcctgatggaagtgcaccaccccacctcagcctctaggctggttctttctcaaaggagacacatggaatggagagctgggtccttatgtatgaattgaaggcagtgggcagcagccaagcagaaccttggagtcagcgatgggaattaggattgaagc

SEQ ID NO: 81 - DNA, 6191 bp, Homo sapiens
gtcagttaaccagaccccagcctgcatccccattgatgaatcaggcagttcctcccgtgcagccgctaagagcaaaggggacctgggagagggtgatgtggtcagtgggcaccatgccggccttgccaaatgctcaggcactctgggtaagcactgtgtaccggctcagatgttcactggctcaggtgtgcaccggctcagatgttcaccggctcaggtgttcactggctcaggtgtgtactggctcaggtgtgcactggctcaggtgtgtaccgtgcactggctcaggtgtgcaccggctcaggtgtgtaccggctcaggtgtgcaccggctcagctgtgcaccggctcagctgttcactggctcaggtgtgtaccggctcaggtgtgcactggctcaggtgtgtaccgtgcactggctcaggcgttcactggctcaggtgtgtaccggctcaggtgtgcaccggctcagctgtgcaccggctcaggtgtgcaccggctcaggtgttcaccggctcaggtgtgcaccagctcaggtgtgtaccgtgcactggctcaggtgtgcaccagctcaggtgttcactggcttaggtgtgcaccggctcagatgtgtaccagctcaggtgtgcaccggctcaggtgtgtaccggctcagatgtgtgccggctcaggtgtgcactggctcaggtgtgcaccagctcagatctgagccagcacaggtctgcaggctcccacaggtcacaacaagaagcaggtgtttctgggcgaggacctgaagcagcaggctggggctgggccaggtcccactgtggctggtggtcagcacacctttgccagcaggcgccacagcacaggtgcccagcccacagcggggcggcagggaatctgctcctggaacctgggttttctgggctggctcccgggggtgttgactgacaggagaaggctgcagaacaagaaggtcgggtttcaggctggcagcctctcctcaattacagggatgctggggtaggccagaacccggtgtcaggtggagtagaagtcacgcttcacgggaggcttctgttttttaagaagtgcctgtgggctggggggtttttggtccagagtctaggggaaggcaaagcttaccaaacagaaagtgtccactccggggtgggggactggggcctcgtctctccgctgggccaggacagggctgtgaggtccagctgcctgctcagctctgggacctgtcctcctgcaggagcccacggccgtgaacatgcacacgggcagatccacatgtcccccgaggaaaaagagagggtcaaggttgagtgtgtgggtgctagggggtgcagaactcacttctaactatgagggttgaggcgggcttcacaggggaggtgggttttgagccaggcctgcagcccggcatctggaagtggcttccaggctctccctgagctctctcctgcaggacacccctgcctgcagatctgcacccccagctccttcctggggacttgatatcatgaccctgcctggcaccccaggggtgaatgctgcacccagccctgagggtttccatctgctgggggcatctgacctgggcaggccagggtgggtgggagggagtccagcgggggaggtgcagggtggccagggggagacactgccctggctggagcctggattcactaggtcatcaccaatgcagggggtcctggctcactggactttgctactagagaaggttggggagctccacatgaaggcaagaaggctggggctcagggtgtaactcatccccggagagcaaccagaaaggccgtcggattgcaacgcagcctgcattgtcctcgctgaacgcctggtcctgtcccacctgcaccggacagcaactgcttcccctccagggcggcccccatcgtcccccaggtgctgcaagagcagtgagacttacccaagacaagtcagaggctttggagctctcgggggcggtggcttctcccaggagccccgtatctgtcagtccccccataaggggaggggagttggcaaggctcctccttgctcccagcgtgaggattgcccctacttttccggcccccacttgccccctccacctgcccttttccctccgggaagccctggaggttttccaagaactctgcgggtcgagggggcagcctatgtggggtggcggggggcctcctgcttgttggatgcccagacgcctacacctttcaccctggggtccagtcggctgatggccatgagagagaagctgagagcaaccagagcccacagctccatgctggtcccccatctgcaaacgctgggccccatgggagctgtgactcggtttccagctcgtcacagggctggccgaggccccggcatgtcaagccatctcaggttgggcaggaatgtggtccgtgttcacatgtgtctctgtgtgtgtgagagagaggggtcagctgggacgctggggtggcagggacagtcctggctcacccctcatcctccctcgacctcgactccctccacatgaggagcccccccttcctggctatcctgtgagttgagcttcctctgctgggagggctttgtcagaggttccctgcggttccagaaggaaagctggctgcagggagggccgggcactggacaccgtgtggctgagcctgtggcgggggctgcacagctgggttcccagcccccctccttgtccccaccccaccgcactgggaggccctgctgaggggccagagtccggctgcaggtcccacgggtgggggtggggcccctcattagcactgcagctgacactgagggcttccacctcgctaattgattaaactgtttagaaaccaggccggcgtggtgggaattggccccggccgggctgtccgctccccttctgtgcaggcagcggcccccggagttcatcagtcaggccggttggtggggtcccggccctggctgccctcgggaacccttctttgctcctttgtgcggtcaaaatggtgagggtcctgagaggagctggtgagaccccggggtcctctcctccctgaccactcactgggcgagcatggagggaggcctactgtgcacgggcatgttcctgggaacctgcctgctgggattaaacccgcccttgtgaaggacggcaggtgggtcactcaataccaggaggggcacggggctgtgagcagaggcccgagagccttctgaggcggcaccgggtgctcctgggccctgctctcctgggatttgttgtgcctgtgacctcagcctcttccttcctctcctgtgggattcccccaacaccccctcccctcctgccattccttcccccaccaggccccatgcctcccctccccagtgccccctacccccaggtcttccctctaggacatcagcctgggctgtgggtcttggtctcccacagagactgagtcctgggagaagggcagagccttggttcccagtgcagcccctgtgccagcctgcagtgggcaccggttcagccggtgcacactgggtcctgcccccacctgaggagcggcctggggcctgatcagccctgctggtgtctggcctgcagccagcaccggctctgctattcacacttggttacaggtgggtgcccatcccagcagcctcggagcagagtgggtcgggctccggaggtgggggcggccactaacagcaggaggtcgtggcagtgcggctatggcaggggttctgaggggcggaaggcaggggcgggacgtggggacgcagacctgcagggaggacgccggctcacccagcagggaggggatggccgcccagggacccccagcctgcccgctctgcttccccgaccgccggggcaggggccccacgggggacgccagggaacgtgaggaatccggagtcaacactgggccactgtgtgctgccagccgggcgggccgtgatttataaagacagcggaggcttggctggtgtcggggcggtgaggtcacggcggccgggggctctggaatttcttcagaagaattttgcttaccaagccacatacttttctagccatcagtttgatcagaggcaagatgaaaaatatgctaaaaaacaaagaaacaaaaatacacccggggggctccggtgagggggaggggcgctgcgggaggggtggagggcccagggaagggtgaggggccgggagccactctgcccggcactctccgcccagaaacagcccaacgcccctttctttccccttttagcactgctgagctggactaaaatgcccaacaaggaactttactaaaaactgaggcaagaaagaaaacacacatgacataaaaatagtcaagggcacattcttgatggtagataactggtctctggccacagcggctgccaggttgggtgtcggccggcgggtctgccagtcccacccataggcactgcacttccctgggccggacagggggtgtggcgggtctgtgggcggggggacaaggttggcaggaccgtgaggggggtggtgggtctgtgggagggggacaaggttggcaggaccgtgaggggggtggcgggtctgtgggcggggggacaaggttggcaggaccgtgaggggggtggtgggtctgtgggagggggacaagggtggcaggaccgtgaggggggtggcgggtctgtgggaggggggacaaggttggcaggaccgtgaggggggtggcgggtctgtgggcaggtggacaagggtggcaggacctgtgagatgatgtgagtgcagcacagtggggctctgtaagaagcgacccgggcagcttgagcaggggcaggctgggcggtgcctacgggtctctgtccaccggagcctctgttcagcccacctcagtgtcgctccggatgtggatagaaggagacactgtctgggccacagaccaggtgcttccttcgtcctgaccacacctgcttctgcccaggagacgctgcaggggctgtgctccccgcccggctactcttgagtggtccccaggctcctcctcctcccggttccacctggagccgtggggctgtgccggggatgcctcgctgcagctgcagctcagggagaactcactgctggagcttctgcctctcccgtgccgtggggccgagccgagctccaccagggtctggacttctgcacgggcagctgtgcttcccagggtcgtggagaggggtccttggtcccagccactgtgtgacctcgaccaggacacttgactttcctgcccccagagggtcttgtctggacctccagagcccccagccttgctcacttggctctgcttctgggcagggtgccctggcattgctgttgctggcacctgccgtgccttggaggggtctccagtgggacctctgagcacggctcttcctgtacttctcagaggtgagcagagggcatttgtgggagaactggaacctggggaggaaaaaccccaaggctggcaaagactccctgcagtctgtccagtgatccactgaggctgagtggtggaggacatggaggccggcccgggaccaggacatggaggccggccagggacctggggaagagagggcctcagtctggtgagaccagcctggtgggtgcctggggaagagagggcctcagtcctgtgagaccagcctggtgggtgcctggggaagagaggccctcagtccggtgaggagaccagcctggtgggtgcaggccacccttgcctgctgtcagggcctgcccttctctccggcctccagctgctttgccccagcgatcaggcgcctgagcttcctcccccgagcctgagtccagctgagctccgtgtggctttcccggtggagcagactctgtctgatttcccaacggctggcgcctcccagggcgtgctccttgccacggaacagccccttggggccaggtgtgtactccaggcagtggcccggcagtgctgggaagtgccggtcatggctgctgcacgtgggttgctgtctgggagagtcctgtggtgtttgctgagggcggaggacaccgaggacagagaatgggcaacttccagggagggcccagatgcagccacgactggggtgcatctgggatacctcgtccagggacactccccaccatggcctggtgcctgtccagcaggaagagcttcagggcagtaggaaggggga

SEQ ID NO: 82 - DNA, 2531 bp, Homo sapiens
tgcactacctgcgcctcagccgcgactacctgcgcgcctggcacagcgaggacgtgtctctgggcgcctggctggcgccggtggacgtccagcgggagcacgacccgcgcttcgacaccgaataccggtcccgcggctgcagcaaccagtacctggtgacgcacaagcagagcctggaggacatgctggagaagcacgcgacgctggcgcgcgagggccgcctgtgcaagcgcgaggtgcagctgcgcctgtcctacgtgtacgactggtccgcgccgccctcgcagtgctgccagagaagggagggcatcccctgagccgccgcggcccggccctccgggacacctgcttcacccggcggcgccttggggcaggtgccgagcgggcgcactacgcccgggccccaaggcccccgtcccgcagccacgcttgtggtcgctgcgtcccggtctgcgtttgggagacccctgggggttgccggggcagcgcgccgtgtccaggtggaggtgcccgttcctggacctcagcgagcctgagccgggcccggccgcacgctgacccccgtgctgtccccgaccggctcacggggctgggctccgatcttccgtgtctcttatcagtggcgtttctcacgtctgcgtctcagatctaacgtggtttcacatcaatccgctttcatgggattttggtctctgtccagtgacttcgtggtaaatgtaactcagtgtttgcttgcgacttatttataaatattgtaagtttgtgtcgatgagtgtaagttggcagtgcgcacgtctcggtttttttacatgatttaaggaaagacttttatgtcagaacttggtgcctgtaccgtcaaccccgctgctgcccgtgtttaaacgcaggagaactttaaaactggccatctatcttttcagtgtacaagtcactgaacccattgtttctttctgaagagactttcctttcaaggcttcccatgggtccgcgccacacagggccggtgctgctttatttcagactctgccccaggttccaggaatccgaaccccggagtgctgacgcggttccccaacttccgccttaagaaaacaggaccagccggcaccaggcccgtctctcacgtactttaacacatccttgaaagcccctcgtttaatgagaaaagcgaacactgcggtccttgccaaagtaaaatgaagctgccccaggacaaggggttaccatgagctccctggagtccgacgcgggttttctctctgggggacctgggtggtccccgctgtggtctttgttgtcccactttgggaccgggtccagtctggggtctagtctcgagcatcagggtcaggctcggggcagggctgggttaggctccgggtcagtcttgccatgggtttgggagcaggtttgggttacttgcgtttgaaggcagcagtggtctcaggaggaagaaacgggggcgggagagagtggtgatctgtggtcagtgggtcagtgacctgcacggtgattctcccacctccaaaaggtaggggtgggactggaggcgtccctaggtcaggccgttgagttcgagctccgatgggccaccttgaatccaggactgaccgcccgtgtgtgcacagtttgttcttggacgaggactcgtgaggatcgagggctggggaccccggtgtgagcaggatggggccctgccctcccgtgggagttgtggactcgagcccaggggctgcccgtcacagcggtgtcccaggtccctgccatccgattttacctgggatgtcttctctggagtttggaattgcttgaggaaccctgcgtgtgcttggagaggccagagggcttgctgagaaccccatggacagtggagagcgggattcgaaccaagggctggactcccacacctctggcctgcgtcgcccagttctttgtggctctgaagaattggccgctgtggaaaagagcaaatgtccgagacccccaacaggaagagtctaaaaatccagtttgcaaccacttctgacctacaaaaaaatggaaatttagtgtttttcagcctaagacattaaatttcatatcagaacaaagcctgccccaggctgaccctccccagccgtaccgtggtgaacgggttcagaggatacgtgggctgaaggctgggcctcgggagggctgggggcttccagagccggggcagctgcagctctctctggtctcacctggaacttgccctgtagatcctccctgccctgcggctccaatcgaccgtgcacgggccgtggcatccgtcccccaggcgtccttccctggtcttagcttgtacagctccccacccacccaggtactcggttcccggagaccagggccaaaccaggaggccctcgggagatggggggtcaccgaattcatttccatgtgggaacttgggatacaaaacagccaactcttcctcagccacacggatgtttctcctctagtggccccgagaacctaccatggaggggacagtgtcagggctggacgggcac

SEQ ID NO: 83 - DNA, 30 bp, Artificial sequence
  Reverse DNA Primer
tctgcggctgacctggcctccacgtctcac

SEQ ID NO: 84 - DNA, 30 bp, Artificial Sequence
  REVERSE DNA PRIMER
ctacccgtctcccaccccctctccccaccc

SEQ ID NO: 85 - DNA, 30 bp, Artificial Sequence
  FORWARD DNA PRIMER
ccctaaactcctccctatcccttctcaatc

SEQ ID NO: 86 - DNA, 28 bp, Artificial Sequence
  FORWARD DNA PRIMER
aaaaaaaacctcatttcctccccaaagc

SEQ ID NO: 87 - DNA, 32 bp, Artificial Sequence
  FORWARD DNA PRIMER
agttcctaaacaactatgagctaaagtatcag

SEQ ID NO: 88 - DNA, 34 bp, Artificial Sequence
  REVERSE DNA PRIMER
cttttaagtgtgaagagttaagaagtatcatgtc

SEQ ID NO: 89 - DNA, 30 bp, Artificial Sequence
  FORWARD DNA PRIMER
ttgatgtttatgtccagattttctcttccc

SEQ ID NO: 90 - DNA, 30 bp, Artificial Sequence
  REVERSE DNA PRIMER
gaatctcaaaatgcttaactccaaaaccag

SEQ ID NO: 91 - DNA, 30 bp, Artificial Sequence
  FORWARD DNA PRIMER
cagagcatagtcaagagaggcgcattttcc

SEQ ID NO: 92 - DNA, 30 bp, Artificial Sequence
  REVERSE DNA PRIMER
aagagcccctaaattagccccgtagaaacc

SEQ ID NO: 93 - DNA, 31 bp, Artificial Sequence
  FORWARD DNA PRIMER
gcaaagacaatgcaaaaaacactttacatgg

SEQ ID NO: 94 - DNA, 34 bp, Artificial Sequence
  REVERSE DNA PRIMER
gcctgatataggtatattcagagagctacagaag

SEQ ID NO: 95 - DNA, 30 bp, Artificial Sequence
  FORWARD DNA PRIMER
actcccttttggataatcaaaatgctcaac

SEQ ID NO: 96 - DNA, 31 bp, Artificial Sequence
  REVERSE DNA PRIMER
gcaaaattacctttcaaatgtgtacttgctc

SEQ ID NO: 97 - DNA, 30 bp, Artificial
  FORWARD DNA PRIMER
ttgaaatatggtacaaagaaggggttggag

SEQ ID NO: 98 - DNA, 30 bp, Artificial Sequence
  FORWARD DNA PRIMER
cttgaagtccttgccgaagaaaaatagttg

SEQ ID NO: 99 - DNA, 32 bp, Artificial Sequence
  FORWARD DNA PRIMER
gctgactcaagaactgtagcattgagtgtaag

SEQ ID NO: 100 - DNA, 32 bp, Artificial Sequence
  REVERSE DNA PRIMER
ggggaatgcaagcatattatatgagcagaagg

SEQ ID NO: 101 - DNA, 31 bp, ARTIFICIAL
  FORWARD DNA PRIMER
gcaaaggacctctttaatgcttatcagccac

SEQ ID NO: 102 - DNA, 30 bp, ARTIFICIAL
  REVERSE DNA PRIMER
ggtgagagctatggaaagcctctcctattg

SEQ ID NO: 103 - DNA, 32 bp, ARTIFICIAL
  FORWARD DNA PRIMER
ttccagccccacctgctcaggcagcctctatg

SEQ ID NO: 104 - DNA, 31 bp, Artificial Sequence
  REVERSE DNA PRIMER
gccagcacagcctcctgtcttagccctgtcc

SEQ ID NO: 105 - DNA, 30 bp, Artificial Sequence
  FORWARD DNA PRIMER
gcgagaaatgcctccctattccccaggagc

SEQ ID NO: 106 - DNA, 30 bp, Artificial Sequence
  REVERSE DNA PRIMER
tcccagaactttgcctgttgcccatgccac

SEQ ID NO: 107 - DNA, 30 bp, Artificial Sequence
  FORWARD DNA PRIMER
agcagctccagagcagggaacccacctcac

SEQ ID NO: 108 - DNA, 30 bp, Artificial Sequence
  REVERSE DNA PRIMER
gtgtccacaccaggcagcgtccaactcagc

SEQ ID NO: 109 - DNA, 30 bp, Artificial Sequence
  FORWARD DNA PRIMER
atgagggaggagtggggagaggaagtgaag

SEQ ID NO: 110 - DNA, 30 bp, Artificial Sequence
  REVERSE DNA PRIMER
actacctggtgtccagtacccaaatccagc

SEQ ID NO: 111 - DNA, 30 bp, Artificial Sequence
  FORWARD DNA PRIMER
ccctctttctgaacaccccccggcagacac

SEQ ID NO: 112 - DNA, 30 bp, Artificial Sequence; identical to SEQ ID NO: 111
  REVERSE DNA PRIMER

SEQ ID NO: 113 - DNA, 30 bp, Artificial Sequence
  FORWARD DNA PRIMER
tctgctctcctgtgccaagcgtcaatatgg

SEQ ID NO: 114 - DNA, 29 bp, Artificial Sequence
  REVERSE DNA PRIMER
acctctctgggtctctctcctcctcactg

SEQ ID NO: 115 - DNA, 33 bp, Artificial Sequence
  FORWARD DNA PRIMER
gcatttctcagaataatgaatggcaggaaatac

SEQ ID NO: 116 - DNA, 30 bp, Artificial Sequence
  REVERSE DNA PRIMER
gtgcatgtttcaagacattctcagattgtg

SEQ ID NO: 117 - DNA, 30 bp, Artificial Sequence
  FORWARD DNA PRIMER
caagttggtaaatggaggcattatatggag

SEQ ID NO: 118 - DNA, 30 bp, Artificial Sequence
  REVERSE DNA PRIMER
agtcacgtatcaagtggaaataaaatcgtc

SEQ ID NO: 119 - DNA, 30 bp, Artificial Sequence
  REVERSE DNA PRIMER
acaacaggacaatgcatacaaccacgaaac

SEQ ID NO: 120 - DNA, 30 bp, Artificial Sequence
  REVERSE DNA  PRIMER
tcattagaatgaaagggagccacagagcag

SEQ ID NO: 121 - DNA, 30 bp, Artificial Sequence
  FORWARD DNA PRIMER
agctccaggtaactctcaggccagcagccc

SEQ ID NO: 122 - DNA, 32 bp, Artificial Sequence
  REVERSE DNA PRIMER
aaggaggaagtggaagctcagcccaggcagtg

SEQ ID NO: 123 - DNA, 31 bp, Artificial Sequence
  FORWARD DNA PRIMER
tgctgaccgagcacatacacaattcagtgac

SEQ ID NO: 124 - DNA, 35 bp, Artificial Sequence
  REVERSE DNA PRIMER
agggtctctgctaacgtagtgaaaatacgcaaatg

SEQ ID NO: 125 - DNA, 30 bp, Artificial Sequence
  FORWARD DNA PRIMER
ctgagcagccaccctggatgctcctgcacg

SEQ ID NO: 126 - DNA, 30 bp, Artificial Sequence
  REVERSE DNA PRIMER
ctctggccctcggcccattgccacctcaac

SEQ ID NO: 127 - DNA, 30 bp, Artificial Sequence
  FORWARD DNA PRIMER
acagaagcaagcagaagtacagaaccagag

SEQ ID NO: 128 - DNA, 30 bp, Artificial Sequence
  REVERSE DNA PRIMER
tttctccctcctagatgatcgacttgggac

SEQ ID NO: 129 - DNA, 30 bp, Artificial Sequence
  REVERSE DNA PRIMER
caccatctgcatcttacatcttattccacc

SEQ ID NO: 130 - DNA, 30 bp, Artificial Sequence
  REVERSE DNA PRIMER
aagttaattggagggaaatggctgtaaagg

SEQ ID NO: 131 - DNA, 32 bp, Artificial Sequence
  REVERSE DNA PRIMER
gagttaagctcagctcactctgtggcactacc

SEQ ID NO: 132 - DNA, 32 bp, Artificial Sequence
  FORWARD DNA PRIMER
ggaagtgtctgtggtttgccagctcctgttct

SEQ ID NO: 133 - DNA, 30 bp, Artificial Sequence
  REVERSE DNA PRIMER
gattctgacccttgcccagcctacgtctcg

SEQ ID NO: 134 - DNA, 30 bp, Artificial Sequence
  REVERSE DNA PRIMER
tgacccacaatctttcccttctggcaccac

SEQ ID NO: 135 - DNA, 34 bp, Artificial Sequence
  FORWARD DNA PRIMER
gatgtttctaactatacctttatgtgtttttcct

SEQ ID NO: 136 - DNA, 32 bp, Artificial Sequence
  REVERSE DNA PRIMER
gctcttcctaccaagttatcttcatctattcg

SEQ ID NO: 137 - DNA, 31 bp, Artificial Sequence
  FORWARD DNA PRIMER
ccagatactggtctcattcttgggcagtttc

SEQ ID NO: 138 - DNA, 32 bp, ARTIFICIAL
  REVERSE DNA PRIMER
ccgagtttgactttcactcactcacctagatg

SEQ ID NO: 139 - DNA, 30 bp, Artificial Sequence
  FORWARD DNA PRIMER
aatgaaagggatacgtttgcgtctgtcctg

SEQ ID NO: 140 - DNA, 30 bp, Artificial Sequence
  REVERSE DNA PRIMER
ggtaaagttcttcccctggctcttcacaac

SEQ ID NO: 141 - DNA, 30 bp, Artificial Sequence
  FORWARD DNA PRIMER
attttagtgaagaaacttgctgtggagtcg

SEQ ID NO: 142 - DNA, 30 bp, Artificial Sequence
  REVERSE DNA PAPER
aagaagaaggaaagaacaagaaaagcccag

SEQ ID NO: 143 - DNA, 32 bp, Artificial Sequence
  FORWARD DNA PRIMER
ccacacccagccaacagcagacgtgatggaag

SEQ ID NO: 144 - DNA, 31 bp, Artificial Sequence
  Reverse DNA Primer
ctgaggagacaggtgggacagaggggcagac

SEQ ID NO: 145 - DNA, 30 bp, Artificial Sequence
  FORWARD DNA PRIMER
gctcctccccacacctgaccctgccctcac

SEQ ID NO: 146 - DNA, 30 bp, Artificial Sequence
  REVERSE DNA PRIMER
gagctggcccgttttgccacctgtcacccc

SEQ ID NO: 147 - DNA, 30 bp, Artificial Sequence
  FORWARD DNA PRIMER
caacccgagagatgagccctgcgtccactg

SEQ ID NO: 148 - DNA, 30 bp, Artificial Sequence
  REVERSE DNA PRIMER
cacctgcgtcttcaagccctaatgggcacc

SEQ ID NO: 149 - DNA, 30 bp, Artificial Sequence
  FORWARD DNA PRIMER
aatgaagaaatgaatctctctccttggacg

SEQ ID NO: 150 - DNA, 30 bp, Artificial Sequence
  REVERSE DNA PRIMER
tttatcatgtggcaggcaattaaatgacag

SEQ ID NO: 151 - DNA, 30 bp, Artificial Sequence
  FORWARD DNA PRIMER
gtgtccccaggcagagttaagaaaagaagc

SEQ ID NO: 152 - DNA, 33 bp, Artificial Sequence
  REVERSE DNA PRIMER
gcaggagtgaaacaacaaaaaatacagccagtc

SEQ ID NO: 153 - DNA, 30 bp, Artificial Sequence
  FORWARD DNA PRIMER
tactccttccttccttccctcaaccctgac

SEQ ID NO: 154 - DNA, 30 bp, Artificial Sequence
  REVERSE DNA PRIMER
tttgggcagagtgtggatggagaagattgg

SEQ ID NO: 155 - DNA, 30 bp, Artificial Sequence
  FORWARD DNA PRIMER
ttcagaaggtagagttggaggatcataggc

SEQ ID NO: 156 - DNA, 30 bp, Artificial Sequence
  REVERSE DNA PRIMER
tccccacagagtaaacagtaggaaggaaag

SEQ ID NO: 157 - DNA, 31 bp, Artificial Sequence
  FORWARD DNA PRIMER
cacaaaaagattaaaacacaatcttgtgagc

SEQ ID NO: 158 - DNA, 32 bp, Artificial Sequence
  REVERSE DNA PRIMER
actcatcctttattcttctagtaagaattgcc

SEQ ID NO: 159 - DNA, 30 bp, Artificial Sequence
  FORWARD DNA PRIMER
tgcctgctgactgagggggatggccggaac

SEQ ID NO: 160 - DNA, 30 bp, Artificial Sequence
  REVERSE DNA PRIMER
ggctgtgggtgtgcgggataggggaggctc

SEQ ID NO: 161 - DNA, 30 bp, Artificial Sequence
  FORWARD DNA PRIMER
tccttgctgcactacctacccatgcaggcg

SEQ ID NO: 162 - DNA, 30 bp, Artificial Sequence
  REVERSE DNA PRIMER
ggtcaccgggaggaagccacacatctgacg

SEQ ID NO: 163 - DNA, 32 bp, Artificial Sequence
  FORWARD DNA PRIMER
tcttagaacatgtgacagaatcaaaaaattcc

SEQ ID NO: 164 - DNA, 32 bp, Artificial Sequence; identical to SEQ ID NO: 163
  FORWARD DNA PRIMER

SEQ ID NO: 165 - DNA, 30 bp, Artificial Sequence
  FORWARD DNA PRIMER
tttcagacggtcgagtgacagtccaaacgg

SEQ ID NO: 166 - DNA, 30 bp, Artificial Sequence
  REVERSE DNA PRIMER
ggaggctctgctttccagccagatgtaagg

SEQ ID NO: 167 - DNA, 32 bp, Artificial Sequence
  FORWARD DNA PRIMER
gcatacatctccgacactaggaaagacacgac

SEQ ID NO: 168 - DNA, 30 bp, Artificial Sequence
  REVERSE DNA PAPER
attggcctttcagcttgcccaaacacaaac

SEQ ID NO: 169 - DNA, 32 bp, Artificial Sequence
  FORWARD DNA PRIMER
cttaaaatatccagtctcagttttgtttcctc

SEQ ID NO: 170 - DNA, 30 bp, Artificial Sequence
  REVERSE DNA PRIMER
ttaaatgcaactcaaaagaagaaaggtctc

SEQ ID NO: 171 - DNA, 31 bp, Artificial Sequence
  FORWARD DNA PRIMER
ccttttttttgtcacctagtatttgcaacac

SEQ ID NO: 172 - DNA, 30 bp, Artificial Sequence
  REVERSE DNA PRIMER
ctaaaacccataaattgaccgaacactctc

SEQ ID NO: 173 - DNA, 30 bp, Artificial Sequence
  FORWARD DNA PRIMER
gggatagatgatggtttgttgtaatttgag

SEQ ID NO: 174 - DNA, 35 bp, Artificial Sequence
  REVERSE DNA PRIMER
gtctctagataatctaataatatccacttcccaag

SEQ ID NO: 175 - DNA, 31 bp, Artificial Sequence
  FORWARD DNA PRIMER
gccacgcacttccctgctgtttgaaagaccc

SEQ ID NO: 176 - DNA, 30 bp, Artificial Sequence
  Reverse DNA Primer
gtgtttgtcaccccactcctgctcctgccc

SEQ ID NO: 177 - DNA, 30 bp, Artificial Sequence
  FORWARD DNA PRIMER
gtgtcggttctccaccaccacgatgagccc

SEQ ID NO: 178 - DNA, 30 bp, Artificial Sequence
  REVERSE DNA PRIMER
tcccgcctagcagagttgctgtctggcaag

SEQ ID NO: 179 - DNA, 30 bp, Artificial Sequence
  FORWARD DNA PRIMER
agttctctgcttcttccttgttttctctcc

SEQ ID NO: 180 - DNA, 30 bp, Artificial Sequence
  REVERSE DNA PRIMER
tccctttttgcttctctgtgttgtgatttc

SEQ ID NO: 181 - DNA, 30 bp, Artificial Sequence
  FORWARD DNA PRIMER
tcggataaaagcagaagcagagagagcagg

SEQ ID NO: 182 - DNA, 30 bp, Artificial Sequence
  REVERSE DNA PRIMER
agccccctcctaaaggctgtcacctataag

SEQ ID NO: 183 - DNA, 30 bp, Artificial Sequence
  FORWARD DNA PRIMER
atcctttccttttttgccttcttcctcatc

SEQ ID NO: 184 - DNA, 30 bp, Artificial Sequence
  REVERSE DNA PRIMER
cttctttcctccccatcttctccttcttag

SEQ ID NO: 185 - DNA, 30 bp, Artificial Sequence
  FORWARD DNA PRIMER
gacaggttggggatctagagagctggggag

SEQ ID NO: 186 - DNA, 30 bp, Artificial Sequence
  REVERSE DNA PRIMER
aaagggggtgttagtgaggggccacaaaag

SEQ ID NO: 187 - DNA, 30 bp, Artificial Sequence
  FORWARD DNA PRIMER
gcaatcagatttctctcaaaccacgaacac

SEQ ID NO: 188 - DNA, 30 bp, Artificial Sequence
  REVERSE DNA PRIMER
tttatcaggatatgcgttttcctccaaccc

SEQ ID NO: 189 - DNA, 33 bp, Artificial Sequence
  FORWARD DNA PRIMER
ccttaacaaacaaacagaaaaaaaagaaaggag

SEQ ID NO: 190 - DNA, 31 bp, Artificial Sequence
  REVERSE DNA PRIMER
agtcccaatatttgaacctaaatgcaaaaag

SEQ ID NO: 191 - DNA, 30 bp, Artificial Sequence
  FORWARD DNA PRIMER
atcttgttgcatcctgagagaaacagaatc

SEQ ID NO: 192 - DNA, 30 bp, Artificial Sequence
  REVERSE DNA PRIMER
caggcatctacttgagaactgacaaactac

SEQ ID NO: 193 - DNA, 30 bp, Artificial Sequence
  FORWARD DNA PRIMER
tgagaatgtgattgccgttctgaaaacacc

SEQ ID NO: 194 - DNA, 34 bp, Artificial Sequence
  REVERSE DNA PRIMER
tcttttctgtgtgcttgattcttgcagatacagc

SEQ ID NO: 195 - DNA, 30 bp, Artificial Sequence
  FORWARD DNA PRIMER
ggagaaggggagtttgctggggagacgagg

SEQ ID NO: 196 - DNA, 30 bp, Artificial Sequence
  REVERSE DNA PRIMER
acacaatggaaacaatggggagggtgggcg

SEQ ID NO: 197 - DNA, 30 bp, Artificial Sequence
  FORWARD DNA PRIMER
acctgccctgccacctctgttctccctgcc

SEQ ID NO: 198 - DNA, 35 bp, Artificial Sequence
  REVERSE DNA PRIMER
cgcctttgagtcaaccaagccccaagatgcacacc

SEQ ID NO: 199 - DNA, 30 bp, Artificial Sequence
  FORWARD DNA PRIMER
accactaagagcccctgtcaccctccagcc

SEQ ID NO: 200 - DNA, 30 bp, Artificial Sequence
  REVERSE DNA PRIMER
ttccccattccccagtccaacaccccctcc

SEQ ID NO: 201 - DNA, 30 bp, Artificial Sequence
  FORWARD DNA PRIMER
cagatggagacactctccctgggaaatgcc

SEQ ID NO: 202 - DNA, 30 bp, Artificial Sequence
  REVERSE DNA PRIMER
ttttgccttcctgctgcatgaccagctaac

SEQ ID NO: 203 - DNA, 30 bp, Artificial Sequence
  FORWARD DNA PRIMER
ctctctgctccacctctggctttgacgacg

SEQ ID NO: 204 - DNA, 30 bp, Artificial Sequence
  REVERSE DNA PRIMER
agactgcctcccctcccctaacccagaatg

SEQ ID NO: 205 - DNA, 30 bp, Artificial Sequence
  FORWARD DNA PRIMER
agtgcccaggaaagaccaggaaaatacaag

SEQ ID NO: 206 - DNA, 31 bp, Artificial Sequence
  Reverse DNA Primer
gggaaatagtagcgtaagctgtcaactccag

SEQ ID NO: 207 - DNA, 34 bp, Artificial Sequence
  FORWARD DNA PRIMER
tccatttcctgccatctaagcaatgcagacacag

SEQ ID NO: 208 - DNA, 33 bp, Artificial Sequence
  REVERSE DNA PRIMER
tggactgcttgctggtcgcttacatcactttac

SEQ ID NO: 209 - DNA, 30 bp, Artificial Sequence
  FORWARD DNA PRIMER
tcagaggggggctggacattgaatgtgaac

SEQ ID NO: 210 - DNA, 30 bp, Artificial Sequence
  REVERSE DNA PRIMER
gtcaccataggacacagacaggaagtgggg

SEQ ID NO: 211 - DNA, 30 bp, Artificial Sequence
  FORWARD DNA PRIMER
tagaaataacgaccaaaagcctcccctgtg

SEQ ID NO: 212 - DNA, 30 bp, Artificial Sequence
  REVERSE DNA PRIMER
ttcaagctgtcagggacatcatgttgagag

SEQ ID NO: 213 - DNA, 30 bp, Artificial Sequence
  FORWARD DNA PRIMER
tttgtatgttattaccctcgttgtgccatc

SEQ ID NO: 214 - DNA, 30 bp, Artificial Sequence
  REVERSE DNA PRIMER
tctcagcctcagaaaatgcttatgttgaag

SEQ ID NO: 215 - DNA, 30 bp, Artificial Sequence
  FORWARD DNA PRIMER
ttttttccctcctggcctcactcttgcaac

SEQ ID NO: 216 - DNA, 30 bp, Artificial Sequence
  REVERSE DNA PRIMER
atagaaggaagcaggacaacggggacagac

SEQ ID NO: 217 - DNA, 30 bp, Artificial Sequence
  FORWARD DNA PRIMER
cggaagtcaacagtcactgacgagtcggag

SEQ ID NO: 218 - DNA, 30 bp, Artificial Sequence
  REVERSE DNA PRIMER
agagtatagggaccagcaggaacacggagg

SEQ ID NO: 219 - DNA, 30 bp, Artificial Sequence
  FORWARD DNA PRIMER
gcaccagcccttaccttcctcccttcacag

SEQ ID NO: 220 - DNA, 30 bp, Artificial Sequence
  REVERSE DNA PRIMER
atatggtaggtgctcaccacatgcaggccc

SEQ ID NO: 221 - DNA, 30 bp, Artificial Sequence
  FORWARD DNA PRIMER
cctttctctacaccctcccacctgctgctc

SEQ ID NO: 222 - DNA, 30 bp, Artificial Sequence
  REVERSE DNA PRIMER
cacccacctctccctgcctctagtctcttc

SEQ ID NO: 223 - DNA, 30 bp, Artificial Sequence
  FORWARD DNA PRIMER
ccctaccccagatcctgaggattcacatag

SEQ ID NO: 224 - DNA, 30 bp, Artificial Sequence
  REVERSE DNA PRIMER
gggacagtcagaaacatctctgaaaccctg

SEQ ID NO: 225 - DNA, 33 bp, Artificial Sequence
  FORWARD DNA PRIMER
gctcagtgctctcccgctctcctgcttctcttc

SEQ ID NO: 226 - DNA, 35 bp, Artificial Sequence
  REVERSE DNA PRIMER
actcagcctctaatcagcctctctgctccacccac

SEQ ID NO: 227 - DNA, 30 bp, Artificial Sequence
  FORWARD DNA PRIMER
taatgtatgcccacaaatctccagcgaccc

SEQ ID NO: 228 - DNA, 30 bp, Artificial Sequence
  REVERSE DNA PRIMER
tccagcaccatctctgaacaactacatgcc

SEQ ID NO: 229 - DNA, 30 bp, Artificial Sequence
  FORWARD DNA PRIMER
tctaagaccaagtcgctacactcttaactg

SEQ ID NO: 230 - DNA, 30 bp, Artificial Sequence
  REVERSE DNA PRIMER
cttctttcaaccataaaagccttcctcctc

SEQ ID NO: 231 - DNA, 30 bp, Artificial Sequence
  FORWARD DNA PRIMER
ttcagcgccagcctcttcgctccgtccaag

SEQ ID NO: 232 - DNA, 30 bp, Artificial Sequence
  REVERSE DNA PRIMER
tggtcaggtgtgggtcaggagaccccagcc

SEQ ID NO: 233 - DNA, 30 bp, Artificial Sequence
  FORWARD DNA PRIMER
gggtctcacatgtagcattcctgggcacac

SEQ ID NO: 234 - DNA, 30 bp, Artificial Sequence
  REVERSE DNA PRIMER
gtcctcccattcccatccctatccccactg

SEQ ID NO: 235 - DNA, 30 bp, Artificial Sequence
  FORWARD DNA PRIMER
caggtaagggagatgagacctccagacaac

SEQ ID NO: 236 - DNA, 30 bp, Artificial Sequence
  REVERSE DNA PRIMER
ccaaatacagacacagcctcaaccccattc

SEQ ID NO: 237 - DNA, 30 bp, Artificial Sequence
  FORWARD DNA PRIMER
cgcaggaaataggcaaacacacactggaag

SEQ ID NO: 238 - DNA, 30 bp, Artificial Sequence
  FORWARD DNA PRIMER
ggaccctacactggatgggttttagcagtc

SEQ ID NO: 239 - DNA, 30 bp, Artificial Sequence
  FORWARD DNA PRIMER
atccacagctttgatctagggaaaataaac

SEQ ID NO: 240 - DNA, 30 bp, Artificial Sequence
  REVERSE DNA PRIMER
tgtgttggaaatgcaacttaaattgaactg

SEQ ID NO: 241 - DNA, 31 bp, Artificial Sequence
  FORWARD DNA PRIMER
tatagacacgtgacaaagtagctgaaagacc

SEQ ID NO: 242 - DNA, 30 bp, Artificial Sequence
  REVERSE DNA PRIMER
tctgtttctgtgtatgactgcaatttaacc

SEQ ID NO: 243 - DNA, 30 bp, Artificial Sequence
  FORWARD DNA PRIMER
catgctaaattcatgggccatattttcaac

SEQ ID NO: 244 - DNA, 30 bp, Artificial Sequence
  REVERSE DNA PRIMER
gatgcaaaatgttcatctcacatcacaatc

SEQ ID NO: 245 - DNA, 3026 bp, Homo sapiens
  misc_feature (1843)..(1843): n is a, c, t or g
caatcagatttctctcaaaccacgaacacaggggtcggtatctgaggcgccggcaccagacacggcagggtctgagtgctccctgacaagcgatgatgcgcaggcttggagccatgccagtgacacgcctaggaaagttcacgcaccgcccagcacgcctgcgcatgcctgttcccgctccctggtgccccgggcgcctgcctgtcccggctcccatgggtgctgggtgtgtggaagctccggccccctcgggctgggttcattggggtcctcctgtgtggtcagtggactctgtacccccacagcacctgaggggtggctgacactgctttcccagctgctgcaggggctcagggaacacaggtgaccccacgtctctaccgagaatgagcacaccaacacctctcagaagacagctgcagcctgcagagggcagtggaccccacccaggcccacggtgtggacggctctgcctcggtctctgctgagccaggcccagagggaccccaggtgagcagcaaaccccccaggcctgggctagcaccggggtaacccttcctgctcagcacctgttcacctgtcccctctgctggtggcctcctgtcctcccgctctgggctcagcagcagccccgtggagaggccctgccaccaccccgccctgctggagacaggcctcctacgcgggctcctgcagccggtcgccctgggcctcctagaagccggggatcctctgctgaccaccggcagaaaacgtgcttctcaagctgcaggtgattcaccagtagtgggcaaggaactgaatgtggtgattactgcggagtcagcaaaacccgcgtgagaacgggcagctgagggcctgccgggtgagggaagcctcacggttcctgtttcatgagtttgctgtgagtgcacacgaggctgtggctgtggagtgtgcaacagtccacgcgtgcctgcgtgtgctcatgtgcgtgtgtccaccagcttgtgtgcacgcatatgagcgagtgcgttttgctcccagcttggtcgcagcgacggcgcagggaaccccgggtgaggccgaggaccgggaagggaggagggggctccgacccatcggacttaggggagccccgggtccgagacgccgcctctgtcccttcaagagtcgagcctggcgcacagggcagggacgcgggtccacaccggccggcagctcgttcccgcccatactcgggtacgccgctgcgaccccgcccgcctggcctgcgacgacgctcagggccagcgggggtgacggtcccagaggcagaggcgccgcagccccagagtccccatccctgcgcggaccggcaaccccagtgcaccaagaggccctaacaccgagcccccagcaccgagtccccagcaccgggccctcagcaccgagtccccagcaccgagtccccagcaccgagtccccagcaccgagcccgcccctctggttcccccgcccgcccctctccgcgcctcaccgggtccgctcctggacgcgctcctctgggatgcagcttctccgcgccccggagccccaggaaaatgaaagacacgagagggaggggccagggaggaggcgcggacccgcgcgggacccacctcccagatgaggaaggagctgggtttacgggaagcctccaagtttcgggaaccacccgcgttcacaacaagcgtgacggtgaatttattattttcacgggaggccagcactcgcggttcacgctaaaggaagcaggaaagccgccgggagcatttttccaggagagttcgtgcctgggcgggtccgagcatgcgtgcggcggcgttccccgcggggctgtttnatgccgctcctggaggcctcgagtctgtgcacggggcgagctgggcggccgagtgggccgcggggagggagggcggggggcggccccagatgcctgggagtgcgcgggcagagtgagctggacccccggatgcagaggccctttcataaaagcgcgcagagcagaggagtgatgtcccccagctcccccgcagaggtcctgcacctgcggcctgggcttcagcgtcctgcggcccctgcggaggtgctggcctggccagcccgggaggaggggcccagcctgttggggcaggagattggggtgcgggtagaaggctccaagacgcatccgggccgggaacccacagacatcccaggtgggcaggaggtggctcgaggaggcctggaggacccggcgcctggcggggtggcaggcgggccacgtcctccactagaacccgagggggcacgcgggcaggtgcgggcggggtcaaggatgaccaggtatcttcgggacactaggaggaggccccacaggctgcagtcacgtgagtgggcaagtccccaccgggcagatgatgggggacactggggcgtgggcaatgcccccagtttcatggaagagaggaagaagcagaaccaaactccgggaaaccctcaaatgtggggaatggacggagcagggccagactggacgctgaaccttggagcctgcagctcagccatcagacccagggtccagaggtgggtggcacagaacaaagtcccccgggatgttccaaaagagaaactgtcgccaaattggcaggtgaaacacagcctgtcatcctcccagcaagacggcaccatggccggggcacagaggtcagattccccagcccccgccctcgggaaaccccagccaccctggctgccagtgagatgctggagagggggctgaaatcccacctgcccacgtcctctgcacagaggggcttgtccccgaggccacatcccccagcagccacagcttccttctccttttttcctgcctactagatctctcaactcagagggggctgcagttcctgggggcaggggggtccggctgcttaggcaggagcacctgcaccgtgaggctctggagggcagctgaaggctggcaggcttttgtcccgtgaggggacaccactgggggttggaggaaaacgcatatcctgataaag

SEQ ID NO: 246 - DNA, 2368 bp, Homo sapiens
aatcttgttgcatcctgagagaaacagaatccaaacggatgttggccagggtattattcaaggaggtcagatcatctgtgtgtttggtaagggtatctgtgcaagtggtcctgacttcatttagattgctggtcagcgtccgcaggtggtgggctgtgtaactgatattgctaatgatgttcacaatatccgtctcaaagagctggaagcgttcctccagttggttgaacttgatggctgttctattctctgcatctttgtgtaagtcctgcaggtctttcaggttctgctcgttggcttgagagatagtggtgatgttctccatctgacctgtgaatgagttgagctggctgttcatatcctccagggtgtcgttgttggctttggccaacgcagagttgttggcagccagcgtctgcaagctctgcactttctccttcagccaatccgtgtccttcttggcttgaagaaaaacctgctgcagattttgaaagtcgttcttgattcgctggatagcctggcttgtgtcatccacagaccgctgcagattcgtgatgaggttcctctgctgcacctgggtcaggttcaggttgttgaggttcatgatgaccacattatgagaatacatttggttctgcagattgccctggagcacgctggtatcttgctgcagattcgtgacatagccattatacgcctggagggttttgtttacagtggtgatgaggaaagagttattctccaaagtttctttcaattgactctgcctgtccaccagagcatccccgctcgcctgtaacttctccagcgtatccttgttcttgctggttttttctgtaatctcacgaagttgctgacggagatctagaatgtctgatctgaaggtggagagttctgagttggtgctgatagctttcttcccagtttggtcacctggaataagaaatatctgtgacttatattggtggtatggagaagtgttcaggcaaggccaaagatcccgaacacacttaatcggtatgcactgtattttagatgcaaaattggcagtataagcggacagctctgcattagtaaaatgtacatatctattaaaactgggtcctggggaatcggaaaagaagctcagaactaggaatgacaaacttggctgaacatttttctcaaagagggagggggaatttactagattttagggcagtgggcaggctgtcaagaagaaactaaccttttaaatttcccaaatttttttttaatgaaagcaaaaatcaaggaatagaatatgctaggatctttcactttataacttaatttctacaattctatgtagtttaaagtatttcaaaaatgctcagtaaattcctatttatgtgacagtttttaataaagggtatttgtgttttttttcagtcaggattgatcttcagatattatttggcacataatagttttcttggcaggacttaattccaaaactgacccttaactttaaaatttaagcatttgaattaaatcatgaggggagactcaacatgcaacacaaaaattgaatgtccttccgggtgaatggggagtttatagcaacatcattctaagaagctgtggtcatttatgtagagtcaggggatttcatggtttagtcttgtcacagattacctaattttttcaggtcactttccactgctgtgagcttgtcatcataggtttggcgagatgtttccatgccacctgtgacattgtccattttctctacaactaagatttggaaaatgatgcattagtatacatatctgctcatattttatttttcagtttcaaaacaagagatcatttcattatggaacaaaggaaacagattgaacgaaaacagtgtaactgaaatcaaatataggaaagaaaagccatctttttggaaaaataacttacttgtcacaaaacccaggggtacaatttacttagttgagaattgtatgttcttaactattcttatgattctgtaatgccttggatgtttcagaaatcatttggaactaatttaaaaattttcatgcattttagaagtccctaatctgctatttcctatattaatttccatagatgaaggcaaggcacactgtgataatttacaaaatgttgtcactcatcagcttccctaacattcttggcaggtgggactcatttacctagaaaaggattccattggcaaggaaaacccagctcaattctatatacaaaatcggcatagaaaggttgcaaagtcaagagtgtctgccactttctgttatgagttccaccacaaggccctgaaaatctgctttttgttagtgacaactgattctgtagtttgtcagttctcaagtagatgcct

SEQ ID NO: 247 - DNA, 2022 bp, Homo sapiens
gcctccagcaacctctgtctgagttccccaaagcttgcagaaatccacatagtggatcctggggtgataatgtcctaccttggaggccctgaggaaataaaaccagctggagatagtaagatcccgccttaccagctagctggaactacccaactttccacaggatacaatcctggccatgtgctcccagaaatcatttccctccgattgccagcactcttgcctactacgaacctttctttctccttccctacttctgccacgccacctcctgctaccgcctttgacacgccacctctccctacgtgtcggggagggtacagagcctctggaggcagcatggtgggaagggaaggcactcaccagggtcagtccggatgccacatcctgcacagcggtaattctgcttggccacggcaattttcctcctgaggaagggtaaggacagggcattggcacagagcagctgcgtgagaccttggaggtgtgaaggagtgagcacacatacatacagctccagttaagtatgggaagagaggggaattcacctacattttagttggacaaaaatgaacctattgggagagctaactccatataagatttaggtctaggcagtcactctgcccagtaaggaaccacacattctgtacaaatataaggaatgagatgtggtaaaggagagagaatgacaggagagaagagcatccatctatcttagaaagagaagaaaaaccagcaagcccacacaactactgggaggaaagctacaggttgggaatgccagcaaaacaaaacccgcctcgtttccaattagctccaggaattaagagtaagaaacgaaggaccaaatggacgacgccccccctctgcctttaaatgaagagaacggtgtgggaaggacagctggaggcagggacaagtgggtgagacgaaaaccctgacaatccaaagaggacggatctgtgctccaaagggcacagacactggccactcacgttggggctggatgaacattaaaaattatctgaggccggggcggggcccactccaagttgccacgaacacgaatccgcagcttgtagatgtcagcgtgctgcccgtcatccggtgagatgggcagtgagtcaggaatgggcaggagctgcaggaggaaagcacagttggggtaagctcgtgtcagtgtgctgcccgtcatctggtgagatgggcagtgagtcagggatgggcaggagaaaaacacagttggggtaagttcacacggacgggcttgagaaacagaaatgcgggacccttttggccatgacagagcataatgagtgaaagacatttcaggaacaccacaggataagggcttcagggaacctcagaaacaaccaggaggcgccaaggtactacaagtgagggccgtgggttccaagaagcaaacagaaacagcctaccagggcagtggccccacggctcatgctgtccctgcacccatcccaggacccttgctgtgccagtgtgtttcatgccttaaagacaactgcagagcaaagaatccaagcgatttacttttgcgtagtgtctccgaggtggtcacaaaccaaacatgactgagtctggcgagcagtcacgtgaataaggaccgcgaacgcgccgtcatctctgctctgacaaggtgagcaagcattcactcgttcatttatcacttgacacattgtaatgaatggcttccacgagtaaggggggaacacccaggctcattccagactagggacatgtgacgaaggaaaacaaggtcacagaggctcacgatggcccctgggtaggaagaagagctaaggacctaccttctgaggggcatcatgctccgggacaagccactccagctccgaggcggctggaagctgcatcccctcaaactgcttcaggagccccatggccaccgcctcagcagacgtggagtgcaggaagcagtgggagctggaaaggggagaatcaaggacggctgaacacagggaaaggatgggcgatgcg

SEQ ID NO: 248 - DNA, 2152 bp, Homo sapiens
actatcttcatctctcttcctataccccccattgacacgtgaatcagcgtttctcagaatactgcaggtttggagtgtgtgtggcggaggagggcggagcagcgtggaaggtggagaggtgggcggtgtcggggatatcagcagggcagtgggcattggaggggtgcccttggcctcagccacagggccgttccagagccctgcgtgggcgaggccagggcggcgcgtgatggtgccctccgagaagcactgggaccagcaggaaaggctgcctgccggtgcgcaggaaaagggaagagagccggggaattgctttttgacccgtaagggagcgtttcttggtggatggggaaatcaaaaaattgactacggtgtagtcagctacatcgtgtaccaattttcaaataccggtgagatcagtaaaaagagaaagggaaggagatcacagatagcatgaaaccaagccatcaataatgaaagtaccactggttactgagcagcgtctgcttctaactgactttgctgggggaggggcgggacaggtacaagcaaaaacagcaacgacagcgcagcagttgcttcatgtgagtaataattgaatggtacgaggctcttccacattcatgtattgaaggcccaagtgcggccaaggtctccctggttcctgaggtttgtttcatgctgggttccttatactccagatgtcgggagggaccctcaggggccgaggtgcccacacctgtgctccctgcatgacagacttcctggggtcttggctcccagtctgtcctcatcctctacacacacccaaatgtggaagtcacccccagcttgagtgaatcccacaccctcagaccattggccatgatattacgtgtgttgcaaaatatcaaggattcagctgagaggctctcgcagtggacggctcagaggccgagtcacacactgcccaggctttccctggggggccctggcccgggggccccctgccttaagatgcccttcctctcctccctcagtctcccactgtcttcaactcgggccctcactctgcttatcatagaccccaaaatgcctctgctcaaacaaatggcttgacctgttagcgatatagaaaagtgagcggatcctttgaacatgttcgtttctccttttctccacccaccctgcgccgtttcccatttctctaagtgcctggaatgtgtggagagtctcctgatgatatgatgccagctgtgcccagctccctggaacacaacatagggaattaaccagtgtgttcctctttcctccgttagtgaaaatgagtactatttaataatgcagtgacacaggatttgttgctgttgcagcacttgcatggccatgctcaccttcacaccacgcggaggccaaaggcattgttccctcagctgcggccctctcccctcagcagccctggccattccaccatggtgtagtcctcctgcccttctccatccttctgaatcccattctgccagctccagggctgcacgccctctggaatgaccacccgcagctagcccaagctgctcctgctgtttattttctttgcactttgtttaattatttcccacatcttggtcctctctccttgatttcagatggattgctgaagacagagtgtatttgtggctccgctcaggctgtacacagacaggggcactcagcatccgtgggtcgtatttcattctagggccaggagcgcgggctactgcgtcagtgggaaagacgtggagatgagttcatatttacctatttcatggtgaaatctgcaaggtccctaaggcaatggctttcttgaatggtgacagcaactgatgagtctgaaaaatctttgtgtctcacttaggatttttgcacagctggtttcataattcagttattttgatacaaaagcgttctgctctaattagtaaaaaaagaccaggcgatagtgtttgcctcttgttaggtggctgccccatccatgcctttcatttctggagtaggtgcccaggaaatgtttactgagttgcaccagtgaatgaactcatgatgccgggattagaaggggaagcccttggagcctccttctgccccagttctcagcgtccctggtgttcagtaagtattagctggtcagtggagt

SEQ ID NO: 249 - DNA, 2271 bp, Homo sapiens
catttctcagaataatgaatggcaggaaataccatagttaattaataattgactggtttgtaattatgtgctatctacacccataaagaaattgagaagctcataaaatgcacatataaataagagttaattatgtgaataagtttaaatgtttttatgacaatttaaaattattttacttttataagacttccatgtaggtactagcactttcattaatgtgcttgctatttttcacttaaatttttatctctatgaaaacctaacaccttcgagaaacggattcatgtgcacgtttctgttgctaaactgtggcaggaacatcagaccttaataagagaagggtgaggaaccacaactgcatatgtagtattcacagtaggagaaaagtgatactaatataccatgtagaaaaaaagcacaacaaaataagataccatttagcacacacagacaaacatgtttgctgctttgtttcttgtgactgacagacgctcttacttactccgagtctttgaggtaataactgcttggaagatggccgaagaggaggtgttgacatgcaagagtggctattttaaaggagcacgaaccatgggctaataagcgcctgcgatgtggccacttcaagcccacatgctgccagcaccatgtcctcgtctggcgtggacatccaagggcggaggaagagctgaaccctccacaaaggttccatttgtatgcagaaacaatgtccacagtaggcgagggttttctttaaaatcattagcgtagctaaatttcaaagttcaagtaaaaattgttttttacagattgggaagtcctcttccgttgtacccatcagcagaaggtgtgtgtgttcaaggcaaagcgatcagaattgagtgcagaattgacctctgtcggaatgttccgcatcctaggtctcctgtccctcgctgccactgcgaagtttgctggagacagactgtgccttcacggtcagacaatgccctcctggactcttctggctttgtaatgtgcctgctcttcagccagacggggccttctggaaggagtgaaggccagtagtcagagatgctggtgcaaacctatgctctgtcattcccagactcggtgttcttgggtgaatcctctccctgtctgttttctgggaataataagaacctgtcacttctgtctttgcgggctgctgtgaggatggtttgctatgctgtaatatgaaaggaccatgcagatgataaaatgacccacagaaaaagctggtattctcattatcatcatttaaaatactacaggtgaactttctgtgtaagtagaggttctttgcagaaacatttttgttttaaatttttgaaaagactttatccttgaacagaatatgtggcagagggatttgtccgtattcatgtctcattacaaacatctcttctggttaaaaatgcaaatgcagctgacaggagaggacagatgcttggctagaagccttctgactgtcatcctcagctgcccctcagcagtaactacaaagcctgcttcctcaaaagctactcctggtatttgctgggttgtgccctcttcttttttttttcttctttttttgctttatgcacaaagtgagcagcacaaaggcatgatctcatggccattgtagcatgggcaactttgggttaaattgctttggtctctatttaatttggttatttttctcccacatgcttttgcactgtccggaaaatgagctttttcatgattactctcagtgtgctgagactagtcagcagcgttgaaagattctttgtttttgcacagccagcccagggctcacggacacactttaatatcctgcatccacactcccttttcctttgtgtgtaaattcccgagaatgaaggaaccgttttaccccctcatgtttcaggatgctttgctaaggcgagaacctcacagtacatgaaagcacctgtagggctcctgtctgaggagccacccacctatgtctgcatccagtccgctcctttacaagattaaagtggcccggctgagacactgctttttagaaggtaagttacactcagaaaagtcttatctgaaaaatcgtgtttgactgttaacagatctaatgttattctttaaaaaaatatagtccaacttatagaaatttctcattgagagactatctaaacagtgaacagtgaccaaacacaagtcctctgttagggtaggaacagccgcacaatcacaatctgagaatgtcttgaaacatgcaca

SEQ ID NO: 250 - DNA, 2949 bp, Homo sapiens
aaactgtgtcctgacacccccagacctgctggccagcagggaggggcctctcagcatctgggctttctccttgctcagggaacaggagcacagctctgagaactaaggatgggggtaagtgagctaggccctcaaggcagggcacttactaggtggaaaaaacagcctggaagctcatgggcatgaaaatgaggtccatggagagagcttcctctgtggcccagaaactagaagctggaacagccatgtggaactgtgcagcagcccagaacaggatatgggggcctaagtcacagcagaccagtgagaggagaaagctgacctcagattgcagatctgtataaagaaaagtagggtggcgggggagccttgggttcaaattctggaacaggagggacaaagaagggcagggaattggtggtgatgagtaggtaccacttctggggaagatgacagagcaactggacctgaaaaactctcgacttacctaaaatatcaattacagccagtgacaaagaattcacgccacacaactcattaccaatcaaacaaactactatggttatctcaaaccaaacgtcactttacttttttggtaacttttcattataataataaactctattcatgaatatgcagcctccataatcttctcccttgtaacaaacgtgcagtccgttcacaagctgtaaaaacaagcccaaacccaagacatcacaagaggcaagagcagtggcagtgagaagggagcctgtaaaggatgtttcaaaggagggtcccaggctatgtggccactggatgtaggcagtgagctgagtccaggctttcggtctgggaagtggcagaggctgagacaatggccaaagaggagttggagaggaaactatgctcggtttcactcctgccagcccaacagcctattccctggtgtgaatcaactggtgtttgatcaactttgatcgctggctgaaggctttcccacaagcagcacagtcatagggcttcaccccagtgtgaatcctctggtgctggatgaggaccgaacgctgactgaaggctttcccacactcactgcatttgtaggggcgctcgcccgtgtggattatctgatgctgaatgaggtgtgagctctggctgaagcccttaccacattcaacacaggtgtagggtttttccccagtatgaactttctggtggtgaatgagatttgagcttcggttgaaggctttaccacactggttacattcatggggcttcagcccattatgaatcctctgatgctgaatgagggttgagctctggctgaaggtttttccacattcagtacattcatagggcttctctccagtgtggactcgctggtgaaggatgaggttggagctgcgaccaaaggtcttcccacactcgtggcaggcgtagggcttgtcgcctgtgtgcacgccctggtgctgaatgagggctgagctgtggctgaaggccttcccacagacactgcatctgtacggcttctctcccgtgtggatgatctggtgctttcggagcactgagctataactaaaggcttttccacatacattacacacgtgaggcttttctccagtgtgaattctccgatgctgaataaggctggagctctgactaaatgctttcccacagtcactgcacttatagggcttctctccagtgtgaaccctgtggtgcttaatgaggttggagacccgactgaagggcttgccacaatcattacactcataaggcttctctccagtgtggaccctctggtgcttcctcaggtgtgcactctggctgaaggctttcccacactcgccacactcaaaaggcttctctcctgtgtgagtcctgtggtgtttgatgaggtttgagcttcgcctgaaggccttcccacactcactgcacacatacggtttctccccagaatggattctttgatgttggatgaggtttgagctccgcctaaaagccttcccacattcattgcattcatagggcttctcactcatgtgagacttttggtgctttttaaggctcgagttctggctgaaggcttttccacattcattacacatataaggcctctcactgctgtggtgactctgatgcctagaaaagtctgagtgccctcggaaggctttcccacattcgctgcactggtaagctttctcactcatatgagatcgatgacggtttttaagaactgagttctggctgaaggttttcccacaatcatcacacataaaggaagcctccccagtgtggactatttgacgctgaataaggtcaggatttccttggaaggttttcccacactcattacatatgagtggactttcagctgtgggaaccccctcatgaccagttaggtccacactgtgctggaaactctggccacccatgtcatatggatgtggcctctcttctgtagggatttcctgacatgccatcaggtttgggctcagactgaagcgactgtcaaaaccattacagtccagatctttctcccctaaggggcccctaaggagccccatggcagctggtgtgaagtccccctcctgggagagggactgtggcagcctcctgccttcggggactccccagtctctttctgatacatcatcacacagatctccaagctcgggtacctgggaaacatcaccagcatagttttctgatatttctgcctgtgattccaaatcttcatgaatgtcttccttgtgaagaaactccttgtcttcagtcctggtgtcacaatctgaaacaataaatagaatatcacttggaaggcagtgctgcagcaggagcaggaacatagacagtcacagttgcacccactaactgtggaggaggcaaggggagcaggggatcctctggggtggcagtccagatcagagggcatcagggaggggtgggaggagcactgggtgattaggc

SEQ ID NO: 251 - DNA, 1754 bp, Homo sapiens
cactccatccctcctggaaaaggactggaccccaattcccaccattgcttttttgggacccattatcttccttagcttcctatgcatctacagggtagtctgggcttcacttcctcagtgtccctgtatgaaattaggtggatatagattagtctgatgtaggaatatcacactgtactaaggtttagtttgtatgttattctctcaagtaactgatctttcaatccaactaaacacttcctatgtgctttaaggtggtgggaattacaagcatagcaagttatgattggtcacggatttctttcctctttaaatggtgacctactgcccattgtacctactcaaagcaactttctttaggaaaaaagaccacagtctactttcctaagcataaactcagttctcattccacctctaccacctgcaagatttgttaggcttaagcagtcccttaacttctttgagtgtttgttgccttgcctacttcattggaagtaaggctctggaacagggaaggtttgcctccataagactaaaagttatgctaatataagagactagcaaaatgggagacatattcagctctcttcttgtggggaataccttgcccttgaccaaaagccttgtcccagaaagagccgtgtgggtgttggctttgtgcccaacatgtggctcctctgccatgattgatggcttcatttaagaaacaggttttaggattttttcccctaaaatcttattcctgttaattatcatggatcaactttaccttagctcgtttaatacacagtcacctggtataaaagcatgtgaaaacccccagggatcgtaaccacatttatgcattgagaaaagagagtgaggccaagattttgagatgtgttcaaatgcaagaagcttttaaaatgcaaagtattctaaaactgttgaaagttgaagctaactgttgttcccttgttgaaggtaaaaagtaaagcatttttaggaaagcacttttccttatgtgtctaatatttgggaactgcataggagaacagtttaataggaaccctgatattgacagtaagatatattcttaatgtagtaaccagacccagggcagaatttgcaaacccatggtaggcatacaggtggctgaagaagaatcgggacagcaagatctcactgagatgcaattccattcctccatttgatacagattaagatttctgaaaaagaccatcctcctaaaccctcatggactctgcagataatatgaggccagaaaatgaataattcccaactcttgctatctcgttactggccagtgtgtctggcttcgctgagtgtgtgccttctgaagcgtaccctataattattcagcaggtatagtccagttcgtcctacttactttagcaagattacctttcttttatttttcctgtgaaaatccttctcttccttctttcctcctttgtctttcctctttgttaactttttaaatctaaagtgccttgaaaaacttgtttacatagtagtaagaaggaaaatgttgacttgtgctatcctgggaaccttgaccttcctgcattatggataaatcatttccctgcaggtggaagtggaaaattgcagatagaaccacattgactcacattctccttctacttccatttgagtgagcaccaagtatgcatcacgacttgagattataaagttggcttaatgatgagacaggtttctcagtcgggttttccattggctcgaagttcaca

(19) United States
(12) Patent Application Publication
(10) Pub. No.: US 2004/0161773 A1
(43) Pub. Date: Aug. 19, 2004

(54) Subtelomeric DNA probes and method of producing the same

(75) Inventors: Peter K. Rogan, Overland Park, KS (US); Joan H. M. Knoll, Overland Park, KS (US)

Correspondence Address: HOVEY WILLIAMS LLP, 2405 GRAND BLVD., SUITE 400, KANSAS CITY, MO, 64108, US

(73) Assignee: THE CHILDREN'S MERCY HOSPITAL

(21) Appl. No.: 10/676,248

(22) Filed: Sep. 30, 2003

Related U.S. Application Data
(60) Provisional application No. 60/415,345, filed on Sep. 30, 2002.
(60) Provisional application No. 60/484,494, filed on Jul. 2, 2003.

(51) Int. Cl. C12Q 1/68; C07H 21/04

(52) U.S. Cl. 435/6; 536/24.3

(57) ABSTRACT

The present invention provides subtelomeric probes and primer pairs which can be used to develop subtelomeric probes as well as methods of making and using the same. Advantageously, the probes are located in close proximity to the telomere of a chromosome and are generally much smaller than currently available probes.

### RELATED APPLICATION

This application claims the benefit of application serial No. 60/415,345, filed on Sep. 30, 2002, and application serial No. 60/484,494, filed on Jul. 2, 2003. Additionally, the content and teachings of each of these provisional applications is hereby incorporated by reference herein.

### SEQUENCE LISTING

This application contains a sequence listing in both paper format and on two identical CD-ROM's filed herewith. The sequence listing on paper is identical to the sequence listing on the two CD-ROM's and all are expressly incorporated by reference herein.

### BACKGROUND OF THE INVENTION

1. Field of the Invention

The present invention is concerned with chromosomal ends and subtelomeres and the detection of chromosomal rearrangements occurring in the subtelomeric regions of chromosomes. More particularly, the present invention is concerned with probes that can be used to identify such chromosomal rearrangements in medical and cancer genetic diagnoses. Still more particularly, the present invention is concerned with single copy probes effective for hybridizing to a single location in the genome wherein hybridization analysis will indicate whether the chromosome has undergone any rearrangment at the telomere or subtelomere region. Still more particularly, the present invention is concerned with single copy probes that are useful for detecting a broader spectrum of abnormal chromosomal termini than currently detectable with existing cloned probes, providing insight into how the telomere and subtelomere regions of chromosomes are organized, correlating how the sequences of these chromosomal regions are related to each other and to other chromosomal regions, correlating rearrangements with specific clinical effects, and characterizing breakpoints in rare chromosomal rearrangements that are genetically balanced and unbalanced. Finally, the present invention is concerned with methods of making such probes.

2. Description of the Prior Art

Chromosomes are the DNA-containing cellular structures of organisms and are visible as a morphological entity only during cell division. Chromosomes consist of two chromatids.

Each pair of chromatids form a homolog, each having a short arm (the p arm), a long arm (the q arm), a centromere connecting the long arm to the short arm, and a telomere at each end. After pretreatment of the chromosomes with chemicals or heat, each of the arms exhibits alternating light and dark banding patterns that are a function of chromatin condensation. G-banding is in common use in clinical cytogenetics. R-banding or reverse band is occasionally used and is the reverse pattern of light and dark G-bands. G-banded chromosomes will be referred to in this application.

The centromere is a specialized protein-DNA structure in human chromosomes that binds the chromatids together and is responsible for accurate segregation of chromosomes in somatic cells and germ cells. The centromere is often visible as a constricted region in the chromosome and its position is responsible for determining whether the chromosome is metacentric, submetacentric, or acrocentric. In metacentric chromosomes, the length of the p arm (or short arm) is roughly equal to the length of the q arm (or long arm). In submetacentric chromosomes, the length of the p arm is somewhat less than the length of the q arm. In acrocentric chromosomes, the length of the p arm is much shorter than the length of the q arm. It is known that acrocentric chromosomes have a specialized short arm comprised of highly repetitive DNA sequences and multiple copies of genes for ribosomal RNA.

Telomeres are specialized protein-DNA structures that demarcate the ends of each chromatid in a chromosome. Typically, the telomeres are located in a light G-band which are gene rich and contain a lower density of repetitive sequences as compared to the dark G-band regions. Because of their location in the light G-bands, exchanges and rearrangements between the terminal ends (the telomeres) of chromosomes are difficult to detect visually. While telomeres are not chromosome-specific, the subtelomeric or telomere-associated repeat sequences immediately adjacent to them and also located in the light-staining G-bands can be chromosome-specific. The telomeres themselves are composed of a TG-rich repeat of 3-20 kb in length, which in vertebrates is (TTAGGG)ₙ. This array is required to maintain chromosome stability by preventing end-to-end chromosome fusions and exonucleolytic degradation. Additionally, telomeres are needed for replication of DNA and have an important role in maintaining cell longevity. Immediately adjacent to the TTAGGG tandem repeats are families of complex repetitive DNA of up to several kilobases (kb) in length. These sequences tend to be present on multiple chromosomes, and are confined to the subtelomeric regions. Naturally occurring mutations in humans reveal that chromosomes lacking these repeats can be inherited normally, suggesting that these sequences have no important biological role. Sequence analysis of DNA adjacent to the 4p, 16, and 22q telomeres revealed interstitial degenerate (TTAGGG)ₙ repeats dividing the subtelomeric regions into distal and proximal subdomains with different degrees of sequence similarity to other chromosome ends. The proximal subtelomeric sequence contains long sequences common to a small number of chromosomes and the distal subtelomeric sequences contain the previously described short complex repeats common to many chromosomes. Additionally, chromosome-specific low-copy repeats or duplicons (i.e. paralogs) can occur in multiple regions of the human genome including the subtelomeric regions. Trask et al identified members of the olfactory receptor gene family within a large segment of DNA that is duplicated and has high similarity near many human telomeres. Intra- and interchromosomal recombination between different duplicons in this gene family leads to chromosomal rearrangements. The similarity between non-allelic copies of highly related sequences (>95% homology) has made the subtelomeric domains extremely difficult to analyze at the molecular level.

Subtle chromosomal rearrangements involving a gain or loss of the subtelomeric regions (neighboring sequences) have been observed in 0-10% of individuals with idiopathic mental retardation and other inherited clinical abnormalities. Other applications of subtelomeric probes include investigation of individuals with recurrent spontaneous miscarriages and infertility, characterization of constitutional and acquired chromosomal abnormalities, selected cases of preimplantation diagnosis, and diagnosis of abnormalities using interphase cells obtained either for chorionic villus sampling or early amniocentesis.

Cytogenetically defined terminal deletions occur by three mechanisms: telomere regeneration or healing, retention of the original telomere producing interstitial deletions, and formation of derivative chromosomes by obtaining a different telomeric sequence, ie. telomere capture, through cytogenetic rearrangement. Because the majority of telomeric deletions are probably stabilized by telomere regeneration, this suggests that the maximum number of terminal deletions should be detected using probes that are as close to the telomere as possible.

Due to the small size of these rearrangements and the presence of pale staining bands at the ends of most chromosomes, the rearrangements are often not detectable by routine cytogenetic methods that include G-banding or R-banding. Instead, they are detected by DNA probe hybridization to chromosomes and fluorescence microscopy in a technique referred to as fluorescence in situ hybridization (or FISH) or by microsatellite analyses. Unlike microsatellite analyses which require that parental and/or other family members be studied in addition to the patient, FISH requires only the patient sample to detect the abnormality. Conventional FISH probes are generally between 60,000 and 170,000 base pairs in length with an average of about 110,000 base pairs in length (rather than 5 million base pairs which is the average size of a chromosomal band) and usually come from a portion of one chromosomal band. Therefore, FISH can detect abnormalities not seen by routine cytogenetic methods. The probe hybridizes only to the homologous DNA sequences near the end of the chromosome arm. In normal individuals, there are 2 copies of the sequence (one from each parent) and thus, 2 sites of hybridization (one per chromosome of each homologous pair) in each cell. In patients with unbalanced terminal chromosome rearrangements, there is a deviation in either the copy number or location of the sequence, such that deletions are detected by the absence of hybridization from the end of the cognate chromosome and trisomies are detected by the presence of an additional hybridization signal on another chromosome. The chromosomal location of the hybridizations is immediately apparent from cytogenetic characterization of the chromosomes, enabling both balanced and unbalanced translocations to be detected.

Given the highly repetitive telomere structure and the fact that all current approaches rely on the presence of unique sequence to investigate subtelomeric regions, there is a tradeoff using current assays between sensitivity and specificity. Sensitivity is defined as having a probe that detects the smallest deletions (ie. close to the chromosomal end), and specificity is defined as a probe that contains only sequences from a particular chromosome. Probes containing complex repeats in the distal telomeric and subtelomeric domain may lie closer to the end of the chromosome, but lack the specificity of single copy probes (such probes can be used to assess the integrity of multiple or all telomeres simultaneously). Current “chromosome-specific” probes capable of detecting specific subtelomeric regions are generally large, and usually do not lie in the distal subtelomeric interval. Due to their larger size, these conventional FISH probes have a greater likelihood of containing low frequency paralogous sequences found on other chromosomes (and hybridizations to such chromosomal targets cannot be suppressed by addition of Cₒt 1 DNA). In order to select cloned probe sequences that do not have paralogous copies on other chromosomes, conventional FISH probes must be comprised of locus specific segments. Sequences meeting these criteria are often a considerable distance from the telomere. Deletions that occur between the sequence recognized by the probe and the telomere cannot be detected with such probes. Thus, assays that use large chromosome-specific telomeric probes compromise the sensitivity of the assay, as more distal terminal rearrangements will fail to be detected.

The first generation of chromosome-specific FISH probes for each telomere (except the acrocentric p arms) were cosmids, fosmids, bacteriophage, P1, PAC clones derived from half YACS (Yeast Artificial Chromosomes), which possess large intact terminal fragments of human chromosomes. These clones are composed of clusters of single copy sequences interspersed with repetitive sequences on chromosomes. There is a paucity of chromosomal sequences with this genomic organization the ends of several chromosomes as a result of the high frequencies of paralogous sequences (often seen on multiple chromosomes) in the terminal bands of chromosomes and the relatively high densities of telomere associated repetitive sequences. Half YACS were not available for 1p, 5p, 6p, 9p, 12p, 15q, and 20q telomeres and these ends were derived by screening genomic libraries with the most telomeric markers on the human radiation hybrid map. Consequently the physical distance between these clones and the cognate telomeres was unknown. It is now known that some of the subtelomeric commercially-available probes used in conventional FISH are not located near the telomeres but rather several hundred kilobases from the end. Interphase mapping has since shown that the commercially-available 9p clone is <1.2-1.5 Mb from the telomere and the commercially-available 12p clone is >800 kb from the telomere, whereas the commercially-available 15q clone maybe ˜100 kb from the telomere. The distances for some commercially-available 1p, 5p, 6p, 11q, 19p, and Yp clones are still unknown. Large gap sizes between clones and the corresponding telomere, genomic polymorphism in hybridization patterns and cross-hybridization has prompted the development of a second generation set of telomere specific clones. While these clones are in the vicinity are of the telomere, substantial distances to the ends of the chromosomes remain. Some of the commercially available probes are so far from the telomere that they do not even reside in the terminal light-staining band region of the chromosome. For example, based on the coordinate of the sequence tag site (STS) in a commercial 14qtel probe, the probe is located in 14q32.32, a dark G-band, and is therefore closer to the centromere than any probe that would be contained in the terminal light band. These clones have large inserts, which assure that hybridization intensities are adequate, however they may fail to detect deletions of sequences contained within the probes themselves or of sequences closer to the telomere itself.

In conventional FISH, the DNA probes contain large genomic intervals (from ˜50 to several hundred kilobases) which consist of both unique and repetitive synthetic DNA. Because repetitive DNA has a widespread distribution, it can interfere with the detection of chromosome-specific abnormalities. As a result, methods have been developed to suppress the repetitive DNA and prevent binding of repetitive sequences to chromosomal DNA. One such method involves preannealing these repetitive sequences in the probe with an excess of unlabeled repetitive DNA, so that only the probe's unique sequences hybridize to the chromosome.

Conventional probes suffer from many deficiencies including the fact that they are unsequenced and therefore, their locations have not been accurately determined in chromosomes. By comparison of the sequences of available sequence tagged sites (STS) contained within these probes, it has been demonstrated that several of these probes contain sequences that are considerable distances from the telomere (millions of base pairs). The lengths of the conventional probes themselves have only been approximately determined and the STS could occur anywhere within the probe. This means that the precise location of the probe can only be determined within a window spanning equal distances corresponding to the approximate length of the probe both proximal and distal of the STS. Furthermore, some of these conventional probes were derived by complementation of half-YACs (which lacking telomeres) functionally for the presence of sequences that serve as telomeres. In fact, several of these synthetic DNA clones do not contain the actual telomeres of a number of chromosome arms. Telomere-like sequences (which may have served as telomeres in lineages ancestral to humans) can be found at multiple internal locations in human chromosomes, and these sequences may have been selected for in the complementation studies that were developed to retrieve human telomeres and associated single copy sequences.

Furthermore, the coordinates of several conventional probes cannot be determined because the sequence tagged sites (STS) reported by Vysis, Inc. and by Knight et al. correspond to their internal laboratory designations, rather than being assigned by the public Human Genome Organization nomenclature committee. Unless these laboratory-based STSs were deposited in the genome database, GenBank, or other public databases, the laboratory designations of these STSs cannot be related to publicly assigned STSs. Accordingly, due to these obstacles, the locations of several of these STSs have not been determined in public sources. Therefore, synthetic clones presumed to contain subtelomeric sequences cannot be anchored on the reference genome sequence by these STSs and their location in the genome cannot be confirmed except by microscopic visualization of these probes. Such microscopic visualization lacks the very high resolution that can now be achieved by direct mapping onto the human genome reference sequence. The inability to map several of the available subtelomeric probes that are in common use in cytogenetic laboratories has potentially adverse consequences for patients with chromosomal abnormalities involving the terminal bands of chromosomes. If these probes consist of sequences that are localized considerable distances from the ends of the chromosomes (like the 14qter and 16pter commercial probes), then it will not be possible to determine whether the failure to detect an abnormality is due to the position of the probe on the chromosome, the size of the rearranged chromosomal region or both of these factors. This is the case for subtelomeric probes available for chromosomes 1p, 5p, 6p,11q, 19p, Yp, Yq. For such probes, it would not even be possible to determine if the failure to detect an abnormality is due to a false negative finding (ie. an error) using the probe. This situation is unacceptable practice for a reagent commonly used for clinical diagnosis of disease and an application for a medical diagnostic device based on them would be rejected by the US Food and Drug Administration based on current guidelines. Of course, the probes are labeled for research use only. Moreover, it is not even possible for one skilled in the art to investigate the locations of several of these probes because the clones from which they were derived are no longer available. This means that these conventional cloned reagents which are in common use cannot be subjected to quality control standards by independent researchers, despite the fact that these reagents are commonly used for detection of clinical abnormalities. Since the completion of the human genome reference sequence, several companies that produced genomic reagents for human genome mapping and characterization have discontinued support for these products or no longer maintain them, due to lack of demand. One of these companies that produced cloned synthetics for detection of subtelomeric rearrangements is no longer in business and the company that acquired them discontinued support for this product line 2 years ago. Accordingly, one thing that is needed in the art is a set of probes that are precisely localized and are derived from available genome sequences which are essentially perpetually available.

Finally, it has been shown that prior art probes suffer from cross hybridization to other locations in the genome in addition to the location of interest. This occurs because many synthetic DNA probes for subtelomeric analysis are not sequenced and therefore, it is not possible to verify by sequence analysis of the human genome that the DNA sequences contained in them do not have paralogous sequences at other distant locations on the same or other chromosomes. Consequently, several of these probes have been found to cross-hybridize to other chromosomes. The manufacturer (Vysis, Inc.) discloses that the following probes cross-hybridize to other chromosomes in their product literature:

| Probe | Cross Hybridization Location |
|---|---|
| 3q | 2p |
| 4p | 17p |
| 8q | 11p |
| 10p | 12p |
| 11p | 16p/17p/20p |
| 16q | 4q/9q/10p/16p/18p |
| 17p | 11p |

Additionally, the Xp and Yp share homology and a single probe that detects both is available. Similarly, a single probe to detect both Xq and Yq is available as they share homology.

A hypothetical example can be used to describe the potential adverse consequences of such cross-hybridization. Suppose a parent contains a cryptic chromosome rearrangement that was a translocation between chromosomes 10p and 12p and this translocation is transmitted to her offspring in an unbalanced manner, such that one of the 10p sequences is missing and the 12p sequence is duplicated. Using the 10p probe, the normal copy chromosome 10p crosshybridizes to a single chromosome 12p, this would suggest that a translocation between these chromosomes had occurred. Because of the loss of 10p sequences from the other homologous chromosome, there would be only one hybridization evident each on chromosomes 10p and 12p. However, a chromosome 12 probe would hybridize to three copies of this chromosome (the normal and duplicated copies), which would be inconsistent with the results found with the 10p probe. Unequivocal interpretation of both findings would require unnecessarily complex (and ultimately, incorrect) explanations. Accordingly, what is needed in the art are probes that do not cross-hybridize. Such probes would clearly and simply demonstrate the presence of the translocation and the unbalanced nature of the karyotype.

Currently the two most common techniques for studying subtelomeric regions are 1) FISH of probes (BAC, PAC, P1, YAC and other large synthetic clones) mapped to terminal chromosomal bands, and 2) the use of polymorphic microsatellite markers mapped to the subtelomeric region. For the first technique, a number of disadvantages are observed. First, cross-hybridization of certain subtelomeric probes is evident, some polymorphisms resulting in deletions have been detected and not all of the probes are as close to the chromosomal termini as reported such that they would not be able to detect smaller subtelomeric rearrangements. Table 3 shows the distance of the common commercial probes used in clinical diagnosis from the end of the chromosome.

For the second technique that involves use of polymorphic microsatellite analysis, one disadvantage is that the markers must discriminate between chromosomes (ie. be informative) and most of the informative markers are located a relatively long distance from the telomere. As a result, small deletions could be easily missed by this method. An additional disadvantage is that DNA samples from the patient's parents are required.

Other molecular techniques have been developed and used for assessing subtelomeric regions. The multiplex amplifiable probe hybridization (MAPH) allows assessment of copy number at specific loci. This technique relies on correct genomic placement of currently mapped genetic loci/STSs and will miss small deletions if the loci/STSs have been placed in a wrong position within the chromosomal end. For example, D16S3400 was originally placed within 300 kb of the chromosomal end but we have placed it more than 3000 kb from the chromosomal end using the April 2003 version of the genome sequence (see table 3).

Multiplex ligation dependent probe amplification (MLPA) is conceptually similar to MAPH, except that it is less tedious and simpler to perform on specimens from patients. Like MAPH, determination of sequence copy number in the specimen is dictated by an initial hybridization of probe to purified patient genomic DNA. Instead of measuring the amount of hybridized sequence with a secondary probe that is related to a target sequence, MLPA achieves specificity for the hybridization target by ligation of very short sequences homologous to the target in vitro. Read out occurs by PCR amplification of the annealed, hybridized probes using universal primers in vector sequences adjacent to the complement of the genomic target. Both approaches, however, depend on prior knowledge of the single copy nature of the genomic target sequence in normal individuals, since the abnormalities is detected by determining the ratio of hybridization in normal and abnormal targets. This approach contrasts with the method of the instant invention, in which the single copy properties of a sequence are established during the development of the probe. This is not a trivial difference, since the presence of paralogous sequences in the genome related to the probe could result in false positive detection and distort the copy number ratio determined with the probe sequence. Given the very short lengths of the homologous genomic sequence contained in the MLPA probes, one skilled in the art would have to have prior knowledge of the single copy nature of the gene region from which the probe were derived, in order to be confident that paralogous targets were not present in the genome. Finally, while MLSPA is simpler to perform than MAPH, a substantial up front effort is required to clone a pair of genomic sequences in phage vectors by synthetic techniques prior to testing patient specimens. Such cloning steps are unnecessary in the art of the present invention.

Array based comparative genomic hybridization (CGH) has been used used to survey subtelomeric rearrangements. This technique has the advantage of surveying multiple regions of the genome simultaneously, however it has a number of pitfalls that are not inherent in the present invention. For detection of unbalanced rearrangements, large cloned synthetic DNA probes in the telomeric region are required. (a) Several of these probes are not close to the telomere (b) the large size of these probes precludes the detection of small rearrangements, and (c) terminal chromosome rearrangements that overlap a portion of the sequence homologous to the probe will be scored as intact (ie. false negative results) (d) hybridization of repetitive sequences in these probes must be blocked, typically with an excess of Cot1 DNA. Variability in the batches of Cpt1 DNA and in the efficiency of this blocking procedure has been shown to compromise the laboratory-to-laboratory reproducibility of this procedure, which makes it less suitable for clinical or research testing.

Most of these techniques do not detect balanced translocations which is needed for identifying parental carriers of these rearrangements that could result in additional offspring with unbalanced chromosome complements and clinical abnormalities. Conventional FISH probes will detect these rearrangements if the chromosome breakpoint is contained within sequences homologous to the probe or if the probe is known to be distal to the breakpoint. The likelihood that a subtelomeric probe would detect such a rearrangement is quite low, since the probe is relatively small (100-300 kb) compared to the potentially large region in which the break might occur (several megabases) and generally has not been precisely localized within the chromosomal interval. By contrast, the breakpoint for such rearrangements can be identified by systematic hybridization of an array of single copy probes derived from this chromosomal band (Knoll and Rogan Am J Med Genet 2003, the teachings and content of which are hereby incorporated by reference), whose positions in the genome are determined during the development of these probes.

### SUMMARY OF THE INVENTION

The present invention overcomes the deficiencies of the prior art and provides a distinct advance in the state of the art. In particular, the present approach develops unique sequence, single copy hybridization probes that are considerably smaller and generally closer to the chromosome ends than available corresponding cloned probes for detection of subtelomeric abnormalities. Preferably, each probe is specific for a single chromosome arm. Additionally, the probe must be of sufficient length for detection, preferably by fluorescence microscopy, array comparative genomic hybridization or related techniques. The probes of the present invention preferably have lengths less than 25 kb, more preferably between about 25 base pairs and about 15 kb, still more preferably between about 50 base pairs and about 12 kb, still more preferably between about 60 base pairs to about 10 kb, even more preferably between about 70 base pairs and about 9 kb, still more preferably between about 80 base pairs and about 8 kb, still more preferably between about 90 base pairs and about 7 kb, still more preferably between about 100 base pairs and about 6 kb, still more preferably between about 250 base pairs and about 5 kb, still more preferably between about 500 base pairs and about 4.5 kb, more preferably between about 1 kb and about 4 kb, and most preferably between about 1.5 kb and about 3.5 kb. Such preferred probes are up to 100× smaller than the currently available probes. Advantageously, these small probes can be designed to exclude hybridization to low copy paralogous sequences on other chromosomes. Due to their size and the relative abundance of paralogous sequences in these regions, larger cloned probes, such as those that are currently commercially-available, are more likely to contain sequences with paralogs on other chromosomes. Such larger probes have greater potential to compromise specificity, and therefore might not be ideal for distinguishing the subtelomeric region of a particular chromosome from other genomic sequences. The requirement for hybridizing larger probes provides one explanation as to why these clones are comprised of genomic sequences that lie further away from the telomere and why some contain paralogous, cross-hybridizing sequences. Moreover, the isolated short genomic intervals recognized by single copy probes permit the identification of specific hybridization intervals that are closer to the ends of chromosomes than available synthetic DNA probes that are presently used for detection of subtelomeric rearrangements. Hybridization of probes of the present invention is detectable regardless of whether the entire probe or only a portion of the probe is bound to the chromosome. Therefore, the extent of a chromosomal region gain or loss that involves only a portion of the probe sequence may not be recognized by the prior art probes but will be recognized by the probes of the present invention. The shorter probes of the present invention will thereby produce fewer misdiagnoses (false negative results for chromosome deletions, for example) when analyzing the genomes of patients whose breakpoints occur within the chromosomal sequences spanned by the hybridized probe.

Probe design for single copy hybridization should permit generation of considerably smaller probes that are closer to the chromosomal ends than are currently available. Generally, the method comprises searching a moving window beginning at the terminal nucleotide on a chromosome end on the human genome sequence database (i.e., Public Consortium Celera Genomics Data Bases) to identify single copy intervals in the terminal chromosomal band. Preferably the single copy interval is the single copy interval in the subtelomeric region that is closest to the telomere. Preferably, the single copy interval is within about 8000 kb of the terminal nucleotide of the telomere of the chromosome, more preferably it is within about 7000 kb of such a terminal nucleotide, still more preferably it is within about 6000 kb of such a terminal nucleotide, even more preferably it is within about 5000 kb of such a terminal nucleotide, more preferably it is within about 3500 kb of such a terminal nucleotide, still more preferably it is within about 2500 kb of such a terminal nucleotide, even more preferably it is within about 1500 kb of such a terminal nucleotide, more preferably it is within about 1000 kb of such a terminal nucleotide, even more preferably it is within about 800 kb of such a terminal nucleotide, more preferably it is within about 600 kb of such a terminal nucleotide, more preferably it is within about 500 kb of such a terminal nucleotide, still more preferably it is within about 400 kb of such a terminal nucleotide, even more preferably it is within about 300 kb of such a terminal nucleotide, still more preferably it is within about 200 kb of such a terminal nucleotide, and most preferably it is within about 100 kb of such a terminal nucleotide. The method may then comprise the step of verifying that the identified interval is in fact a single copy sequence and is found only in that interval. Such verification can take place either computationally or experimentally and a preferred method includes both forms of verification. Experimental confirmation or verification can be accomplished through conventional techniques including experimentally hybridizing the single copy sequence to chromosomes. Computational verification can occur by conventional computer-based techniques for searching genomes including analyses with BLAT or BLAST software. However, other equally suitable techniques for genome-wide computational sequence comparisons would also verify the single copy nature of potential probes. Single copy sequences are then sorted by length and primers are designed for some of the intervals (preferably those greater than 1.5 kb in length because they can be reliably visualized by FISH and those closest to the telomere but in the subtelomere region). Primers developed during such an approach would indicate to those of skill in the art that the desired sequences could be developed using conventional techniques and publicly available knowledge including the publicly available genome databases. This is because the coordinates of the primers can be found in the genome databases and then these primers can be used to generate the sequence of interest. Furthermore, the developed sequence can be verified by comparison to the genome drafts. Primers developed by the present invention and their locations are provided herein.

Single copy probe technology, such as that disclosed in U.S. Ser. No. 09/573,080 (filed May 16, 2000) and Ser. No. 09/854,867 (filed May 14, 2001) (the teachings and content of both applications is hereby incorporated by reference) is appropriate for developing subtelomeric sequences, since the majority of probes hybridize only to the correct chromosomal location in the majority of chromosomes. es single copy probes can be designed, amplified, purified and labeled in parallel. For probes that do not hybridize to a single location, when related sequences are missing from the draft genome sequence, alternative primers were developed for these loci or neighboring loci. Probes that show hybridization to multiple loci can also be bisected into two or more parts to determine which component hybridizes to paralogous loci or repetitive sequences. Such bisection involves development of internal primers, possibly new end primers and hybridization of the new products to chromosomes. Unlike other chromosomal regions, the subtelomeric intervals of many chromosomes present some unusual challenges in the design of single copy probes. While these regions are quite gene-rich, there has been considerable exchange and duplication of genetic material between the terminal sequences of different chromosomes.

In more detail, subtelomeric single copy probes are developed using computer software-based design of DNA probe sequences corresponding to subtelomeric intervals. This involves identification of most subtelomeric single copy intervals, then comparison of these intervals with the genome draft to verify the sequence interval is not present at other locations in the human genome sequence. Because the human genome sequence is considered to be more accurate as additional data are incorporated in more recent versions of the sequence, currently designed probes are compared to these versions of genome sequence to determine if coordinates of designed probes remain within 300 kb of the end of the chromosome. If large amounts of additional sequence (>300 kb) have been added to the telomeric end of the draft sequence of a chromosome since the production of a probe, new probes that are closer to the chromosomal ends are designed from the newly established subtelomeric interval.

Next, fragments are synthesized using PCR-amplification with multiple pairs of primer sets for each subtelomeric region. Other approaches or direct synthesis of single copy probes would also be feasible (see U.S. Pat. No. 6,521,427, the teachings and content of which are hereby incorporated by reference), however, these methods are more suited for high volume probe production than the instant methods. The majority of designed probes can be amplified and amplification can be optimized to produce a single homogeneous PCR product. Infrequently, no amplification is observed for a set of primers. This necessitates that the PCR amplification conditions be carefully optimized, and primer and amplification product sequences are re-examined to determine if they exhibit homology to sequences on other chromosomes. If PCR amplification is still not achieved, alternative primer sets unique to this locus are prepared and the amplification procedure is repeated.

Once amplification reactions are optimized, then multiple (or a single large volume) reactions are performed in parallel to obtain adequate product for hybridization. The product is either isolated by gel electrophoresis and purified by column centrifugation or by non-denaturing high performance liquid chromatography (DHPLC) purification of reaction mixtures. The product is then labeled by nick translation, purified and hybridized to normal metaphase chromosomes from two individuals (at least one male) and analyzed by fluorescence microscopy. If hybridization efficiency is low (due to low specific activity of incorporation of the modified nucleotide), the probe is relabeled and the chromosomal hybridization is repeated. Multiple single copy probes from adjacent intervals may be combined to increase hybridization signal intensities.

For probes that hybridize to multiple sites, several alternative methods are available. One such method involves bisecting the primary product into two or more derived products, which are synthesized, labeled and hybridized. If information in the genome sequence database reveals which probe sequences contain potential paralogous copies, the probe is bisected to exclude such sequences. The genome sequence from the region is examined for its location and sequence content in multiple versions of the genome draft as the genome draft is continually being updated with new information. If both bisected components continue to cross-hybridize, a single copy probe is designed from the adjacent proximally-located genomic interval. Alternatively or additionally, the primary product is also preannealed with Cₒt 1 DNA to determine if hybridization to multiple chromosomal loci can be reduced or eliminated. If this procedure results in a chromosome-specific subtelomeric hybridization pattern, it indicates that the probe contains a highly reiterated sequence that was not detected during probe design. In this circumstance, a single copy probe is designed from the adjacent proximally-located single copy genomic interval.

The present invention therefore finds great utility in detecting chromosomal rearrangements. It has recently been estimated that chromosomal rearrangements resulting in an imbalance in DNA sequences near the ends of chromosomes may account for up to 10% of individuals with idiopathic mental retardation and other clinical findings. Specialized chromosome testing such as conventional fluorescence in situ hybridization (FISH) involving DNA probes from these chromosomal regions is required to detect these abnormalities. Now that the human genome sequence has become available, we have recognized that a substantial number of the commercial DNA probes that are commonly used to detect these rearrangements are not found at the ends of the chromosomes. Many of the probes of the present invention are closer to the ends of chromosomes than the currently available probes, thereby allowing identification of some patients with terminal rearrangements of human chromosomes that may not be identifiable with currently available commercial probes. Probes produced in this way are useful for: (a) detecting a broader spectrum of abnormal chromosomal termini than currently detectable with existing cloned probes (b) providing insight into how these chromosomal regions are organized and (c) how the sequences of these chromosomal regions are related to each other and to other chromosomal regions. We have previously used human genome sequences to directly develop single copy probes targeted to a wide variety of chromosomal regions for fluorescence in situ hybridization (scFISH) (U.S. Ser. No. 09/854,867, filed May 14, 2001) (the teachings and content of which is hereby incorporated by reference). Such probes may also be useful in detecting previously unrecognized terminal rearrangements in some patients.

The present invention also provides a streamlined process for producing arrays of single copy probes. Arrays of multiple single copy probes can be designed to cover the same target sizes as conventional recombinant probes, however, other unique applications of these arrays increase the resolution of delineating abnormalities. scProbe arrays can either be used to simultaneously detect targets from multiple chromosomal regions or from a single continuous genomic interval and the automated production of single copy probe arrays is a high throughput process. Such a process was used to simultaneously develop single copy probes from all euchromatic chromosomal termini. Such arrays can also be used for precise delineation of translocation, the deletion, and other rearrangement boundary breakpoints in subtelomeres. For example, multiple probes have been developed from chromosome 9q34 and different subsets of these probes have been hybridized in combination in order to examine the ABL1 chromosomal breakpoints in chronic myelogeneous leukemia (CML) and to detect upstream ABL1 deletions that are associated with early blast crisis (Knoll and Rogan, Sequence-Based In Situ Detection of Chromosomal Abnormalities at High Resolution, Am. J. Med. Gen. 121A:245-257 (2003)).

One aspect of the present invention is that the single copy probes of the present invention (with the exception of chromosomes 3p and 19q) are located in the generally light-staining terminal G-bands of the chromosome. This is significant because in routine clinical cytogenetic analysis, metaphase chromosomes are banded and examined microscopically to look for alterations in chromosome number or chromosome structure. Chromosome pairs are aligned according to size and banding pattern. This alignment is called the karyotype and it is the standard and basic method for examining the integrity of all chromosomes in a cell. In a normal human cell, there are 46 chromosomes, 22 pairs of autosomes (numbered 1 through 22) and one pair of sex chromosomes (XX in females and XY in males). Chromosomes are paired and arranged in the karyotype from largest to smallest in size and according to placement of their centromere and the subsequent designation of the chromosome as metacentric, submetacentric, or acrocentric. Each chromosome contains DNA (unique single copy, repetitive dispersed and highly reiterated DNA) and protein. The centromeres of each chromosome and the majority of the chromosome Y long arm contain heterochromatin which is comprised of repetitive DNA that is transcriptionally inactive. The short arms of acrocentric chromosomes also have highly repetitive DNA in addition to multiple copies of genes for ribosomal RNA. The telomeres of chromosomes contain short telomere-specific DNA repeat sequences (TTAGGG)ₙ that function to cap and protect the ends of the chromosome. Adjacent to the telomeric regions, are subtelomeric regions which are comprised in part of chromosome specific DNA sequences and telomere associated repeats (FIG. 16). Exceptions to chromosome specificity of the subtelomeric regions include the short arms of acrocentric chromosomes, the long arm of the Y chromosome which contains heterochromatin and shares homology with the end of the X chromosome long arm.

When chromosomes are pretreated with methods that could involve heat or chemicals each of the 22 autosomes and the sex chromosomes have a characteristic banded pattern that uniquely identifies that chromosome. The bands are dark and light staining structures on metaphase chromosomes and serve as chromosome specific landmarks. It is onto these structures that cloned DNA sequences have been mapped. They provide reference points for localizing and ordering nucleic acid probes, sequence tagged sites, ESTs, DNA contigs, genes, etc that otherwise could not be referenced as no single chromosome has been sequenced in its entirety due to the repetitive nature of centromeric regions, heterochromatic regions and acrocentric short arms.

The commonly used banding pattern in clinical cytogenetics is referred to as G-banding and this banding is often achieved by pretreating chromosomes with trypsin followed by staining them with Geimsa but other methods of treatment such as staining with fluorescent dyes (such as but not limited to 4,6-diamidino-2-phenylindole) also yield chromosome specific banding patterns. R-banding are reverse banding is the reversed pattern of light and dark G-bands. Chromosomes captured at different times of the cell cycle, i.e., metaphase versus prometaphase, results in chromosomes with more or fewer visible bands.

Chromosome anomalies identified by karyotyping of banded chromosomes are described using the International System for Cytogenetic Nomenclature (ISCN), first introduced in 1971 and published in 1972, with the 1995 version in current usage around the world (ISCN, 1995). This nomenclature is the universal language for cytogeneticists and clinicians to describe chromosomal abnormalities so that findings can be communicated to one another and other clinical professionals without the need to provide a karyotype each time. The ISCN also provides a reference for chromosome band resolution. The ISCN defines 3 different levels of band resolution by the number of visible bands; 400, 550, and 850 bands per haploid karyotype. A typical high-resolution cytogenetic study will have a band-resolution of at least 550 bands. At this level of resolution, the terminal G-bands are light staining for all chromosomes except chromosomes 3p, 19q and Yp. Chromosomal bands for many regions separate into light and/or dark staining sub-bands as the resolution increases. At the 850 band level, chromosome Yp also has a light staining terminal band, the terminal chromosome 3p band (ie. 3p26) separates into three small sub-bands—two dark (3p26.1, 3p26.3) and one light (3p26.2), and the terminal chromosome 19 band (19q13.4) separates into three small sub-bands—two dark (19q13.41, 19q13.43) and one light band (19q13.42). As a result of the chromosomal ends being light staining and thus appearing the same for most chromosomes, any exchanges (i.e., translocations) between only these terminal chromosomal bands or within those chromosomal regions would not be recognized by routine cytogenetic analysis. Such a physical characteristic requires the utilization of other molecular methods, such as fluorescence in situ hybridization (FISH) with chromosome specific nucleic acid probes, in order to identify terminal chromosomal band rearrangements.

The structural definitions provided by this nomenclature allows probes (including genes) to be mapped to chromosomal bands (which are an average size of 5 million base pairs) by those of skill in the art. Advantageously, ISCN banding notation, although imprecise, is stable. Moreover, the human genome sequence is only interpretable by reference to this banded chromosome scaffold. In fact, the sequence is not complete because limitations of technology has not permitted sequencing of (a) centromere and heterochromatin and (b) acrocentric chromosomes (13,14,15,18,21,22) p arm sequences. As a result, the existing array of human genome contigs can unequivocally be placed on this scaffold by reference to the banding information. Otherwise, one without knowledge of the genome sequence, might think, for example, that position 1 of chromosome 21 in either the public or private human genome sequence databases actually begins at the beginning of the p arm, which is not correct.. Accordingly, in order to accurately and consistently describe where sequences are located, one must use the coordinate and the sequence together as using either the sequence or the coordinate alone as the structural feature that links the probes together, would lead to erroneous results.

Another aspect of the present invention provides methods for the application of single copy products for solid phase hybridization of subtelomeric chromosomal sequences. One skilled in the art can appreciate that single copy nucleic acid products synthesized by the instant method can be stably attached to solid surface by covalent chemical or electrostatic charge neutralization, and subsequently hybridized to a solution composed of a mixture of labeled nucleic acids. Typically, the substrate will be a microscope slide, however other surfaces, for example columns, capillaries or chips may also be used. The nucleic acid mixtures may be comprised of purified DNA complete genomes, a set of synthetic clones, DNA fragments, PCR products or a library of cDNA or cRNA. An array of single copy probes of the art may be used as targets for comparative genomic hybridization (CGH) methods. This array would be advantageous for detection of subtelomeric rearrangements compared to current arrays based on synthetic genomic clones. The hybridization reaction of labeled genomic DNA to arrays of synthetic genomic clones requires the addition of a reagent repetitive DNA sequences for blocking repeat sequence hybridization, also known as Cot 1 DNA. The array CGH technique offers an alternative approach for simultaneous identification of monosomy and trisomy of the subtelomeric regions of chromosomes. This is based on comparing the relative intensities of hybridization of a normal and a patient genomic sequences, each labeled with a different fluorescent moiety. In a recent multicenter study of array CGH based on cloned probes (Carter et al. Cytometry 49:43-48, 2002), the teachings and content of which are incorporated by reference herein), variability in suppression of repetitive sequence hybridization in these clones was shown to be the most common explanation for lack of reproducibility between laboratories working with the same batch of labeled genomic probes and clones. The failure to completely suppress repeat sequence hybridization introduced errors in measurements of the normal/abnormal fluorescence intensity ratios. This source of error would not be present using arrays comprised of single copy products, since it would not be necessary to add blocking reagent to the hybridization reaction. In addition, delineation of the boundaries of the imbalanced chromosomal region would be more precise using CGH arrays comprised of single copy products since the locations of these probes on the chromosome have been precisely defined at the nucleotide sequence level, in contrast with many synthetic genomic probes that have been traditionally used for array CGH and FISH analysis of subtelomeric rearrangements.

In another aspect of the present invention, a method of using the probes and correlating them with clinical phenotypes is provided. Subtelomeric regions have been studied by conventional FISH with synthetic DNA probes in individuals with cytogenetically normal chromosomes (at ≧550 band resolution) identify a molecular defect. These regions have also been studied in some individuals with visible cytogenetic abnormalities to further characterize the abnormality. The normal chromosome study population includes 1) those with infertility or multiple pregnancy loss; and 2) individuals with mental retardation in which the common causes of mental retardation have been excluded and the cause remains unknown (ie. idiopathic mental retardation). For the cytogenetically normal patient populations, the subtelomeric results of these studies did not demonstrate any increase in abnormalities in individuals with multiple pregnancy losses or infertility. However, for those individuals with a diagnosis of idiopathic mental retardation, subtelomeric abnormalities were found in ˜0.5% with mild mental retardation, and in ˜5% (range of 0-10%) of those with moderate to severe mental retardation and other clinical abnormalities. For the moderately to severely retarded individuals, different studies report a wide range in the frequency of subtelomeric abnormalities. This is probably related to ascertainment bias as a result of the relatively nonspecific clinical criteria that were used to define the subtelomeric study population. The best clinical indicators for performing subtelomeric analysis in moderately to severely retarded individuals included a positive family history of mental retardation, growth retardation (prenatal and postnatal), dysmorphic facies and one or more other nonfacial dysmorphic features and/or congenital abnormalities.

Mental retardation is the common feature in most if not all patients with subtelomeric abnormalities resulting in genetic imbalances. There are few subtelomeric deletions that result in a specific set of clinical features that can direct the clinician towards a diagnosis. The majority of patients with subtelomere abnormalities currently lack a characteristic set of clinical findings. For these patients, the subtelomere defect is generally loss of the region (ie. deletion or monosomy) or loss of one region and gain of another chromosomal end due to an unbalanced reciprocal translocation (ie. partial monosomy for one chromosome and partial trisomy for another chromosome). Given the number of chromosomes and the number of subtelomeric regions, there are a very large number of different combinations of partial monosomy and partial trisomy for different subtelomeric regions. It seems likely that the rather substantial number of potential chromosome rearrangements would result in an equally diverse set of clinical phenotypes. There are several other factors that could also give rise to the clinical variability. They include: 1) the amount (and genetic content) of the terminal band or bands that are lost in deletions given the length of the terminal chromosomal bands (several million base pairs), 2) plus the size of the chromatin loss and gain in unbalanced translocations and 3) variable unmasking of recessive alleles on homologs. For most subtelomeric abnormalities, the number of patients with similar abnormalities reported is limited and for some subtelomeric regions, no cases have been reported. In about half of patients, the subtelomere rearrangements appear to be de novo. The remaining half are inherited from transmission of an abnormal chromosome or chromosomes from a carrier parent. A sufficient number of patients with such rearrangements will have to be ascertained in order to identify common clinical findings; because of the imprecise localization of currently available probes and the clinical variability seen in patients, and it is unlikely that it will be possible to diagnose specific chromosome imbalances based on clinical findings. Therefore, the only practical strategy for analyzing this group of patients is a comprehensive examination of all subtelomeric regions. After the abnormal subtelomeric region or regions are identified, the size of the imbalance (and the specific genes involved) could be further characterized by testing with a set of different probes derived from that terminal chromosomal band.

For the few subtelomeric deletions that result in a specific set of clinical features that direct the diagnosis, a specific subtelomeric probe will be adequate to confirm the diagnosis. A set of probes for the specific subtelomeric region will delineate the size or length of the deletion that defines the specific clinical findings in a given patient. Several well characterized syndromes result from deletion of only a portion of a terminal chromosomal band include monosomy 1p36 syndrome (chromosome 1p deletion), Wolf-Hirschom syndrome (chromosome 4p deletion), Cri-du-chat syndrome (chromosome 5p deletion) and Miller-Dieker syndrome (chromosome 17p deletion). Nevertheless, patients with these syndromes have a constellation of clinical findings some of which are variable, depending on deletion size and other genetic factors including unmasking of one or more recessive genes.

In addition, to the inherited or constitutional chromosome abnormalities, acquired chromosome abnormalities as observed in some cancers including leukemia can be surveyed with the subtelomeric probes to detect subtle rearrangements or to further characterize cytogenetically visible abnormalities.

In another aspect of the present invention, a subtelomeric probe useful for detecting chromosomal rearrangements is provided. The probe generally comprises a single copy DNA sequence having a length of less than 25 kb and more preferably less than 10 kb wherein the sequence is capable of hybridizing to the terminal G-band or R-band of an arm of a single chromosome. When G-banding is used, the terminal band is light-staining and when R-banding is used, the terminal band is dark staining. Chromosome arms for this invention aspect include 1p, 1q, 2p, 2q, 3p, 4p, 4q, 5p, 5q, 6p, 6q, 7p, 7q, 8p, 8q, 9p, 9q, 10p, 10q, 11p, 11q, 12p, 12q, 13q, 14p, 14q, 15p, 15q, 16p, 16q, 17p, 17q, 18q, 19p, 19q, 20p, 20q, 21p, 21q, 22p, 22q, Xp, Xq, and Yp. Exemplary probes are generally selected from the group consisting of 1-3, 5-23, 26-36, 38-57, 59-61, 63-67, 69-82, and 245-251. Preferably, the probe is within 8000 kb of the telomere of the chromosome. In this respect, exemplary probes include 1-3, 5-23, 26-36, 38-57, 59-61, 63-67, 69-82, and 245-251. More preferably, the probe is within 300 kb of the telomere of the chromosome. In this respect, probes selected from the group consisting of SEQ ID NOS. 36, 80, 46, 47, 49, 51, 56, 248, 57, 78, 59, 75, 76, 74, 63, 250, 251, 66, 65, 67, 4, 3, 1, 9, 6, 11, 10, 17, 20, 19, 18, 21, 81, 26, 29, 28, 31, 32, 43, 42, 41, 40, 44, 45, and 70 are preferred. Moreover, preferred probes are either labeled or modified to attach to a surface.

In another aspect of the present invention, a method of developing single copy DNA sequence probes from subtelomeric regions of chromosomes is provided. The probes are capable of hybridizing to a single location in the genome of an individual and the method generally comprises the steps of searching the DNA sequence of the chromosome on a nucleotide-by-nucleotide basis beginning at the terminal nucleotide for a single copy interval of at least 500 base pairs in length that is closest to said terminal nucleotide, identifying a single copy interval, synthesizing the identified single copy interval, and using the synthesized single copy interval as a probe. Preferred methods include the step of verifying computationally or experimentally that the identified single copy interval is represented at a single genomic location or where paralogous sequences are closely linked so that only a single signal is detected. In this respect, it is preferred that the single copy sequence is labeled. Additionally, it is preferred that the identifying step includes verifying both computationally and experimentally. Preferred methods of computational verification include using software to determine that the probe sequence is located at a single position in the genome. Preferred methods of experimental verification include rehybridizing the single copy probe to the chromosome and visualizing said probe on the terminal band and correct arm of the chromosome. Preferred single copy intervals are selected from the group consisting of SEQ ID NOS.1-3, 5-23, 26-36, 38-57, 59-61, 63-67, 69-82, and 245-251. The method may also include the step of preannealing the single copy probe with highly repetitive DNA.

In yet another aspect of the present invention, a synthetic single copy polynucleotide for identifying chromosomal rearrangements is provided. The polynucleotide is preferably located within 8,000 kb of the terminal nucleotide of a chromosome and is capable of hybridizing to a single location on a specific chromosome when no chromosomal rearrangement has occurred. Preferred polynucleotides have a length of less than 25 kb and are found in the terminal G-band or R-band of said specific chromosome. Preferred polynucleotides are selected from the group consisting of SEQ ID NOS.1-3, 5-23, 26-36, 38-57, 59-61, 63-67, 69-82, and 245-251. Particularly preferred polynucleotides are located within about 300 kb of the terminal nucleotide of a specific chromosome. Particularly preferred polynucleotides include polynucleotides selected from the group consisting of SEQ ID NOS.36, 80, 46, 47, 49, 51, 56, 248, 57, 78, 59, 75, 76, 74, 63, 250, 251, 66, 65, 67, 4, 3, 1, 9, 6, 11, 10, 17, 20, 19, 18, 21, 81, 26, 29, 28, 31, 32, 43, 42, 41, 40, 44, 45, and 70. It is preferred that the polynucleotides are either labeled or chemically modified to attach to a surface.

In another aspect of the present invention, an oligonucleotide primer pair used for deriving single copy probes that can detect chromosomal rearrangements is provided. The primers are preferably selected from the group consisting of SEQ ID NOS. 83-244.

In yet another aspect of the present invention, an improved synthetic DNA probe operable for detecting chromosomal rearrangements is provided. The probe includes a DNA sequence capable of hybridizing to a location on a chromosome arm. The improvement of the probe is that the probe has a length of less than 25 kb. Additionally, the improvement is that the probe is a single copy sequence with at least a portion of the probe being located closer to the end of a telomere on a chromosome than a clone selected from the group consisting of cosmids, fosmids, bacteriophage, P1, and PAC clones derived from half YACS. Preferably, the entire probe is located closer to the end of a telomere on a chromosome than the previously referenced clones. Preferred chromosome arms for this aspect of the present invention include an arm selected from the group consisting of 2p, 3p, 7p, 8p, 10p, 11p, 16p, Xp, Yp, 1q, 3q, 4q, 6q, 7q, 8q, 9q, 10q, 12q, 13q, 14q, 15q, 16q, 17q, 18q, 20q, 22q, and Xq. Preferably the probe is located within 8,000 kb of the terminal nucleotide of the telomere of a chromosome. Still more preferably, the probe is located within 300 kb of the terminal nucleotide of the telomere of a chromosome. In preferred forms, the probe is located in the terminal G-band or R-band of said chromosome. Preferred probes for this aspect of the invention include probes selected from the group consisting of SEQ ID NOS.46, 47, 49, 56, 78, 59, 64, 249, 2, 4, 3, 5, 9, 11, 20, 19, 21, 81, 246, 70, 72, 73,36, 80, 247, 50, 57, 75, 76, 74, 63, 250, 66, 65, 67, 1, 6, 10, 12, 16, 15, 13, 14, 17, 18, 81, 245, 26, 31, 32, 43, 42, 41, 40, 44, and 45.

In another aspect of the present invention, a method of screening an individual for cytogenetic abnormalities is provided. The individual should be diagnosed with idiopathic mental retardation based on a common set of clinical findings. Additionally, the individual should exhibit at least one clinical abnormality associated with idiopathic mental retardation. The method generally comprises the steps of screening the genome of the individual using a plurality of hybridization probes, wherein each of the probes has a length of less than about 25 kb, and detecting hybridization patterns of the probes, wherein the hybridization patterns will indicate cytogenetic abnormalities in the individual's genome. Preferably, at least one probe from each chromosome arm should be used in the assay. However, in some situations, only certain chromosome arms will need to be assayed because the clinical abnormality or the common set of clinical findings maybe associated with a subset of the entire set of chromosome arms. The method may further include the step of associating the hybridization patterns with specific clinical abnormalities. Preferably, the probes are single copy probes meaning that they are either represented at a single genomic location or where paralogous sequences are closely linked so that only a single hybridization signal is detected.

In another aspect of the present invention, a method of delineating the extent of a chromosome imbalance is provided. The method generally includes the steps of assaying a chromosome arm using a plurality of hybridization probes having a length of less than about 25 kb, detecting hybridization patterns of the probes on the arm, and comparing the hybridization patterns with a standard genome map of the arm in order to delineate the extent of a chromosome imbalance. Such a method may be performed on a plurality of chromosome arms. The arm(s) assayed maybe selected due to a common set of clinical findings for the individual or the clinical abnormality may be associated with one or more arms. The method may further include the step of correlating imbalances on the arm with a medical condition. Preferred medical conditions include idiopathic mental retardation and cancer.

### BRIEF DESCRIPTION OF THE DRAWINGS

The patent or application file contains at least one drawing, in the form of photographs, executed in color. Copies of this patent or patent application publication with color drawing(s) will be provided by the Office upon request and payment of the necessary fee.

FIG. 1 is a series of twelve photographs depicting various probes hybridizing to specific chromosome locations on various chromosomes. These images are enlarged in FIGS. 2-13;

FIG. 2 is a photograph of a 2.6 kb probe hybridizing to chromosome 5q;

FIG. 3 is a photograph of a 2.5 kb probe hybridizing to chromosome 7q;

FIG. 4 is a photograph of a 2.2 and a 2.4 kb probe hybridizing to chromosome 9q;

FIG. 5 is a photograph of a 3.2 kb probe hybridizing to chromosome 13q;

FIG. 6 is a photograph of a 3.8 and a 1.8 kb probe hybridizing to chromosome 14q;

FIG. 7 is a photograph of a 2.6 kb probe hybridizing to chromosome 17p;

FIG. 8 is a photograph of a 2.5 kb probe hybridizing to chromosome 18q;

FIG. 9 is a photograph of a 2.0 kb probe hybridizing to chromosome 19q;

FIG. 10 is a photograph of a 2.6 kb probe hybridizing to chromosome 20p;

FIG. 11 is a photograph of a 2.1, 3.0 and a 3.7 kb probe hybridizing to chromosome 20q;

FIG. 12 is a photograph of a 3.5 kb probe hybridizing to chromosome 22q;

FIG. 13 is a photograph of a 2.5 kb probe hybridizing to chromosome Xq; and

FIG. 14 is a photograph of a 2.3 kb probe hybridizing to chromosome 19q.

FIG. 15 is a series of photographs of various probes localized on specific chromosomal arms;

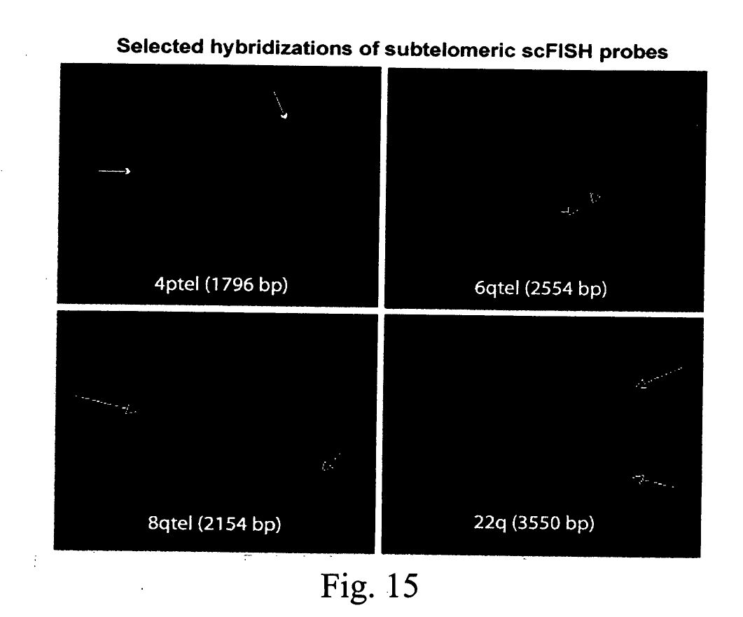

FIG. 16 is a schematic drawing of the structure of a chromosome end depicting the location of single copy probes in relation to the telomere;

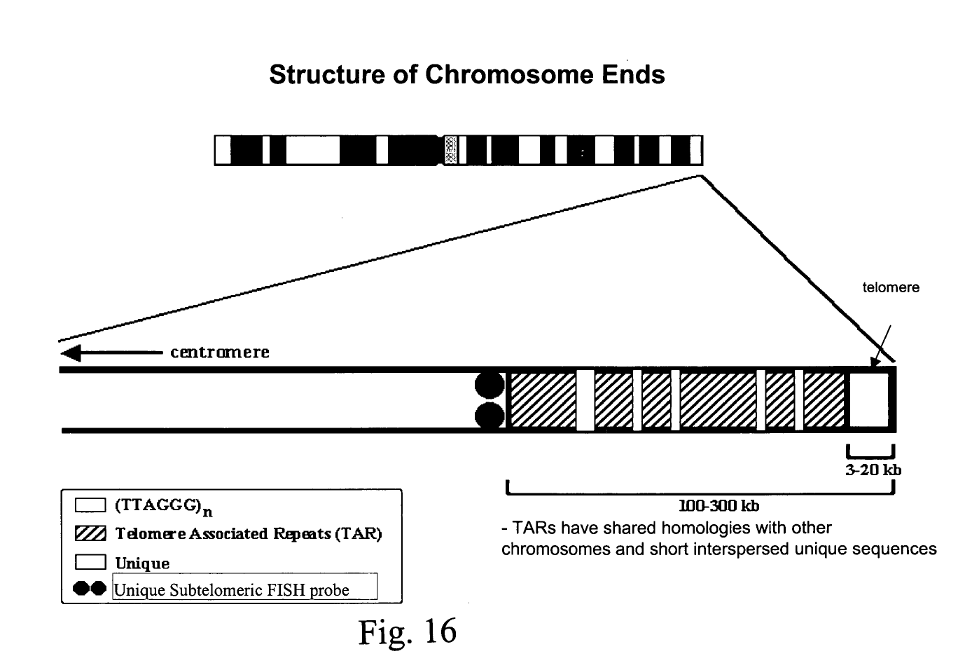

FIG. 17 is a schematic drawing of various gene locations in the 13q arm and their relation to a prior art probe and to a single copy probe in accordance with the present invention;

FIG. 18 is a photograph of a single copy chromosome 18q probe (2530 bp in length) hybridized to a metaphase spread with an abnormal or derivative chromosome 6 and normal chromosome 18; and

FIG. 19 is a photograph of two single copy subtelomeric probes for chromosomes 14q (1984 bp) and 3p (2093 bp) hybridized to normal metaphase cells.

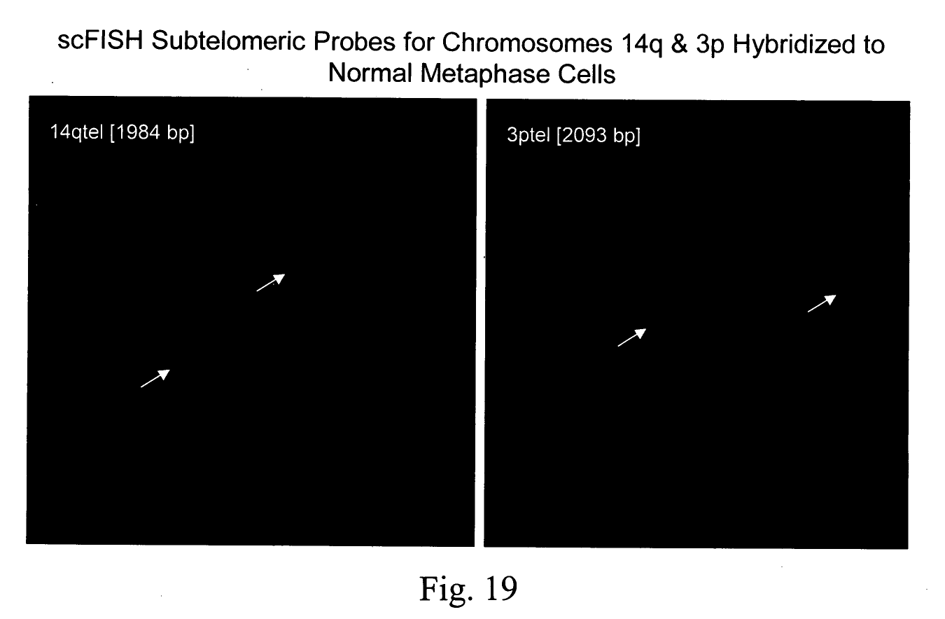

### DETAILED DESCRIPTION OF THE PREFERRED EMBODIMENT

The following examples set forth preferred embodiments of the present invention. It is to be understood that these examples are provided by way of illustration and nothing therein should be taken as a limitation upon the overall scope of the invention.

### EXAMPLE 1

This example describes the process of developing single copy probes in accordance with the present invention.

Materials and Methods:

Development of Subtelomeric Single Copy FISH Probes for all Human Chromosomes and Testing Them by Hybridizing Them to Normal Human Chromosomes.

Probe design. Probe sequences are designed and verified from the April 2001, June 2002 and November 2002 human genome drafts, and the Celera Genomics human genome sequence as described previously (Rogan et al, Sequence-Based Designs of Single-Copy Genomic DNA Probes for Fluorescence In Situ Hybridization, 11 Genome Research, 1086-1094 (2001) the contents and teachings of which are hereby incorporated by reference). The primary objective is to select single copy probes that recognize a single genomic location adjacent to the telomeres of each euchromatic chromosomal arm. This poses unique challenges for chromosomal termini that have evolved by paralogous duplication events. Paralogous non-allelic duplications are detected by comparing the sequences of target single copy intervals with the remainder of the genome. The BLAT server at the National Laboratory of Medicine is used to test for similarities to other non-allelic sequences in the public human genome draft, whereas the Celera sequence is searched locally on a Sun workstation using BLAST. Non-allelic sequence blocks of <500 bp in length and/or <80% sequence identity are not considered as potential sites for cross-hybridization, because such sequence similarities would not be detectable by FISH.

Single copy intervals are sought within successive 100 kb intervals from each chromosome end. If a single copy interval of at least ˜1.8 kb in length can be located within the first 100 kb of subtelomeric sequence (and which does not computationally cross-hybridize elsewhere in the genome), then this interval is selected as a probe. Otherwise, adjacent 100 kb genomic intervals are searched for candidate single copy probe sequences until adequate probe(s) can be identified. The majority of the previously developed single copy probes are within 200 kb of the telomere. Although a longer chromosomal probe is generally desired, a probe of 1.5 kb can generally be developed from a 1.8 kb single copy interval and visualized by FISH.

Probe generation, labeling and FISH. A single DNA fragment for each chromosomal region is amplified using long PCR procedures with Pfx-Taq (Invitrogen, Inc). Experimental optimization involved running a series of PCR reactions, each with a different annealing temperature bracketing the predicted annealing temperatures of the primers, to determine the highest possible temperature that produced a homogeneous-sized amplification product. Specificity was also optimized by varying the concentration of PCR enhancer solution according to the manufacturer's recommendations. If no amplification is achieved with a given primer set under a range of temperatures and enhancer concentrations, an alternative adjacent single copy interval is selected for probe development. The fragments are then isolated by conventional techniques including column purification or gel electrophoresis to remove any potentially contaminating repetitive sequences and purified from low temperature agarose using Micro-spin columns (Millipore) or by preparative non-denaturing high performance liquid chromatography (Transgenomic, Omaha Nebr.). The probe fragments are then directly labeled by nick translation using a modified or directly-labeled nucleotide (eg, digoxigenin-dNTP, fluorochrome-dNTP,etc). The labeled probes are denatured and hybridized to fixed, denatured chromosomal preparations immobilized on microscope slides. The probes are hybridized to chromosomes of two individuals according to conventional FISH methods (Knoll and Lichter, In Situ Hybridization to Metaphase Chromosomes and Interphase Nuclei, Current Protocols in Human Genetics, Vol. 1, Unit 4.3 (eds. N. C. Dracopoli et al.) (1994) the teachings and content of which are hereby incorporated by reference). Probe hybridizations are detected by binding the labeled nucleotide with fluorescently-labeled antibody and viewing with fluorescence microscopy with appropriate filter sets. The total chromosomal DNA is counterstained with 4′,6-diamidino-2-phenylindole (blue) and the hybridized probe signals is visualized with fluorochromes.

Validation. Each autosomal subtelomeric probe hybridizes to a homologous chromosome pair in normal female or male cells (2 signals are expected). Probes from X chromosomes hybridize to a single chromosome in male cells and to 2 chromosomes in females. Probes from the Y chromosome hybridize only to male cells. Parallel hybridizations on two different individuals are performed to confirm chromosome band location. Control hybridizations are performed in parallel with probes that have been previously validated. A minimum of 10 metaphase cells are scored to determine hybridization efficiency for each probe. Generally, conventional FISH probes and single copy FISH probes have hybridization efficiency of at least 90%, more preferably at least 92%, still more preferably at least 94%, still more preferably at least 96%, still more preferably at least 98%, and most preferably 100%.

If a probe indiscriminately hybridizes to many locations on chromosomes, it most likely contains moderately to highly repetitive genomic sequences. Although the present repetitive sequence database is quite comprehensive and this pattern of hybridization is uncommon, it has been observed for a minority of probes. Such a result indicates a repetitive sequence family in the human genome that has not yet been characterized at the DNA sequence level. Based on our previous experience in designing single copy probes, only a minority of probes hybridize non-specifically to non-catalogued, interspersed repetitive sequence families that would be distributed throughout the genome. Probes with genome-wide cross-hybridization or cross-hybridization to highly reiterated sequences can be preannealed to Cₒt 1 DNA. Cross-hybridization can be suppressed or eliminated by preannealing with highly repetitive (ie. Cₒt1) DNA. If the hybridization of single copy sequences within the probe is quenched, then an adjacent single copy interval is selected for probe development.

Characterization of Probes that Hybridize to more than One Chromosomal Region.

In addition to highly-repetitive sequence families in probes that were designed to be single copy, we have unexpectedly observed a pattern of hybridization to a limited set of discrete loci on metaphase chromosomes, in addition to the chromosomal site from which the probe was designed. This hybridization pattern results when the probe contains complex, low-reiteration frequency sequences that are highly-related to sequences on other chromosomes or to other sequences on the same chromosome—these are known as paralogous sequences. This hybridization pattern may arise because the genome sequence is either inaccurate or not yet complete. The human genome sequence, however, is acknowledged to be incomplete, especially in regions containing heterochromatin. Paralogous copies of single copy sequences embedded within such regions are not likely to be comprehensively incorporated in the current genome draft. Other regions of the genome that have not been assembled completely or correctly are indicated in the draft by “gap” intervals. Paralogous or duplicate copies of single copy probes in these regions could also be responsible for unexpected hybridization to non-allelic loci. The software used to select probes is capable of detecting related genomic sequences in silico, however, as the genome sequence is not yet finished, there is always the possibility that a particular probe could anneal to other uncharacterized, related sequences on other chromosomes or the same chromosomes. If cross-hybridization to a discrete pattern of chromosomal loci is not suppressed by preannealing the original probe with highly repetitive DNA (eg. see results for chromosome 16 in Table 1), this indicates that the probe contains one or more paralogous sequences (ie. which are present at low copy) rather than a highly repetitive one.

TABLE 1
Summary of subtelomeric scFISH probes validated
by chromosomal hybridization

| Chromo- | | | Approximate | Actual |
|---|---|---|---|---|
| some | Name | Target | Size | Size |
| 1 | 278592693_278592722F | 1qtel | 1.8 | 1853 |
| | 278594516_278594545R | 1qtel | 1.8 | 1853 |
| 2 | | | | |
| 3 | | | | |
| 4 | 200657614_200657649F | 4qtel | 2.4 | 2426 |
| | 200660008_200660039R | 4qtel | 2.4 | 2426 |
| 5 | 195186729_195186760F | 5qtel | 2.8 | 2795 |
| | 195189493_195189523R | 5qtel | 2.8 | 2795 |
| | 195200011_195200041F | 5qtel | 2.6 | 2661 |
| | 195202642_195202671R | 5qtel | 2.6 | 2661 |
| 6 | | | | |
| 7 | 20273_20302F | 7ptel | 2.9 | 2872 |
| | 23115_23144R | 7ptel | 2.9 | 2872 |
| | 163771088_163771117F** | 7qtel | 2.5 | 2574 |
| | 163773632_163773661R | 7qtel | 2.5 | 2574 |
| 8 | 131014_131044F | 8ptel | 2.3 | 2271 |
| | 133255_133284R | 8ptel | 2.3 | 2271 |
| 9 | 141875348_1418775377F | 9qtel | 2.9 | 2889 |
| | 141878207_141878236R | 9qtel | 2.9 | 2889 |
| | 141889106_141889135F | 9qtel | 2.2 | 2232 |
| | 141891306_141891337R | 9qtel | 2.2 | 2232 |
| | 141871749_141871778F | 9qtel | 2.7 | 2707 |
| | 141874426_141874455R | 9qtel | 2.7 | 2707 |
| | 141897247_141897276F | 9qtel | 2.3 | 2278 |
| | 141899495_141899524R | 9qtel | 2.3 | 2278 |
| 10 | 230747_230779F*\hat{ }⁺ | 10ptel | 2.1 | 2132 |
| | 232879_232848R | 10ptel | 2.1 | 2132 |
| | 185297_185326F*⁺ | 10ptel | 2 | 2051 |
| | 187348_187319R | 10ptel | 2 | 2051 |
| | 201244_201278F*⁺ | 10ptel | 3.2 | 3203 |
| | 204448_204479R | 10ptel | 3.2 | 3203 |
| | 20032_20062F*\hat{ }⁺ | 10ptel | 2.5 | 2526 |
| | 22558_22527R | 10ptel | 2.5 | 2526 |
| 11 | 16421_16450F | 11ptel | 2.9 | 2884 |
| | 19275_19304R | 11ptel | 2.9 | 2884 |
| | 150509268_150509297F | 11qtel | 2.4 | 2462 |
| | 150511700_150511729R | 11qtel | 2.4 | 2462 |
| | 150528401_150528430F** | 11qtel | 2.5 | 2513 |
| | 150530884_150530913R | 11qtel | 2.5 | 2513 |
| 12 | 159378_159407F | 12ptel | 2 | 1914 |
| | 161259_161291R | 12ptel | 2 | 1914 |
| | 146323815_146323844F | 12qtel | 3.5 | 3456 |
| | 146327241_146327270R | 12qtel | 3.5 | 3456 |
| 13 | 118776702_118776731F | 13qtel | 3.2 | 3209 |
| | 118779881_118779910R | 13qtel | 3.2 | 3209 |
| 14 | 106219634_106219663F | 14qtel | 1.8 | 1866 |
| | 106221410_106221499R | 14qtel | 1.8 | 1866 |
| | 106192496_106192527F | 14qtel | 3.8 | 3839 |
| | 106196305_106196334R | 14qtel | 3.8 | 3839 |
| 15 | | | | |
| 16 | 102168227_102168256F | 16qtel | 2.5 | 2567 |
| | 102170764_102170793R | 16qtel | 2.5 | 2567 |
| | 24259_24288F*** | 16ptel | 5.2 | 5250 |
| | 29479_29508R | 16ptel | 5.2 | 5250 |
| 17 | 589547_589576F | 17ptel | 2.6 | 2593 |
| | 592110_592139R | 17ptel | 2.6 | 2593 |
| | 554691_554720F | 17ptel | 4.9 | 4984 |
| | 559645_559674R | 17ptel | 4.9 | 4984 |
| | 88342552_88342581F | 17qtel | 3 | 3026 |
| | 88345648_8834577R | 17qtel | 3 | 3026 |
| 18 | 344433_344465F* | 18ptel | 2.1 | 2127 |
| | 346559_346529R | 18ptel | 2.1 | 2127 |
| | 83822245_83822274F | 18qtel | 2.5 | 2530 |
| | 83824743_83824774R | 18qtel | 2.5 | 2530 |
| 19 | 24323_24352F | 19ptel | 2 | 2094 |
| | 26382_26416R | 19ptel | 2 | 2094 |
| | 575_604F | 19ptel | 1.8 | 1815 |
| | 2360_2389R | 19ptel | 1.8 | 1815 |
| | 72318330_72318359F | 19qtel | 2.7 | 2721 |
| | 72321021_72321050R | 19qtel | 2.7 | 2721 |
| | 72351418_72351447F | 19qtel | 2.3 | 2399 |
| | 72353787_72353816R | 19qtel | 2.3 | 2399 |
| 20 | 356009_356039F | 20ptel | 2.6 | 2616 |
| | 358594_358624R | 20ptel | 2.6 | 2616 |
| | 400061_400095F** | 20ptel | 2.1 | 2088 |
| | 402116_402148R | 20ptel | 2.1 | 2088 |
| | 64751135_64751104F | 20qtel | 3.1 | 3133 |
| | 64754267_64754238R | 20qtel | 3.1 | 3133 |
| | 64721595_64721624F | 20qtel | 2.1 | 2166 |
| | 64723760_64723731R | 20qtel | 2.1 | 2166 |
| | 64674392_64674424F | 20qtel | 2.9 | 2997 |
| | 64677388_64677354R | 20qtel | 2.9 | 2997 |
| | 64745597_64745626F | 20qtel | 3.7 | 3695 |
| | 64749291_64749262R | 20qtel | 3.7 | 3695 |
| 21 | 44855249_44855278F | 21qtel | 4.3 | 4370 |
| | 44859589_44859618R | 21qtel | 4.3 | 4370 |
| 22 | 47577168_47577197F** | 22qtel | 3.2 | 3239 |
| | 47580377_47580406R | 22qtel | 3.2 | 3239 |
| X | 124934_124963F | Xptel | 1.9 | 1896 |
| | 126829_126800R | Xptel | 1.9 | 1896 |
| | 157753803_157753832F | Xqtel | 2.5 | 2529 |
| | 157756302_157756331R | Xqtel | 2.5 | 2529 |
| Y | 66941_66970F | Yptel | 2.4 | 2446 |
| | 69357_69386R | Yptel | 2.4 | 2446 |
| | 72392_72421F | Yptel | 2 | 2000 |
| | 74362_74391R | Yptel | 2 | 2000 |

*cross-hybridization observed on other chromosomes.
**cross-hybridization may be present; additional verification required.
***cross-hybridization occurred despite Cₒt1 suppression.
\hat{ }hybridization was detected when probe was combined with other 10ptel probes labeled with “\hat{ }”.
⁺hybridization was detected when probe was combined with other 10ptel probes labeled with “⁺”.

Assuming subsequent versions of the genome assembly are more accurate than the April 2001 version, the probe sequence can be compared to more recent versions to determine if additional sequences related to the original probes are present in these versions. To identify paralogs, the probe sequence is compared with the genome drafts, allowing for a lower degree of sequence similarity to the duplicated copies. If the more recent genome sequence drafts reveal the presence of related sequences, two distinct strategies are available for producing chromosome-specific probes where paralogs are present in other bands on this or other chromosomes: (1) bisecting the probe—if the initial probe is sufficiently long—and reamplification of the non-paralogous region of the probe or (2) selecting a different single copy interval not containing any genomic paralogs for probe development. If a related sequence is not identified by sequence analysis, then internal primers are developed to bisect the original probe into sequences that are chromosome-specific.

The original probe can be bisected to determine which component hybridizes to the multiple sites. Bisection of the product occurs by developing internal primers and possibly new end primers (with similar melting temperatures and GC composition) that result in two smaller products. These new products serve as probes for single copy FISH. If cross-hybridization remains after bisection, further dissection of the probe may be possible or a new single copy probe from the neighboring genomic interval is designed and assessed by FISH.

After bisecting the original probe, one of two patterns of hybridization are expected. That is, one product is chromosome-specific and the other hybridizes to other chromosomal regions, or both products still show multiple sites of hybridization. The former pattern localizes the region that contains the repetitive or paralogous sequence, while the latter does not localize the region but rather indicates that the internal primer set spans the repetitive or paralogous sequence.

To date, we can reliably visualize fragments that are 1500 bp or greater in length by fluorescence microscopy. Thus, when a probe is bisected, we endeavor to produce probes that are at least 1500 bp. Shorter probes can also be combined that have a total target size of at least 1500 bp. A probe has been developed with this procedure that detects only chromosome 4p terminal sequences by bisecting a larger probe that cross-hybridizes to paralogous sequences on other chromosomes. Alternative single copy intervals adjacent to the initial cross-hybridizing sequence are selected if the bisected probe cannot be designed to be at least 1.5 kb in length or because of extensive paralogy to non-alleleic sequences that extend throughout the length of the probe sequence.

Ensuring that Probes are Close to the Ends of Chromosomes; and Revising, as Appropriate, Probes Closer to the Chromosomal Ends.

The locations of the probes designed from the April 2001 genome draft are computationally compared to their locations on the more recent genome draft versions. If the position coordinates have shifted further from the end of the chromosome, then new single copy probes closer to the end of the chromosome, were designed from the April 2001 draft, 46 subtelomeric probes that detect single copy targets were validated and an additional 36 subtelomeric single copy probes have been designed from subsequent versions of the genome sequence and mapped. Development of new probes was contingent on the subtelomeric intervals being free of repetitive sequences and paralogs on other chromosomes. By developing probes as close to the ends of chromosomes as possible, we increase the likelihood of detecting terminal rearrangements that would not be evident using existing cloned probes.

Results:

Compared to conventional subtelomeric FISH probes, the subtelomeric single copy probes that we developed in accordance with the present invention detected smaller rearrangements of terminal sequence chromosomes (that result from deletion or unbalanced, cryptic translocations of these genomic regions) than was previoously possible. The present set of probes has been designed to detect all of the euchromatic sequenced subtelomeric regions. Primers have been designed and these primers recognize unique sequences within each subtelomeric region developed and validated as single copy probes for subtelomeric regions of chromosomes 1, 3, 5q, 7, 8, 9q, 10p, 11, 14q, 16q, 17, 19, 20q, Xp, and Yp. (See Table 2 ). Because these sequences are unique and the corresponding human genome sequence is publicly available, the primers themselves define one and only one product in the genome. Therefore, some of the primers listed in SEQ ID NOS 83-244 are equivalent to the products listed in SEQ ID NOS 1-3, 5-23, 26-36, 38-57, 59-61, 63-67, 69-82, and 245-251.

TABLE 2

Primer sequences and locations

| | | | | |
|---|---|---|---|---|
| | Chromosome | | | |
| | coordinate Range (forward, reverse primer)* | | | Exper- |
| | Length | Tₘ | Tₘ | imentally |
| Band | Sequences of Primer Pair | computed | predicted | Optimized Tₘ |

| | | | | | | |
|---|---|---|---|---|---|---|
| 1ptel | | | | | | |
| Range | 17994_18023F, 20024_19995R | | | | | |
| Length | 2031 | | | | | |
| Forward | TCTGCGGCTGACCTGGCCTCCACGTCTCAC | SEQ ID 1 | 69.5 | 69.65 | | |
| Reverse | CTACCCGTCTCCCACCCCCTCTCCCCACCC | SEQ ID 2 | 69.8 | | 78.2 | |
| Optimal Tm | | | | | | 78.2 |
| 1ptel | | | | | | |
| Range | 20726_20755F, 22139_22168R | | | | | |
| Length | 1433 | | | | | |
| Forward | CCCTAAACTCCTCCCTATCCCTTCTCAATC | SEQ ID 3 | 59.1 | 59.05 | | |
| Reverse | AAAAAAAACCTCATTTCCTCCCCAAAGC | SEQ ID 4 | 59.0 | | 66.8 | |
| Optimal Tm | | | | | 66.8 | |
| 1qtel | | | | | | |
| Range | 278615828_278615859F, 278617891_278617924R | | | | | |
| Length | 2097 | | | | | |
| Forward | AGTTCCTAAACAACTATGAGCTAAAGTATCAG | SEQ ID 5 | 55.3 | 55.3 | | |
| Reverse | CTTTTAAGTGTGAAGAGTTAAGAAGTATCATGTC | SEQ ID 6 | 55.3 | | 58.4 | |
| Optimal Tm | | | | | | 58.4 |
| 1qtel | | | | | | |
| Range | 278592693_278592722F, 278594516_278594545R | | | | | |
| Length | 1853 | | | | | |
| Forward | TTGATGTTTATGTCCAGATTTTCTCTTCCC | SEQ ID 7 | 55.9 | 55.95 | | |
| Reverse | GAATCTCAAAATGCTTAACTCCAAAACCAG | SEQ ID 8 | 56.0 | | 61.8 | |
| Optimal Tm | | | | | | 61.8 |
| 2ptel | | | | | | |
| Range | 78433_78462F, 80517_80546R | | | | | |
| Length | 2114 | | | | | |
| Forward | CAGAGCATAGTCAAGAGAGGCGCATTTTCC | SEQ ID 9 | 61.4 | 61.45 | | |
| Reverse | AAGAGCCCCTAAATTAGCCCCGTAGAAACC | SEQ ID 10 | 61.5 | | 66.8 | |
| Optimal Tm | | | | | | 66.8 |
| 2ptel | | | | | | |
| Range | 61604_61634F, 64223_64256R | | | | | |
| Length | 2653 | | | | | |
| Forward | GCAAAGACAATGCAAAAAACACTTTACATGG | SEQ ID 11 | 57.6 | 57.6 | | |
| Reverse | GCCTGATATAGGTATATTCAGAGAGCTACAGAAG | SEQ ID 12 | 57.6 | | 61.8 | |
| Optimal Tm | | | | | | 61.8 |
| 2qtel | | | | | | |
| Range | 247101356_247101385F, 247104869_247104899R | | | | | |
| Length | 3544 | | | | | |
| Forward | ACTCCCTTTTGGATAATCAAAATGCTCAAC | SEQ ID 13 | 56.7 | 56.7 | | |
| Reverse | GCAAAATTACCTTTCAAATGTGTACTTGCTC | SEQ ID 14 | 56.7 | | 61.8 | |
| Optimal Tm | | | | | | 61.8 |
| 4qtel | | | | | | |
| Range | 200662680_200662709F, 200664537_200664508R | | | | | |
| Length | 1857 | | | | | |
| Forward | TTGAAATATGGTACAAAGAAGGGGTTGGAG | SEQ ID 15 | 57.3 | 57.35 | | |
| Reverse | CTTGAAGTCCTTGCCGAAGAAAAATAGTTG | SEQ ID 16 | 57.4 | | 64.6 | |
| Optimal Tm | | | | | | 64.6 |
| 4qtel | | | | | | |
| Range | 200657614_200657649F, 200660008_200660039R | | | | | |
| Length | 2426 | | | | | |
| Forward | GCTGACTCAAGAACTGTAGCATTGAGTGTAAG | SEQ ID 17 | 59.5 | 59.5 | | |
| Reverse | GGGGAATGCAAGCATATTATATGAGCAGAAGG | SEQ ID 18 | 59.5 | | 64.6 | |
| Optimal Tm | | | | | 64.6 | |
| 5qtel | | | | | | |
| Range | 195200011_195200041F, 195202642_195202671R | | | | | |
| Length | 2661 | | | | | |
| Forward | GCAAAGGACCTCTTTAATGCTTATCAGCCAC | SEQ ID 19 | 60.1 | 60.15 | | |
| Reverse | GGTGAGAGCTATGGAAAGCCTCTCCTATTG | SEQ ID 20 | 60.0 | | 66.8 | |
| Optimal Tm | | | | | | 66.8 |
| 5qtel | | | | | | |
| Range | 195186729_195186760F, 195189493_195189523R | | | | | |
| Length | 2795 | | | | | |
| Forward | TTCCAGCCCCACCTGCTCAGGCAGCCTCTATG | SEQ ID 21 | 68.7 | 68.4 | | |
| Reverse | GCCAGCACAGCCTCCTGTCTTAGCCCTGTCC | SEQ ID 22 | 68.1 | | 75.5 | |
| Optimal Tm | | | | | | 75.5 |
| 5qtel | | | | | | |
| Range | 195129480_195129509F, 195131860_195131889R | | | | | |
| Length | 2410 | | | | | |
| Forward | GCGAGAAATGCCTCCCTATTCCCCAGGAGC | SEQ ID 23 | 65.3 | 66.65 | | |
| Reverse | TCCCAGAACTTTGCCTGTTGCCCATGCCAC | SEQ ID 24 | 66.2 | | 68.1 | |
| Optimal Tm | | | | | | |
| 7ptel | | | | | | |
| Range | 20273_20302F, 23115_23144R | | | | | |
| Length | 2872 | | | | | |
| Forward | AGCAGCTCCAGAGCAGGGAACCCACCTCAC | SEQ ID 25 | 67.8 | 67.8 | | |
| Reverse | GTGTCCACACCAGGCAGCGTCCAACTCAGC | SEQ ID 26 | 67.8 | | 72.1 | |
| Optimal Tm | | | | | | 72.1 |
| 7qtel | | | | | | |
| Range | 163817881_163817910F, 163821021_163821050R | | | | | |
| Length | 3170 | | | | | |
| Forward | ATGAGGGAGGAGTGGGGAGAGGAAGTGAAG | SEQ ID 27 | 63.3 | 63.1 | | |
| Reverse | ACTACCTGGTGTCCAGTACCCAAATCCAGC | SEQ ID 28 | 62.9 | | 68.5 | |
| Optimal Tm | | | | | | 68.5 |
| 7qtel | | | | | | |
| Range | 163771088_163771117F, 163773632_163773661R | | | | | |
| Length | 2574 | | | | | |
| Forward | CCCTCTTTCTGAACACCCCCCGGCAGACAC | SEQ ID 29 | 66.9 | 66.5 | | |
| Reverse | TGGCAGGCTGTCCTGGTCGTATTCGAGGTC | SEQ ID 30 | 66.1 | | 61.8 | |
| Optimal Tm | | | | | | |
| 8ptel | | | | | | |
| Range | 163906_163935F, 165984_165955R | | | | | |
| Length | 2079 | | | | | |
| Forward | TCTGCTCTCCTGTGCCAAGCGTCAATATGG | SEQ ID 31 | 63.7 | 63.9 | | |
| Reverse | ACCTCTCTGGGTCTCTCTCCTCCTCACTG | SEQ ID 32 | 64.1 | | 68.1 | |
| Optimal Tm | | | | | | 68.1 |
| 8ptel | | | | | | |
| Range | 131014_131044F, 133255_133284R | | | | | |
| Length | 2271 | | | | | |
| Forward | GCATTTCTCAGAATAATGAATGGCAGGAAATAC | SEQ ID 33 | 57.5 | 57.6 | | |
| Reverse | GTGCATGTTTCAAGACATTCTCAGATTGTG | SEQ ID 34 | 57.7 | | 61.8 | |
| Optimal Tm | | | | | | 61.8 |
| 9ptel | | | | | | |
| Range | 190285_190314F, 192338_192367R | | | | | |
| Length | 2083 | | | | | |
| Forward | CAAGTTGGTAAATGGAGGCATTATATGGAG | SEQ ID 35 | 56.3 | 56.3 | | |
| Reverse | AGTCACGTATCAAGTGGAAATAAAATCGTC | SEQ ID 36 | 56.3 | | 61.8 | |
| Optimal Tm | | | | | | 61.8 |
| 9qtel | | | | | | |
| Range | 141875348_1418775377F, | | | | | |
| | 141878207_141878236R | | | | | |
| Length | 2889 | | | | | |
| Forward | ACAACAGGACAATGCATACAACCACGAAAC | SEQ ID 37 | 60.4 | 60.35 | | |
| Reverse | TCATTAGAATGAAAGGGAGCCACAGAGCAG | SEQ ID 38 | 60.3 | | 66.8 | |
| Optimal Tm | | | | | | 66.8 |
| 9qtel | | | | | | |
| Range | 141889106_141889135F, 141891306_141891337R | | | | | |
| Length | 2232 | | | | | |
| Forward | AGCTCCAGGTAACTCTCAGGCCAGCAGCCC | SEQ ID 39 | 67.6 | 67.55 | | |
| Reverse | AAGGAGGAAGTGGAAGCTCAGCCCAGGCAGTG | SEQ ID 40 | 67.5 | | 72.1 | |
| Optimal Tm | | | | | | 72.1 |
| 9qtel | | | | | | |
| Range | 141878644_141878674F, 141881106_141881140R | | | | | |
| Length | 2497 | | | | | |
| Forward | TGCTGACCGAGCACATACACAATTCAGTGAC | SEQ ID 41 | 62.6 | 62.3 | | |
| Reverse | AGGGTCTCTGCTAACGTAGTGAAAATACGCAAATG | SEQ ID 42 | 62.0 | | 63.2-68.5 | |
| Optimal Tm | | | | | | 63.2-68.5 |
| 9qtel | | | | | | |
| Range | 141871749_141871778F, 141874426_141874455R | | | | | |
| Length | 2707 | | | | | |
| Forward | CTGAGCAGCCACCCTGGATGCTCCTGCACG | SEQ ID 43 | 68.9 | 68.95 | | |
| Reverse | CTCTGGCCCTCGGCCCATTGCCACCTCAAC | SEQ ID 44 | 69 | | 64.6 | |
| Optimal Tm | | | | | | 64.6 |
| 9qtel | | | | | | |
| Range | 141897247_141897276F, 141899495_141899524R | | | | | |
| Length | 2278 | | | | | |
| Forward | ACAGAAGCAAGCAGAAGTACAGAACCAGAG | SEQ ID 45 | 60.4 | 60.45 | | |
| Reverse | TTTCTCCCTCCTAGATGATCGACTTGGGAC | SEQ ID 46 | 60.5 | | 58.4 | |
| Optimal Tm | | | | | | 58.4 |
| 9qtel | | | | | | |
| Range | 141928044_141928073F, 141930725_141930750R | | | | | |
| Length | 2711 | | | | | |
| Forward | CACCATCTGCATCTTACATCTTATTCCACC | SEQ ID 47 | 57.8 | 57.75 | | |
| Reverse | AAGTTAATTGGAGGGAAATGGCTGTAAAGG | SEQ ID 48 | 57.7 | | 61.8 | |
| Optimal Tm | | | | | | 61.8 |
| 10ptel | | | | | | |
| Range | 230747_230779F, 232879_232848R | | | | | |
| Length | 2132 | | | | | |
| Forward | GAGTTAAGCTCAGCTCACTCTGTGGCACTACC | SEQ ID 49 | | 64 | | |
| Reverse | GGAAGTGTCTGTGGTTTGCCAGCTCCTGTTCT | SEQ ID 50 | | | 64 | |
| Optimal Tm | | | | | | 64 |
| Range | 185297_185326F, 187348_187319R | | | | | |
| Length | 2051 | | | | | |
| Forward | GATTCTGACCCTTGCCCAGCCTACGTCTCG | SEQ ID 51 | | 64 | | |
| Reverse | TGACCCACAATCTTTCCCTTCTGGCACCAC | SEQ ID 52 | | | 64 | |
| Optimal Tm | | | | | | 64 |
| Range | 201244_201278F, 204448_204479R | | | | | |
| Length | 3203 | | | | | |
| Forward | GATGTTTCTAACTATACCTTTATGTGTTTTTCCT | SEQ ID 53 | | 57 | | |
| Reverse | GCTCTTCCTACCAAGTTATCTTCATCTATTCG | SEQ ID 54 | | | 57 | |
| Optimal Tm | | | | | | 57 |
| Range | 20032_20062F, 22558_22527R | | | | | |
| Length | 2526 | | | | | |
| Forward | CCAGATACTGGTCTCATTCTTGGGCAGTTTC | SEQ ID 55 | | 61 | | |
| Reverse | CCGAGTTTGACTTTCACTCACTCACCTAGATG | SEQ ID 56 | | | 61 | |
| Optimal Tm | | | | | | 61 |
| 10qtel | | | | | | |
| Range | 144785104_144785133F, 144786894_144786923R | | | | | |
| Length | 1820 | | | | | |
| Forward | AATGAAAGGGATACGTTTGCGTCTGTCCTG | SEQ ID 57 | 61.1 | 61.05 | | |
| Reverse | GGTAAAGTTCTTCCCCTGGCTCTTCACAAC | SEQ ID 58 | 61 | | 66.8 | |
| Optimal Tm | | | | | | 66.8 |
| 10qtel | | | | | | |
| Range | 144752659_144752688F, 144756387_144756416R | | | | | |
| Length | 3758 | | | | | |
| Forward | ATTTTAGTGAAGAAACTTGCTGTGGAGTCG | SEQ ID 59 | 58.1 | 58.05 | | |
| Reverse | AAGAAGAAGGAAAGAACAAGAAAAGCCCAG | SEQ ID 60 | 58.0 | | 66.8 | |
| Optimal Tm | | | | | | 66.8 |
| 10qtel | | | | | | |
| Range | 144746646_144746677F, 144751955_144751985R | | | | | |
| Length | 5340 | | | | | |
| Forward | CCACACCCAGCCAACAGCAGACGTGATGGAAG | SEQ ID 61 | 67.2 | 67.1 | | |
| Reverse | CTGAGGAGACAGGTGGGACAGAGGGGCAGAC | SEQ ID 62 | 67.0 | | 68.1 | |
| Optimal Tm | | | | | | 68.1 |
| 11ptel | | | | | | |
| Range | 16421_16450F, 19275_19304R | | | | | |
| Length | 2884 | | | | | |
| Forward | GCTCCTCCCCACACCTGACCCTGCCCTCAC | SEQ ID 63 | 69.4 | 69.45 | | |
| Reverse | GAGCTGGCCCGTTTTGCCACCTGTCACCCC | SEQ ID 64 | 69.5 | | 75.5 | |
| Optimal Tm | | | | | | 75.5 |
| 11qtel | | | | | | |
| Range | 150509268_150509297F, 150511700_150511729R | | | | | |
| Length | 2462 | | | | | |
| Forward | CAACCCGAGAGATGAGCCCTGCGTCCACTG | SEQ ID 65 | 66.9 | 66.5 | | |
| Reverse | CACCTGCGTCTTCAAGCCCTAATGGGCACC | SEQ ID 66 | 66.1 | | 72.1 | |
| Optimal Tm | | | | | | 72.1 |
| 11qtel | | | | | | |
| Range | 150528401_150528430F, 150530884_150530913R | | | | | |
| Length | 2513 | | | | | |
| Forward | AATGAAGAAATGAATCTCTCTCCTTGGACG | SEQ ID 67 | 57.2 | 57.1 | | |
| Reverse | TTTATCATGTGGCAGGCAATTAAATGACAG | SEQ ID 68 | 57.0 | | 61.8 | |
| Optimal Tm | | | | | | 61.8 |
| 12ptel | | | | | | |
| Range | 159378_159407F, 161259_161291R | | | | | |
| Length | 1914 | | | | | |
| Forward | GTGTCCCCAGGCAGAGTTAAGAAAAGAAGC | SEQ ID 69 | 61.2 | 61.15 | | |
| Reverse | GCAGGAGTGAAACAACAAAAAATACAGCCAGTC | SEQ ID 70 | 60.9 | | 66.8 | |
| Optimal Tm | | | | | | 66.8 |
| 12ptel | | | | | | |
| Range | 186089_186118F, 189015_189044R | | | | | |
| Length | 2956 | | | | | |
| Forward | TACTCCTTCCTTCCTTCCCTCAACCCTGAC | SEQ ID 71 | 62 | 62 | | |
| Reverse | TTTGGGCAGAGTGTGGATGGAGAAGATTGG | SEQ ID 72 | 62.0 | | 68.5 | |
| Optimal Tm | | | | | | 68.5 |
| 12qtel | | | | | | |
| Range | 146323815_146323844F, 146327241_146327270R | | | | | |
| Length | 3456 | | | | | |
| Forward | TTCAGAAGGTAGAGTTGGAGGATCATAGGC | SEQ ID 73 | 59.1 | 59.2 | | |
| Reverse | TCCCCACAGAGTAAACAGTAGGAAGGAAAG | SEQ ID 74 | 59.3 | | 61.8 | |
| Optimal Tm | | | | | | 61.8 |
| 12qtel | | | | | | |
| Range | 146336097_146336127F, 146338576_146338607R | | | | | |
| Length | 2511 | | | | | |
| Forward | CACAAAAAGATTAAAACACAATCTTGTGAGC | SEQ ID 75 | 55.5 | 55.5 | | |
| Reverse | ACTCATCCTTTATTCTTCTAGTAAGAATTGCC | SEQ ID 76 | 55.5 | | 55.5 | |
| Optimal Tm | | | | | | 55.5 |
| 13qtel | | | | | | |
| Range | 118776702_118776731F, 118779881_118779910R | | | | | |
| Length | 3209 | | | | | |
| Forward | TGCCTGCTGACTGAGGGGGATGGCCGGAAC | SEQ ID 77 | 69.6 | 69.65 | | |
| Reverse | GGCTGTGGGTGTGCGGGATAGGGGAGGCTC | SEQ ID 78 | 69.7 | | 64.6-75.5 | |
| Optimal Tm | | | | | | 64.6-75.5 |
| 13qtel | | | | | | |
| Range | 118764062_118764091F, 118767129_118767158R | | | | | |
| Length | 3097 | | | | | |
| Forward | TCCTTGCTGCACTACCTACCCATGCAGGCG | SEQ ID 79 | 66.8 | 66.85 | | |
| Reverse | GGTCACCGGGAGGAAGCCACACATCTGACG | SEQ ID 80 | 66.9 | | 64.8 | |
| Optimal Tm | | | | | | 64.8 |
| 14qtel | | | | | | |
| Range | 106231822_106231855F, 106234034_106234063R | | | | | |
| Length | 2242 | | | | | |
| Forward | TCTTAGAACATGTGACAGAATCAAAAAATTCC | SEQ ID 81 | 55.4 | 55.35 | | |
| Reverse | TTTAAGAGAATGAAAGTCATACCTGTAGCC | SEQ ID 82 | 55.3 | | 58.4 | |
| Optimal Tm | | | | | | 58.4 |
| 14qtel | | | | | | |
| Range | 106219634_106219663F, 106221499_106221470R | | | | | |
| Length | 1866 | | | | | |
| Forward | TTTCAGACGGTCGAGTGACAGTCCAAACGG | SEQ ID 83 | 63.7 | 63.75 | | |
| Reverse | GGAGGCTCTGCTTTCCAGCCAGATGTAAGG | SEQ ID 84 | 63.8 | | 63.2-71.8 | |
| Optimal Tm | | | | | | 63.2-71.8 |
| 14qtel | | | | | | |
| Range | 106192496_106192527F, 106196305_106196334R | | | | | |
| Length | 3839 | | | | | |
| Forward | GCATACATCTCCGACACTAGGAAAGACACGAC | SEQ ID 85 | 61.9 | 62.3 | | |
| Reverse | ATTGGCCTTTCAGCTTGCCCAAACACAAAC | SEQ ID 86 | 62.7 | | 63.2-68.5 | |
| Optimal Tm | | | | | | 63.2-68.5 |
| 15qtel | | | | | | |
| Range | 100651272_100651303F, 100653622_100653593R | | | | | |
| Length | 2351 | | | | | |
| Forward | CTTAAAATATCCAGTCTCAGTTTTGTTTGCTC | SEQ ID 87 | 55.3 | 55.25 | | |
| Reverse | TTAAATGCAACTCAAAAGAAGAAAGGTCTC | SEQ ID 88 | 55.2 | | 61.8 | |
| Optimal Tm | | | | | | 61.8 |
| 15qtel | | | | | | |
| Range | 100655884_100655914F, 100657490_100657461R | | | | | |
| Length | 1607 | | | | | |
| Forward | CCTTTTTTTTGTCACCTAGTATTTGCAACAC | SEQ ID 89 | 56.6 | 56.6 | | |
| Reverse | CTAAAACCCATAAATTGACCGAACACTCTC | SEQ ID 90 | 56.6 | | 61.8 | |
| Optimal Tm | | | | | | 61.8 |
| 15qtel | | | | | | |
| Range | 100596963_100596992F, 100598878_100598844R | | | | | |
| Length | 1916 | | | | | |
| Forward | GGGATAGATGATGGTTTGTTGTAATTTGAG | SEQ ID 91 | 55 | 55 | | |
| Reverse | GTCTCTAGATAATCTAATAATATCCACTTCCCAAG | SEQ ID 92 | 55 | | 55.5 | |
| Optimal Tm | | | | | | 55.5 |
| 16ptel | | | | | | |
| Range | 17530_17560F, 23932_23961R | | | | | |
| Length | 6432 | | | | | |
| Forward | GCCACGCACTTCCCTGCTGTTTGAAAGACCC | SEQ ID 93 | 66.6 | 66.45 | | |
| Reverse | GTGTTTGTCACCCCACTCCTGCTCCTGCCC | SEQ ID 94 | 67.3 | | 72.1 | |
| Optimal Tm | | | | | | 72.1 |
| 16ptel | | | | | | |
| Range | 24259_24288F, 29479_29508R | | | | | |
| Length | 5250 | | | | | |
| Forward | GTGTCGGTTCTCCACCACCACGATGAGCCC | SEQ ID 95 | 67.1 | 66.9 | | |
| Reverse | TCCCGCCTAGCAGAGTTGCTGTCTGGCAAG | SEQ ID 96 | 66.7 | | 68.1 | |
| Optimal Tm | | | | | | 68.1 |
| 16qtel | | | | | | |
| Range | 102168227_102168256F, 102170764_102170793R | | | | | |
| Length | 2567 | | | | | |
| Forward | AGTTCTCTGCTTCTTCCTTGTTTTCTCTCC | SEQ ID 97 | 58.7 | 58.6 | | |
| Reverse | TCCCTTTTTGCTTCTCTGTGTTGTGATTTC | SEQ ID 98 | 58.5 | | 61.8 | |
| Optimal Tm | | | | | | 61.8 |
| 17ptel | | | | | | |
| Range | 589547_589576F, 592110_592139R | | | | | |
| Length | 2593 | | | | | |
| Forward | TCGGATAAAAGCAGAAGCAGAGAGAGCAGG | SEQ ID 99 | 61.7 | 62.2 | | |
| Reverse | AGCCCCCTCCTAAAGGCTGTCACCTATAAG | SEQ ID 100 | 62.7 | | 68.5 | |
| Optimal Tm | | | | | | 68.5 |
| 17ptel | | | | | | |
| Range | 554691_554720F, 559645_559674R | | | | | |
| Length | 4984 | | | | | |
| Forward | ATCCTTTCCTTTTTTGCCTTCTTCCTCATC | SEQ ID 101 | 57.9 | 57.95 | | |
| Reverse | CTTCTTTCCTCCCCATCTTCTCCTTCTTAG | SEQ ID 102 | 58 | | 58.4 | |
| Optimal Tm | | | | | | 58.4 |
| 17qtel | | | | | | |
| Range | 88337031_889337060F, 88339899_88339928R | | | | | |
| Length | 2898 | | | | | |
| Forward | GACAGGTTGGGGATCTAGAGAGCTGGGGAG | SEQ ID 103 | 63.8 | 63.8 | | |
| Reverse | AAAGGGGGTGTTAGTGAGGGGCCACAAAAG | SEQ ID 104 | 63.8 | | 71.8 | |
| Optimal Tm | | | | | | 71.8 |
| 17qtel | | | | | | |
| Range | 88342552_88342581F, 88345577_88345548R | | | | | |
| Length | 3026 | | | | | |
| Forward | GCAATCAGATTTCTCTCAAACCACGAACAC | SEQ ID 105 | 59.1 | 59.1 | | |
| Reverse | TTTATCAGGATATGCGTTTTCCTCCAACCC | SEQ ID 106 | 59.1 | | 66.8 | |
| Optimal Tm | | | | | | 66.8 |
| 18ptel | | | | | | |
| Range | 344433_344465F, 346559_346529R | | | | | |
| Length | 2127 | | | | | |
| Forward | CCTTAACAAACAAACAGAAAAAAAAGAAAGGAG | SEQ ID 107 | 55.6 | 55.6 | | |
| Reverse | AGTCCCAATATTTGAACCTAAATGCAAAAAG | SEQ ID 108 | 55.6 | | 58.4 | |
| Optimal Tm | | | | | | 58.4 |
| 18ptel | | | | | | |
| Range | 335360_335389F, 337727_337697R | | | | | |
| Length | 2368 | | | | | |
| Forward | ATCTTGTTGCATCCTGAGAGAAACAGAATC | SEQ ID 109 | 57.6 | 57.6 | | |
| Reverse | CAGGCATCTACTTGAGAACTGACAAACTAC | SEQ ID 110 | 57.6 | | 61.8 | |
| Optimal Tm | | | | | | 61.8 |
| 18qtel | | | | | | |
| Range | 83822245_83822274F, 83824743_83824774R | | | | | |
| Length | 2530 | | | | | |
| Forward | TGAGAATGTGATTGCCGTTCTGAAAACACC | SEQ ID 111 | 60.2 | 60.05 | | |
| Reverse | TCTTTTCTGTGTGCTTGATTCTTGCAGATACAGC | SEQ ID 112 | 59.9 | | 64.6 | |
| Optimal Tm | | | | | | 64.6 |
| 19ptel | | | | | | |
| Range | 575_604F, 2360_2389R | | | | | |
| Length | 1815 | | | | | |
| Forward | GGAGAAGGGGAGTTTGCTGGGGAGACGAGG | SEQ ID 113 | 66.2 | 66.05 | | |
| Reverse | ACACAATGGAAACAATGGGGAGGGTGGGCG | SEQ ID 114 | 65.9 | | 72.1 | |
| Optimal Tm | | | | | | 72.1 |
| 19ptel | | | | | | |
| Range | 24323_24352F, 26382_26416R | | | | | |
| Length | 2094 | | | | | |
| Forward | ACCTGCCCTGCCACCTCTGTTCTCCCTGCC | SEQ ID 115 | 69.4 | 68.95 | | |
| Reverse | CGCCTTTGAGTCAACCAAGCCCCAAGATGCACACC | SEQ ID 116 | 68.5 | | 61.8 | |
| Optimal Tm | | | | | | 61.8 |
| 19ptel | | | | | | |
| Range | 55302_55331F, 59926_59955R | | | | | |
| Length | 4654 | | | | | |
| Forward | ACCACTAAGAGCCCCTGTCACCCTCCAGCC | SEQ ID 117 | 67.2 | 67.35 | | |
| Reverse | TTCCCCATTCCCCAGTCCAACACCCCCTCC | SEQ ID 118 | 67.5 | | 72.1 | |
| Optimal Tm | | | | | | 72.1 |
| 19qtel | | | | | | |
| Range | 72318330_72318359F, 72321021_72321050R | | | | | |
| Length | 2721 | | | | | |
| Forward | CAGATGGAGACACTCTCCCTGGGAAATGCC | SEQ ID 119 | 63.4 | 63.3 | | |
| Reverse | TTTTGCCTTCCTGCTGCATGACCAGCTAAC | SEQ ID 120 | 63.2 | | 68.5-71.8 | |
| Optimal Tm | | | | | | 68.5-71.8 |
| 19qtel | | | | | | |
| Range | 72351418_72351447F, 72353787_72353816R | | | | | |
| Length | 2399 | | | | | |
| Forward | CTCTCTGCTCCACCTCTGGCTTTGACGACG | SEQ ID 121 | 65.3 | 65.25 | | |
| Reverse | AGACTGCCTCCCCTCCCCTAACCCAGAATG | SEQ ID 122 | 65.2 | | 64.6 | |
| Optimal Tm | | | | | | 64.6 |
| 20ptel | | | | | | |
| Range | 356009_356039F, 358594_358624R | | | | | |
| Length | 2616 | | | | | |
| Forward | AGTGCCCAGGAAAGACCAGGAAAATACAAG | SEQ ID 123 | 61 | 60.75 | | |
| Reverse | GGGAAATAGTAGCGTAAGCTGTCAACTCCAG | SEQ ID 124 | 60.5 | | 66.8 | |
| Optimal Tm | | | | | | 66.8 |
| 20ptel | | | | | | |
| Range | 400061_400095F, 402116_402148R | | | | | |
| Length | 2088 | | | | | |
| Forward | TTCCATTTCCTGCCATCTAAGCAATGCAGACACAG | SEQ ID 125 | 63.7 | 63.7 | | |
| Reverse | TGGACTGCTTGCTGGTCGCTTACATCACTTTAC | SEQ ID 126 | 63.7 | | 63.2-68.5 | |
| Optimal Tm | | | | | | 63.2-68.5 |
| 20qtel | | | | | | |
| Range | 64760349_64760378F, 64762696_64762667R | | | | | |
| Length | 2348 | | | | | |
| Forward | TCAGAGGGGGGCTGGACATTGAATGTGAAC | SEQ ID 127 | 63.5 | 63.3 | | |
| Reverse | GTCACCATAGGACACAGACAGGAAGTGGGG | SEQ ID 128 | 63.1 | | 68.5 | |
| Optimal Tm | | | | | | 68.5 |
| 20qtel | | | | | | |
| Range | 64754684_64754713F, 64759763_64759734R | | | | | |
| Length | 5080 | | | | | |
| Forward | TAGAAATAACGACCAAAAGCCTCCCCTGTG | SEQ ID 129 | 60.4 | 60.4 | | |
| Reverse | TTCAAGCTGTCAGGGACATCATGTTGAGAG | SEQ ID 130 | 60.4 | | 66.8 | |
| Optimal Tm | | | | | | 66.8 |
| 20qtel | | | | | | |
| Range | 64751135_64751104F, 64754267_64754238R | | | | | |
| Length | 3133 | | | | | |
| Forward | TTTGTATGTTATTACCCTCGTTGTGCCATC | SEQ ID 131 | 57.9 | 57.85 | | |
| Reverse | TCTCAGCCTCAGAAAATGCTTATGTTGAAG | SEQ ID 132 | 57.8 | | 64.6 | |
| Optimal Tm | | | | | | 64.6 |
| 20qtel | | | | | | |
| Range | 64745597_64745626F, 64749291_64749262R | | | | | |
| Length | 3695 | | | | | |
| Forward | TTTTTTCCCTCCTGGCCTCACTCTTGCAAC | SEQ ID 133 | 62.7 | 62.8 | | |
| Reverse | ATAGAAGGAAGCAGGACAACGGGGACAGAC | SEQ ID 134 | 62.9 | | 68.5-71.8 | |
| Optimal Tm | | | | | | 68.5-71.8 |
| 20qtel | | | | | | |
| Range | 64737952_64737981F, 64740366_64740337R | | | | | |
| Length | 2415 | | | | | |
| Forward | CGGAAGTCAACAGTCACTGACGAGTCGGAG | SEQ ID 135 | 63.6 | 63.6 | | |
| Reverse | AGAGTATAGGGACCAGCAGGAACACGGAGG | SEQ ID 136 | 63.6 | | 68.5-71.8 | |
| Optimal Tm | | | | | | 68.5-71.8 |
| 20qtel | | | | | | |
| Range | 64733540_64733569F, 64736582_64736553R | | | | | |
| Length | 3043 | | | | | |
| Forward | GCACCAGCCCTTACCTTCCTCCCTTCACAG | SEQ ID 137 | 65.1 | 65.05 | | |
| Reverse | ATATGGTAGGTGCTCACCACATGCAGGCCC | SEQ ID 138 | 65 | | 72.1 | |
| Optimal Tm | | | | | | 72.1 |
| 20qtel | | | | | | |
| Range | 64728344_64728373F, 64733112_64733083R | | | | | |
| Length | 4769 | | | | | |
| Forward | CCTTTCTCTACACCCTCCCACCTGCTGCTC | SEQ ID 139 | 64.7 | 64.25 | | |
| Reverse | CACCCACCTCTCCCTGCCTCTAGTCTCTTC | SEQ ID 140 | 63.8 | | 68.1 | |
| Optimal Tm | | | | | | 68.1 |
| 20qtel | | | | | | |
| Range | 64721595_64721624F, 64723760_64723731R | | | | | |
| Length | 2166 | | | | | |
| Forward | CCCTACCCCAGATCCTGAGGATTCACATAG | SEQ ID 141 | 60.6 | 60.6 | | |
| Reverse | GGGACAGTCAGAAACATCTCTGAAACCCTG | SEQ ID 142 | 60.6 | | 66.8 | |
| Optimal Tm | | | | | | 66.8 |
| 20qtel | | | | | | |
| Range | 64674392_64674424F, 64677388_64677354R | | | | | |
| Length | 2997 | | | | | |
| Forward | GCTCAGTGCTCTCCCGCTCTCCTGCTTCTCTTC | SEQ ID 143 | 67.3 | 67.3 | | |
| Reverse | ACTCAGCCTCTAATCAGCCTCTCTGCTCCACCCAC | SEQ ID 144 | 67.3 | | 75.5 | |
| Optimal Tm | | | | | | 75.5 |
| 21qtel | | | | | | |
| Range | 44855249_44855278F, 44859589_44859618R | | | | | |
| Length | 4370 | | | | | |
| Forward | TAATGTATGCCCACAAATCTCCAGCGACCC | SEQ ID 145 | 62.2 | 62.15 | | |
| Reverse | TCCAGCACCATCTCTGAACAACTACATGCC | SEQ ID 146 | 62.1 | | 68.5-71.8 | |
| Optimal Tm | | | | | | 68.5-71.8 |
| 21qtel | | | | | | |
| Range | 44876898_44876927F, 44878730_44878759R | | | | | |
| Length | 1862 | | | | | |
| Forward | TCTAAGACCAAGTCGCTACACTCTTAACTG | SEQ ID 147 | 58 | 58 | | |
| Reverse | CTTCTTTCAACCATAAAAGCCTTCCTCCTC | SEQ ID 148 | 58 | | 66.8 | |
| Optimal Tm | | | | | | 66.8 |
| 22qtel | | | | | | |
| Range | 47577168_47577197F, 47580377_47580406R | | | | | |
| Length | 3239 | | | | | |
| Forward | TTCAGCGCCAGCCTCTTCGCTCCGTCCAAG | SEQ ID 149 | 68.6 | 68.7 | | |
| Reverse | TGGTCAGGTGTGGGTCAGGAGACCCCAGCC | SEQ ID 150 | 68.8 | | 64.6/72.1 | |
| Optimal Tm | | | | | | 64.6-72.1 |
| 22qtel | | | | | | |
| Range | 47584046_47584075F, 47586361_47586390R | | | | | |
| Length | 2345 | | | | | |
| Forward | GGGTCTCACATGTAGCATTCCTGGGCACAC | SEQ ID 151 | 64.1 | 64.1 | | |
| Reverse | GTCCTCCCATTCCCATCCCTATCCCCACTG | SEQ ID 152 | 64.1 | | 72.1 | |
| Optimal Tm | | | | | | 72.1 |
| 22qtel | | | | | | |
| Range | 47593223_47593252F, 47596743_47596772R | | | | | |
| Length | 3550 | | | | | |
| Forward | CAGGTAAGGGAGATGAGACCTCCAGACAAC | SEQ ID 153 | 61.1 | 61.2 | | |
| Reverse | CCAAATACAGACACAGCCTCAACCCCATTC | SEQ ID 154 | 61.3 | | 66.8 | |
| Optimal Tm | | | | | | 66.8 |
| Xptel | | | | | | |
| Range | 124934_124963F, 126829_126800R | | | | | |
| Length | 1896 | | | | | |
| Forward | CGCAGGAAATAGGCAAACACACACTGGAAG | SEQ ID 155 | 62.0 | 61.95 | | |
| Reverse | GGACCCTACACTGGATGGGTTTTAGCAGTC | SEQ ID 156 | 61.9 | | 68.5 | |
| Optimal Tm | | | | | | 68.5 |
| Xqtel | | | | | | |
| Range | 157753803_157753832F, 157756302_157756331R | | | | | |
| Length | 2529 | | | | | |
| Forward | ATCCACAGCTTTGATCTAGGGAAAATAAAC | SEQ ID 157 | 56 | 56.15 | | |
| Reverse | TGTGTTGGAAATGCAACTTAAATTGAACTG | SEQ ID 158 | 56.3 | | 61.8 | |
| Optimal Tm | | | | | | 61.8 |
| Yptel | | | | | | |
| Range | 66941_66970F, 69357_69386R | | | | | |
| Length | 2446 | | | | | |
| Forward | TATAGACACGTGACAAAGTAGCTGAAAGACC | SEQ ID 159 | 56.6 | 56.45 | | |
| Reverse | TCTGTTTCTGTGTATGACTGCAATTTAACC | SEQ ID 160 | 56.3 | | 61.8 | |
| Optimal Tm | | | | | | 61.8 |
| Yptel | | | | | | |
| Range | 72392_72421F, 74362_74391R | | | | | |
| Length | 2000 | | | | | |
| Forward | CATGCTAAATTCATGGGCCATATTTTCAAC | SEQ ID 161 | 56.3 | 56.3 | | |
| Reverse | GATGCAAAATGTTCATCTCACATCACAATC | SEQ ID 162 | 56.3 | | 61.8 | |
| Optimal Tm | | | | | | 61.8 |

*coordinates from the April, 2001 version of the human genome draft sequence; F: coordinates of forward primer, R: coordinates of reverse primer

Potential probes are densely arrayed across the terminal chromosomal region and coordinates are precisely defined. The probes of the present invention span a range of distances from the telomere of each chromosome arm, generally within the terminal bands of each chromosome. Using individual single-copy probes or these probes in combination, it is possible to delineate the size of the chromosomal region that is involved in the rearrangement with high precision, ie. the length of a gain or loss, the location of a breakpoint of chromosomal translocation or inversion.

Alterations in the short or p-arms of chromosomes 13, 14, 15, 21 and 22 and the long or q-arm of the Y chromosome do not appear to contribute to clinical abnormalities. These regions are comprised predominantly of repetitive sequences and their complete sequences have not been determined. Therefore, probes for these regions were not developed, however, if these chromosomal arms are found to contain unique single copy sequences, the present invention provides a method of developing probes for these regions and applying them.

Table 2 summarizes results of single copy probes for all euchromatic chromosome ends. Probes have been synthesized, hybridized and visualized to the chromosome specific terminal bands for all chromosomes. As stated previously, multiple probes for several chromosomal ends have ben designed and validated. In Table 1, one probe for each of several chromosome terminal bands (11q, 16p, 18p, 20p, and 22q) appear to detect paralogous or repetitive sequence families on other chromosomes. The remaining probes in this table and all additional probes in Table 3 display the chromosomal specificity required for clinical application.

Comparison of Localized scFISH and Recombinant Subtelomeric Probe Locations

| | scFISH probes¹ | | | Recombinant probes² | |
|---|---|---|---|---|---|
| | | SEQ | | Estimated | Approximate |
| | Approx. | ID | Distance from | clone size | distance of STS from |
| | Length(bp) | NO. | Telomere (kb)³ | (kb) | telomere (kb)⁴ |
| 1ptel | 2531* | 82 | 1,045.411-1,047.942 | 90 kb | unknown |
| 1ptel | 3930* | 34 | 1,048.515-1,052.445 | | |
| 1ptel | 3512* | 35 | 1,053.361-1,056.873 | | |
| 1ptel | 2671 | 33 | 3,858.025-3,860.694 | | |
| 1qtel | 1853 | 38 | 7,939.921-7,941.773 | 100 kb | 236 ± 100 |
| 1qtel | 1632* | 36 | 97.847-96.215 | | |
| 1qtel | 2503 | 80 | 89.194-86.692 | | |
| 2ptel | 2653 | 46 | 112.585-115.237 | 175 kb | 322 ± 175 |
| 2qtel | 3355 | 79 | 2,398.933-2,402.287 | 60 kb | 390 ± 46 |
| 3ptel | 2093* | 47 | 181.265-183.325 | 80 kb | 248 ± 80 |
| 3ptel | 1834* | 49 | 199.161-200.994 | | |
| 3qtel | 2953 | 48 | 762.774-765.726 | 95 kb⁶ | 997 ± 95 |
| 3qtel | 2022* | 247 | 595.753-593.731 | | |
| 4ptel | 1796 | 51 | 246.384-248.179; 417.863-419.710⁷ | 145 kb⁶ | (220-292) ± 73 |
| 4qtel | 2426 | 50 | 442.967-445.387 | 130 kb | 930 ± 130 |
| 5ptel | 2189 | 56 | 86.825-89.013 | 191 kb | unknown |
| 5qtel | 2795 | 54 | 2,032.602-2,035.396 | 105 kb | 227 ± 105 |
| 5qtel | 2661 | 55 | 2,019.454-2,022.114 | | |
| 5qtel | 2633* | 52 | 627.290-624.657 | | |
| 5qtel | 1753* | 53 | 422.516-420.763 | | |
| 6ptel | 2152 | 248 | 199.487-201.638 | 80 kb | unknown |
| 6qtel | 2554 | 57 | 175.551-178.104 | 100 kb | (276-282) ± 94 |
| 7ptel | 2872 | 61 | 815.565-818.439 | 60 kb | 218 ± 59 |
| 7ptel | 2434* | 78 | 143.257-145.691 | | |
| 7ptel | 2348* | 59 | 146.749-149.097 | | |
| 7qtel | 2574 | 60 | 1,095.575-1,098.148 | 95 kb | 225 ± 95 |
| 7qtel | 1517* | 75 | 28.945-27.428 | | |
| 7qtel | 1634* | 76 | 5.405-3.771 | | |
| 7qtel | 1865* | 74 | 81.313-79.448 | | |
| 8ptel | 2079 | 64 | 483.728-485.805 | 135 kb | 1,200 ± 135 |
| 8ptel | 2271 | 249 | 455.377-457.645 | | |
| 8qtel | 2154 | 63 | 71.870-74.023 | 100 kb⁶ | 194 ± 100 |
| 8qtel | 2949 | 250 | 145.868-148.816 | | |
| 9ptel | 1754 | 251 | 243.057-244.809 | 115 kb | 140 ± 115⁹ |
| 9qtel | 2232 | 66 | 248.993-251.226 | 95 kb | 223 ± 95 |
| 9qtel | 2707 | 65 | 231.636-234.340 | | |
| 9qtel | 2278 | 67 | 257.634-259.785 | | |
| 10ptel | 2132\hat{ }+ | 5 | 363.852-365.942 | 80 kb⁶ | 328 ± 80 |
| 10ptel | 2051⁺ | 2 | 320.896-322.898 | | |
| 10ptel | 3203⁺ | 4 | 282.669-285.872 | | |
| 10ptel | 2526\hat{ }+ | 3 | 151.566-154.092 | | |
| 10qtel | 1820 | 1 | 184.961-186.780 | 75 kb | 193 ± 75 |
| 11ptel | 2884 | 8 | 1,205.118-1,208.002 | 110 kb⁶ | 290 ± 110 |
| 11ptel | 2489* | 9 | 66.589-69.078 | | |
| 11qtel | 2462 | 7 | 1,781.588-1,784.049 | 160 kb | unknown |
| 11qtel | 2026* | 6 | 33.471-31.445 | | |
| 12ptel | 1914 | 11 | 180.472-182.385 | 100 kb | 0-209 |
| 12qtel | 3456 | 10 | 154.406-157.861 | 165 kb | 180 ± 165 |
| 13qtel | 3209 | 12 | 366.172-369.380 | 75 kb | 2,900 ± 75 |
| 14qtel | 1866 | 16 | 3,155.170-3,157.035 | 160 kb | (4,100-4,200) ± 117 |
| 14qtel | 3839 | 15 | 3,128.031-3,131.869 | | |
| 14qtel | 1984* | 13 | 1,022.102-1,020.118 | | |
| 14qtel | 2617* | 14 | 1,019.175-1,016.558 | | |
| 15qtel | 1607 | 17 | 131.552-133.158 | 100 kb | 420 ± 100 |
| 16ptel | 3361* | 20 | 73.825-77.186 | 110 kb | 3056 ± 110 |
| 16ptel | 2082* | 19 | 56.610-58.692 | | |
| 16qtel | 2567 | 18 | 183.506-186.072 | 110 kb⁶ | 210 ± 110 |
| 17ptel | 2593 | 23 | 895.021-897.613 | 70 kb⁶ | 105 ± 70 |
| 17ptel | 4984 | 22 | 859.347-864.330 | | |
| 17ptel | 2219* | 21 | 101.957-104.176 | | |
| 17qtel | 6191* | 81 | 106.452-100.262 | 160 kb | 750 ± 160 |
| 17qtel | 3026 | 245 | 848.341-871.383 | | |
| 18ptel | 2368 | 246 | 336.408-338.775 | 160 kb | 209 ± 160 |
| 18qtel | 2530 | 26 | 80.057-82.584 | 170 kb | (154-285) ± 40 |
| 19ptel | 1815 | 30 | 1,745.686-1,747.500 | 80 kb | unknown |
| 19ptel | 2094 | 27 | 1,721.659-1,723.752 | | |
| 19ptel | 2400* | 29 | 265.605-268.005 | | |
| 19ptel | 4137* | 28 | 249.688-253.825 | | |
| 19qtel | 2721 | 31 | 121.866-124.586 | 160 kb | 244 ± 160 |
| 19qtel | 2399 | 32 | 88.475-90.874 | | |
| 20ptel | 2616 | 39 | 365.951-368.566 | 160 kb | 0-240 |
| 20qtel | 3133 | 43 | 109.581-112.713 | 140 kb | 62-202 |
| 20qtel | 3695 | 42 | 114.557-118.251 | | |
| 20qtel | 2166 | 41 | 140.088-142.253 | | |
| 20qtel | 2997 | 40 | 186.460-189.456 | | |
| 21qtel | 4370 | 44 | 47.861-52.230 | 170 kb | 0-337 |
| 22qtel | 3550 | 45 | 176.274-178.618 | 80 kb | (161-168) ± 73 |
| Xptel | 1896 | 69 | 2,329.080-2,330.975 | 175 kb | 324 ± 175 |
| | | | | | (X, Y homology)⁸ |
| Xptel | 3700* | 70 | 155.557-159.257 | | |
| Xqtel | 2529 | 71 | 645.399-647.927 | 170 kb | 0-258 |
| Yptel | 2446 | 72 | 2,562.365-2,564.810 | 175 kb | Unknown |
| | | | | | (X, Y homology)⁸ |
| Yptel | 2000 | 73 | 2,567.816-2,569.815 | 170 kb | |
| ¹scFISH probes developed from April 2003 genome draft are labeled with asterisk (*). The remaining probes were from April 01 draft except 1p (Nov 02), 2q, 3q, 4p, 5p, 6, 8q, 9p (June 02). Sequence IDs corresponding to these probes contain the UCSC database version number in the descriptions of these products. | | | | | |
| ²Many of conventional FISH probes were developed by Knight et al. Am. J. Hum. Genet. 67: 320, 2000, and by Abbott Laboratories/Vysis, Inc. | | | | | |
| ³Distance from probe to end of the telomere reported in this table is based on the length of the interval from the probe boundary coordinates to the terminal nucleotide coordinates of each chromosome end in the April 03 version of genome sequence. The computer program BLAT at the Genome Browser website (genome.ucsc.edu) was used to determine these coordinates. Due to inaccuracy in the BLAT algorithm, | | | | | |
| # the coordinates of probe boundaries may differ from the actual coordinates slightly. | | | | | |
| ⁴The position of STS/marker associated with the conventional FISH probe was determined in the April 03 version of the genome sequence. Often a single STS/marker is identified on a clone. There is insufficient information available to determine the positions of STS markers on some of these clones. As a result, error in positioning a probe on the chromosome (ie. ±) is generally the size of the | | | | | |
| # clone provided in: American Journal of Human Genetics 67: p. 320, 2000, and by Abbott/Vysis, Inc. A standard deviation less than the estimated clone size indicates that more than one STS was localized to the clone. | | | | | |

⁶Indicates clones with cross hybridizations to other chromosomes.
⁷Probe recognizes a neighboring paralogous sequence in addition to the known interval.
⁸Reported STS located on X chromosome only, but both commercial probes for sex chromosomes show homology with each other.
⁹Probe detect four paralogs: three of which are on chromosome 9 and one which is on chromosome 2.
unknown = Reported STS/markers could not be placed on genome sequence as they could not be located in all available genome databases or through communication with authors.
\hat{ }hybridization was detected when probe was combined with other 10ptel probes labeled with “\hat{ }”.
⁺hybridization was detected when probe was combined with other 10ptel probes labeled with “+”.

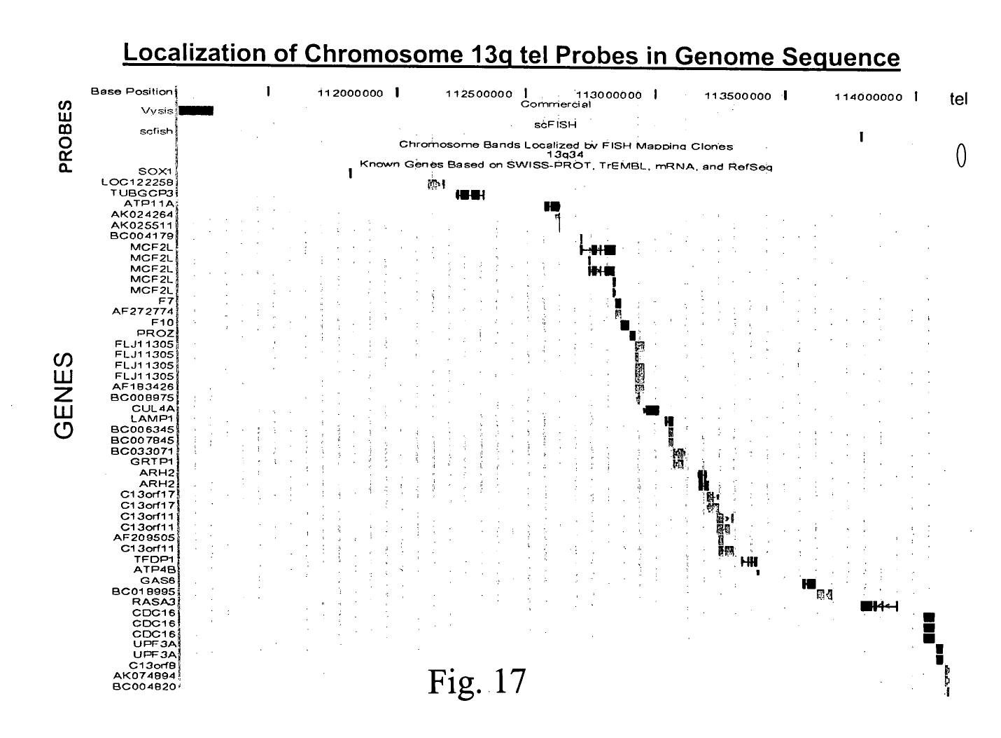

Table 3 compares the location of the corresponding single copy probe with the distance between the end of the available chromosomal sequence and the subtelomeric STS contained within the cloned subtelomeric probe. Commercially available cloned subtelomeric probes (e.g. from Vysis, Inc.) have been positioned on the genome sequence (April 2003 version) based upon one or more sequence tagged sites (STS) contained within them. These STS markers, however, represent a very short interval within the larger cloned segment; therefore, it is not possible to delineate the proximal or distal boundary of the clone from the STS, but the approximate genomic location of the clone can be inferred from the location of the STS. Given the known lengths of a clone and the STS coordinate, it is possible to bracket a range of genomic coordinates covered by that clone. As noted in Table 3, the majority of the single copy probes developed with the present invention are considerably closer to the end of the chromosome than the cognate recombinant probe. The largest differences in distances between the locations of the single copy probes of the present invention and available cloned subtelomeric probes are found for 8pter, 13qter, 14qter, and 16pter where the single copy probes are ˜800 kb or greater closer to the ends of these chromosomes. The distal 8pter interval separating the single copyprobes and conventional probe contains 4 or more genes that, if deleted, would not be detected with the cloned probe but would be detected with the single copy probe. The distal 13qter region (see FIG. 17) contains over 10 confirmed or predicted genes and the distal 14qter contains 3 confirmed genes and 30-40 predicted genes while the 16pter region has more than 200 confirmed and predicted genes. Well-characterized loci in 8p distal to the existing cloned subtelomeric FISH probe, for example, include genes encoding a member of the p53 binding protein family, an interferon induced protein 15 family member, beta-2-like guanine nucleotide-binding protein (which has a role in protein kinase C mediated signaling), and a sequence related to the C5A receptor (which is required for mucosal host cell defense in the lung). The 14qter region that is distal of the cloned subtelomeric probe contains the JAG2 gene, a ligand of the Notch receptor, which has essential roles in craniofacial morphogenesis, limb, thymic development and cochlear hair cell development. It is apparent that loss of a single allele in any of these genes (and others that have not been as thoroughly characterized) will have an adverse clinical outcome. The single copy probes developed for the present invention are the only currently available subtelomeric FISH probes capable of detecting hemizygosity at these loci.

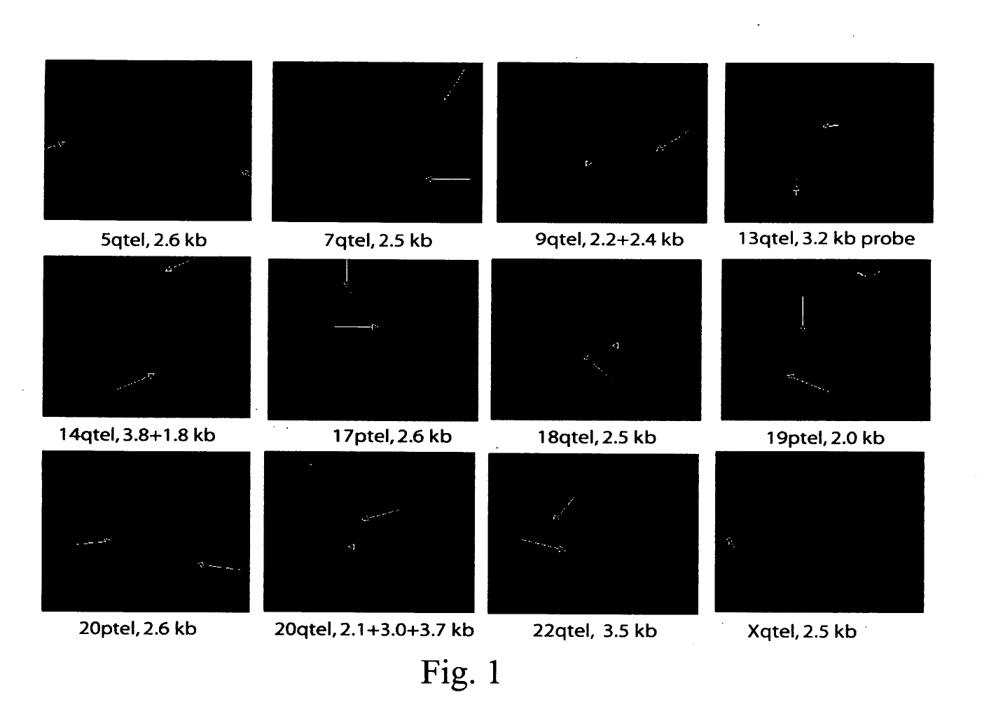

A representative composite panel of 12 subtelomeric single copy probes (or probe combinations) hybridized to normal metaphase chromosomes is shown in FIG. 1. Each panel indicates the telomere detected and the approximate size of the probe (sizes correspond to the “Approximate size” column from Table 1. The arrows indicate the probe hybridizations to the chromosomal ends. Each of the probes specifically hybridize to the homologous chromosome pair from which the sequence is derived.

Table 1 summarizes all of the probes that have been hybridized by September 2002 by chromosome, primer coordinates, chromosome end, approximate and precise sizes of the amplified single copy products. Multiple products from the same subtelomeric region have been individually hybridized except for chromosome 10p, which was hybridized in combination with other 10p probes. As shown in that Table, some probes (e.g. 18ptel) exhibited cross hybridization and some (e.g. 22q) required additional verification prior to ruling out cross hybridization. Furthermore, a 16p probe cross-hybridized despite Cₒt1 suppression.

Table 2 indicates the primers used to amplify each of the probes, the coordinates and the sequences of the primers [derived from the April, 2001 version of the human genome sequence (available online at the genome browser website at the University of California Santa Cruz), and the predicted and then experimentally optimized annealing temperatures for the primers in the amplification reactions that generated the PCR products and the lengths of the amplification products generated with these primers. In general, the optimal annealing temperature was found to lie within 5 degrees C. of the predicted annealing temperature. After optimization of the PCR reaction conditions, all of the products indicated in Table 2 produced single homogenously stained bands by electrophoresis or single sharp peaks in absorbance at a specific timepoint on the DHPLC-Wave system (Transgenomic, Omaha). A subset of these products was labeled and localized to human metaphase chromosomes and are included in Table 3. Table 3 includes the probes from Table 1 that did not cross hybridize to other regions as well as additional probes that we have hybridized to chromosomes since September 2002. The more recently mapped probes have been developed from the April 2003 version of the genome sequence and in many instances are closer to the chromosomal ends. Table 3 gives the precise size of the single copy probe and compares the distance it is from the chromosomal end to that of the synthetic commercial probes.

We observed a number of probes with genomic paralogs detected by molecular cytogenetic analysis, but not by sequence analysis of the April 2001 genome sequence or subsequent version, indicating that the genome sequence is incomplete in the regions containing these paralogous sequences. Complex paralogous domains have also been shown to produce incorrect assemblies of these regions, and this could result in the merging of the paralogous-non-allelic copies into fewer genomic loci. Therefore, probes designed according to this method must be validated by hybridization to normal controls prior to their application to detection of unbalanced rearrangements in patients. This approach may turn out to be useful in identifying potential misassembled regions in future versions of the human genome sequence . Cross-hybridization to unsequenced or incorrectly sequenced genomic regions has precedent (see previous Continuation in Part application; U.S. Ser. No. 09/854,867, the teachings and content of which are hereby incorporated by reference). Previously, we developed probes from two regions, in which closely spaced, highly similar (>95%) paralogous sequences have been localized. The regions include the Down syndrome region on chromosome 21q and the chromosome 16p inversion region for type M4 acute myelogenous leukemia. Both probes hybridized to paralogs on their respective chromosomes but also hybridized to the short arms of acrocentric chromosomes. In these instances, cross-hybridization was suppressed by preannealing with highly repetitive DNA.

Probes with hybridizations to paralogous sequences on other chromosomes or at distant loci (>1 Mb) on the same chromosome compromise the specificity of the assay for detecting abnormalities for the telomere that the probe is designed to detect. In such cases, the sequences in the probe with paralogy to other chromosomal loci have been eliminated. The preferred approaches for eliminating such sequences include (1) selecting and producing alternate probes from the neighboring chromosomal intervals or (2) redesigning probes to eliminate the subsequences that are paralogous to other chromosome loci. Since single copy intervals of suitable size for single copy FISH are densely arranged in the genome, we have generally preferred to develop new probes from adjacent genomic intervals. This approach is less time consuming and less labor intensive than bisecting a probe with paralogous counterparts, however probe bisection, is, in some instances, the only alternative, especially if a probe derived from a particular (small) gene is required. Marked entries in tables 1 and 2 indicate examples of alternate single copy hybridization probes for telomeres where paralogies to other chromosomes had been initially observed.

Discussion:

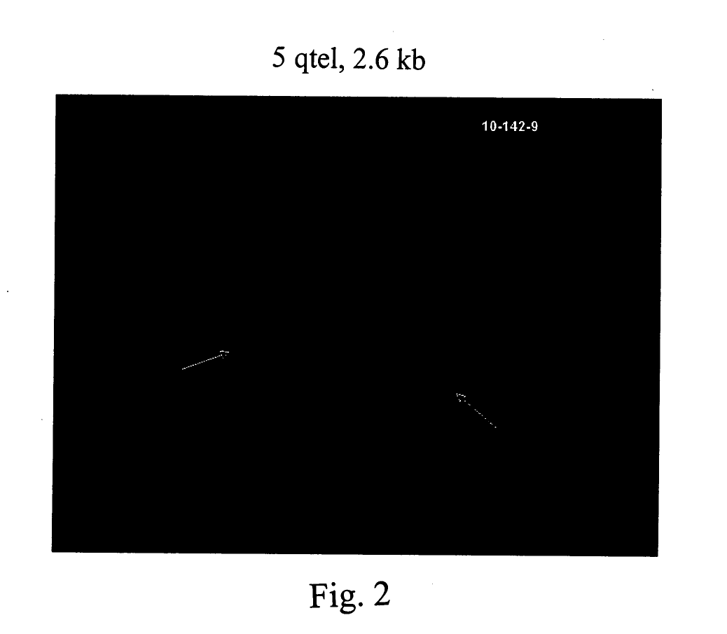

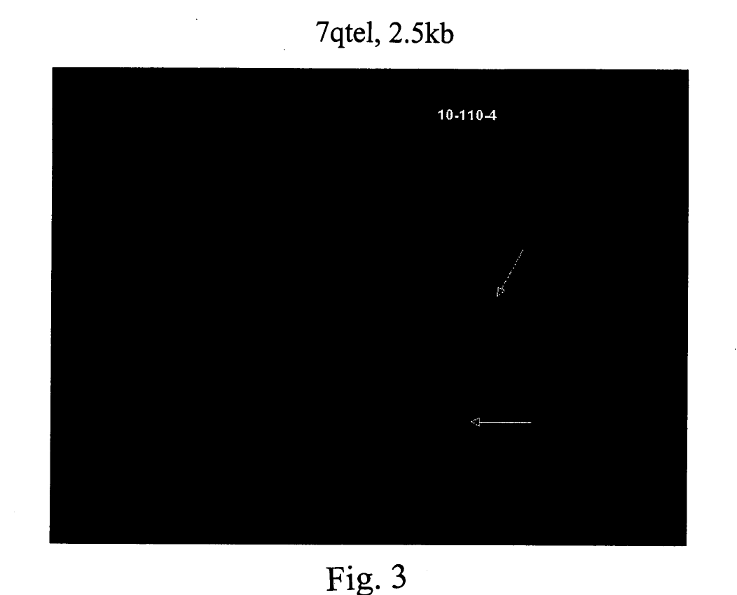

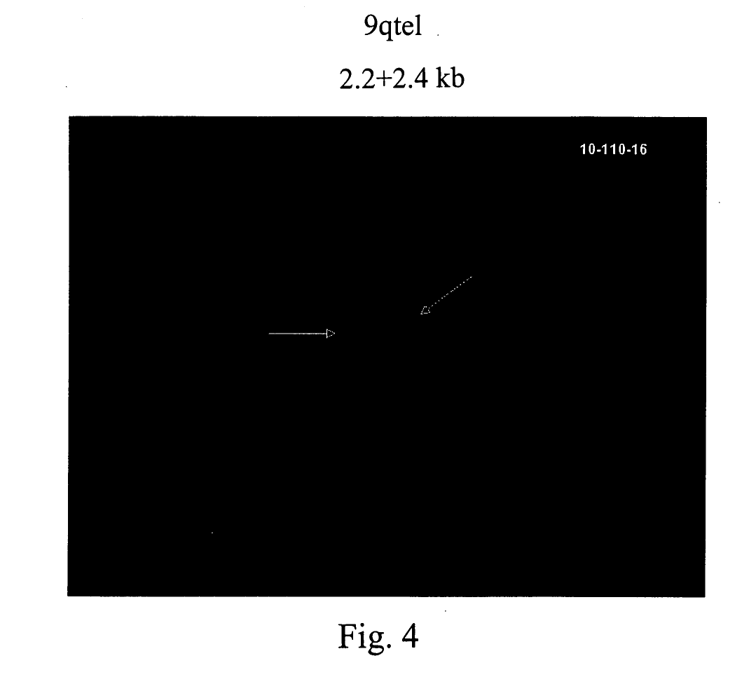

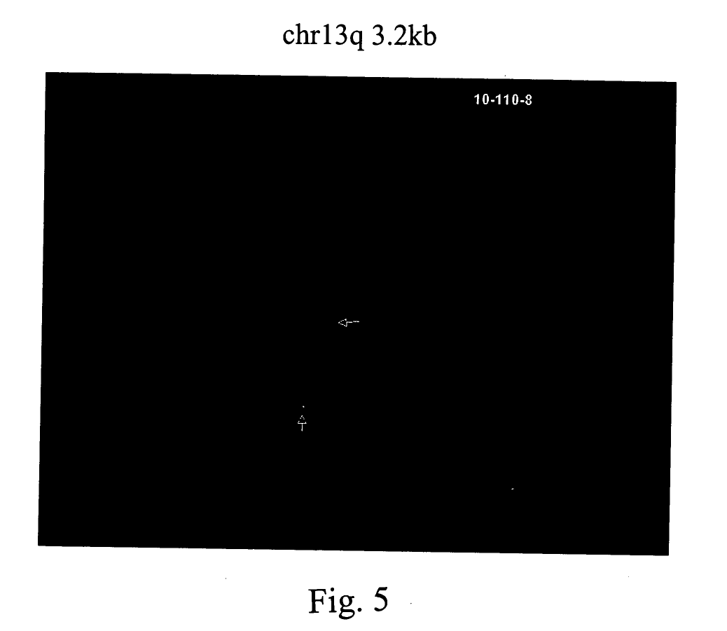

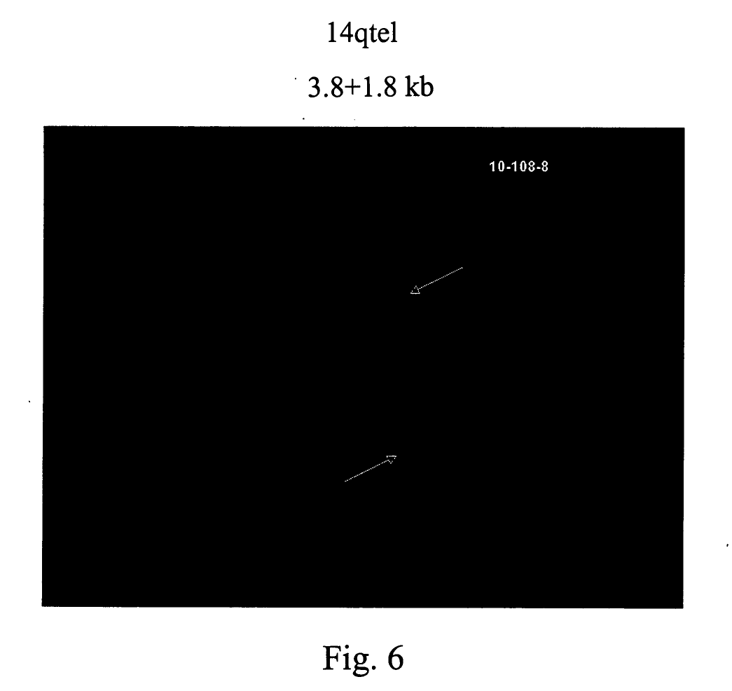

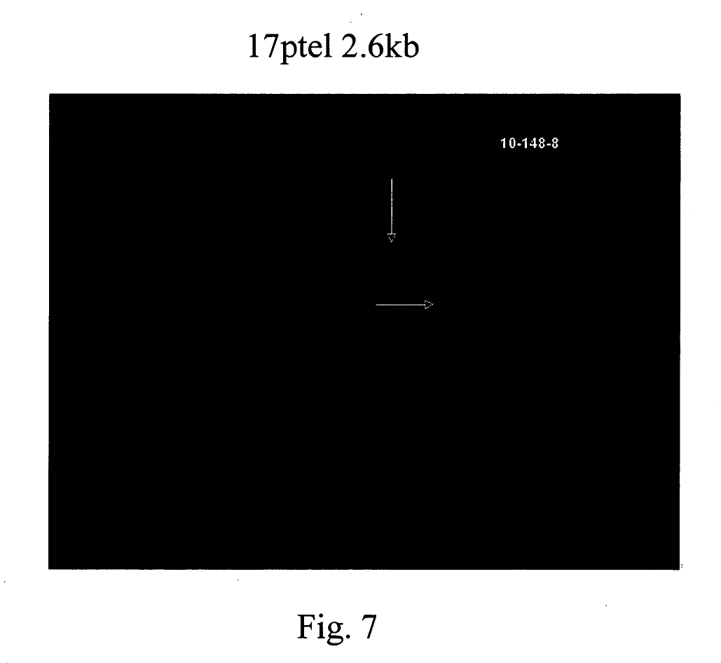

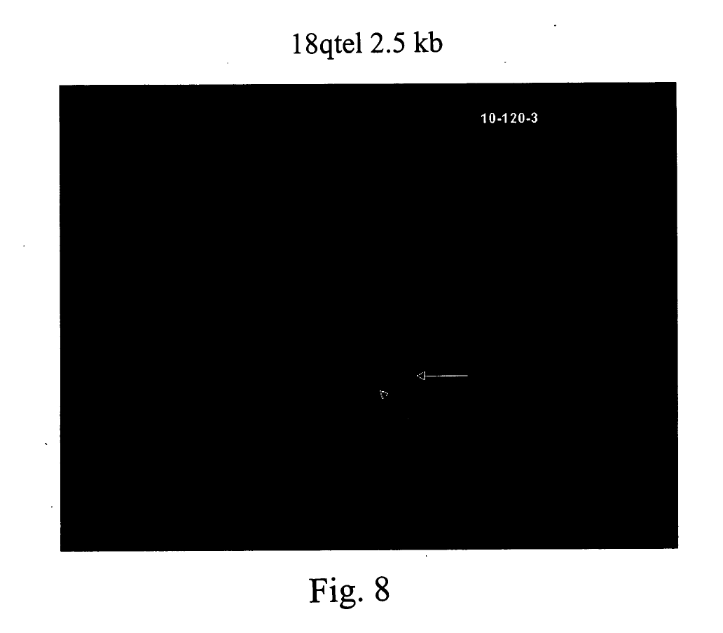

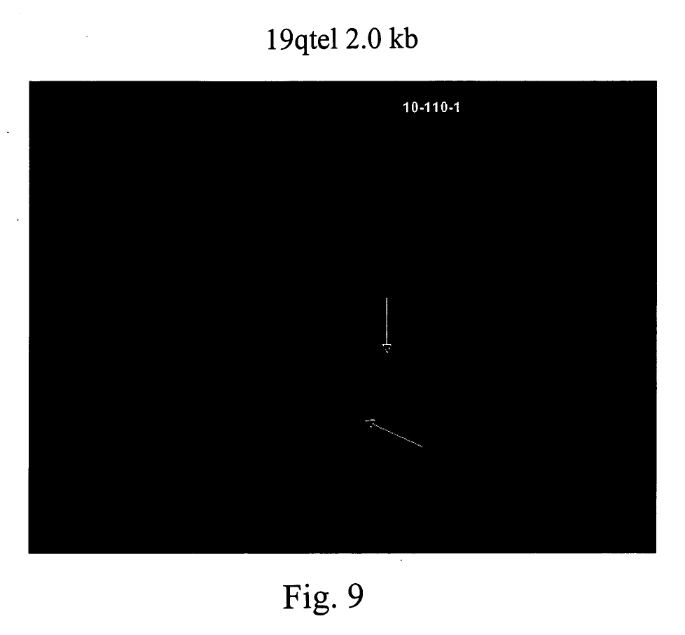

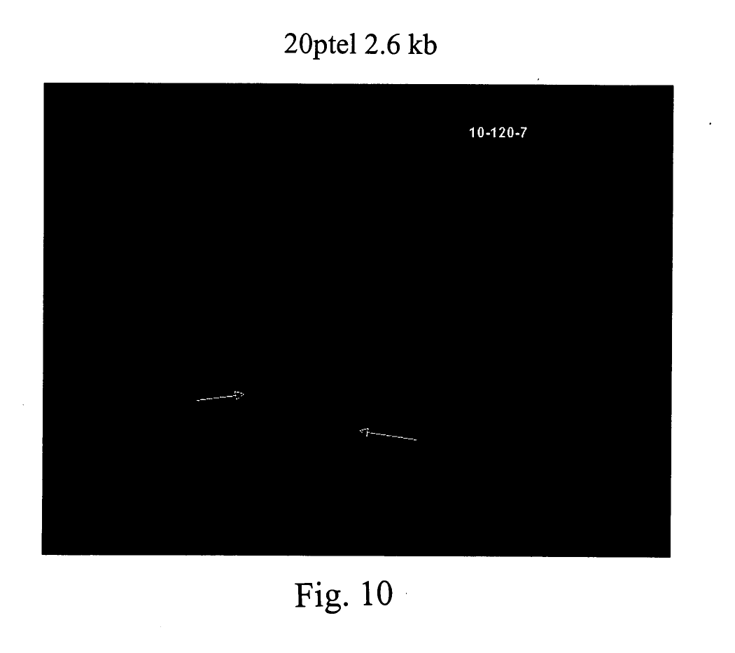

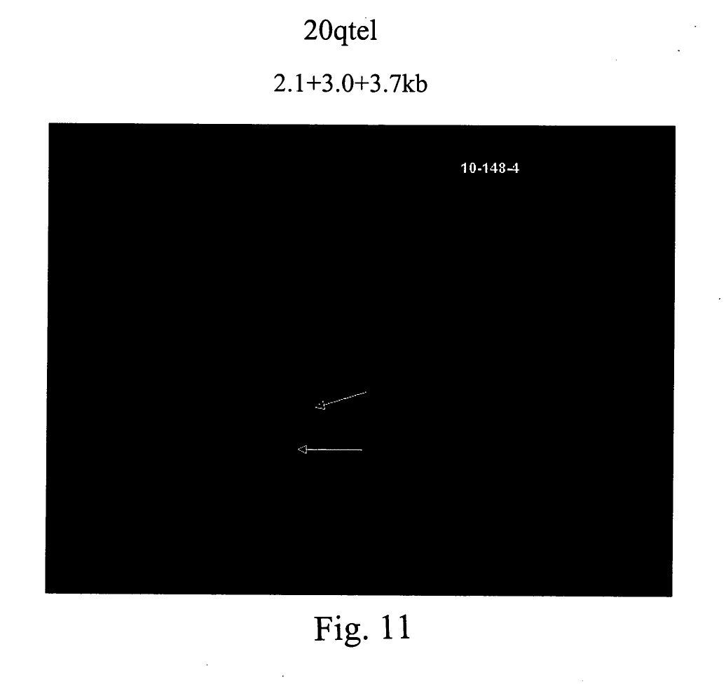

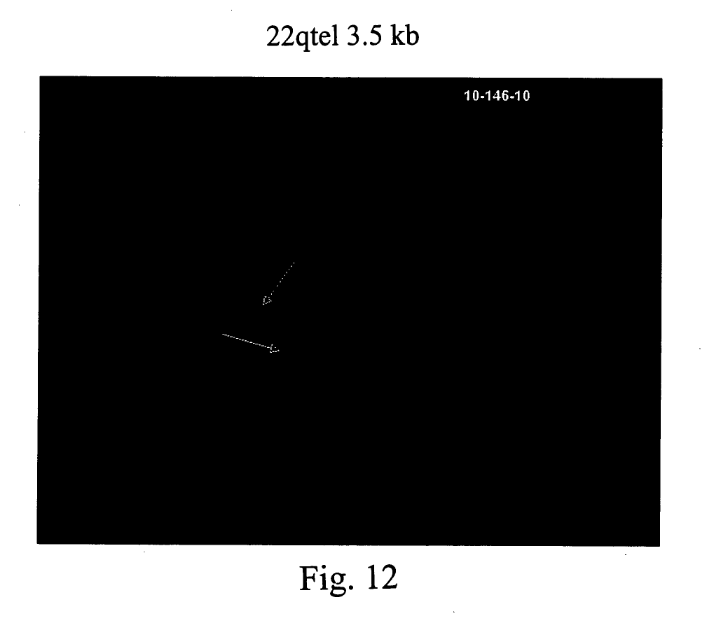

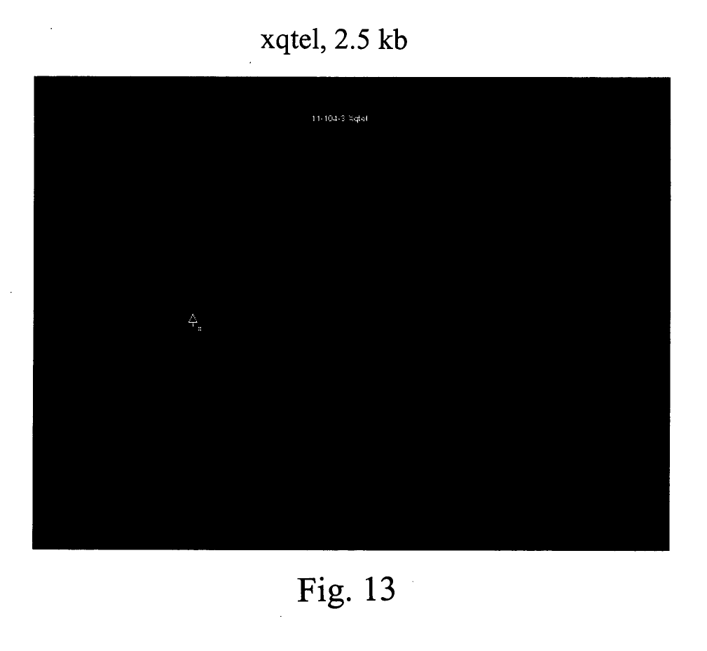

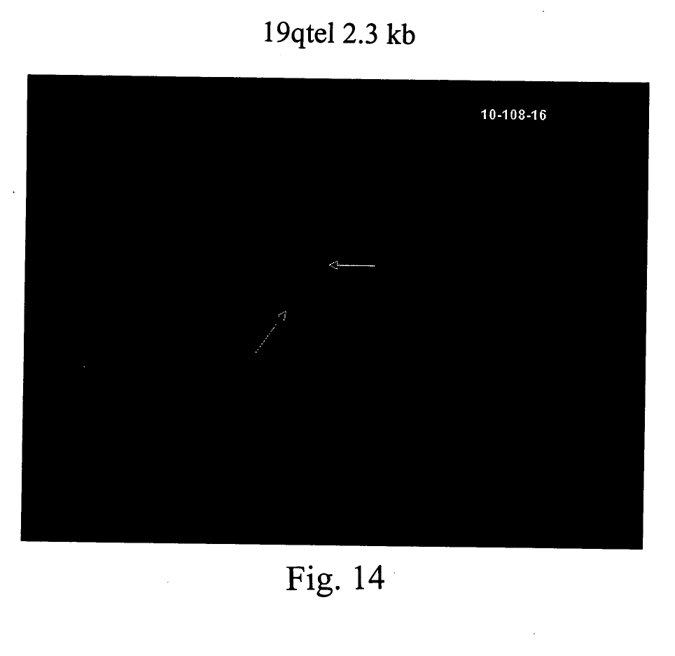

We have developed, tested, and validated a method of producing single copy probes that will detect chromosome rearrangements involving most of the human subtelomeric regions, developed chromosome arm-specific probes for the 42 euchromatic terminal regions and demonstrated that 56 are clearly to the ends of these chromosomes or fall within the range of potential locations for the commonly-used cloned probes but could be closer if the precise locations of the cloned probes could be determined. These single copy probes can therefore detect smaller and more terminal chromosomal imbalances involving subtelomeric sequences than existing probes. We infer that these probes will have greater sensitivity in detecting idiopathic mental retardation and other clinical abnormalities that result from this type of aneuploidy. The location of the probes on the chromosomes is clearly shown in FIGS. 2-13 with FIG. 1 being a compilation of FIGS. 2-13 and was prepared using the raw photos of these Figs. FIG. 14 shows the location of 19qtel which is not represented in FIG. 1.

Thus, the present invention provides methods of determining and developing subtelomeric DNA probes which are smaller than were previously available and usually closer to the telomere. These smaller probes are able to detect smaller mutations, deletions, and rearrangements that larger probes are unable to detect due to their size. Moreover, some mutations, deletions, and rearrangements may actually occur within the sequence of the larger probes and such sequences could not have been detected using the probe but could be detected using the methods and probes of the present invention. The probes of the present invention are able to detect chromosomal rearrangements which are closer to the ends of the chromosomes than was previously possible. This is due to the fact that the probes of the present invention are developed by starting at the very end of each arm of each chromosome and working inward to find one or more unique sequences which are then used to develop corresponding probes. Cross-hybridizing sequences are preferably eliminated computationally, that is to say that sequences identified will be compared to known sequences such that there will be little to no cross hybridization rather than by experimentally determining whether or not you have a probe which cross-hybridizes. Specific examples of subtelomeric probes of the present invention have been developed using the primers identified herein as SEQ ID Nos. 83-244.

### EXAMPLE 2

This example describes the design, synthesis, validation and hybridization of an 18qtel (2530 bp) probe.

Materials and Methods:

A probe from the subtelomeric interval on the long arm of chromosome 18 was developed on Jul. 30, 2001 from the human genome sequence published on Apr. 1, 2001. Sequences from this chromosome were downloaded and analyzed with custom software that was developed to automatically identify prospective single copy intervals and select primer sequences for the polymerase chain reaction. Of course, any method that will identify prospective single copy sequences can be used for purposes of the present invention. A Unix script, integrated single copy FISH, manages the process. The user is requested to provide the version of the human genome sequence from which probes are designed, the coordinates of the chromosomal region and the minimum length of the single copy interval. The minimum length of this interval was chosen to be 1500 nucleotides, based on ease of visualization of FISH probes by fluorescence microscopy. The software will, however, identify single copy intervals of any desired size. An interval containing the terminal 349,999 bp was input and the script retrieved this sequence from the genome browser at the University of California-Santa Cruz website. A Perl program, findirepeatmask.pl then computed the coordinates of all >1500 bp intervals from the output of the RepeatMasker program (Smit A and Green P, University of Washington). The Delila program, xyplo at the ncifcrf website displayed a scatterplot indicating the locations of the single copy intervals. The script then called a series of sequence analysis programs (Wisconsin package; (from accelrys.com), first extracting sequences of each single copy subinterval from the larger sequence, and then selecting oligonucleotide primer sequences optimized for long PCR for each subinterval. The chromosome 18 subinterval from 83,779,017 to 83,879,017 was selected for primer design. Primer selection was performed with a Perl script (primwrapper.pl which executes the Wisconsin program prime) by dynamically decrementing primer annealing temperature, product G/C composition and interval length beginning with the most stringent conditions, as we have previously described (Rogan et al. Genome Research, 11:1086-1094, 2001, the content and teachings of which are incorporated by reference). Design of a set of potential probes in the 350 kb genomic region required ˜1 hour on a 300 MHz Unix workstation. For this chromosome 18 interval, the software offered 25 potential intervals for this long PCR reaction. We selected product 22, which is between 80,057 and 82,584 bp from the end of the given sequence in the “finished” April 2003 genome reference sequence. In the April 2001 sequence, this chromosome 18 sequence was not completed and the probe sequence fell between 43227 and 45756 bp from the end of the available sequence. Even though the RepeatMasker software screens the sequence for repetitive sequence families that are common in the human genome, this software does not detect complex paralogous or low copy number segmental duplicated regions in the genome that do not technically meet the criterion of a repetitive sequence. The single copy composition of this sequence was therefore verified computationally with the BLAT tool at the UCSC Genome Browser website. This tool rapidly determines whether other sequences in the genome are related to a query, and if so the length and the percent similarity of those sequences relative to the query. A script was developed to automate this BLAT procedure for multiple intervals simultaneously. Related sequences less than or equal to 500 bp in length or <1000 bp sequences with more than 30% divergence were unlikely to cross-hybridize to the probe under the hybridization and wash stringency conditions used to detect chromosomal sequences. Sequences that exceeded these thresholds were generally rejected as potential probes, however no such related sequences were detected computationally for the 18q tel region.

The PCR primers that amplify this product consisted of a 30 mer forward and 32 mer reverse strands (SEQ ID NOS 193 and 194). These DNA primers were synthesized by IDT Inc. (Coralville IA), and resuspended in 500 ul of double distilled H₂O then diluted to a working stock concentration of 10 uM. Initially, the primers were tested for their ability to produce an amplification product of the expected size, ie. 2530 bp—based on their respective coordinates in the genome. The test PCR reaction comprised a total of 25 ul and consisted of the forward and reverse primers (each at 0.9 uM), 30 ng of human genomic high molecular weight DNA (stored at 4 deg C.; Promega, Madison Wis.), 1.5 mM MgSO4, 0.625 units of Platinum Pfx polymerase, 10× Reaction buffer, 1.25 mM dNTPs, and 1× PCR Enhancer solution (components and conditions from the manufacturer Invitrogen, Carlsbad Calif.). The initial amplification was carried out at the melting temperature predicted by the primer design program, 60 deg C. Agarose gel electrophoresis revealed the product had the expected size, however additional reaction optimization was needed to obtain a homogeneous product. The Biomek 2000 laboratory automation workstation was used to set up a simultaneously set of parallel reactions for this 18qtel and other products for other subtelomeric regions. For temperature optimization, these parallel reactions were each amplified by PCR at a different annealing temperatures, specifically 53.2,55.5,58.4,61.8,64.6, and 66.8 deg C. on a gradient thermalcycler (MJ Research Alpha) with the same reaction conditions as above, except that the primers were added at 0.3 uM in the optimizing reactions. The thermal cycling conditions were: initial denaturation of genomic template for 2 minutes at 94 deg C., followed by 15 cycles at the above annealing and extension temperatures for 5 minutes and denaturation for 20 minutes. This was followed by an additional 15 cycles at the same temperatures, but the annealing and extension step was increased in duration by 5 minutes per cycle. After a primer extension polishing step at 68 deg C. for 10 minutes, the reaction was chilled and held at 0 deg C. The products were separated by agarose gel electrophoresis and inspected to determine the maximum yield that generated the purest products. The optimum temperature for product of this probe was found to be 64 deg C. The reaction was scaled up to a 200 ul final volume (ie. ˜2 ug) to prepare sufficient amounts of PCR product for labeling and several fluorescence in situ hybridization assays. The product was separated on a preparative agarose gel, the band was excised, and purified using a Montage extraction spin column (Millipore, Watertown Mass.). The eluate from the column was precipitated with ethanol, briefly dessicated, and resuspended in double distilled water at a concentration of 100 ng/ul. Approximately 1 ug of product was recovered. This solution was labeled by nick-translation with either digoxygenin-modified or biotinylated dUTP as described in Rogan et al (2001). This procedure provided sufficient amounts of probe for denaturation and hybridization to 5 slides containing metaphase and interphase chromosomes from normal individuals and patient specimens.

Results:

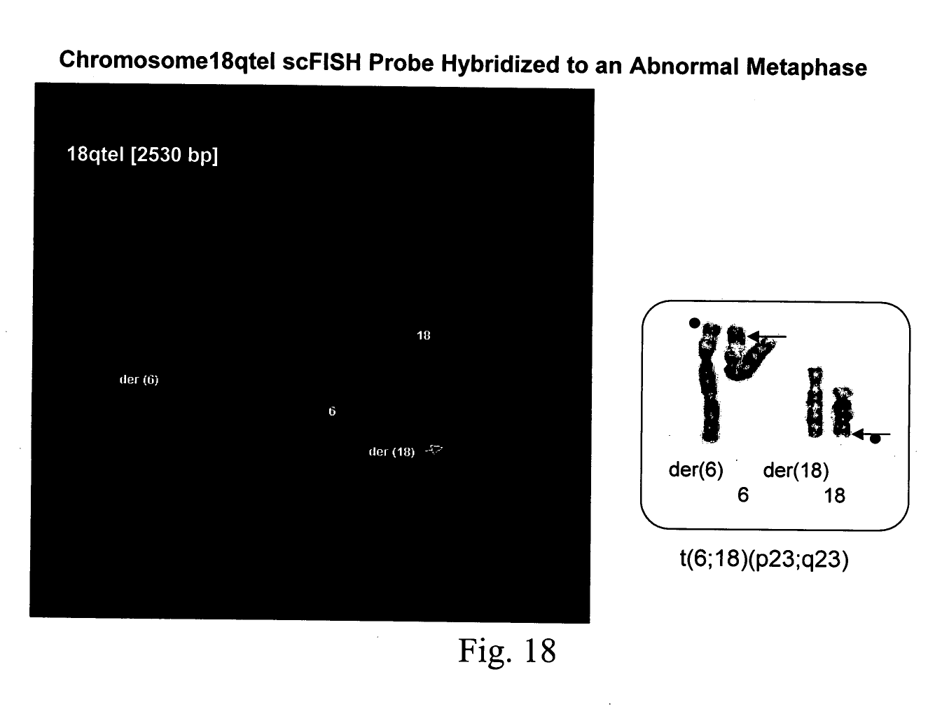

Experimental validation of the probe showed that it did not hybridize to any other chromosomal region in cells from a normal individual with a normal karyotype, consistent with computational prediction that this sequence was present in a single copy in the genome. This probe, having passed both computational and experimental validation, was selected based on its close proximity to the terminus of chromosome 18q for analysis of a patient thought to carry a terminal rearrangement of this chromosome. FIG. 18 shows an example of this probe detecting a translocation of this sequence to the terminal band on the p arm of chromosome 6 in a patient with a 6;18 translocation. In this figure, an 18q subtelomeric probe (2530 bp in length) is hybridized to an abnormal metaphase cell. This cell has a translocation between the short arm of one chromosome 6 and the terminal chromosomal band on one chromosome 18. The locations of the translocation sites are indicated by arrows on the normal G-banded chromosome 6 and normal G-banded chromosome 18. The translocated or derivative (der) G-banded chromosomes 6 and 18 are also included. The position of the 18q probe is indicated in red. The chromosome 18q probe (detected in red) is hybridized to the normal chromosome 18 and the derivative chromosome 6 as shown in the left panel. The derivative chromosome 18 does not hybridize as its subtelomeric region as been exchanged with chromosome 6p genetic material.

I claim:

1. A subtelomeric probe useful for detecting chromosomal rearrangements comprising:
a single copy DNA sequence having a length of less than 25 kb, said sequence being capable of hybridizing to the terminal G-band or R-band of an arm of a single chromosome.

2. The probe of claim 1, said terminal band being light after G-band staining.

3. The probe of claim 1, said terminal band being dark after R-band staining.

4. The probe of claim 1, said arm of said single chromosome being selected from the group consisting of 1p, 1q, 2p, 2q, 3p, 3q, 4p, 4q, 5p, 5q, 6p, 6q, 7p, 7q, 8p, 8q, 9p, 9q, 10p, 10q, 11p, 11q, 12p, 12q, 13q, 14q, 15q, 16p, 16q, 17p, 17q, 18p, 18q, 19p, 19q, 20p, 20q, 21q, 22q, Xp, Xq, and Yp.

5. The probe of claim 1, said probe being selected from the group consisting of SEQ ID NOS. 1-3, 5-23, 26-36, 38-57, 59-61, 63-67, 69-82, and 245-251.

6. The probe of claim 1, said probe having a length of less than 10 kb.

7. The probe of claim 1, said probe being within 8000 kb of the telomere of said chromosome.

8. The probe of claim 7, said probe being selected from the group consisting of SEQ ID NOS. 1-3, 5-23, 26-36, 38-57, 59-61, 63-67, 69-82, and 245-251.

9. The probe of claim 1, said probe being within 300 kb of the telomere of said chromosome.

10. The probe of claim 9, said probe being selected from the group consisting of SEQ ID NOS. 36, 80, 46, 47, 49, 51, 56, 248, 57, 78, 59, 75, 76, 74, 63, 250, 251, 66, 65, 67, 4, 3, 1, 9, 6, 11, 10, 17, 20, 19, 18, 21, 81, 26, 29, 28, 31, 32, 43, 42, 41, 40, 44, 45,and70.

11. The probe of claim 1, said probe being labeled or being modified to attach to a surface.

12. A method of developing single copy DNA sequence probes from subtelomeric regions of chromosomes, said probes being able to hybridize to a single location in the genome, said method comprising the steps of:
searching the DNA sequence of said chromosome on a nucleotide-by-nucleotide basis beginning at the terminal nucleotide for a single copy interval of at least 500 base pairs in length that is closest to said terminal nucleotide;
identifying said single copy interval;
synthesizing said single copy interval; and
using said synthesized single copy interval as said probes.

13. The method of claim 12, said identifying step including the step of verifying computationally or experimentally that said identified single copy interval is represented at a single genomic location or where paralogous sequences are closely linked so that only a single signal is detected.

14. The method of claim 13, said identifying step including verifying computationally and experimentally.

15. The method of claim 13, said computational verification including using software to determine that the probe sequence is located at a single position in the genome.

16. The method of claim 12, said method further including the step of labeling said synthesized single copy sequence.

17. The method of claim 13, said experimental verification including rehybridizing said single copy probe to said chromosome and visualizing said probe on the terminal band and correct arm of said chromosome.

18. The method of claim 12, said single copy interval being selected from the group consisting of SEQ ID NOS. 1-3, 5-23, 26-36, 38-57, 59-61, 63-67, 69-82, and 245-251.

19. The method of claim 12, said method further comprising the step of preannealing said single copy probe with highly repetitive DNA.

20. A synthetic single copy polynucleotide for identifying chromosomal rearrangements, said polynucleotide being located within 8,000 kb of the terminal nucleotide of a chromosome and hybridizing to a single location on a specific chromosome when no chromosomal rearrangement has occurred, said polynucleotide having a length of less than 25 kb.

21. The polynucleotide of claim 20, said polynucleotide being found in the terminal G-band or R-band of said specific chromosome.

22. The polynucleotide of claim 20, said polynucleotide being selected from the group consisting of SEQ ID NOS. 1-3, 5-23, 26-36, 38-57, 59-61, 63-67, 69-82, and 245-251.

23. The polynucleotide of claim 20, said polynucleotide being located within about 300 kb of said terminal nucleotide of said specific chromosome.

24. The polynucleotide of claim 23, said polynucleotide being selected from the group consisting of SEQ ID NOS. 36, 80, 46, 47, 49, 51, 56, 248, 57, 78, 59, 75, 76, 74, 63, 250, 251, 66, 65, 67, 4, 3, 1, 9, 6, 11, 10, 17, 20, 19, 18, 21, 81, 26, 29, 28, 31, 32, 43, 42, 41, 40, 44, 45, and70.

25. The polynucleotide of claim 20, said polynucleotide being labeled or being chemically modified to attach to a surface.

26. An oligonucleotide primer pair used for deriving single copy probes that can detect chromosomal rearrangements, said primers comprising:
a sequence selected from the group consisting of SEQ ID NOS. 83-244.

27. An improved synthetic DNA probe operable for detecting chromosomal rearrangements, said probe including a DNA sequence operable to hybridize to a precise location on a single chromosome arm wherein the improvement comprises a probe of less than 25 kb in length.

28. The improved probe of claim 27, said portion comprising the entire probe.

29. The improved probe of claim 27, said probe having at least a portion thereof being located closer to the end of a telomere on a chromosome arm than a clone selected from the group consisting of cosmids, fosmids, bacteriophage, P1, and PAC clones derived from half YACS, said chromosome arm being selected from the group consisting of 2p, 3p, 5p, 7p, 8p, 10p, 11p, 12p, 16p, 17p, 18p, Xp, Yp, 1q, 3q, 4q, 6q, 7q, 8q, 9q, 10q, 11q, 12q, 13q, 14q, 15q, 16q, 17q, 18q, 19q, 20q, 21q, and 22q.

30. The improved probe of claim 27, said probe being located within 8,000 kb of the terminal nucleotide of the telomere of said chromosome.

31. The improved probe of claim 27, said probe being located within 300 kb of the terminal nucleotide of the telomere of said chromosome.

32. The improved probe of claim 27, said probe being located in the terminal G-band or R-band of said chromosome.

33. The improved probe of claim 27, said probe being selected from the group consisting of SEQ ID NOS. 46, 47, 49, 56, 78, 59, 64, 249, 2, 4, 3, 5, 9, 11, 20, 19, 21, 81, 246, 70, 72, 73, 36, 80, 247, 50, 57, 75, 76, 74, 63, 250, 66, 65, 67, 1, 6, 10, 12, 16, 15, 13, 14, 17, 18, 81, 245, 26, 31, 32, 43, 42, 41, 40, 44, and 45.

34. A method of screening an individual for cytogenetic abnormalities, said individual having either idiopathic mental retardation or mental retardation and at least one other clinical abnormality or cancer said method comprising the steps of:
screening the genome of the individual using a plurality of hybridization probes, each of said probes having a length of less than about 25 kb; and
detecting hybridization patterns of said probes, said hybridization patterns indicating cytogenetic abnormalities in said genome.

35. The method of claim 34, said method further including the step of associating said hybridization patterns with specific clinical abnormalities.

36. The method of claim 34, said probes being represented at a single genomic location or where paralogous sequences are closely linked so that only a single hybridization signal is detected.

37. A method of delineating the extent of a chromosome imbalance comprising the steps of:
assaying a chromosome arm using at least one hybridization probe having a length of less than about 25 kb;
detecting hybridization patterns of said probes on said arm; and
comparing said hybridization patterns with a standard genome map of said arm in order to delineate the extent of a chromosome imbalance.

38. The method of claim 37, said method further including the step of correlating imbalances on said arm with a medical condition selected from the groups consisting of idiopathic mental retardation or cancer.

39. The method of claim 37, said method utilizing a plurality of probes.

40. The method of claim 37, said probe hybridizing to a specific chromosome arm.
